(19) United States
(12) Patent Application Publication
(10) Pub. No.: US 2013/0261173 A1
(43) Pub. Date: Oct. 3, 2013

(54) CYCLODEXTRIN-BASED POLYMERS FOR THERAPEUTIC DELIVERY

(71) Applicants: Thomas C. Crawford, Essex, CT (US); Oliver S. Fetzer, Needham, MA (US); Lawrence Alan Reiter, Mystic, CT (US); Marc Wolfgang, Holliston, MA (US)

(72) Inventors: Thomas C. Crawford, Essex, CT (US); Oliver S. Fetzer, Needham, MA (US); Lawrence Alan Reiter, Mystic, CT (US); Marc Wolfgang, Holliston, MA (US)

(73) Assignee: CERULEAN PHARMA INC., Cambridge, MA (US)

(21) Appl. No.: 13/908,504

(22) Filed: Jun. 3, 2013

Related U.S. Application Data
(63) Continuation of application No. 13/441,477, filed on Apr. 6, 2012, now abandoned.
Application No. 13/441,477 is a continuation of application No. 13/208,703, filed on Aug. 12, 2011.
Application No. 13/208,703 is a continuation-in-part of application No. 12/953,390, filed on Nov. 23, 2010, now abandoned.
(60) Provisional application No. 61/263,749, filed on Nov. 23, 2009.
(60) Provisional application No. 61/391,922, filed on Oct. 11, 2010.

(51) Int. Cl. A61K 47/48

(52) U.S. Cl. CPC A61K 47/48
USPC 514/449; 549/510

(57) ABSTRACT

Methods and compositions relating to CDP-taxane conjugates are described herein.

### CLAIM OF PRIORITY

This application is a continuation of U.S. Ser. No. 13/441,477, filed on Apr. 6, 2012, which is a continuation of U.S. Ser. No. 13/208,703, filed on Aug. 12, 2011, which is a continuation-in-part and claims priority to U.S. Ser. No. 12/953,390, filed Nov. 23, 2010, which claims priority to U.S. Ser. No. 61/263,749, filed Nov. 23, 2009 and U.S. Ser. No. 61/391,922, filed Oct. 11, 2010, the entire contents of each of which are incorporated herein by reference.

### BACKGROUND OF THE INVENTION

Drug delivery of some small molecule therapeutic agents, such as taxane, has been problematic due to their poor pharmacological profiles. These therapeutic agents often have low aqueous solubility, their bioactive forms exist in equilibrium with an inactive form, or high systemic concentrations of the agents lead to toxic side-effects. Some approaches to circumvent the problem of their delivery have been to conjugate the agent directly to a water-soluble polymer such as hydroxypropyl methacrylate (HPMA), polyethyleneglycol, and poly-L-glutamic acid. In some cases, such conjugates have been successful in solubilizing or stabilizing the bioactive form of the therapeutic agent, or achieving a sustained release formulation which circumvents complications associated with high systemic concentrations of the agent.

Another approach to the drug delivery problem has been to form host/guest inclusion complexes between the therapeutic agent and cyclodextrins or derivatives thereof. Cyclodextrins (alpha, beta, and gamma) and their oxidized forms have unique physico-chemical properties such as good water solubility, low toxicity and low immune response. To date, most of the drug delivery studies with cyclodextrins have focused on their ability to form supra-molecular complexes, wherein cyclodextrins form host/guest inclusion complexes with therapeutic molecules and thus alter the physical, chemical, and/or biological properties of these guest molecules.

### SUMMARY OF THE INVENTION

In one aspect, the disclosure features a CDP-taxane conjugate, e.g., a CDP-docetaxel conjugate, a CDP-larotaxel conjugate or CDP-cabazitaxel conjugate, described herein, and methods of making the CDP-taxane conjugates, e.g., a CDP-docetaxel conjugates, a CDP-larotaxel conjugates or CDP-cabazitaxel conjugates, described herein.

In one embodiment, CDP is not biodegradable.

In one embodiment, CDP is biocompatible.

In one embodiment, the CDP-taxane conjugate, e.g., a CDP-docetaxel conjugate, a CDP-larotaxel conjugate or CDP-cabazitaxel conjugate, includes an inclusion complex between a taxane, e.g., docetaxel, larotaxel or cabazitaxel, attached or conjugated to the CDP, e.g., via a covalent linkage or via a linker such as a linker described herein, and another molecule in the CDP. In one embodiment, the CDP-taxane conjugate forms a nanoparticle. In one embodiment, the CDP-taxane conjugate including an inclusion complex forms a nanoparticle. The nanoparticle ranges in size from 10 to 300 nm in diameter, e.g., 10 to 280, 20 to 280, 30 to 250, 30 to 200, 20 to 150, 30 to 100, 20 to 80, 10 to 80, 10 to 70, 20 to 60 or 20 to 50 nm 10 to 70, 10 to 60 or 10 to 50 nm diameter. In one embodiment, the nanoparticle is 20 to 60 nm in diameter. In one embodiment, the composition comprises a population or a plurality of nanoparticles with an average diameter from 10 to 300 nm, e.g., 20 to 280, 15 to 250, 15 to 200, 20 to 150, 15 to 100, 20 to 80, 15 to 80, 15 to 70, 15 to 60 or, 15 to 50, 20 to 50 nm. In one embodiment, the average nanoparticle diameter is from 15 to 60 nm (e.g., 20-60. In one embodiment, the surface charge of the molecule is neutral, or slightly negative. In some embodiments, the zeta potential of the particle surface is from about −80 mV to about 50 mV, about −20 mV to about 20 mV, about −20 mV to about −10 mV, or about −10 mV to about 0.

In an embodiment, the CDP-taxane conjugate complex forms a particle or nanoparticle having a conjugate number described herein. By way of example, a CDP-therapeutic agent conjugate, forms, or is provided in, a particle or nanoparticle having a conjugate number of: 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In an embodiment the conjugate number is 2 to 4 or 2 to 5.

In an embodiment the conjugate number is 1, 2, 3, 4, 5, 6, 7, 8, 9, or 10.

In an embodiment the nanoparticle forms, or is provided in, a preparation of nanoparticles, e.g., a pharmaceutical preparation, wherein at least 40, 50, 60, 70, 80, 90 or 95% of the particles in the preparation have a conjugate number provided herein. In an embodiment the nanoparticle forms, or is provided in, a preparation of nanoparticles, e.g., a pharmaceutical preparation, wherein at least 60% of the particles in the preparation have a conjugate number of 1-5 or 2-5.

In an embodiment, the CDP-taxane conjugate is administered as a nanoparticle or preparation of nanoparticles, e.g., a pharmaceutical preparation, wherein at least 60% of the particles in the preparation have a conjugate number of 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; to 50; 30 to 50; 30 to 40; or 30 to 75.

In one embodiment, the taxane (e.g., docetaxel, paclitaxel, larotaxel or cabazitaxel), conjugated to the CDP is more soluble when conjugated to the CDP, than when not conjugated to the CDP.

In one embodiment, the composition comprises a population, mixture or plurality of CDP-taxane conjugates. In one embodiment, the population, mixture or plurality of CDP-taxane conjugates comprises a plurality of different taxane conjugated to a CDP (e.g., two different taxanes are in the composition such that two different taxanes are attached to a single CDP; or a first taxane is attached to a first CDP and a second taxane is attached to a second CDP and both CDP-taxane conjugates are present in the composition). In one embodiment, the population, mixture or plurality of CDP-taxane conjugates comprises a CDP having a single taxane attached thereto in a plurality of positions (e.g., a CDP has a single taxane attached thereto such that the single taxane for some occurrences is attached through a first position (e.g., a 2′-OH) and for other occurrences is attached through a second position (e.g., a 7-OH) to thereby provide a CDP having a single taxane attached through a plurality of positions on the taxane). In some embodiments, for example, where a third position is available (e.g., a 10-OH), a single taxane can be attached to the CDP through a first, second, and third position (e.g., 2′-OH, 7-OH, and 10-OH). In one embodiment, the population, mixture or plurality of CDP-taxane comprises a first CDP attached to a taxane through a first position (e.g., a 2′-OH) and a second CDP attached to the same taxane through a second position (e.g., a 7-OH) and both CDP-taxane conjugates are present in the composition. In one embodiment, the population, mixture or plurality of CDP-taxane comprises a first CDP attached to a taxane through a first position (e.g., a 2′-OH), a second CDP attached to the same taxane through a second position (e.g., a 7-OH), and a third CDP attached to the same taxane through a third position (e.g., a 10-OH) and all three CDP-taxane conjugates are present in the composition. In some embodiments, a single CDP includes a single taxane attached through a plurality of positions (e.g., the 2′-OH, 7-OH, and/or 10-OH).

In one aspect, the disclosure features a method of treating a proliferative disorder, e.g., a cancer, in a subject, e.g., a human, the method comprises: administering a composition that comprises a CDP-taxane conjugate, e.g., a CDP-docetaxel conjugate, a CDP-larotaxel conjugate and/or a CDP-cabazitaxel conjugate described herein, to a subject in an amount effective to treat the disorder, to thereby treat the proliferative disorder. In an embodiment, the CDP-taxane conjugate comprises a taxane molecule (e.g., docetaxel, paclitaxel, larotaxel and/or cabazitaxel), coupled, e.g., via a linker such as a linker described herein, to a CDP described herein. In an embodiment, the CDP-taxane conjugate comprises a taxane molecule, coupled via a linker shown in FIG. 2 to a CDP moiety, e.g., a CDP described herein. In an embodiment, the CDP-taxane conjugate is a CDP-taxane conjugate shown in FIG. 2.

In one embodiment, the composition is administered in combination with one or more additional anticancer agent, e.g., chemotherapeutic agent, e.g., a chemotherapeutic agent or combination of chemotherapeutic agents described herein, and radiation.

In an embodiment, the method further comprises administering a chemotherapeutic agent as a free agent.

In an embodiment, the taxane associated with the CDP and the free agent are the same chemotherapeutic agent. E.g., the agent is a taxane (e.g., docetaxel, paclitaxel, larotaxel or cabazitaxel).

In an embodiment, the taxane associate with the CDP and the free agent are different chemotherapeutic agents.

In one embodiment, the cancer is a cancer described herein. For example, the cancer can be a cancer of the bladder (including accelerated and metastatic bladder cancer), breast (e.g., estrogen receptor positive breast cancer; estrogen receptor negative breast cancer; HER-2 positive breast cancer; HER-2 negative breast cancer; progesterone receptor positive breast cancer; progesterone receptor negative breast cancer; estrogen receptor negative, HER-2 negative and progesterone receptor negative breast cancer (i.e., triple negative breast cancer); inflammatory breast cancer), colon (including colorectal cancer), kidney (e.g., transitional cell carcinoma), liver, lung (including small and non-small cell lung cancer, lung adenocarcinoma and squamous cell cancer), genitourinary tract, e.g., ovary (including fallopian tube and peritoneal cancers), cervix, prostate, testes, kidney, and ureter, lymphatic system, rectum, larynx, pancreas (including exocrine pancreatic carcinoma), esophagus, stomach, gall bladder, thyroid, skin (including squamous cell carcinoma), brain (including glioblastoma multiforme), head and neck (e.g., occult primary), and soft tissue (e.g., Kaposi's sarcoma (e.g., AIDS related Kaposi's sarcoma), leiomyosarcoma, angiosarcoma, and histiocytoma). Preferred cancers include breast cancer (e.g., metastatic or locally advanced breast cancer), prostate cancer (e.g., hormone refractory prostate cancer), renal cell carcinoma, lung cancer (e.g., non-small cell lung cancer, small cell lung cancer, lung adenocarcinoma, and squamous cell cancer, e.g., unresectable, locally advanced or metastatic non-small cell lung cancer, small cell lung cancer, lung adenocarcinoma, and squamous cell cancer), pancreatic cancer, gastric cancer (e.g., metastatic gastric adenocarcinoma), colorectal cancer, rectal cancer, squamous cell cancer of the head and neck, lymphoma (Hodgkin's lymphoma or non-Hodgkin's lymphoma), renal cell carcinoma, carcinoma of the urothelium, soft tissue sarcoma (e.g., Kaposi's sarcoma (e.g., AIDS related Kaposi's sarcoma), leiomyosarcoma, angiosarcoma, and histiocytoma), gliomas, myeloma (e.g., multiple myeloma), melanoma (e.g., advanced or metastatic melanoma), germ cell tumors, ovarian cancer (e.g., advanced ovarian cancer, e.g., advanced fallopian tube or peritoneal cancer), and gastrointestinal cancer.

In one embodiment, the cancer is resistant to more than one chemotherapeutic agent, e.g., the cancer is a multidrug resistant cancer. In one embodiment, the cancer is resistant to one or more of a platinum based agent, an alkylating agent, an anthracycline and a vinca alkaloid. In one embodiment, the cancer is resistant to one or more of a platinum based agent, an alkylating agent, a taxane and a vinca alkaloid.

In one embodiment, the composition is administered by intravenous administration, e.g., an intravenous administration that is completed in a period equal to or less than 2 hours, 1.5 hours, 1 hour, 45 minutes or 30 minutes. In one embodiment, the composition is administered as a bolus infusion or intravenous push, e.g., over a period of 15 minutes, 10 minutes, 5 minutes or less.

In one embodiment, the CDP-taxane conjugate is a CDP-docetaxel conjugate, e.g., a CDP-docetaxel conjugate described herein, e.g., a CDP-docetaxel conjugate comprising docetaxel, coupled, e.g., via linkers, to a CDP described herein, and e.g., the CDP-docetaxel conjugate is administered to the subject in an amount that includes 60 mg/m² or greater (e.g., 65 mg/m², 70 mg/m², 75 mg/m², 80 mg/m², 85 mg/m², 90 mg/m², 95 mg/m², 100 mg/m², 105 mg/m², 110 mg/m², 115 mg/m², 120 mg/m²) of docetaxel, to thereby treat the disorder. In one embodiment, the conjugate is administered by intravenous administration over a period of about 30 minutes, 45 minutes, 60 minutes, 90 minutes, 120 minutes, 150 minutes or 180 minutes. In one embodiment, the subject is administered at least one additional dose of the conjugate, e.g., the subject is administered at least two, three, four, five, six, seven, eight, nine, ten or eleven additional doses of the conjugate. In one embodiment, the conjugate is administered once every two, three, four, five, six weeks. In another embodiment, the CDP-docetaxel conjugate, e.g., a CDP-docetaxel conjugate described herein, e.g., a CDP-docetaxel conjugate comprising docetaxel, coupled, e.g., via linkers, to a CDP described herein, and e.g., the CDP-docetaxel conjugate is administered to the subject in an amount that includes 30 mg/m² or greater (e.g., 31 mg/m², 33 mg/m², 35 mg/m², 37 mg/m², 40 mg/m², 43 mg/m², 45 mg/m², 47 mg/m², 50 mg/m², 55 mg/m²) of docetaxel, to thereby treat the disorder.

In one embodiment, the conjugate is administered by intravenous administration over a period of about 30 minutes, 45 minutes, 60 minutes, 90 minutes, 120 minutes, 150 minutes or 180 minutes. In one embodiment, the subject is administered at least one additional dose of the conjugate, e.g., the subject is administered at least two, three, four, five, six, seven, eight, nine, ten or eleven additional doses of the conjugate. In one embodiment, the conjugate is administered once a week for three, four, five six, seven weeks, e.g., followed by one, two or three weeks without administration of the CDP-docetaxel conjugate. In one embodiment, the dosing schedule is not changed between doses. For example, when the dosing schedule is once every three weeks, an additional dose (or doses) is administered in three weeks. In one embodiment, when at least one additional dose is administered, the additional dose (or additional doses) is administered in an amount such that the conjugate includes 60 mg/m² or greater (e.g., 65 mg/m², 70 mg/m², 75 mg/m², 80 mg/m², 85 mg/m², 90 mg/m², 95 mg/m², 100 mg/m², 105 mg/m², 110 mg/m², 115 mg/m², 120 mg/m²) of docetaxel. In one embodiment, when at least one additional dose is administered, the additional dose (or additional doses) is administered by intravenous administration over a period equal to or less than about 30 minutes, 45 minutes, 60 minutes, 90 minutes, 120 minutes, 150 minutes or 180 minutes. In an embodiment, the CDP-docetaxel conjugate comprises docetaxel, coupled via a linker shown in FIG. 2, to a CDP described herein. In an embodiment, the CDP-docetaxel conjugate is a CDP-docetaxel conjugate shown in FIG. 2.

In one embodiment, the CDP-taxane conjugate is a CDP-docetaxel conjugate, e.g., a CDP-docetaxel conjugate described herein, e.g., a CDP-docetaxel conjugate comprising docetaxel, coupled, e.g., via linkers, to a CDP described herein, and the conjugate is administered to the subject in an amount of the composition that includes 60 mg/m² or greater (e.g., 65 mg/m², 70 mg/m², 75 mg/m², 80 mg/m², 85 mg/m², 90 mg/m², 95 mg/m², 100 mg/m², 105 mg/m², 110 mg/m², 115 mg/m², 120 mg/m²) of docetaxel, administered by intravenous administration over a period equal to or less than about 30 minutes, 45 minutes, 60 minutes, 90 minutes, 120 minutes, 150 minutes or 180 minutes, for at least two, three, four, five or six doses, wherein the subject is administered a dose of the conjugate once every two, three, four, five or six weeks.

In one embodiment, the CDP-taxane conjugate is a CDP-docetaxel conjugate, e.g., a CDP-docetaxel conjugate described herein, e.g., a CDP-docetaxel conjugate comprising docetaxel, coupled, e.g., via linkers, to a CDP described herein, and the conjugate is administered to the subject in an amount of the composition that includes 30 mg/m² or greater (e.g., 31 mg/m², 33 mg/m², 35 mg/m², 37 mg/m², 40 mg/m², 43 mg/m², 45 mg/m², 47 mg/m², 50 mg/m², 55 mg/m²) of docetaxel, administered by intravenous administration over a period equal to or less than about 30 minutes, 45 minutes, 60 minutes, 90 minutes, 120 minutes, 150 minutes or 180 minutes, for at least two, three, four, five or six doses, wherein the subject is administered a dose of the conjugate once a week for two, three four, five, six doses, e.g., followed by one, two or three weeks without administration of the CDP-docetaxel conjugate.

In one embodiment, the composition includes a CDP-docetaxel conjugate, e.g., a CDP-docetaxel conjugate described herein, e.g., a CDP-docetaxel conjugate comprising docetaxel, coupled, e.g., via linkers, to a CDP described herein, and at least two, three, four, five, six, seven, eight, nine, ten or eleven doses are administered to the subject and each dose is an amount of the composition that includes 60 mg/m² or greater (e.g., 65 mg/m², 70 mg/m², 75 mg/m², 80 mg/m², 85 mg/m², 90 mg/m², 95 mg/m², 100 mg/m², 105 mg/m², 110 mg/m², 115 mg/m², 120 mg/m²) of docetaxel, to thereby treat the disorder. In one embodiment, the dose is administered once every two, three, four, five, six, seven or eight weeks. In one embodiment, a dose is administered once every three weeks. In one embodiment, the composition includes a CDP-docetaxel conjugate, e.g., a CDP-docetaxel conjugate described herein, e.g., a CDP-docetaxel conjugate comprising docetaxel, coupled, e.g., via linkers, to a CDP described herein, and at least two, three, four, five, six, seven, eight, nine, ten or eleven doses are administered to the subject and each dose is an amount of the composition that includes 30 mg/m² or greater (e.g., 31 mg/m², 33 mg/m², 35 mg/m², 37 mg/m², 40 mg/m², 43 mg/m², 45 mg/m², 47 mg/m², 50 mg/m², 55 mg/m²) of docetaxel, to thereby treat the disorder. In one embodiment, the dose is administered once a week for two, three, four, five, six, seven weeks, e.g., followed by one, two, three weeks without administration of the CDP-docetaxel conjugate. In one embodiment, each dose is administered by intravenous administration over a period of about 30 minutes, 45 minutes, 60 minutes, 90 minutes, 120 minutes, 150 minutes or 180 minutes. In one embodiment, the dosing schedule is not changed between doses. For example, when the dosing schedule is once every three weeks, an additional dose (or doses) is administered in three weeks.

In one embodiment, the CDP-taxane conjugate is a CDP-paclitaxel conjugate, e.g., a CDP-paclitaxel conjugate described herein and, e.g., a CDP-paclitaxel conjugate comprising paclitaxel, coupled, e.g., via linkers, to a CDP described herein, and, e.g., the conjugate is administered in an amount that includes 135 mg/m² or greater (e.g., 140 mg/m², 145 mg/m², 150 mg/m², 155 mg/m², 160 mg/m², 165 mg/m², 170 mg/m², 175 mg/m², 180 mg/m², 185 mg/m², 190 mg/m², 195 mg/m², 200 mg/m², 210 mg/m², 220 mg/m², 230 mg/m², 240 mg/m², 250 mg/m², 260 mg/m²) of paclitaxel, to thereby treat the disorder. In one embodiment, the CDP-paclitaxel conjugate is administered by intravenous administration over a period equal to or less than about 30 minutes, 45 minutes, 60 minutes, 90 minutes, 120 minutes, 150 minutes or 180 minutes. In one embodiment, the subject is administered at least one additional dose of the conjugate, e.g., the subject is administered at least two, three, four, five, six, seven, eight, nine or ten additional doses of the conjugate. In one embodiment, the CDP-paclitaxel conjugate is administered once every one, two, three, four, five or six weeks. In one embodiment, the dosing schedule is not changed between doses. For example, when the dosing schedule is once every three weeks, an additional dose (or doses) is administered in three weeks. In one embodiment, when at least one additional dose is administered, the additional dose (or additional doses) is administered in an amount that includes 135 mg/m² or greater (e.g., 140 mg/m², 145 mg/m², 150 mg/m², 155 mg/m², 160 mg/m², 165 mg/m², 170 mg/m², 175 mg/m², 180 mg/m², 185 mg/m², 190 mg/m², 195 mg/m², 200 mg/m², 210 mg/m², 220 mg/m², 230 mg/m², 240 mg/m², 250 mg/m², 260 mg/m²) of paclitaxel. In one embodiment, when at least one additional dose is administered, the additional dose (or additional doses) is administered by intravenous administration over a period equal to or less than about 30 minutes, 45 minutes, 60 minutes, 90 minutes, 120 minutes, 150 minutes or 180 minutes. In an embodiment, the CDP-paclitaxel conjugate comprises paclitaxel, coupled via a linker shown in FIG. 2 to a CDP described herein. In an embodiment, the CDP-paclitaxel conjugate is a CDP-paclitaxel conjugate shown in FIG. 2.

In one embodiment, the CDP-taxane conjugate includes a CDP-paclitaxel conjugate, e.g., a CDP-paclitaxel conjugate described herein, e.g., a CDP-paclitaxel conjugate comprising paclitaxel, coupled, e.g., via linkers, to a CDP described herein, and the conjugate is administered to the subject in an amount that includes 135 mg/m² or greater (e.g., 140 mg/m², 145 mg/m², 150 mg/m², 155 mg/m², 160 mg/m², 165 mg/m², 170 mg/m², 175 mg/m², 180 mg/m², 185 mg/m², 190 mg/m², 195 mg/m², 200 mg/m², 210 mg/m², 220 mg/m², 230 mg/m², 240 mg/m², 250 mg/m², 260 mg/m²) of paclitaxel, administered by intravenous administration over a period equal to or less than about 30 minutes, 45 minutes, 60 minutes, 90 minutes, 120 minutes, 150 minutes or 180 minutes, for at least two, three, four, five, six, seven or eight doses, wherein the subject is administered a dose of the conjugate once every one, two, three, four, five or six weeks.

In one embodiment, the CDP-taxane conjugate is a CDP-paclitaxel conjugate, e.g., a CDP-paclitaxel conjugate described herein, e.g., a CDP-paclitaxel conjugate comprising paclitaxel, coupled, e.g., via linkers, to a CDP described herein, and at least two, three, four, five, six, seven, eight, nine or ten doses are administered to the subject and each dose is an amount that includes 135 mg/m² or greater (e.g., 140 mg/m², 145 mg/m², 150 mg/m², 155 mg/m², 160 mg/m², 165 mg/m², 170 mg/m², 175 mg/m², 180 mg/m², 185 mg/m², 190 mg/m², 195 mg/m², 200 mg/m², 210 mg/m², 220 mg/m², 230 mg/m², 240 mg/m², 250 mg/m², 260 mg/m²) of paclitaxel, to thereby treat the disorder.

In one embodiment, the dose is administered once every one, two, three, four, five, six, seven or eight weeks. In one embodiment, a dose is administered once every three weeks. In one embodiment, each dose is administered by intravenous administration over a period equal to or less than about 30 minutes, 45 minutes, 60 minutes, 90 minutes, 120 minutes, 150 minutes or 180 minutes. In one embodiment, the dosing schedule is not changed between doses. For example, when the dosing schedule is once every three weeks, an additional dose (or doses) is administered in three weeks.

In one embodiment, the CDP-taxane conjugate is a CDP-cabazitaxel conjugate described herein, e.g., a CDP-cabazitaxel conjugate comprising cabazitaxel, coupled, e.g., directly or via linker, to a CDP described herein, and the CDP-cabazitaxel conjugate is administered to the subject in an amount that includes 5 mg/m² or greater (e.g., 10 mg/m², 12 mg/m², 15 mg/m², 20 mg/m², 25 mg/m², 30 mg/m², 35 mg/m², 40 mg/m², 45 mg/m², 50 mg/m², 55 mg/m², or 60 mg/m²) of cabazitaxel, to thereby treat the disorder. In one embodiment, the conjugate, particle or composition is administered by intravenous administration over a period of about 30 minutes, 45 minutes, 60 minutes, 90 minutes, 120 minutes, 150 minutes or 180 minutes. In one embodiment, the subject is administered at least one additional dose of the conjugate, particle or composition, e.g., the subject is administered at least two, three, four, five, six, seven, eight, nine, ten or eleven additional doses of the conjugate, particle or composition. In one embodiment, the conjugate, particle or composition is administered once every one, two, three, four, five, six weeks. In one embodiment, the dosing schedule is not changed between doses. For example, when the dosing schedule is once every three weeks, an additional dose (or doses) is administered in three weeks. In one embodiment, when at least one additional dose is administered, the additional dose (or additional doses) is administered in an amount such that the conjugate, particle or composition includes 5 mg/m² or greater (e.g., 10 mg/m², 12 mg/m², 15 mg/m², 20 mg/m², 25 mg/m², 30 mg/m², 35 mg/m², 40 mg/m², 45 mg/m², 50 mg/m², 55 mg/m², or 60 mg/m²) of cabazitaxel. In one embodiment, when at least one additional dose is administered, the additional dose (or additional doses) is administered by intravenous administration over a period equal to or less than about 30 minutes, 45 minutes, 60 minutes, 90 minutes, 120 minutes, 150 minutes or 180 minutes.

In one embodiment, the CDP-taxane conjugate is a CDP-cabazitaxel conjugate described herein, e.g., a CDP-cabazitaxel conjugate comprising cabazitaxel, coupled, e.g., directly or via linker, to a CDP described herein, and the CDP-cabazitaxel conjugate is administered to the subject in an amount of the composition that includes 5 mg/m² or greater (e.g., 10 mg/m², 12 mg/m², 15 mg/m², 20 mg/m², 25 mg/m², 30 mg/m², 35 mg/m², 40 mg/m², 45 mg/m², 50 mg/m², 110 mg/m², 55 mg/m², or 60 mg/m²) of cabazitaxel, administered by intravenous administration over a period equal to or less than about 30 minutes, 45 minutes, 60 minutes, 90 minutes, 120 minutes, 150 minutes or 180 minutes, for at least one, two, three, four, five or six doses, wherein the subject is administered a dose of the conjugate, particle or composition once every two, three, four, five or six weeks.

In one embodiment, the CDP-taxane conjugate is a CDP-cabazitaxel conjugate described herein, e.g., a CDP-cabazitaxel conjugate comprising cabazitaxel, coupled, e.g., directly or via linker, to a CDP described herein, and at least two, three, four, five, six, seven, eight, nine, ten or eleven doses are administered to the subject and each dose is an amount of the composition that includes 5 mg/m² or greater (e.g., 10 mg/m², 12 mg/m², 15 mg/m², 20 mg/m², 25 mg/m², 30 mg/m², 35 mg/m², 40 mg/m², 45 mg/m², 50 mg/m², 55 mg/m², or 60 mg/m²) of cabazitaxel, to thereby treat the disorder. In one embodiment, the dose is administered once every one, two, three, four, five, six, seven or eight weeks. In one embodiment, a dose is administered once every three weeks. In one embodiment, each dose is administered by intravenous administration over a period of about 30 minutes, 45 minutes, 60 minutes, 90 minutes, 120 minutes, 150 minutes or 180 minutes. In one embodiment, the dosing schedule is not changed between doses. For example, when the dosing schedule is once every three weeks, an additional dose (or doses) is administered in three weeks.

In one embodiment, the CDP-taxane conjugate, e.g., a CDP-taxane conjugate comprising a taxane molecule (e.g., a docetaxel, paclitaxel, larotaxel and/or cabazitaxel molecule), coupled, e.g., via linkers, to a CDP described herein, is administered once every three weeks in combination with one or more additional chemotherapeutic agent that is also administered once every three weeks. In one embodiment, the CDP-taxane conjugate is administered once every three weeks in combination with one or more of the following chemotherapeutic agents: a vinca alkaloid (e.g., vinblastine, vincristine, vindesine and vinorelbine); an alkylating agent (e.g., cyclophosphamide, dacarbazine, melphalan, ifosfamide, temozolomide); a topoisomerase inhibitor (e.g., topotecan, irinotecan, etoposide, teniposide, lamellarin D, SN-38, camptothecin (e.g., CRLX101, formerly known as IT-101)); a platinum-based agent (e.g., cisplatin, carboplatin, oxaliplatin), an antibiotic (e.g., mitomycin, actinomycin, bleomycin), an antimetabolite (e.g., an antifolate, a purine analogue, a pyrimidine analogue (e.g., capecitabine)); an anthracycline (e.g., doxorubicin, daunorubicin, epirubicin, idarubicin, mitoxantrone, valrubicin); a steroid (e.g., prednisone or prednisolone) and a taxane (e.g., paclitaxel, docetaxel, larotaxel or cabazitaxel).

In one embodiment, the CDP-taxane conjugate, e.g., a CDP-taxane conjugate comprising a taxane molecule, coupled, e.g., via a linker, to a CDP described herein, is administered once every two weeks in combination with one or more additional chemotherapeutic agent that is administered orally. In one embodiment, the CDP-taxane conjugate is administered once every two weeks in combination with one or more of the following chemotherapeutic agents: capecitabine, estramustine, erlotinib, rapamycin, SDZ-RAD, CP-547632; AZD2171, sunitinib, sorafenib and everolimus.

In an embodiment, the CDP-taxane conjugate complex forms a particle or nanoparticle having a conjugate number described herein. By way of example, a CDP-therapeutic agent conjugate, forms, or is provided in, a particle or nanoparticle having a conjugate number of: 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In an embodiment the conjugate number is 2 to 4 or 2 to 5.

In an embodiment the conjugate number is 1, 2, 3, 4, 5, 6, 7, 8, 9, or 10.

In an embodiment the nanoparticle forms, or is provided in, a preparation of nanoparticles, e.g., a pharmaceutical preparation, wherein at least 40, 50, 60, 70, 80, 90 or 95% of the particles in the preparation have a conjugate number provided herein. In an embodiment the nanoparticle forms, or is provided in, a preparation of nanoparticles, e.g., a pharmaceutical preparation, wherein at least 60% of the particles in the preparation have a conjugate number of 1-5 or 2-5.

In an embodiment, the CDP-taxane conjugate is administered as a nanoparticle or preparation of nanoparticles, e.g., a pharmaceutical preparation, wherein at least 60% of the particles in the preparation have a conjugate number of 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In yet another aspect, the invention features a method of identifying a subject, e.g., a human, having a proliferative disorder, e.g., cancer, for treatment with a CDP-taxane conjugate, e.g., a CDP-docetaxel conjugate, a CDP-paclitaxel conjugate, a CDP-larotaxel conjugate and/or a CDP-cabazitaxel described herein, the method comprising identifying a subject having a proliferative disorder who has received an anticancer agent; and administering a composition comprising a CDP-taxane conjugate, e.g., a CDP-docetaxel conjugate, a CDP-paclitaxel conjugate, a CDP-larotaxel conjugate and/or a CDP-cabazitaxel described herein, to a subject, e.g., a human, in an amount effective to treat the disorder, to thereby treat the proliferative disorder.

In another aspect, the disclosure features a method of treating a chemotherapeutic sensitive, a chemotherapeutic refractory, a chemotherapeutic resistant, and/or a relapsed cancer. The method comprises: administering a composition comprising a CDP-taxane conjugate, e.g., a CDP-docetaxel conjugate, a CDP-paclitaxel conjugate, a CDP-larotaxel conjugate and/or a CDP-cabazitaxel described herein, to a subject, e.g., a human, in an amount effective to treat the disorder, to thereby treat the proliferative disorder.

In an embodiment, the CDP-taxane conjugate comprises a taxane molecule (e.g., a docetaxel, paclitaxel, larotaxel and/or cabazitaxel molecule), coupled, e.g., via a linker such as a linker described herein, to a CDP described herein. In an embodiment, the CDP-taxane conjugate comprises a taxane molecule (e.g., a docetaxel, paclitaxel, larotaxel and/or cabazitaxel molecule), coupled via a linker shown in FIG. 2 to a CDP moiety, e.g., a CDP described herein. In an embodiment, the CDP-taxane conjugate is a CDP-taxane conjugate shown in FIG. 2.

In one embodiment, the cancer is refractory to, resistant to and/or relapsed during or after, treatment with, one or more of: an anthracycline (e.g., doxorubicin, daunorubicin, epirubicin, idarubicin, mitoxantrone, valrubicin), an alkylating agent (e.g., cyclophosphamide, dacarbazine, melphalan, ifosfamide, temozolomide), an antimetabolite (e.g., an antifolate, a purine analogue, a pyrimidine analogue (e.g., capecitabine)), a vinca alkaloid (e.g., vinblastine, vincristine, vindesine, vinorelbine), a topoisomerase inhibitor (e.g., topotecan, irinotecan, etoposide, teniposide, lamellarin D, SN-38, camptothecin (e.g., CRLX101)), a taxane (e.g., docetaxel, paclitaxel, larotaxel or cabazitaxel) and a platinum-based agent (e.g., cisplatin, carboplatin, oxaliplatin). In one embodiment, the cancer is resistant to more than one chemotherapeutic agent, e.g., the cancer is a multidrug resistant cancer. In one embodiment, the cancer is resistant to one or more of a platinum based agent, an alkylating agent, an anthracycline and a vinca alkaloid. In one embodiment, the cancer is resistant to one or more of a platinum based agent, an alkylating agent, a taxane and a vinca alkaloid. In one embodiment, the CDP-taxane conjugate (e.g., a CDP-cabazitaxel conjugate) is administered to a subject who cancer is refractory to, resistant to and/or has relapsed during or after treatment with a taxane (e.g., docetaxel or paclitaxel).

In one embodiment, the CDP-taxane conjugate is administered in combination with a second chemotherapeutic agent, e.g., a chemotherapeutic agent described herein. For example, the CDP-taxane conjugate can be administered in combination with a vinca alkaloid (e.g., vinblastine, vincristine, vindesine, vinorelbine), a steroid (e.g., prednisone or prednisolone) and/or a platinum-based agent (e.g., cisplatin, carboplatin, oxaliplatin).

In one embodiment, the cancer is a cancer described herein. For example, the cancer can be a cancer of the bladder (including accelerated and metastatic bladder cancer), breast (e.g., estrogen receptor positive breast cancer; estrogen receptor negative breast cancer; HER-2 positive breast cancer; HER-2 negative breast cancer; progesterone receptor positive breast cancer; progesterone receptor negative breast cancer; estrogen receptor negative, HER-2 negative and progesterone receptor negative breast cancer (i.e., triple negative breast cancer); inflammatory breast cancer), colon (including colorectal cancer), kidney (e.g., transitional cell carcinoma), liver, lung (including small and non-small cell lung cancer, lung adenocarcinoma and squamous cell cancer), genitourinary tract, e.g., ovary (including fallopian tube and peritoneal cancers), cervix, prostate (e.g., hormone refractory prostate cancer), testes, kidney, and ureter, lymphatic system, rectum, larynx, pancreas (including exocrine pancreatic carcinoma), esophagus, stomach, gall bladder, thyroid, skin (including squamous cell carcinoma), brain (including glioblastoma multiforme), head and neck (e.g., occult primary), and soft tissue (e.g., Kaposi's sarcoma (e.g., AIDS related Kaposi's sarcoma), leiomyosarcoma, angiosarcoma, and histiocytoma). Preferred cancers include breast cancer (e.g., metastatic or locally advanced breast cancer), prostate cancer (e.g., hormone refractory prostate cancer), renal cell carcinoma, lung cancer (e.g., non-small cell lung cancer, small cell lung cancer, lung adenocarcinoma, and squamous cell cancer, e.g., unresectable, locally advanced or metastatic non-small cell lung cancer, small cell lung cancer, lung adenocarcinoma, and squamous cell cancer), pancreatic cancer, gastric cancer (e.g., metastatic gastric adenocarcinoma), colorectal cancer, rectal cancer, squamous cell cancer of the head and neck, lymphoma (Hodgkin's lymphoma or non-Hodgkin's lymphoma), renal cell carcinoma, carcinoma of the urothelium, soft tissue sarcoma (e.g., Kaposi's sarcoma (e.g., AIDS related Kaposi's sarcoma), leiomyosarcoma, angiosarcoma, and histiocytoma), gliomas, myeloma (e.g., multiple myeloma), melanoma (e.g., advanced or metastatic melanoma), germ cell tumors, ovarian cancer (e.g., advanced ovarian cancer, e.g., advanced fallopian tube or peritoneal cancer), and gastrointestinal cancer.

In one embodiment, the composition includes a CDP-docetaxel conjugate, e.g., a CDP-docetaxel conjugate described herein, e.g., a CDP-docetaxel conjugate comprising docetaxel molecules, coupled, e.g., via linkers, to a CDP described herein.

In one embodiment, the CDP-docetaxel conjugate is administered at a dose and/or dosing schedule described herein.

In one embodiment, the composition includes a CDP-paclitaxel conjugate, e.g., a CDP-paclitaxel conjugate described herein, e.g., a CDP-paclitaxel conjugate comprising paclitaxel molecules, coupled, e.g., via linkers, to a CDP described herein. In one embodiment, the CDP-paclitaxel conjugate is administered at a dose and/or dosing schedule described herein.

In one embodiment, the composition includes a CDP-larotaxel conjugate, e.g., a CDP-larotaxel conjugate described herein, e.g., a CDP-larotaxel conjugate comprising larotaxel molecules, coupled, e.g., via linkers, to a CDP described herein. In one embodiment, the CDP-larotaxel conjugate is administered at a dose and/or dosing schedule described herein.

In one embodiment, the composition includes a CDP-cabazitaxel conjugate, e.g., a CDP-cabazitaxel conjugate described herein, e.g., a CDP-cabazitaxel conjugate comprising cabazitaxel molecules, coupled, e.g., via linkers, to a CDP described herein.

In one embodiment, the CDP-cabazitaxel conjugate is administered at a dose and/or dosing schedule described herein.

In an embodiment, the CDP-taxane conjugate complex forms a particle or nanoparticle having a conjugate number described herein. By way of example, a CDP-therapeutic agent conjugate, forms, or is provided in, a particle or nanoparticle having a conjugate number of: 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In an embodiment the conjugate number is 2 to 4 or 2 to 5.

In an embodiment the conjugate number is 1, 2, 3, 4, 5, 6, 7, 8, 9, or 10.

In an embodiment the nanoparticle forms, or is provided in, a preparation of nanoparticles, e.g., a pharmaceutical preparation, wherein at least 40, 50, 60, 70, 80, 90 or 95% of the particles in the preparation have a conjugate number provided herein. In an embodiment the nanoparticle forms, or is provided in, a preparation of nanoparticles, e.g., a pharmaceutical preparation, wherein at least 60% of the particles in the preparation have a conjugate number of 1-5 or 2-5.

In an embodiment, the CDP-taxane conjugate is administered as a nanoparticle or preparation of nanoparticles, e.g., a pharmaceutical preparation, wherein at least 60% of the particles in the preparation have a conjugate number of 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In yet another aspect, the invention features a method of treating metastatic or locally advanced breast cancer in a subject, e.g., a human. The method comprises: administering a CDP-taxane conjugate, e.g., a CDP-docetaxel conjugate, a CDP-paclitaxel conjugate, a CDP-larotaxel conjugate and/or a CDP-cabazitaxel conjugate described herein, to a subject in an amount effective to treat the cancer, to thereby treat the cancer.

In an embodiment, the CDP-taxane conjugate comprises taxane molecules (e.g., docetaxel, paclitaxel, larotaxel and/or cabazitaxel molecules), coupled, e.g., via a linker such as a linker described herein, to a CDP moiety, e.g., a CDP described herein. In an embodiment, the CDP-taxane conjugate comprises a taxane (e.g., docetaxel, paclitaxel, larotaxel and/or cabazitaxel), coupled via a linker shown in FIG. 2 to a CDP moiety, e.g., a CDP described herein. In an embodiment, the CDP-taxane conjugate is a CDP-taxane conjugate shown in FIG. 2.

In one embodiment, the breast cancer is estrogen receptor positive breast cancer; estrogen receptor negative breast cancer; HER-2 positive breast cancer; HER-2 negative breast cancer; progesterone receptor positive breast cancer; progesterone receptor negative breast cancer; estrogen receptor negative, HER-2 negative and progesterone receptor negative breast cancer (i.e., triple negative breast cancer) or inflammatory breast cancer.

In one embodiment, the CDP-taxane conjugate is administered in combination with a HER-2 pathway inhibitor, e.g., a HER-2 inhibitor or a HER-2 receptor inhibitor. For example, the CDP-taxane conjugate is administered with trastuzumab.

In some embodiments, the CDP-taxane conjugate is administered in combination with a second chemotherapeutic agent. For example, the CDP-taxane conjugate is administered in combination with a vascular endothelial growth factor (VEGF) pathway inhibitor, e.g., a VEGF inhibitor (e.g., bevacizumab) or VEGF receptor inhibitor (e.g., CP-547632 and AZD2171). In one embodiment, the CDP-taxane conjugate is administered in combination with bevacizumab.

In some embodiments, the CDP-taxane conjugate is administered in combination with an anthracycline (e.g., daunorubicin, doxorubicin, epirubicin, valrubicin and idarubicin).

In some embodiments, the CDP-taxane conjugate is administered in combination with an anti-metabolite, e.g., an antifolate (e.g., floxuridine, pemetrexed) or pyrimidine analogue (e.g., 5FU)).

In some embodiments, the CDP-taxane conjugate is administered in combination with an anthracycline (e.g., daunorubicin, doxorubicin, epirubicin, valrubicin and idarubicin) and an anti-metabolite (e.g., floxuridine, pemetrexed, 5FU).

In some embodiments, the CDP-taxane conjugate is administered in combination with a platinum-based agent (e.g., cisplatin, carboplatin, oxaliplatin).

In some embodiments, the CDP-taxane conjugate is administered in combination with an mTOR inhibitor. Non-limiting examples of mTOR inhibitors include rapamycin, everolimus, AP23573, CCI-779 and SDZ-RAD.

In some embodiments, the CDP-taxane conjugate is administered in combination with a vinca alkaloid (e.g., vinblastine, vincristine, vindesine, vinorelbine).

In some embodiments, the CDP-taxane conjugate is administered in combination with an antibiotic (e.g., mitomycin, actinomycin, bleomycin).

In some embodiments, the CDP-taxane conjugate is administered in combination with an alkylating agent (e.g., cyclophosphamide, dacarbazine, melphalan, ifosfamide, temozolomide).

In one embodiment, the CDP-taxane conjugate is a CDP-docetaxel conjugate, e.g., a CDP-docetaxel conjugate described herein, e.g., a CDP-docetaxel conjugate comprising docetaxel, coupled, e.g., via linkers, to a CDP described herein. In an embodiment, the CDP-docetaxel conjugate comprises docetaxel, coupled via a linker shown in FIG. 2 to a CDP described herein. In an embodiment, the CDP-docetaxel conjugate is a CDP-docetaxel conjugate shown in FIG. 2.

In one embodiment, the CDP-docetaxel conjugate is administered at a dose and/or dosing schedule described herein.

In one embodiment, the CDP-taxane conjugate is a CDP-paclitaxel conjugate, e.g., a CDP-paclitaxel conjugate described herein, e.g., a CDP-paclitaxel conjugate comprising paclitaxel, coupled, e.g., via linkers, to a CDP described herein. In an embodiment, the CDP-paclitaxel conjugate comprises paclitaxel, coupled via a linker shown in FIG. 2 to a CDP described herein. In an embodiment, the CDP-paclitaxel conjugate is a CDP-paclitaxel conjugate shown in FIG. 2.

In one embodiment, the CDP-paclitaxel conjugate is administered at a dose and/or dosing schedule described herein.

In one embodiment, the CDP-taxane conjugate is a CDP-larotaxel conjugate, e.g., a CDP-larotaxel conjugate described herein, e.g., a CDP-larotaxel conjugate comprising larotaxel, coupled, e.g., via linkers, to a CDP described herein. In an embodiment, the CDP-larotaxel conjugate comprises larotaxel, coupled via a linker shown in FIG. 2 to a CDP described herein. In an embodiment, the CDP-larotaxel conjugate is a CDP-larotaxel conjugate shown in FIG. 2.

In one embodiment, the CDP-larotaxel conjugate is administered at a dose and/or dosing schedule described herein.

In one embodiment, the CDP-taxane conjugate is a CDP-cabazitaxel conjugate, e.g., a CDP-cabazitaxel conjugate, e.g., a CDP-cabazitaxel conjugate comprising cabazitaxel, coupled, e.g., via linkers, to a CDP described herein. In an embodiment, the CDP-cabazitaxel conjugate comprises cabazitaxel, coupled via a linker shown in FIG. 2 to a CDP described herein. In an embodiment, the CDP-cabazitaxel conjugate is a CDP-cabazitaxel conjugate shown in FIG. 2.

In one embodiment, the CDP-cabazitaxel conjugate is administered at a dose and/or dosing schedule described herein.

In an embodiment, the CDP-taxane conjugate complex forms a particle or nanoparticle having a conjugate number described herein. By way of example, a CDP-therapeutic agent conjugate, forms, or is provided in, a particle or nanoparticle having a conjugate number of: 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In an embodiment the conjugate number is 2 to 4 or 2 to 5.

In an embodiment the conjugate number is 1, 2, 3, 4, 5, 6, 7, 8, 9, or 10.

In an embodiment the nanoparticle forms, or is provided in, a preparation of nanoparticles, e.g., a pharmaceutical preparation, wherein at least 40, 50, 60, 70, 80, 90 or 95% of the particles in the preparation have a conjugate number provided herein. In an embodiment the nanoparticle forms, or is provided in, a preparation of nanoparticles, e.g., a pharmaceutical preparation, wherein at least 60% of the particles in the preparation have a conjugate number of 1-5 or 2-5.

In an embodiment, the CDP-taxane conjugate is administered as a nanoparticle or preparation of nanoparticles, e.g., a pharmaceutical preparation, wherein at least 60% of the particles in the preparation have a conjugate number of 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In yet another aspect, the invention features a method of treating metastatic or locally advanced breast cancer, e.g. a breast cancer described herein, in a subject, e.g., a human. The method comprises:

providing a subject who has metastatic or locally advanced breast cancer and has been treated with a chemotherapeutic agent which did not effectively treat the cancer (e.g., the subject has a chemotherapeutic refractory, a chemotherapeutic resistant and/or a relapsed cancer) or which had an unacceptable side effect (e.g., the subject has a chemotherapeutic sensitive cancer), and

administering a CDP-taxane conjugate, e.g., a CDP-docetaxel conjugate, a CDP-paclitaxel conjugate, a CDP-larotaxel conjugate and/or a CDP-cabazitaxel conjugate described herein, to a subject in an amount effective to treat the cancer, to thereby treat the cancer.

In an embodiment, the CDP-taxane conjugate comprises taxane molecules (e.g., docetaxel, paclitaxel, larotaxel and/or cabazitaxel molecules), coupled, e.g., via a linker such as a linker described herein, to a CDP moiety, e.g., a CDP described herein. In an embodiment, the CDP-taxane conjugate comprises a taxane (e.g., docetaxel, paclitaxel, larotaxel and/or cabazitaxel), coupled via a linker shown in FIG. 2 to a CDP moiety, e.g., a CDP described herein. In an embodiment, the CDP-taxane conjugate is a CDP-taxane conjugate shown in FIG. 2.

In one embodiment, the cancer is refractory to, resistant to, and/or relapsed with treatment with one or more of: a taxane, an anthracycline, a vinca alkaloid (e.g., vinblastine, vincristine, vindesine and vinorelbine), an alkylating agent (e.g., cyclophosphamide, dacarbazine, melphalan, ifosfamide, temozolomide) and a platinum-based agent (e.g., cisplatin, carboplatin, oxaliplatin). In one embodiment, the cancer is refractory to, resistant to, and/or relapsed with treatment with one or more of: an anthracycline and an alkylating agent, and a CDP-taxane conjugate is administered to the subject.

In one embodiment, the cancer is a multidrug resistant cancer.

In one embodiment, the composition is administered in combination with a pyrimidine analogue, e.g., a pyrimidine analogue described herein (e.g., capecitabine).

In one embodiment, the CDP-taxane conjugate is a CDP-docetaxel conjugate, e.g., a CDP-docetaxel conjugate described herein, e.g., a CDP-docetaxel conjugate comprising docetaxel, coupled, e.g., via linkers, to a CDP described herein. In an embodiment, the CDP-docetaxel conjugate comprises docetaxel, coupled via a linker shown in FIG. 2 to a CDP described herein. In an embodiment, the CDP-docetaxel conjugate is a CDP-docetaxel conjugate shown in FIG. 2.

In one embodiment, the CDP-docetaxel conjugate is administered at a dose and/or dosing schedule described herein.

In one embodiment, the CDP-taxane conjugate is a CDP-paclitaxel conjugate, e.g., a CDP-paclitaxel conjugate described herein, e.g., a CDP-paclitaxel conjugate comprising paclitaxel, coupled, e.g., via linkers, to a CDP described herein. In an embodiment, the CDP-paclitaxel conjugate comprises paclitaxel, coupled via a linker shown in FIG. 2 to a CDP described herein. In an embodiment, the CDP-paclitaxel conjugate is a CDP-paclitaxel conjugate shown in FIG. 2.

In one embodiment, the CDP-paclitaxel conjugate is administered at a dose and/or dosing schedule described herein.

In one embodiment, the CDP-taxane conjugate is a CDP-larotaxel conjugate, e.g., a CDP-larotaxel conjugate described herein, e.g., a CDP-larotaxel conjugate comprising larotaxel, coupled, e.g., via linkers, to a CDP described herein. In an embodiment, the CDP-larotaxel conjugate comprises larotaxel, coupled via a linker shown in FIG. 2 to a CDP described herein. In an embodiment, the CDP-larotaxel conjugate is a CDP-larotaxel conjugate shown in FIG. 2.

In one embodiment, the CDP-larotaxel conjugate is administered at a dose and/or dosing schedule described herein.

In one embodiment, the CDP-taxane conjugate is a CDP-cabazitaxel conjugate, e.g., a CDP-cabazitaxel conjugate described herein, e.g., a CDP-cabazitaxel conjugate comprising cabazitaxel, coupled, e.g., via linkers, to a CDP described herein. In an embodiment, the CDP-cabazitaxel conjugate comprises cabazitaxel, coupled via a linker shown in FIG. 2 to a CDP described herein. In an embodiment, the CDP-cabazitaxel conjugate is a CDP-cabazitaxel conjugate shown in FIG. 2.

In one embodiment, the CDP-cabazitaxel conjugate is administered at a dose and/or dosing schedule described herein.

In an embodiment, the CDP-taxane conjugate complex forms a particle or nanoparticle having a conjugate number described herein. By way of example, a CDP-therapeutic agent conjugate, forms, or is provided in, a particle or nanoparticle having a conjugate number of: 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In an embodiment the conjugate number is 2 to 4 or 2 to 5.

In an embodiment the conjugate number is 1, 2, 3, 4, 5, 6, 7, 8, 9, or 10.

In an embodiment the nanoparticle forms, or is provided in, a preparation of nanoparticles, e.g., a pharmaceutical preparation, wherein at least 40, 50, 60, 70, 80, 90 or 95% of the particles in the preparation have a conjugate number provided herein. In an embodiment the nanoparticle forms, or is provided in, a preparation of nanoparticles, e.g., a pharmaceutical preparation, wherein at least 60% of the particles in the preparation have a conjugate number of 1-5 or 2-5.

In an embodiment, the CDP-taxane conjugate is administered as a nanoparticle or preparation of nanoparticles, e.g., a pharmaceutical preparation, wherein at least 60% of the particles in the preparation have a conjugate number of 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In yet another aspect, the invention features a method of treating hormone refractory prostate cancer in a subject, e.g., a human. The method comprises: administering a CDP-taxane conjugate, e.g., a CDP-docetaxel conjugate, a CDP-paclitaxel conjugate, a CDP-larotaxel conjugate and/or a CDP-cabazitaxel conjugate described herein, to a subject in an amount effective to treat the cancer, to thereby treat the cancer.

In an embodiment, the CDP-taxane conjugate comprises taxane molecules (e.g., docetaxel, paclitaxel, larotaxel and/or cabazitaxel molecules), coupled, e.g., via a linker such as a linker described herein, to a CDP moiety, e.g., a CDP described herein. In an embodiment, the CDP-taxane conjugate comprises a taxane (e.g., docetaxel, paclitaxel, larotaxel and/or cabazitaxel), coupled via a linker shown in FIG. 2 to a CDP moiety, e.g., a CDP described herein. In an embodiment, the CDP-taxane conjugate is a CDP-taxane conjugate shown in FIG. 2.

In one embodiment, the CDP-taxane conjugate is administered in combination with prednisone or prednisolone, e.g., prednisone or prednisolone at a dose of 5 mg, 10 mg or 15 mg).

In one embodiment, the CDP-taxane conjugate is administered in combination with estramustine.

In one embodiment, the CDP-taxane conjugate is administered in combination with an anthracenedione (e.g., mitoxantrone) and prednisone or prednisolone, e.g., prednisone or prednisolone at a dose of 5 mg, 10 mg or 15 mg).

In one embodiment, the CDP-taxane conjugate is administered in combination with a vascular endothelial growth factor (VEGF) pathway inhibitor, e.g., a VEGF inhibitor (e.g., bevacizumab) or VEGF receptor inhibitor (e.g., CP-547632 and AZD2171).

In one embodiment, the CDP-taxane conjugate is administered in combination with an mTOR inhibitor. Non-limiting examples of mTOR inhibitors include rapamycin, everolimus, AP23573, CCI-779, and SDZ-RAD.

In one embodiment, the CDP-taxane conjugate is administered in combination with a platinum-based agent (e.g., cisplatin, carboplatin, oxaliplatin).

In one embodiment, the CDP-taxane conjugate is a CDP-docetaxel conjugate, e.g., a CDP-docetaxel conjugate described herein, e.g., a CDP-docetaxel conjugate comprising docetaxel, coupled, e.g., via linkers, to a CDP described herein. In an embodiment, the CDP-docetaxel conjugate comprises docetaxel, coupled via a linker shown in FIG. 2 to a CDP described herein. In an embodiment, the CDP-docetaxel conjugate is a CDP-docetaxel conjugate shown in FIG. 2.

In one embodiment, the CDP-docetaxel conjugate is administered at a dose and/or dosing schedule described herein.

In one embodiment, the CDP-taxane conjugate is a CDP-paclitaxel conjugate, e.g., a CDP-paclitaxel conjugate described herein, e.g., a CDP-paclitaxel conjugate comprising paclitaxel, coupled, e.g., via linkers, to a CDP described herein. In an embodiment, the CDP-paclitaxel conjugate comprises paclitaxel, coupled via a linker shown in FIG. 2 to a CDP described herein. In an embodiment, the CDP-paclitaxel conjugate is a CDP-paclitaxel conjugate shown in FIG. 2.

In one embodiment, the CDP-paclitaxel conjugate is administered at a dose and/or dosing schedule described herein.

In one embodiment, the CDP-taxane conjugate is a CDP-larotaxel conjugate, e.g., a CDP-larotaxel conjugate described herein, e.g., a CDP-larotaxel conjugate comprising larotaxel, coupled, e.g., via linkers, to a CDP described herein. In an embodiment, the CDP-larotaxel conjugate comprises larotaxel, coupled via a linker shown in FIG. 2 to a CDP described herein. In an embodiment, the CDP-larotaxel conjugate is a CDP-larotaxel conjugate shown in FIG. 2.

In one embodiment, the CDP-larotaxel conjugate is administered at a dose and/or dosing schedule described herein.

In one embodiment, the CDP-taxane conjugate is a CDP-cabazitaxel conjugate, e.g., a CDP-cabazitaxel conjugate described herein, e.g., a CDP-cabazitaxel conjugate comprising cabazitaxel, coupled, e.g., via linkers, to a CDP described herein. In an embodiment, the CDP-cabazitaxel conjugate comprises cabazitaxel, coupled via a linker shown in FIG. 2 to a CDP described herein. In an embodiment, the CDP-cabazitaxel conjugate is a CDP-cabazitaxel conjugate shown in FIG. 2.

In one embodiment, the CDP-cabazitaxel conjugate is administered at a dose and/or dosing schedule described herein.

In an embodiment, the CDP-taxane conjugate complex forms a particle or nanoparticle having a conjugate number described herein. By way of example, a CDP-therapeutic agent conjugate, forms, or is provided in, a particle or nanoparticle having a conjugate number of: 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In an embodiment the conjugate number is 2 to 4 or 2 to 5.

In an embodiment the conjugate number is 1, 2, 3, 4, 5, 6, 7, 8, 9, or 10.

In an embodiment the nanoparticle forms, or is provided in, a preparation of nanoparticles, e.g., a pharmaceutical preparation, wherein at least 40, 50, 60, 70, 80, 90 or 95% of the particles in the preparation have a conjugate number provided herein. In an embodiment the nanoparticle forms, or is provided in, a preparation of nanoparticles, e.g., a pharmaceutical preparation, wherein at least 60% of the particles in the preparation have a conjugate number of 1-5 or 2-5.

In an embodiment, the CDP-taxane conjugate is administered as a nanoparticle or preparation of nanoparticles, e.g., a pharmaceutical preparation, wherein at least 60% of the particles in the preparation have a conjugate number of 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In yet another aspect, the invention features a method of treating hormone refractory prostate cancer in a subject, e.g., a human. The method comprises:

providing a subject who has hormone refractory prostate cancer and has been treated with a chemotherapeutic agent that did not effectively treat the cancer (e.g., the subject has a chemotherapeutic refractory, chemotherapeutic resistant and/or relapsed cancer) or who had unacceptable side effect (e.g., the subject has a chemotherapeutic sensitive cancer), and

administering a CDP-taxane conjugate, e.g., a CDP-docetaxel conjugate, a CDP-paclitaxel conjugate, a CDP-larotaxel conjugate and/or a CDP-cabazitaxel conjugate described herein, to a subject in an amount effective to treat the cancer, to thereby treat the cancer.

In an embodiment, the CDP-taxane conjugate comprises taxane molecules (e.g., docetaxel, paclitaxel, larotaxel and/or cabazitaxel molecules), coupled, e.g., via a linker such as a linker described herein, to a CDP moiety, e.g., a CDP described herein. In an embodiment, the CDP-taxane conjugate comprises a taxane (e.g., docetaxel, paclitaxel, larotaxel and/or cabazitaxel), coupled via a linker shown in FIG. 2 to a CDP moiety, e.g., a CDP described herein. In an embodiment, the CDP-taxane conjugate is a CDP-taxane conjugate shown in FIG. 2.

In one embodiment, the subject has been treated with a taxane (e.g., docetaxel or paclitaxel) that did not effectively treat the cancer (e.g., the subject has a taxane refractory, taxane resistant and/or relapsed cancer), and the subject is administered a CDP-taxane conjugate (e.g., a CDP-cabazitaxel conjugate and/or a CDP-larotaxel conjugate).

In one embodiment, the CDP-taxane conjugate is a CDP-docetaxel conjugate, e.g., a CDP-docetaxel conjugate described herein, e.g., a CDP-docetaxel conjugate comprising docetaxel, coupled, e.g., via linkers, to a CDP described herein. In an embodiment, the CDP-docetaxel conjugate comprises docetaxel, coupled via a linker shown in FIG. 2 to a CDP described herein. In an embodiment, the CDP-docetaxel conjugate is a CDP-docetaxel conjugate shown in FIG. 2.

In one embodiment, the CDP-docetaxel conjugate is administered at a dose and/or dosing schedule described herein.

In one embodiment, the CDP-taxane conjugate is a CDP-paclitaxel conjugate, e.g., a CDP-paclitaxel conjugate described herein, e.g., a CDP-paclitaxel conjugate comprising paclitaxel, coupled, e.g., via linkers, to a CDP described herein. In an embodiment, the CDP-paclitaxel conjugate comprises paclitaxel, coupled via a linker shown in FIG. 2 to a CDP described herein. In an embodiment, the CDP-paclitaxel conjugate is a CDP-paclitaxel conjugate shown in FIG. 2.

In one embodiment, the CDP-paclitaxel conjugate is administered at a dose and/or dosing schedule described herein.

In one embodiment, the CDP-taxane conjugate is a CDP-larotaxel conjugate, e.g., a CDP-larotaxel conjugate described herein, e.g., a CDP-larotaxel conjugate comprising larotaxel, coupled, e.g., via linkers, to a CDP described herein. In an embodiment, the CDP-larotaxel conjugate comprises larotaxel, coupled via a linker shown in FIG. 2 to a CDP described herein. In an embodiment, the CDP-larotaxel conjugate is a CDP-larotaxel conjugate shown in FIG. 2.

In one embodiment, the CDP-larotaxel conjugate is administered at a dose and/or dosing schedule described herein.

In one embodiment, the CDP-taxane conjugate is a CDP-cabazitaxel conjugate, e.g., a CDP-cabazitaxel conjugate described herein, e.g., a CDP-cabazitaxel conjugate comprising cabazitaxel, coupled, e.g., via linkers, to a CDP described herein. In an embodiment, the CDP-cabazitaxel conjugate comprises cabazitaxel, coupled via a linker shown in FIG. 2 to a CDP described herein. In an embodiment, the CDP-cabazitaxel conjugate is a CDP-cabazitaxel conjugate shown in FIG. 2.

In one embodiment, the CDP-cabazitaxel conjugate is administered at a dose and/or dosing schedule described herein.

In one embodiment, the CDP-taxane conjugate is administered in combination with prednisone or prednisolone, e.g., prednisone or prednisolone at a dose of 5 mg, 10 mg or 15 mg).

In an embodiment, the CDP-taxane conjugate complex forms a particle or nanoparticle having a conjugate number described herein. By way of example, a CDP-therapeutic agent conjugate, forms, or is provided in, a particle or nanoparticle having a conjugate number of: 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In an embodiment the conjugate number is 2 to 4 or 2 to 5.

In an embodiment the conjugate number is 1, 2, 3, 4, 5, 6, 7, 8, 9, or 10.

In an embodiment the nanoparticle forms, or is provided in, a preparation of nanoparticles, e.g., a pharmaceutical preparation, wherein at least 40, 50, 60, 70, 80, 90 or 95% of the particles in the preparation have a conjugate number provided herein. In an embodiment the nanoparticle forms, or is provided in, a preparation of nanoparticles, e.g., a pharmaceutical preparation, wherein at least 60% of the particles in the preparation have a conjugate number of 1-5 or 2-5.

In an embodiment, the CDP-taxane conjugate is administered as a nanoparticle or preparation of nanoparticles, e.g., a pharmaceutical preparation, wherein at least 60% of the particles in the preparation have a conjugate number of 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In yet another aspect, the invention features a method of treating metastatic or advanced ovarian cancer (e.g., peritoneal or fallopian tube cancer) in a subject, e.g., a human. The method comprises: administering a CDP-taxane conjugate, e.g., a CDP-docetaxel conjugate, a CDP-larotaxel conjugate and/or a CDP-cabazitaxel conjugate described herein, to a subject in an amount effective to treat the cancer, to thereby treat the cancer.

In an embodiment, the CDP-taxane conjugate comprises taxane molecules (e.g., docetaxel, paclitaxel, larotaxel and/or cabazitaxel molecules), coupled, e.g., via a linker such as a linker described herein, to a CDP moiety, e.g., a CDP described herein. In an embodiment, the CDP-taxane conjugate comprises a taxane (e.g., docetaxel, paclitaxel, larotaxel and/or cabazitaxel), coupled via a linker shown in FIG. 2 to a CDP moiety, e.g., a CDP described herein. In an embodiment, the CDP-taxane conjugate is a CDP-taxane conjugate shown in FIG. 2.

In one embodiment, the CDP-taxane conjugate is administered in combination with a platinum-based agent (e.g., cisplatin, carboplatin, oxaliplatin).

In one embodiment, the CDP-taxane conjugate is administered in combination with an alkylating agent (e.g., cyclophosphamide, dacarbazine, melphalan, ifosfamide, temozolomide).

In one embodiment, the CDP-taxane conjugate is administered in combination with a platinum-based agent (e.g., cisplatin, carboplatin, oxaliplatin) and an alkylating agent (e.g., cyclophosphamide, dacarbazine, melphalan, ifosfamide, temozolomide).

In one embodiment, the CDP-taxane conjugate is administered in combination with one or more of: an anti-metabolite, e.g., an antifolate (e.g., pemetrexed, floxuridine, raltitrexed) or pyrimidine analog (e.g., capecitabine, cytrarabine, gemcitabine, 5-fluorouracil); an alkylating agent (e.g., cyclophosphamide, dacarbazine, melphalan, ifosfamide, temozolomide); a topoisomerase inhibitor (e.g., etoposide, topotecan, irinotecan, tenoposide, lamellarin D, SN-38); a platinum based agent (carboplatin, cisplatin, oxaliplatin); a vinca alkaloid (e.g., vinblastine, vincristine, vindesine, vinorelbine). In one embodiment, the composition is administered in combination with one or more of: capecitabine, cyclophosphamide, etoposide, gemcitabine, ifosfamide, irinotecan, melphalan, oxaliplatin, vinorelbine, vincristine and pemetrexed.

In one embodiment, the CDP-taxane conjugate is administered in combination with a vascular endothelial growth factor (VEGF) pathway inhibitor, e.g., a VEGF inhibitor or VEGF receptor inhibitor. In one embodiment, the VEGF inhibitor is bevacizumab. In another embodiment, the VEGF receptor inhibitor is selected from CP-547632 and AZD2171.

In one embodiment, the CDP-taxane conjugate is administered in combination with an mTOR inhibitor, e.g., rapamycin, everolimus, AP23573, CCI-779 or SDZ-RAD.

In one embodiment, the CDP-taxane conjugate is a CDP-docetaxel conjugate, e.g., a CDP-docetaxel conjugate described herein, e.g., a CDP-docetaxel conjugate comprising docetaxel, coupled, e.g., via linkers, to a CDP described herein. In an embodiment, the CDP-docetaxel conjugate comprises docetaxel, coupled via a linker shown in FIG. 2 to a CDP described herein. In an embodiment, the CDP-docetaxel conjugate is a CDP-docetaxel conjugate shown in FIG. 2.

In one embodiment, the CDP-docetaxel conjugate is administered at a dose and/or dosing schedule described herein.

In one embodiment, the CDP-taxane conjugate is a CDP-paclitaxel conjugate, e.g., a CDP-paclitaxel conjugate described herein, e.g., a CDP-paclitaxel conjugate comprising paclitaxel, coupled, e.g., via linkers, to a CDP described herein. In an embodiment, the CDP-paclitaxel conjugate comprises paclitaxel, coupled via a linker shown in FIG. 2 to a CDP described herein. In an embodiment, the CDP-paclitaxel conjugate is a CDP-paclitaxel conjugate shown in FIG. 2.

In one embodiment, the CDP-paclitaxel conjugate is administered at a dose and/or dosing schedule described herein.

In one embodiment, the CDP-taxane conjugate is a CDP-larotaxel conjugate, e.g., a CDP-larotaxel conjugate described herein, e.g., a CDP-larotaxel conjugate comprising larotaxel, coupled, e.g., via linkers, to a CDP described herein. In an embodiment, the CDP-larotaxel conjugate comprises larotaxel, coupled via a linker shown in FIG. 2 to a CDP described herein. In an embodiment, the CDP-larotaxel conjugate is a CDP-larotaxel conjugate shown in FIG. 2.

In one embodiment, the CDP-larotaxel conjugate is administered at a dose and/or dosing schedule described herein.

In one embodiment, the CDP-taxane conjugate is a CDP-cabazitaxel conjugate, e.g., a CDP-cabazitaxel conjugate described herein, e.g., a CDP-cabazitaxel conjugate comprising cabazitaxel, coupled, e.g., via linkers, to a CDP described herein. In an embodiment, the CDP-cabazitaxel conjugate comprises cabazitaxel, coupled via a linker shown in FIG. 2 to a CDP described herein. In an embodiment, the CDP-cabazitaxel conjugate is a CDP-cabazitaxel conjugate shown in FIG. 2.

In one embodiment, the CDP-cabazitaxel conjugate is administered at a dose and/or dosing schedule described herein.

In an embodiment, the CDP-taxane conjugate complex forms a particle or nanoparticle having a conjugate number described herein. By way of example, a CDP-therapeutic agent conjugate, forms, or is provided in, a particle or nanoparticle having a conjugate number of: 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In an embodiment the conjugate number is 2 to 4 or 2 to 5.

In an embodiment the conjugate number is 1, 2, 3, 4, 5, 6, 7, 8, 9, or 10.

In an embodiment the nanoparticle forms, or is provided in, a preparation of nanoparticles, e.g., a pharmaceutical preparation, wherein at least 40, 50, 60, 70, 80, 90 or 95% of the particles in the preparation have a conjugate number provided herein. In an embodiment the nanoparticle forms, or is provided in, a preparation of nanoparticles, e.g., a pharmaceutical preparation, wherein at least 60% of the particles in the preparation have a conjugate number of 1-5 or 2-5.

In an embodiment, the CDP-taxane conjugate is administered as a nanoparticle or preparation of nanoparticles, e.g., a pharmaceutical preparation, wherein at least 60% of the particles in the preparation have a conjugate number of 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In yet another aspect, the invention features a method of treating metastatic or advanced ovarian cancer (e.g., peritoneal or fallopian tube cancer) in a subject, e.g., a human. The method comprises:

providing a subject who has advanced ovarian cancer and has been treated with a chemotherapeutic agent that did not effectively treat the cancer (e.g., the subject has a chemotherapeutic refractory, a chemotherapeutic resistant and/or a relapsed cancer) or who had an unacceptable side effect (e.g., the subject has a chemotherapeutic sensitive cancer), and

administering a composition comprising a CDP-taxane conjugate, e.g., a CDP-docetaxel conjugate, a CDP-larotaxel conjugate and/or a CDP-cabazitaxel conjugate described herein, to a subject in an amount effective to treat the cancer, to thereby treat the cancer.

In an embodiment, the CDP-taxane conjugate comprises taxane molecules (e.g., docetaxel, paclitaxel, larotaxel and/or cabazitaxel molecules), coupled, e.g., via a linker such as a linker described herein, to a CDP moiety, e.g., a CDP described herein. In an embodiment, the CDP-taxane conjugate comprises a taxane (e.g., docetaxel, paclitaxel, larotaxel and/or cabazitaxel), coupled via a linker shown in FIG. 2 to a CDP moiety, e.g., a CDP described herein. In an embodiment, the CDP-taxane conjugate is a CDP-taxane conjugate shown in FIG. 2.

In one embodiment, the subject has been treated with a platinum-based agent that did not effectively treat the cancer (e.g., the subject has been treated with cisplatin, carboplatin or oxaliplatin which did not effectively treat the cancer). In one embodiment, the subject has been treated with cisplatin or carboplatin which did not effectively treat the cancer. In one embodiment, the subject has been treated with a taxane (e.g., docetaxel or paclitaxel) which did not effectively treat the cancer.

In one embodiment, the CDP-taxane conjugate is administered in combination with a pyrimidine analog, e.g., capecitabine or gemcitabine.

In one embodiment, the CDP-taxane conjugate is administered in combination with capecitabine and gemcitabine.

In one embodiment, the CDP-taxane conjugate is administered in combination with an anthracycline, e.g., daunorubicin, doxorubicin, epirubicin, valrubicin and idarubicin. In one embodiment, the anthracycline is doxorubicin, e.g., liposomal doxorubicin.

In one embodiment, the CDP-taxane conjugate is administered in combination with a topoisomerase I inhibitor, e.g., irinotecan, topotecan, tenoposide, lamellarin D, SN-38, camptothecin (e.g., CRLX101). In one embodiment the topoisomerase I inhibitor is topotecan. In another embodiment, the topoisomerase I inhibitor is irinotecan or etoposide.

In one embodiment, the CDP-taxane conjugate is administered in combination with one or more of: an anti-metabolite, e.g., an antifolate (e.g., pemetrexed, floxuridine, raltitrexed) or pyrimidine analog (e.g., capecitabine, cytrarabine, gemcitabine, 5FU); an alkylating agent (e.g., cyclophosphamide, dacarbazine, melphalan, ifosfamide, temozolomide); a platinum based agent (carboplatin, cisplatin, oxaliplatin); and a vinca alkaloid (e.g., vinblastine, vincristine, vindesine, vinorelbine). In one embodiment, the CDP-taxane conjugate is administered in combination with one or more of: capecitabine, cyclophosphamide, etoposide, gemcitabine, ifosfamide, irinotecan, melphalan, oxaliplatin, vinorelbine, vincristine and pemetrexed.

In one embodiment, the CDP-taxane conjugate is a CDP-docetaxel conjugate, e.g., a CDP-docetaxel conjugate described herein, e.g., a CDP-docetaxel conjugate comprising docetaxel, coupled, e.g., via linkers, to a CDP described herein. In an embodiment, the CDP-docetaxel conjugate comprises docetaxel, coupled via a linker shown in FIG. 2 to a CDP described herein. In an embodiment, the CDP-docetaxel conjugate is a CDP-docetaxel conjugate shown in FIG. 2.

In one embodiment, the CDP-docetaxel conjugate is administered at a dose and/or dosing schedule described herein.

In one embodiment, the CDP-taxane conjugate is a CDP-paclitaxel conjugate, e.g., a CDP-paclitaxel conjugate described herein, e.g., a CDP-paclitaxel conjugate comprising paclitaxel, coupled, e.g., via linkers, to a CDP described herein. In an embodiment, the CDP-paclitaxel conjugate comprises paclitaxel, coupled via a linker shown in FIG. 2 to a CDP described herein. In an embodiment, the CDP-paclitaxel conjugate is a CDP-paclitaxel conjugate shown in FIG. 2.

In one embodiment, the CDP-paclitaxel conjugate is administered at a dose and/or dosing schedule described herein.

In one embodiment, the CDP-taxane conjugate is a CDP-larotaxel conjugate, e.g., a CDP-larotaxel conjugate described herein, e.g., a CDP-larotaxel conjugate comprising larotaxel, coupled, e.g., via linkers, to a CDP described herein. In an embodiment, the CDP-larotaxel conjugate comprises larotaxel, coupled via a linker shown in FIG. 2 to a CDP described herein. In an embodiment, the CDP-larotaxel conjugate is a CDP-larotaxel conjugate shown in FIG. 2.

In one embodiment, the CDP-larotaxel conjugate is administered at a dose and/or dosing schedule described herein.

In one embodiment, the CDP-taxane conjugate is a CDP-cabazitaxel conjugate, e.g., a CDP-cabazitaxel conjugate described herein, e.g., a CDP-cabazitaxel conjugate comprising cabazitaxel, coupled, e.g., via linkers, to a CDP described herein. In an embodiment, the CDP-cabazitaxel conjugate comprises cabazitaxel, coupled via a linker shown in FIG. 2 to a CDP described herein. In an embodiment, the CDP-cabazitaxel conjugate is a CDP-cabazitaxel conjugate shown in FIG. 2.

In one embodiment, the CDP-cabazitaxel conjugate is administered at a dose and/or dosing schedule described herein.

In an embodiment, the CDP-taxane conjugate complex forms a particle or nanoparticle having a conjugate number described herein. By way of example, a CDP-therapeutic agent conjugate, forms, or is provided in, a particle or nanoparticle having a conjugate number of: 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In an embodiment the conjugate number is 2 to 4 or 2 to 5.

In an embodiment the conjugate number is 1, 2, 3, 4, 5, 6, 7, 8, 9, or 10.

In an embodiment the nanoparticle forms, or is provided in, a preparation of nanoparticles, e.g., a pharmaceutical preparation, wherein at least 40, 50, 60, 70, 80, 90 or 95% of the particles in the preparation have a conjugate number provided herein. In an embodiment the nanoparticle forms, or is provided in, a preparation of nanoparticles, e.g., a pharmaceutical preparation, wherein at least 60% of the particles in the preparation have a conjugate number of 1-5 or 2-5.

In an embodiment, the CDP-taxane conjugate is administered as a nanoparticle or preparation of nanoparticles, e.g., a pharmaceutical preparation, wherein at least 60% of the particles in the preparation have a conjugate number of 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In yet another aspect, the invention features a method of treating non-small cell lung cancer (e.g., unresectable, locally advanced or metastatic non-small cell lung cancer) in a subject, e.g., a human. The method comprises: administering a CDP-taxane conjugate, e.g., a CDP-docetaxel conjugate, a CDP-paclitaxel conjugate, a CDP-larotaxel conjugate and/or a CDP-cabazitaxel conjugate described herein, to a subject in an amount effective to treat the cancer, to thereby treat the cancer.

In an embodiment, the CDP-taxane conjugate comprises taxane molecules (e.g., docetaxel, paclitaxel, larotaxel and/or cabazitaxel molecules), coupled, e.g., a via linker such as a liner described herein, to a CDP moiety, e.g., a CDP described herein. In an embodiment, the CDP-taxane conjugate comprises a taxane (e.g., docetaxel, paclitaxel, larotaxel and/or cabazitaxel), coupled via a linker shown in FIG. 2 to a CDP moiety, e.g., a CDP described herein. In an embodiment, the CDP-taxane conjugate is a CDP-taxane conjugate shown in FIG. 2.

In one embodiment, the CDP-taxane conjugate is administered in combination with a vascular endothelial (VEGF) pathway inhibitor, e.g., a VEGF inhibitor or VEGF receptor inhibitor. In one embodiment, the VEGF inhibitor is bevacizumab. In another embodiment, the VEGF receptor inhibitor is selected from CP-547632 and AZD2171.

In one embodiment, the CDP-taxane conjugate is administered in combination with an epidermal growth factor (EGF) pathway inhibitor, e.g., an EGF inhibitor or EGF receptor inhibitor. In one embodiment, the EGF receptor inhibitor is cetuximab, erlotinib, or gefitinib.

In one embodiment, the CDP-taxane conjugate is administered in combination with a platinum-based agent (e.g., cisplatin, carboplatin, oxaliplatin). In one embodiment, the CDP-taxane conjugate is administered in combination with a platinum-based agent (e.g., cisplatin, carboplatin, oxaliplatin) and a nucleoside analog (e.g., gemcitabine). In one embodiment, the CDP-taxane conjugate is administered in combination with a platinum-based agent (e.g., cisplatin, carboplatin, oxaliplatin) and an anti-metabolite, e.g., an antifolate (e.g., floxuridine, pemetrexed) or pyrimidine analogue (e.g., 5FU). In one embodiment, the CDP-taxane conjugate is administered in combination with a platinum-based agent (e.g., cisplatin, carboplatin, oxaliplatin) and a vinca alkaloid (e.g., vinblastine, vincristine, vindesine, vinorelbine).

In one embodiment, the CDP-taxane conjugate is administered in combination with a vinca alkaloid (e.g., vinblastine, vincristine, vindesine, vinorelbine).

In one embodiment, the CDP-taxane conjugate is administered in combination with an alkylating agent (e.g., cyclophosphamide, dacarbazine, melphalan, ifosfamide, temozolomide).

In one embodiment, the CDP-taxane conjugate is administered in combination with an mTOR inhibitor, e.g., rapamycin, everolimus, AP23573, CCI-779 or SDZ-RAD.

In one embodiment, the CDP-taxane conjugate, either alone or with any of the combinations described herein, is administered in combination with radiation.

In one embodiment, the CDP-taxane conjugate is a CDP-docetaxel conjugate, e.g., a CDP-docetaxel conjugate described herein, e.g., a CDP-docetaxel conjugate comprising docetaxel, coupled, e.g., via linkers, to a CDP described herein. In an embodiment, the CDP-docetaxel conjugate comprises docetaxel, coupled via a linker shown in FIG. 2 to a CDP described herein. In an embodiment, the CDP-docetaxel conjugate is a CDP-docetaxel conjugate shown in FIG. 2.

In one embodiment, the CDP-docetaxel conjugate is administered at a dose and/or dosing schedule described herein.

In one embodiment, the CDP-taxane conjugate is a CDP-paclitaxel conjugate, e.g., a CDP-paclitaxel conjugate described herein, e.g., a CDP-paclitaxel conjugate comprising paclitaxel, coupled, e.g., via linkers, to a CDP described herein. In an embodiment, the CDP-paclitaxel conjugate comprises paclitaxel, coupled via a linker shown in FIG. 2 to a CDP described herein. In an embodiment, the CDP-paclitaxel conjugate is a CDP-paclitaxel conjugate shown in FIG. 2.

In one embodiment, the CDP-paclitaxel conjugate is administered at a dose and/or dosing schedule described herein.

In one embodiment, the CDP-taxane conjugate is a CDP-larotaxel conjugate, e.g., a CDP-larotaxel conjugate described herein, e.g., a CDP-larotaxel conjugate comprising larotaxel, coupled, e.g., via linkers, to a CDP described herein. In an embodiment, the CDP-larotaxel conjugate comprises larotaxel, coupled via a linker shown in FIG. 2 to a CDP described herein. In an embodiment, the CDP-larotaxel conjugate is a CDP-larotaxel conjugate shown in FIG. 2.

In one embodiment, the CDP-larotaxel conjugate is administered at a dose and/or dosing schedule described herein.

In one embodiment, the CDP-taxane conjugate is a CDP-cabazitaxel conjugate, e.g., a CDP-cabazitaxel conjugate described herein, e.g., a CDP-cabazitaxel conjugate comprising cabazitaxel, coupled, e.g., via linkers, to a CDP described herein. In an embodiment, the CDP-cabazitaxel conjugate comprises cabazitaxel, coupled via a linker shown in FIG. 2 to a CDP described herein. In an embodiment, the CDP-cabazitaxel conjugate is a CDP-cabazitaxel conjugate shown in FIG. 2.

In one embodiment, the CDP-cabazitaxel conjugate is administered at a dose and/or dosing schedule described herein.

In an embodiment, the CDP-taxane conjugate complex forms a particle or nanoparticle having a conjugate number described herein. By way of example, a CDP-therapeutic agent conjugate, forms, or is provided in, a particle or nanoparticle having a conjugate number of: 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In an embodiment the conjugate number is 2 to 4 or 2 to 5.

In an embodiment the conjugate number is 1, 2, 3, 4, 5, 6, 7, 8, 9, or 10.

In an embodiment the nanoparticle forms, or is provided in, a preparation of nanoparticles, e.g., a pharmaceutical preparation, wherein at least 40, 50, 60, 70, 80, 90 or 95% of the particles in the preparation have a conjugate number provided herein. In an embodiment the nanoparticle forms, or is provided in, a preparation of nanoparticles, e.g., a pharmaceutical preparation, wherein at least 60% of the particles in the preparation have a conjugate number of 1-5 or 2-5.

In an embodiment, the CDP-taxane conjugate is administered as a nanoparticle or preparation of nanoparticles, e.g., a pharmaceutical preparation, wherein at least 60% of the particles in the preparation have a conjugate number of 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In yet another aspect, the invention features a method of treating unresectable, advanced or metastatic non-small cell lung cancer in a subject, e.g., a human. The method comprises:

providing a subject who has unresectable, advanced or metastatic non-small cell lung cancer and has been treated with a chemotherapeutic agent that did not effectively treat the cancer (e.g., the subject has a chemotherapeutic refractory, a chemotherapeutic resistant and/or a relapsed cancer) or who had an unacceptable side effect (e.g., the subject has a chemotherapeutic sensitive cancer), and

administering a CDP-taxane conjugate, e.g., a CDP-docetaxel conjugate, a CDP-paclitaxel conjugate, a CDP-larotaxel conjugate and/or a CDP-cabazitaxel conjugate described herein, to a subject in an amount effective to treat the cancer, to thereby treat the cancer.

In an embodiment, the CDP-taxane conjugate comprises taxane molecules (e.g., docetaxel, paclitaxel, larotaxel and/or cabazitaxel molecules), coupled, e.g., via a linker such as a linker described herein, to a CDP moiety, e.g., a CDP described herein. In an embodiment, the CDP-taxane conjugate comprises a taxane (e.g., docetaxel, paclitaxel, larotaxel and/or cabazitaxel), coupled via a linker shown in FIG. 2 to a CDP moiety, e.g., a CDP described herein. In an embodiment, the CDP-taxane conjugate is a CDP-taxane conjugate shown in FIG. 2.

In one embodiment, the subject has been treated with a vascular endothelial growth factor (VEGF) pathway inhibitor (e.g., a VEGF inhibitor or VEGF receptor inhibitor) which did not effectively treat the cancer (e.g., the subject has been treated with bevacizumab CP-547632 or AZD2171 which did not effectively treat the cancer).

In one embodiment, the subject has been treated with an endothelial growth factor (EGF) pathway inhibitor (e.g., an EGF inhibitor or an EGF receptor inhibitor) which did not effectively treat the cancer (e.g., the subject has been treated with cetuximab, erlotinib, gefitinib which did not effectively treat the cancer).

In one embodiment, the subject has been treated with a platinum-based agent which did not effectively treat the cancer (e.g., the subject has been treated with cisplatin, carboplatin or oxaliplatin which did not effectively treat the cancer).

In one embodiment, the subject has been treated with a taxane (e.g., docetaxel or paclitaxel) which did not effectively treat the cancer.

In one embodiment, the CDP-taxane conjugate is administered in combination with an anti-metabolite, e.g., an antifolate, e.g., floxuridine, pemetrexed or pyrimidine analogue (e.g., 5FU).

In one embodiment, the CDP-taxane conjugate is administered in combination with an EGF pathway inhibitor, e.g., an EGF inhibitor or EGF receptor inhibitor. The EGF receptor inhibitor can be, e.g., cetuximab, erlotinib or gefitinib.

In one embodiment, the CDP-taxane conjugate is a CDP-docetaxel conjugate, e.g., a CDP-docetaxel conjugate described herein, e.g., a CDP-docetaxel conjugate comprising docetaxel, coupled, e.g., via linkers, to a CDP described herein. In an embodiment, the CDP-docetaxel conjugate comprises docetaxel, coupled via a linker shown in FIG. 2 to a CDP described herein. In an embodiment, the CDP-docetaxel conjugate is a CDP-docetaxel conjugate shown in FIG. 2.

In one embodiment, the CDP-docetaxel conjugate is administered at a dose and/or dosing schedule described herein.

In one embodiment, the CDP-taxane conjugate is a CDP-paclitaxel conjugate, e.g., a CDP-paclitaxel conjugate described herein, e.g., a CDP-paclitaxel conjugate comprising paclitaxel, coupled, e.g., via linkers, to a CDP described herein. In an embodiment, the CDP-paclitaxel conjugate comprises paclitaxel, coupled via a linker shown in FIG. 2 to a CDP described herein. In an embodiment, the CDP-paclitaxel conjugate is a CDP-paclitaxel conjugate shown in FIG. 2.

In one embodiment, the CDP-paclitaxel conjugate is administered at a dose and/or dosing schedule described herein.

In one embodiment, the CDP-taxane conjugate is a CDP-larotaxel conjugate, e.g., a CDP-larotaxel conjugate described herein, e.g., a CDP-larotaxel conjugate comprising larotaxel, coupled, e.g., via linkers, to a CDP described herein. In an embodiment, the CDP-larotaxel conjugate comprises larotaxel, coupled via a linker shown in FIG. 2 to a CDP described herein. In an embodiment, the CDP-larotaxel conjugate is a CDP-larotaxel conjugate shown in FIG. 2.

In one embodiment, the CDP-larotaxel conjugate is administered at a dose and/or dosing schedule described herein.

In one embodiment, the CDP-taxane conjugate is a CDP-cabazitaxel conjugate, e.g., a CDP-cabazitaxel conjugate described herein, e.g., a CDP-cabazitaxel conjugate comprising cabazitaxel, coupled, e.g., via linkers, to a CDP described herein. In an embodiment, the CDP-cabazitaxel conjugate comprises cabazitaxel, coupled via a linker shown in FIG. 2 to a CDP described herein. In an embodiment, the CDP-cabazitaxel conjugate is a CDP-cabazitaxel conjugate shown in FIG. 2.

In one embodiment, the CDP-cabazitaxel conjugate is administered at a dose and/or dosing schedule described herein.

In an embodiment, the CDP-taxane conjugate complex forms a particle or nanoparticle having a conjugate number described herein. By way of example, a CDP-therapeutic agent conjugate, forms, or is provided in, a particle or nanoparticle having a conjugate number of: 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In an embodiment the conjugate number is 2 to 4 or 2 to 5.

In an embodiment the conjugate number is 1, 2, 3, 4, 5, 6, 7, 8, 9, or 10.

In an embodiment the nanoparticle forms, or is provided in, a preparation of nanoparticles, e.g., a pharmaceutical preparation, wherein at least 40, 50, 60, 70, 80, 90 or 95% of the particles in the preparation have a conjugate number provided herein. In an embodiment the nanoparticle forms, or is provided in, a preparation of nanoparticles, e.g., a pharmaceutical preparation, wherein at least 60% of the particles in the preparation have a conjugate number of 1-5 or 2-5.

In an embodiment, the CDP-taxane conjugate is administered as a nanoparticle or preparation of nanoparticles, e.g., a pharmaceutical preparation, wherein at least 60% of the particles in the preparation have a conjugate number of 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In yet another aspect, the invention features a method of treating multiple myeloma in a subject, e.g., a human. The method comprises: administering a composition comprising a CDP-taxane conjugate, e.g., a CDP-docetaxel conjugate, a CDP-paclitaxel conjugate, a CDP-larotaxel conjugate and/or a CDP-cabazitaxel conjugate described herein, to a subject in an amount effective to treat the myeloma, to thereby treat the myeloma.

In an embodiment, the CDP-taxane conjugate comprises taxane molecules (e.g., docetaxel, paclitaxel, larotaxel and/or cabazitaxel molecules), coupled, e.g., via a linker such as a linker described herein, to a CDP moiety, e.g., a CDP described herein. In an embodiment, the CDP-taxane conjugate comprises a taxane (e.g., docetaxel, paclitaxel, larotaxel and/or cabazitaxel), coupled via a linker shown in FIG. 2 to a CDP moiety, e.g., a CDP described herein. In an embodiment, the CDP-taxane conjugate is a CDP-taxane conjugate shown in FIG. 2.

In one embodiment, the CDP-taxane conjugate is administered as a primary treatment for multiple myeloma.

In one embodiment, the CDP-taxane conjugate is administered in combination with dexamethasone. In one embodiment, the CDP-taxane conjugate is further administered in combination with an anthracycline (e.g., daunorubicin, doxorubicin (e.g., liposomal doxorubicin), epirubicin, valrubicin and idarubicin), thalidomide or thalidomide derivative (e.g., lenalidomide).

In one embodiment, the CDP-taxane conjugate is administered in combination with a proteasome inhibitor (e.g., bortezomib) and dexamethasone. In one embodiment, the CDP-taxane conjugate is further administered in combination with an anthracycline (e.g., daunorubicin, doxorubicin (e.g., liposomal doxorubicin), epirubicin, valrubicin and idarubicin), thalidomide or thalidomide derivative (e.g., lenalidomide).

In one embodiment, the CDP-taxane conjugate is administered in combination with a vinca alkaloid (e.g., vinblastine, vincristine, vindesine and vinorelbine) and dexamethasone. In one embodiment, the CDP-taxane conjugate is further administered in combination with an anthracycline (e.g., daunorubicin, doxorubicin (e.g., liposomal doxorubicin), epirubicin, valrubicin and idarubicin).

In one embodiment, the CDP-taxane conjugate is administered in combination with thalidomide or thalidomide derivative (e.g., lenalidomide).

In one embodiment, after the subject has received a primary treatment, e.g., a primary treatment described herein, the subject is further administered a high dose treatment. For example, the subject can be administered a high dose treatment of dexamethasone, an alkylating agent (e.g., cyclophosphamide or melphalan) and/or a CDP-taxane conjugate described herein.

In one embodiment, after the primary treatment, e.g., after the primary treatment and the high dose treatment, stem cells are transplanted into the subject. In one embodiment, a subject who has received a stem cell transplant is administered thalidomide. In one embodiment, the subject is further administered a corticosteroid (e.g., prednisone).

In one embodiment, the CDP-taxane conjugate is administered in combination with a vascular endothelial growth factor (VEGF) pathway inhibitor, e.g., a VEGF inhibitor or VEGF receptor inhibitor. In one embodiment, the VEGF inhibitor is bevacizumab. In one embodiment, the VEGF receptor inhibitor is selected from CP-547632 and AZD2171.

In some embodiments, the CDP-taxane conjugate is administered in combination with an mTOR inhibitor. Non-limiting examples of mTOR inhibitors include rapamycin, everolimus, AP23573, CCI-779 and SDZ-RAD.

In one embodiment, the CDP-taxane conjugate is a CDP-docetaxel conjugate, e.g., a CDP-docetaxel conjugate described herein, e.g., a CDP-docetaxel conjugate comprising docetaxel, coupled, e.g., via linkers, to a CDP described herein. In an embodiment, the CDP-docetaxel conjugate comprises docetaxel, coupled via a linker shown in FIG. 2 to a CDP described herein. In an embodiment, the CDP-docetaxel conjugate is a CDP-docetaxel conjugate shown in FIG. 2.

In one embodiment, the CDP-docetaxel conjugate is administered at a dose and/or dosing schedule described herein.

In one embodiment, the CDP-taxane conjugate is a CDP-paclitaxel conjugate, e.g., a CDP-paclitaxel conjugate described herein, e.g., a CDP-paclitaxel conjugate comprising paclitaxel, coupled, e.g., via linkers, to a CDP described herein. In an embodiment, the CDP-paclitaxel conjugate comprises paclitaxel, coupled via a linker shown in FIG. 2 to a CDP described herein. In an embodiment, the CDP-paclitaxel conjugate is a CDP-paclitaxel conjugate shown in FIG. 2.

In one embodiment, the CDP-paclitaxel conjugate is administered at a dose and/or dosing schedule described herein.

In one embodiment, the CDP-taxane conjugate is a CDP-larotaxel conjugate, e.g., a CDP-larotaxel conjugate described herein, e.g., a CDP-larotaxel conjugate comprising larotaxel, coupled, e.g., via linkers, to a CDP described herein. In an embodiment, the CDP-larotaxel conjugate comprises larotaxel, coupled via a linker shown in FIG. 2 to a CDP described herein. In an embodiment, the CDP-larotaxel conjugate is a CDP-larotaxel conjugate shown in FIG. 2.

In one embodiment, the CDP-larotaxel conjugate is administered at a dose and/or dosing schedule described herein.

In one embodiment, the CDP-taxane conjugate is a CDP-cabazitaxel conjugate, e.g., a CDP-cabazitaxel conjugate described herein, e.g., a CDP-cabazitaxel conjugate comprising cabazitaxel, coupled, e.g., via linkers, to a CDP described herein. In an embodiment, the CDP-cabazitaxel conjugate comprises cabazitaxel, coupled via a linker shown in FIG. 2 to a CDP described herein. In an embodiment, the CDP-cabazitaxel conjugate is a CDP-cabazitaxel conjugate shown in FIG. 2.

In one embodiment, the CDP-cabazitaxel conjugate is administered at a dose and/or dosing schedule described herein.

In an embodiment, the CDP-taxane conjugate complex forms a particle or nanoparticle having a conjugate number described herein. By way of example, a CDP-therapeutic agent conjugate, forms, or is provided in, a particle or nanoparticle having a conjugate number of: 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In an embodiment the conjugate number is 2 to 4 or 2 to 5.

In an embodiment the conjugate number is 1, 2, 3, 4, 5, 6, 7, 8, 9, or 10.

In an embodiment the nanoparticle forms, or is provided in, a preparation of nanoparticles, e.g., a pharmaceutical preparation, wherein at least 40, 50, 60, 70, 80, 90 or 95% of the particles in the preparation have a conjugate number provided herein. In an embodiment the nanoparticle forms, or is provided in, a preparation of nanoparticles, e.g., a pharmaceutical preparation, wherein at least 60% of the particles in the preparation have a conjugate number of 1-5 or 2-5.

In an embodiment, the CDP-taxane conjugate is administered as a nanoparticle or preparation of nanoparticles, e.g., a pharmaceutical preparation, wherein at least 60% of the particles in the preparation have a conjugate number of 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In yet another aspect, the invention features a method of treating multiple myeloma in a subject, e.g., a human, the method comprising:

providing a subject who has multiple myeloma and has been treated with a chemotherapeutic agent that did not effectively treat the myeloma (e.g., the subject has a chemotherapeutic refractory myeloma, a chemotherapeutic resistant myeloma and/or a relapsed myeloma) or who had an unacceptable side effect (e.g., the subject has a chemotherapeutic sensitive myeloma), and

administering a CDP-taxane conjugate, e.g., a CDP-docetaxel conjugate, a CDP-paclitaxel conjugate, a CDP-larotaxel conjugate and/or a CDP-cabazitaxel conjugate described herein, to a subject in an amount effective to treat the myeloma, to thereby treat the myeloma.

In an embodiment, the CDP-taxane conjugate comprises taxane molecules (e.g., docetaxel, paclitaxel, larotaxel and/or cabazitaxel molecules), coupled, e.g., via a linker such as a linker described herein, to a CDP moiety, e.g., a CDP described herein. In an embodiment, the CDP-taxane conjugate comprises a taxane (e.g., docetaxel, paclitaxel, larotaxel and/or cabazitaxel), coupled via a linker shown in FIG. 2 to a CDP moiety, e.g., a CDP described herein. In an embodiment, the CDP-taxane conjugate is a CDP-taxane conjugate shown in FIG. 2.

In one embodiment, the subject has been treated with a proteosome inhibitor, e.g., bortezomib, which did not effectively treat the myeloma (e.g., the subject has a bortezomib refractory, a bortezomib resistant and/or relapsed myeloma).

In one embodiment, the subject has been treated with an anthracycline (e.g., daunorubicin, doxorubicin, epirubicin, valrubicin or idarubicin) which did not effectively treat the cancer (e.g., the subject has a doxorubicin refractory, a doxorubicin resistant and/or a relapsed myeloma).

In one embodiment, the subject has been treated with a thalidomide or thalidomide derivative (e.g., lenalidomide) which did not effectively treat the myeloma (e.g., the subject has thalidomide or thalidomide derivative refractory, thalidomide or thalidomide derivative resistant and/or a relapsed myeloma).

In one embodiment, the subject has been treated with a taxane (e.g., docetaxel or paclitaxel) which did not effectively treat the myeloma.

In one embodiment, the CDP-taxane conjugate is administered in combination with an anthracycline (e.g., daunorubicin, doxorubicin (e.g., liposomal doxorubicin), epirubicin, valrubicin and idarubicin). In one embodiment, the CDP-taxane conjugate is administered in combination with an anthracycline (e.g., daunorubicin, doxorubicin (e.g., liposomal doxorubicin), epirubicin, valrubicin and idarubicin) and a proteosome inhibitor, e.g., bortezomib.

In another embodiment, the CDP-taxane conjugate is administered in combination with a proteosome inhibitor, e.g., bortezomib.

In one embodiment, the CDP-taxane conjugate is administered in combination with thalidomide or a thalidomide derivative (e.g. lenalidomide) and dexamethasone.

In one embodiment, the CDP-taxane conjugate is administered in combination with dexamethaxone and cyclophosphamide. In one embodiment, the CDP-taxane conjugate is further administered in combination with a topoisomerase inhibitor (e.g., etoposide, topotecan, irinotecan, tenoposide, SN-38, lamellarin D) and/or a platinum based agent (carboplatin, cisplatin, oxaliplatin). In one embodiment, the CDP-taxane conjugate is further administered in combination with an anthracycline (e.g., daunorubicin, doxorubicin (e.g., liposomal doxorubicin), epirubicin, valrubicin and idarubicin).

In one embodiment, the CDP-taxane conjugate is a CDP-docetaxel conjugate, e.g., a CDP-docetaxel conjugate described herein, e.g., a CDP-docetaxel conjugate comprising docetaxel, coupled, e.g., via linkers, to a CDP described herein. In an embodiment, the CDP-docetaxel conjugate comprises docetaxel, coupled via a linker shown in FIG. 2 to a CDP described herein. In an embodiment, the CDP-docetaxel conjugate is a CDP-docetaxel conjugate shown in FIG. 2.

In one embodiment, the CDP-docetaxel conjugate is administered at a dose and/or dosing schedule described herein.

In one embodiment, the CDP-taxane conjugate is a CDP-paclitaxel conjugate, e.g., a CDP-paclitaxel conjugate described herein, e.g., a CDP-paclitaxel conjugate comprising paclitaxel, coupled, e.g., via linkers, to a CDP described herein. In an embodiment, the CDP-paclitaxel conjugate comprises docetaxel, coupled via a linker shown in FIG. 2 to a CDP described herein. In an embodiment, the CDP-paclitaxel conjugate is a CDP-docetaxel conjugate shown in FIG. 2.

In one embodiment, the CDP-paclitaxel conjugate is administered at a dose and/or dosing schedule described herein.

In one embodiment, the CDP-taxane conjugate is a CDP-larotaxel conjugate, e.g., a CDP-larotaxel conjugate described herein, e.g., a CDP-larotaxel conjugate comprising larotaxel, coupled, e.g., via linkers, to a CDP described herein. In an embodiment, the CDP-larotaxel conjugate comprises larotaxel, coupled via a linker shown in FIG. 2 to a CDP described herein. In an embodiment, the CDP-larotaxel conjugate is a CDP-larotaxel conjugate shown in FIG. 2.

In one embodiment, the CDP-larotaxel conjugate is administered at a dose and/or dosing schedule described herein.

In one embodiment, the CDP-taxane conjugate is a CDP-cabazitaxel conjugate, e.g., a CDP-cabazitaxel conjugate described herein, e.g., a CDP-cabazitaxel conjugate comprising cabazitaxel, coupled, e.g., via linkers, to a CDP described herein. In an embodiment, the CDP-cabazitaxel conjugate comprises cabazitaxel, coupled via a linker shown in FIG. 2 to a CDP described herein. In an embodiment, the CDP-cabazitaxel conjugate is a CDP-cabazitaxel conjugate shown in FIG. 2.

In one embodiment, the CDP-cabazitaxel conjugate is administered at a dose and/or dosing schedule described herein.

In an embodiment, the CDP-taxane conjugate complex forms a particle or nanoparticle having a conjugate number described herein. By way of example, a CDP-therapeutic agent conjugate, forms, or is provided in, a particle or nanoparticle having a conjugate number of: 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In an embodiment the conjugate number is 2 to 4 or 2 to 5.

In an embodiment the conjugate number is 1, 2, 3, 4, 5, 6, 7, 8, 9, or 10.

In an embodiment the nanoparticle forms, or is provided in, a preparation of nanoparticles, e.g., a pharmaceutical preparation, wherein at least 40, 50, 60, 70, 80, 90 or 95% of the particles in the preparation have a conjugate number provided herein. In an embodiment the nanoparticle forms, or is provided in, a preparation of nanoparticles, e.g., a pharmaceutical preparation, wherein at least 60% of the particles in the preparation have a conjugate number of 1-5 or 2-5.

In an embodiment, the CDP-taxane conjugate is administered as a nanoparticle or preparation of nanoparticles, e.g., a pharmaceutical preparation, wherein at least 60% of the particles in the preparation have a conjugate number of 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In yet another aspect, the invention features a method of treating AIDS-related Kaposi's Sarcoma in a subject, e.g., a human. The method comprises: administering a CDP-taxane conjugate, e.g., a CDP-docetaxel conjugate, a CDP-paclitaxel conjugate, a CDP-larotaxel conjugate and/or a CDP-cabazitaxel conjugate described herein, to a subject in an amount effective to treat the sarcoma, to thereby treat the sarcoma.

In an embodiment, the CDP-taxane conjugate comprises taxane molecules (e.g., docetaxel, paclitaxel, larotaxel and/or cabazitaxel molecules), coupled, e.g., via a linker such as a linker described herein, to a CDP moiety, e.g., a CDP described herein. In an embodiment, the CDP-taxane conjugate comprises a taxane (e.g., docetaxel, paclitaxel, larotaxel and/or cabazitaxel), coupled via a linker shown in FIG. 2 to a CDP moiety, e.g., a CDP described herein. In an embodiment, the CDP-taxane conjugate is a CDP-taxane conjugate shown in FIG. 2.

In one embodiment, the CDP-taxane conjugate is administered in combination with an antiviral agent, e.g., a nucleoside or a nucleotide reverse transcriptase inhibitor, a non-nucleoside reverse transcriptase inhibitor, a protease inhibitor, an integrase inhibitor, and entry or fusion inhibitor, a maturation inhibitor, or a broad spectrum inhibitor. Examples of nucleoside reverse transcriptase inhibitors include zidovudine, didanosine, zalcitabine, stavudine, lamivudine, abacavir, emtricitabine and apricitabine. Nucleotide reverse transcriptase include, e.g., tenofovir and adefovir. Examples of a non-nucleoside reverse transcriptase inhibitor include efavirenz, nevirapine, delavirdine and etravirine. Protease inhibitors include, e.g., saquinavir, ritonavir, indinavir, nelfinavir and amprenavir. An exemplary integrase inhibitor is raltegravir. Examples of entry inhibitors and fusion inhibitors include maraviroc and enfuvirtide. Maturation inhibitors include, e.g., bevirimat and vivecon.

In one embodiment, the CDP-taxane conjugate is administered in combination with cryosurgery. In one embodiment, CDP-taxane conjugate is administered in combination alitretinoin.

In one embodiment, the CDP-taxane conjugate is administered in combination with an anthracycline (e.g., daunorubicin, doxorubicin (e.g., liposomal doxorubicin), epirubicin, valrubicin and idarubicin). In one embodiment, the CDP-taxane conjugate is further administered with a vinca alkaloid (e.g., vinblastine, vincristine, vindesine and vinorelbine) and an antibiotic (e.g., actinomycin, bleomycin, hydroxyurea and mitomycin).

In one embodiment, the CDP-taxane conjugate is administered in combination with a taxane (e.g., paclitaxel or docetaxel). In one embodiment, the CDP-taxane conjugate is further administered with a vinca alkaloid (e.g., vinblastine, vincristine, vindesine and vinorelbine).

In one embodiment, the CDP-taxane is administered in combination with a vinca alkaloid (e.g., vinblastine, vincristine, vindesine and vinorelbine).

In some embodiments, the CDP-taxane conjugate is administered in combination with a vascular endothelial growth factor (VEGF) pathway inhibitor, e.g., a VEGF inhibitor (e.g., bevacizumab) or VEGF receptor inhibitor (e.g., CP-547632 and AZD2171). In one embodiment, the CDP-taxane conjugate is administered in combination with bevacizumab.

In some embodiments, the CDP-taxane conjugate is administered in combination with an mTOR inhibitor. Non-limiting examples of mTOR inhibitors include rapamycin, everolimus, AP23573, CCI-779 and SDZ-RAD.

In one embodiment, the CDP-taxane conjugate is a CDP-docetaxel conjugate, e.g., a CDP-docetaxel conjugate described herein, e.g., a CDP-docetaxel conjugate comprising docetaxel, coupled, e.g., via linkers, to a CDP described herein. In an embodiment, the CDP-docetaxel conjugate comprises docetaxel, coupled via a linker shown in FIG. 2 to a CDP described herein. In an embodiment, the CDP-docetaxel conjugate is a CDP-docetaxel conjugate shown in FIG. 2.

In one embodiment, the CDP-docetaxel conjugate is administered at a dose and/or dosing schedule described herein.

In one embodiment, the CDP-taxane conjugate is a CDP-paclitaxel conjugate, e.g., a CDP-paclitaxel conjugate described herein, e.g., a CDP-paclitaxel conjugate comprising paclitaxel, coupled, e.g., via linkers, to a CDP described herein. In an embodiment, the CDP-paclitaxel conjugate comprises paclitaxel, coupled via a linker shown in FIG. 2 to a CDP described herein. In an embodiment, the CDP-paclitaxel conjugate is a CDP-paclitaxel conjugate shown in FIG. 2.

In one embodiment, the CDP-paclitaxel conjugate is administered at a dose and/or dosing schedule described herein.

In one embodiment, the CDP-taxane conjugate is a CDP-larotaxel conjugate, e.g., a CDP-larotaxel conjugate described herein, e.g., a CDP-larotaxel conjugate comprising larotaxel, coupled, e.g., via linkers, to a CDP described herein. In an embodiment, the CDP-larotaxel conjugate comprises larotaxel, coupled via a linker shown in FIG. 2 to a CDP described herein. In an embodiment, the CDP-larotaxel conjugate is a CDP-larotaxel conjugate shown in FIG. 2.

In one embodiment, the CDP-larotaxel conjugate is administered at a dose and/or dosing schedule described herein.

In one embodiment, the CDP-taxane conjugate is a CDP-cabazitaxel conjugate, e.g., a CDP-cabazitaxel conjugate described herein, e.g., a CDP-cabazitaxel conjugate comprising cabazitaxel, coupled, e.g., via linkers, to a CDP described herein. In an embodiment, the CDP-cabazitaxel conjugate comprises cabazitaxel, coupled via a linker shown in FIG. 2 to a CDP described herein. In an embodiment, the CDP-cabazitaxel conjugate is a CDP-cabazitaxel conjugate shown in FIG. 2.

In one embodiment, the CDP-cabazitaxel conjugate is administered at a dose and/or dosing schedule described herein.

In an embodiment, the CDP-taxane conjugate complex forms a particle or nanoparticle having a conjugate number described herein. By way of example, a CDP-therapeutic agent conjugate, forms, or is provided in, a particle or nanoparticle having a conjugate number of: 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In an embodiment the conjugate number is 2 to 4 or 2 to 5.

In an embodiment the conjugate number is 1, 2, 3, 4, 5, 6, 7, 8, 9, or 10.

In an embodiment the nanoparticle forms, or is provided in, a preparation of nanoparticles, e.g., a pharmaceutical preparation, wherein at least 40, 50, 60, 70, 80, 90 or 95% of the particles in the preparation have a conjugate number provided herein. In an embodiment the nanoparticle forms, or is provided in, a preparation of nanoparticles, e.g., a pharmaceutical preparation, wherein at least 60% of the particles in the preparation have a conjugate number of 1-5 or 2-5.

In an embodiment, the CDP-taxane conjugate is administered as a nanoparticle or preparation of nanoparticles, e.g., a pharmaceutical preparation, wherein at least 60% of the particles in the preparation have a conjugate number of 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In yet another aspect, the invention features a method of treating AIDS-related Kaposi's Sarcoma, in a subject, e.g., a human. The method comprises:

providing a subject who has AIDS-related Kaposi's Sarcoma and has been treated with a chemotherapeutic agent which did not effectively treat the sarcoma (e.g., the subject has a chemotherapeutic refractory, a chemotherapeutic resistant and/or a relapsed sarcoma) or which had an unacceptable side effect (e.g., the subject has a chemotherapeutic sensitive sarcoma), and

administering a CDP-taxane conjugate, e.g., a CDP-docetaxel conjugate, a CDP-paclitaxel conjugate, a CDP-larotaxel conjugate and/or a CDP-cabazitaxel conjugate described herein, to a subject in an amount effective to treat the cancer, to thereby treat the cancer.

In an embodiment, the CDP-taxane conjugate comprises taxane molecules (e.g., docetaxel, paclitaxel, larotaxel and/or cabazitaxel molecules), coupled, e.g., via a linker such as a linker described herein, to a CDP moiety, e.g., a CDP described herein. In an embodiment, the CDP-taxane conjugate comprises a taxane (e.g., docetaxel, paclitaxel, larotaxel and/or cabazitaxel), coupled via a linker shown in FIG. 2 to a CDP moiety, e.g., a CDP described herein. In an embodiment, the CDP-taxane conjugate is a CDP-taxane conjugate shown in FIG. 2.

In one embodiment, the sarcoma is refractory to, resistant to, and/or relapsed with treatment with one or more of: a taxane (e.g., paclitaxel and docetaxel), an anthracycline, a vinca alkaloid (e.g., vinblastine, vincristine, vindesine and vinorelbine) and an anthracycline (e.g., daunorubicin, doxorubicin, epirubicin, valrubicin and idarubicin).

In one embodiment, the cancer is a multidrug resistant sarcoma.

In one embodiment, the CDP-taxane conjugate is a CDP-docetaxel conjugate, e.g., a CDP-docetaxel conjugate described herein, e.g., a CDP-docetaxel conjugate comprising docetaxel, coupled, e.g., via linkers, to a CDP described herein. In an embodiment, the CDP-docetaxel conjugate comprises docetaxel, coupled via a linker shown in FIG. 2 to a CDP described herein. In an embodiment, the CDP-docetaxel conjugate is a CDP-docetaxel conjugate shown in FIG. 2.

In one embodiment, the CDP-docetaxel conjugate is administered at a dose and/or dosing schedule described herein.

In one embodiment, the CDP-taxane conjugate is a CDP-paclitaxel conjugate, e.g., a CDP-paclitaxel conjugate described herein, e.g., a CDP-paclitaxel conjugate comprising paclitaxel, coupled, e.g., via linkers, to a CDP described herein. In an embodiment, the CDP-paclitaxel conjugate comprises paclitaxel, coupled via a linker shown in FIG. 2 to a CDP described herein. In an embodiment, the CDP-paclitaxel conjugate is a CDP-paclitaxel conjugate shown in FIG. 2.

In one embodiment, the CDP-paclitaxel conjugate is administered at a dose and/or dosing schedule described herein.

In one embodiment, the CDP-taxane conjugate is a CDP-larotaxel conjugate, e.g., a CDP-larotaxel conjugate described herein, e.g., a CDP-larotaxel conjugate comprising larotaxel, coupled, e.g., via linkers, to a CDP described herein. In an embodiment, the CDP-larotaxel conjugate comprises larotaxel, coupled via a linker shown in FIG. 2 to a CDP described herein. In an embodiment, the CDP-larotaxel conjugate is a CDP-larotaxel conjugate shown in FIG. 2.

In one embodiment, the CDP-larotaxel conjugate is administered at a dose and/or dosing schedule described herein.

In one embodiment, the CDP-taxane conjugate is a CDP-cabazitaxel conjugate, e.g., a CDP-cabazitaxel conjugate described herein, e.g., a CDP-cabazitaxel conjugate comprising cabazitaxel, coupled, e.g., via linkers, to a CDP described herein. In an embodiment, the CDP-cabazitaxel conjugate comprises cabazitaxel, coupled via a linker shown in FIG. 2 to a CDP described herein. In an embodiment, the CDP-cabazitaxel conjugate is a CDP-cabazitaxel conjugate shown in FIG. 2.

In one embodiment, the CDP-cabazitaxel conjugate is administered at a dose and/or dosing schedule described herein.

In an embodiment, the CDP-taxane conjugate complex forms a particle or nanoparticle having a conjugate number described herein. By way of example, a CDP-therapeutic agent conjugate, forms, or is provided in, a particle or nanoparticle having a conjugate number of: 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In an embodiment the conjugate number is 2 to 4 or 2 to 5.

In an embodiment the conjugate number is 1, 2, 3, 4, 5, 6, 7, 8, 9, or 10.

In an embodiment the nanoparticle forms, or is provided in, a preparation of nanoparticles, e.g., a pharmaceutical preparation, wherein at least 40, 50, 60, 70, 80, 90 or 95% of the particles in the preparation have a conjugate number provided herein. In an embodiment the nanoparticle forms, or is provided in, a preparation of nanoparticles, e.g., a pharmaceutical preparation, wherein at least 60% of the particles in the preparation have a conjugate number of 1-5 or 2-5.

In an embodiment, the CDP-taxane conjugate is administered as a nanoparticle or preparation of nanoparticles, e.g., a pharmaceutical preparation, wherein at least 60% of the particles in the preparation have a conjugate number of 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In yet another aspect, the invention features a method of treating gastric cancer in a subject, e.g., a human. The method comprises: administering a CDP-taxane conjugate, e.g., a CDP-docetaxel conjugate, a CDP-paclitaxel conjugate, a CDP-larotaxel conjugate and/or a CDP-cabazitaxel conjugate described herein, to a subject in an amount effective to treat the cancer, to thereby treat the cancer.

In an embodiment, the CDP-taxane conjugate comprises taxane molecules (e.g., docetaxel, paclitaxel, larotaxel and/or cabazitaxel molecules), coupled, e.g., via a linker such as a linker described herein, to a CDP moiety, e.g., a CDP described herein. In an embodiment, the CDP-taxane conjugate comprises a taxane (e.g., docetaxel, paclitaxel, larotaxel and/or cabazitaxel), coupled via a linker shown in FIG. 2 to a CDP moiety, e.g., a CDP described herein. In an embodiment, the CDP-taxane conjugate is a CDP-taxane conjugate shown in FIG. 2.

In one embodiment, the gastric cancer is gastroesophageal junction adenocarcinoma.

In one embodiment, the CDP-taxane conjugate is administered prior to surgery, after surgery or before and after surgery to remove the cancer.

In one embodiment, the CDP-taxane conjugate is administered in combination with one or more of an anthracycline (e.g., daunorubicin, doxorubicin (e.g., liposomal doxorubicin), epirubicin, valrubicin and idarubicin), a platinum-based agent (e.g., cisplatin, carboplatin, oxaliplatin) and an anti-metabolite, e.g., an antifolate (e.g., floxuridine, pemetrexed) or pyrimidine analogue (e.g., 5FU)).

In some embodiments, the CDP-taxane conjugate is administered in combination with an anti-metabolite, e.g., an antifolate (e.g., floxuridine, pemetrexed) or pyrimidine analogue (e.g., capecitabine, 5FU)). In one embodiment, the CDP-taxane conjugate is further administered with a taxane (e.g., paclitaxel or docetaxel).

In one embodiment, the CDP-taxane conjugate is administered in combination with radiation.

In some embodiments, the CDP-taxane conjugate is administered in combination with a vascular endothelial growth factor (VEGF) pathway inhibitor, e.g., a VEGF inhibitor (e.g., bevacizumab) or VEGF receptor inhibitor (e.g., CP-547632 and AZD2171). In one embodiment, the CDP-taxane conjugate is administered in combination with bevacizumab.

In some embodiments, the CDP-taxane conjugate is administered in combination with an mTOR inhibitor. Non-limiting examples of mTOR inhibitors include rapamycin, everolimus, AP23573, CCI-779 and SDZ-RAD.

In one embodiment, the CDP-taxane conjugate is a CDP-docetaxel conjugate, e.g., a CDP-docetaxel conjugate described herein, e.g., a CDP-docetaxel conjugate comprising docetaxel, coupled, e.g., via linkers, to a CDP described herein. In an embodiment, the CDP-docetaxel conjugate comprises docetaxel, coupled via a linker shown in FIG. 2 to a CDP described herein. In an embodiment, the CDP-docetaxel conjugate is a CDP-docetaxel conjugate shown in FIG. 2.

In one embodiment, the CDP-docetaxel conjugate is administered at a dose and/or dosing schedule described herein.

In one embodiment, the CDP-taxane conjugate is a CDP-paclitaxel conjugate, e.g., a CDP-paclitaxel conjugate described herein, e.g., a CDP-paclitaxel conjugate comprising paclitaxel, coupled, e.g., via linkers, to a CDP described herein. In an embodiment, the CDP-paclitaxel conjugate comprises paclitaxel, coupled via a linker shown in FIG. 2 to a CDP described herein. In an embodiment, the CDP-paclitaxel conjugate is a CDP-paclitaxel conjugate shown in FIG. 2.

In one embodiment, the CDP-paclitaxel conjugate is administered at a dose and/or dosing schedule described herein.

In one embodiment, the CDP-taxane conjugate is a CDP-larotaxel conjugate, e.g., a CDP-larotaxel conjugate described herein, e.g., a CDP-larotaxel conjugate comprising larotaxel, coupled, e.g., via linkers, to a CDP described herein. In an embodiment, the CDP-larotaxel conjugate comprises larotaxel, coupled via a linker shown in FIG. 2 to a CDP described herein. In an embodiment, the CDP-larotaxel conjugate is a CDP-larotaxel conjugate shown in FIG. 2.

In one embodiment, the CDP-larotaxel conjugate is administered at a dose and/or dosing schedule described herein.

In one embodiment, the CDP-taxane conjugate is a CDP-cabazitaxel conjugate, e.g., a CDP-cabazitaxel conjugate described herein, e.g., a CDP-cabazitaxel conjugate comprising cabazitaxel, coupled, e.g., via linkers, to a CDP described herein. In an embodiment, the CDP-cabazitaxel conjugate comprises cabazitaxel, coupled via a linker shown in FIG. 2 to a CDP described herein. In an embodiment, the CDP-cabazitaxel conjugate is a CDP-cabazitaxel conjugate shown in FIG. 2.

In one embodiment, the CDP-cabazitaxel conjugate is administered at a dose and/or dosing schedule described herein.

In an embodiment, the CDP-taxane conjugate complex forms a particle or nanoparticle having a conjugate number described herein. By way of example, a CDP-therapeutic agent conjugate, forms, or is provided in, a particle or nanoparticle having a conjugate number of: 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In an embodiment the conjugate number is 2 to 4 or 2 to 5.

In an embodiment the conjugate number is 1, 2, 3, 4, 5, 6, 7, 8, 9, or 10.

In an embodiment the nanoparticle forms, or is provided in, a preparation of nanoparticles, e.g., a pharmaceutical preparation, wherein at least 40, 50, 60, 70, 80, 90 or 95% of the particles in the preparation have a conjugate number provided herein. In an embodiment the nanoparticle forms, or is provided in, a preparation of nanoparticles, e.g., a pharmaceutical preparation, wherein at least 60% of the particles in the preparation have a conjugate number of 1-5 or 2-5.

In an embodiment, the CDP-taxane conjugate is administered as a nanoparticle or preparation of nanoparticles, e.g., a pharmaceutical preparation, wherein at least 60% of the particles in the preparation have a conjugate number of 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In yet another aspect, the invention features a method of treating gastric cancer, e.g. a gastric cancer described herein such as gastroesophageal junction adenocarcinoma, in a subject, e.g., a human. The method comprises:

providing a subject who has gastric cancer and has been treated with a chemotherapeutic agent which did not effectively treat the cancer (e.g., the subject has a non-resectable cancer, a chemotherapeutic refractory, a chemotherapeutic resistant and/or a relapsed cancer) or which had an unacceptable side effect (e.g., the subject has a chemotherapeutic sensitive cancer), and

administering a CDP-taxane conjugate, e.g., a CDP-docetaxel conjugate, a CDP-paclitaxel conjugate, a CDP-larotaxel conjugate and/or a CDP-cabazitaxel conjugate described herein, to a subject in an amount effective to treat the cancer, to thereby treat the cancer.

In an embodiment, the CDP-taxane conjugate comprises taxane molecules (e.g., docetaxel, paclitaxel, larotaxel and/or cabazitaxel molecules), coupled, e.g., via a linker such as a linker described herein, to a CDP moiety, e.g., a CDP described herein. In an embodiment, the CDP-taxane conjugate comprises a taxane (e.g., docetaxel, paclitaxel, larotaxel and/or cabazitaxel), coupled via a linker shown in FIG. 2 to a CDP moiety, e.g., a CDP described herein. In an embodiment, the CDP-taxane conjugate is a CDP-taxane conjugate shown in FIG. 2.

In one embodiment, the cancer is refractory to, resistant to, and/or relapsed with treatment with one or more of: a taxane (e.g., paclitaxel and docetaxel), an anthracycline (e.g., daunorubicin, doxorubicin, epirubicin, valrubicin and idarubicin), an anti-metabolite, e.g., an antifolate (e.g., floxuridine, pemetrexed) or pyrimidine analogue (e.g., capecitabine, 5FU)), and a platinum-based agent (e.g., cisplatin, carboplatin, oxaliplatin).

In one embodiment, the cancer is a multidrug resistant cancer.

In one embodiment, the CDP-taxane conjugate is administered in combination with a pyrimidine analogue, e.g., a pyrimidine analogue described herein (e.g., capecitabine and 5FU).

In one embodiment, the CDP-taxane conjugate is administered in combination with a platinum-based agent (e.g., cisplatin, carboplatin, oxaliplatin). In one embodiment, the CDP-taxane conjugate is further administered in combination with a pyrimidine analogue, e.g., a pyrimidine analogue described herein (e.g., capecitabine and 5FU). In another embodiment, the CDP-taxane conjugate is further administered in combination with a topoisomerase inhibitor (e.g., etoposide, topotecan, irinotecan, tenoposide, SN-38, lamellarin D).

In one embodiment, the CDP-taxane conjugate is administered in combination with a topoisomerase inhibitor (e.g., etoposide, topotecan, irinotecan, tenoposide, SN-38, lamellarin D). In one embodiment, the CDP-taxane conjugate is further administered in combination with a pyrimidine analogue, e.g., a pyrimidine analogue described herein (e.g., capecitabine and 5FU).

In some embodiments, the CDP-taxane conjugate is administered in combination with a taxane (e.g., paclitaxel and docetaxel). In one embodiment, the CDP-taxane conjugate is further administered in combination with a pyrimidine analogue, e.g., a pyrimidine analogue described herein (e.g., capecitabine and 5FU).

In one embodiment, the CDP-taxane conjugate is a CDP-docetaxel conjugate, e.g., a CDP-docetaxel conjugate described herein, e.g., a CDP-docetaxel conjugate comprising docetaxel, coupled, e.g., via linkers, to a CDP described herein. In an embodiment, the CDP-docetaxel conjugate comprises docetaxel, coupled via a linker shown in FIG. 2 to a CDP described herein. In an embodiment, the CDP-docetaxel conjugate is a CDP-docetaxel conjugate shown in FIG. 2.

In one embodiment, the CDP-docetaxel conjugate is administered at a dose and/or dosing schedule described herein.

In one embodiment, the CDP-taxane conjugate is a CDP-paclitaxel conjugate, e.g., a CDP-paclitaxel conjugate described herein, e.g., a CDP-paclitaxel conjugate comprising paclitaxel, coupled, e.g., via linkers, to a CDP described herein. In an embodiment, the CDP-paclitaxel conjugate comprises paclitaxel, coupled via a linker shown in FIG. 2 to a CDP described herein. In an embodiment, the CDP-paclitaxel conjugate is a CDP-paclitaxel conjugate shown in FIG. 2.

In one embodiment, the CDP-paclitaxel conjugate is administered at a dose and/or dosing schedule described herein.

In one embodiment, the CDP-taxane conjugate is a CDP-larotaxel conjugate, e.g., a CDP-larotaxel conjugate described herein, e.g., a CDP-larotaxel conjugate comprising larotaxel, coupled, e.g., via linkers, to a CDP described herein. In an embodiment, the CDP-larotaxel conjugate comprises larotaxel, coupled via a linker shown in FIG. 2 to a CDP described herein. In an embodiment, the CDP-larotaxel conjugate is a CDP-larotaxel conjugate shown in FIG. 2.

In one embodiment, the CDP-larotaxel conjugate is administered at a dose and/or dosing schedule described herein.

In one embodiment, the CDP-taxane conjugate is a CDP-cabazitaxel conjugate, e.g., a CDP-cabazitaxel conjugate described herein, e.g., a CDP-cabazitaxel conjugate comprising cabazitaxel, coupled, e.g., via linkers, to a CDP described herein. In an embodiment, the CDP-cabazitaxel conjugate comprises cabazitaxel, coupled via a linker shown in FIG. 2 to a CDP described herein. In an embodiment, the CDP-cabazitaxel conjugate is a CDP-cabazitaxel conjugate shown in FIG. 2.

In one embodiment, the CDP-cabazitaxel conjugate is administered at a dose and/or dosing schedule described herein.

In an embodiment, the CDP-taxane conjugate complex forms a particle or nanoparticle having a conjugate number described herein. By way of example, a CDP-therapeutic agent conjugate, forms, or is provided in, a particle or nanoparticle having a conjugate number of: 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In an embodiment the conjugate number is 2 to 4 or 2 to 5.

In an embodiment the conjugate number is 1, 2, 3, 4, 5, 6, 7, 8, 9, or 10.

In an embodiment the nanoparticle forms, or is provided in, a preparation of nanoparticles, e.g., a pharmaceutical preparation, wherein at least 40, 50, 60, 70, 80, 90 or 95% of the particles in the preparation have a conjugate number provided herein. In an embodiment the nanoparticle forms, or is provided in, a preparation of nanoparticles, e.g., a pharmaceutical preparation, wherein at least 60% of the particles in the preparation have a conjugate number of 1-5 or 2-5.

In an embodiment, the CDP-taxane conjugate is administered as a nanoparticle or preparation of nanoparticles, e.g., a pharmaceutical preparation, wherein at least 60% of the particles in the preparation have a conjugate number of 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In yet another aspect, the invention features a method of treating a soft tissue sarcoma (e.g., non-resectable, advanced, metastatic or relapsed soft tissue sarcoma) in a subject, e.g., a human. The method comprises: administering a CDP-taxane conjugate, e.g., a CDP-docetaxel conjugate, a CDP-paclitaxel conjugate, a CDP-larotaxel conjugate and/or a CDP-cabazitaxel conjugate described herein, to a subject in an amount effective to treat the sarcoma, to thereby treat the sarcoma.

In an embodiment, the CDP-taxane conjugate comprises taxane molecules (e.g., docetaxel, paclitaxel, larotaxel and/or cabazitaxel molecules), coupled, e.g., via a linker such as a linker described herein, to a CDP moiety, e.g., a CDP described herein. In an embodiment, the CDP-taxane conjugate comprises a taxane (e.g., docetaxel, paclitaxel, larotaxel and/or cabazitaxel), coupled via a linker shown in FIG. 2 to a CDP moiety, e.g., a CDP described herein. In an embodiment, the CDP-taxane conjugate is a CDP-taxane conjugate shown in FIG. 2.

In one embodiment, the soft tissue sarcoma is rhabdomyosarcoma, leiomyosarcoma, hemangiosarcoma, lymphangiosarcoma, synovial sarcoma, neurofibrosarcoma, liposarcoma, fibrosarcoma, malignant fibrous histiocytoma and dermatofibrosarcoma.

In one embodiment, the CDP-taxane conjugate is administered in combination with an anthracycline, e.g., daunorubicin, doxorubicin (e.g., liposomal doxorubicin), epirubicin, valrubicin and idarubicin.

In one embodiment, the CDP-taxane conjugate is administered in combination with an alkylating agent (e.g., cyclophosphamide, dacarbazine, melphalan, ifosfamide, temozolomide). In one embodiment, the CDP-taxane conjugate is further administered in combination with mesna. In one embodiment, the CDP-taxane conjugate is further administered in combination with an anthracycline, e.g., daunorubicin, doxorubicin (e.g., liposomal doxorubicin), epirubicin, valrubicin and idarubicin.

In one embodiment, the CDP-taxane conjugate is administered in combination with an anti-metabolite, e.g., an antifolate (e.g., pemetrexed, floxuridine, raltitrexed) or pyrimidine analog (e.g., capecitabine, cytrarabine, gemcitabine, 5FU).

In one embodiment, the CDP-taxane conjugate is administered in combination with a vinca alkaloid (e.g., vinblastine, vincristine, vindesine, vinorelbine).

In some embodiments, the CDP-taxane conjugate is administered in combination with a vascular endothelial growth factor (VEGF) pathway inhibitor, e.g., a VEGF inhibitor (e.g., bevacizumab) or VEGF receptor inhibitor (e.g., CP-547632 and AZD2171). In one embodiment, the CDP-taxane conjugate is administered in combination with bevacizumab.

In some embodiments, the CDP-taxane conjugate is administered in combination with an mTOR inhibitor. Non-limiting examples of mTOR inhibitors include rapamycin, everolimus, AP23573, CCI-779 and SDZ-RAD.

In one embodiment, the CDP-taxane conjugate is a CDP-docetaxel conjugate, e.g., a CDP-docetaxel conjugate described herein, e.g., a CDP-docetaxel conjugate comprising docetaxel, coupled, e.g., via linkers, to a CDP described herein. In an embodiment, the CDP-docetaxel conjugate comprises docetaxel, coupled via a linker shown in FIG. 2 to a CDP described herein. In an embodiment, the CDP-docetaxel conjugate is a CDP-docetaxel conjugate shown in FIG. 2.

In one embodiment, the CDP-docetaxel conjugate is administered at a dose and/or dosing schedule described herein.

In one embodiment, the CDP-taxane conjugate is a CDP-paclitaxel conjugate, e.g., a CDP-paclitaxel conjugate described herein, e.g., a CDP-paclitaxel conjugate comprising paclitaxel, coupled, e.g., via linkers, to a CDP described herein. In an embodiment, the CDP-paclitaxel conjugate comprises paclitaxel, coupled via a linker shown in FIG. 2 to a CDP described herein. In an embodiment, the CDP-paclitaxel conjugate is a CDP-paclitaxel conjugate shown in FIG. 2.

In one embodiment, the CDP-paclitaxel conjugate is administered at a dose and/or dosing schedule described herein.

In one embodiment, the CDP-taxane conjugate is a CDP-larotaxel conjugate, e.g., a CDP-larotaxel conjugate described herein, e.g., a CDP-larotaxel conjugate comprising larotaxel, coupled, e.g., via linkers, to a CDP described herein. In an embodiment, the CDP-larotaxel conjugate comprises larotaxel, coupled via a linker shown in FIG. 2 to a CDP described herein. In an embodiment, the CDP-larotaxel conjugate is a CDP-larotaxel conjugate shown in FIG. 2.

In one embodiment, the CDP-larotaxel conjugate is administered at a dose and/or dosing schedule described herein.

In one embodiment, the CDP-taxane conjugate is a CDP-cabazitaxel conjugate, e.g., a CDP-cabazitaxel conjugate described herein, e.g., a CDP-cabazitaxel conjugate comprising cabazitaxel, coupled, e.g., via linkers, to a CDP described herein. In an embodiment, the CDP-cabazitaxel conjugate comprises cabazitaxel, coupled via a linker shown in FIG. 2 to a CDP described herein. In an embodiment, the CDP-cabazitaxel conjugate is a CDP-cabazitaxel conjugate shown in FIG. 2.

In one embodiment, the CDP-cabazitaxel conjugate is administered at a dose and/or dosing schedule described herein.

In an embodiment, the CDP-taxane conjugate complex forms a particle or nanoparticle having a conjugate number described herein. By way of example, a CDP-therapeutic agent conjugate, forms, or is provided in, a particle or nanoparticle having a conjugate number of: 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In an embodiment the conjugate number is 2 to 4 or 2 to 5.

In an embodiment the conjugate number is 1, 2, 3, 4, 5, 6, 7, 8, 9, or 10.

In an embodiment the nanoparticle forms, or is provided in, a preparation of nanoparticles, e.g., a pharmaceutical preparation, wherein at least 40, 50, 60, 70, 80, 90 or 95% of the particles in the preparation have a conjugate number provided herein. In an embodiment the nanoparticle forms, or is provided in, a preparation of nanoparticles, e.g., a pharmaceutical preparation, wherein at least 60% of the particles in the preparation have a conjugate number of 1-5 or 2-5.

In an embodiment, the CDP-taxane conjugate is administered as a nanoparticle or preparation of nanoparticles, e.g., a pharmaceutical preparation, wherein at least 60% of the particles in the preparation have a conjugate number of 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In yet another aspect, the invention features a method of treating a soft tissue sarcoma, in a subject, e.g., a human. The method comprises:

providing a subject who has a soft tissue sarcoma and has been treated with a chemotherapeutic agent which did not effectively treat the sarcoma (e.g., the subject has a chemotherapeutic refractory, a chemotherapeutic resistant and/or a relapsed sarcoma) or which had an unacceptable side effect (e.g., the subject has a chemotherapeutic sensitive sarcoma), and

administering a CDP-taxane conjugate, e.g., a CDP-docetaxel conjugate, a CDP-paclitaxel conjugate, a CDP-larotaxel conjugate and/or a CDP-cabazitaxel conjugate described herein, to a subject in an amount effective to treat the sarcoma, to thereby treat the sarcoma.

In an embodiment, the CDP-taxane conjugate comprises taxane molecules (e.g., docetaxel, paclitaxel, larotaxel and/or cabazitaxel molecules), coupled, e.g., via a linker such as a linker described herein, to a CDP moiety, e.g., a CDP described herein. In an embodiment, the CDP-taxane conjugate comprises a taxane (e.g., docetaxel, paclitaxel, larotaxel and/or cabazitaxel), coupled via a linker shown in FIG. 2 to a CDP moiety, e.g., a CDP described herein. In an embodiment, the CDP-taxane conjugate is a CDP-taxane conjugate shown in FIG. 2.

In one embodiment, the sarcoma is refractory to, resistant to, and/or relapsed with treatment with one or more of: a taxane (e.g., paclitaxel and docetaxel), an anthracycline (e.g., doxorubicin, daunorubicin, epirubicin, idarubicin, mitoxantrone, valrubicin), a vinca alkaloid (e.g., vinblastine, vincristine, vindesine and vinorelbine) and an alkylating agent (e.g., cyclophosphamide, dacarbazine, melphalan, ifosfamide, temozolomide).

In one embodiment, the sarcoma is a multidrug resistant cancer.

In one embodiment, the soft tissue sarcoma is rhabdomyosarcoma, leiomyosarcoma, hemangiosarcoma, lymphangiosarcoma, synovial sarcoma, neurofibrosarcoma, liposarcoma, fibrosarcoma, malignant fibrous histiocytoma and dermatofibrosarcoma.

In one embodiment, the CDP-taxane conjugate is administered in combination with an anthracycline, e.g., daunorubicin, doxorubicin (e.g., liposomal doxorubicin), epirubicin, valrubicin and idarubicin.

In one embodiment, the CDP-taxane conjugate is administered in combination with an alkylating agent (e.g., cyclophosphamide, dacarbazine, melphalan, ifosfamide, temozolomide). In one embodiment, the CDP-taxane conjugate is further administered in combination with mesna. In one embodiment, the CDP-taxane conjugate is further administered in combination with an anthracycline, e.g., daunorubicin, doxorubicin (e.g., liposomal doxorubicin), epirubicin, valrubicin and idarubicin.

In one embodiment, the CDP-taxane conjugate is administered in combination with an anti-metabolite, e.g., an antifolate (e.g., pemetrexed, floxuridine, raltitrexed) or pyrimidine analog (e.g., capecitabine, cytrarabine, gemcitabine, 5FU).

In one embodiment, the CDP-taxane conjugate is administered in combination with a vinca alkaloid (e.g., vinblastine, vincristine, vindesine, vinorelbine).

In some embodiments, the CDP-taxane conjugate is administered in combination with a vascular endothelial growth factor (VEGF) pathway inhibitor, e.g., a VEGF inhibitor (e.g., bevacizumab) or VEGF receptor inhibitor (e.g., CP-547632 and AZD2171). In one embodiment, the CDP-taxane conjugate is administered in combination with bevacizumab.

In some embodiments, the CDP-taxane conjugate is administered in combination with an mTOR inhibitor. Non-limiting examples of mTOR inhibitors include rapamycin, everolimus, AP23573, CCI-779 and SDZ-RAD.

In one embodiment, the CDP-taxane conjugate is a CDP-docetaxel conjugate, e.g., a CDP-docetaxel conjugate described herein, e.g., a CDP-docetaxel conjugate comprising docetaxel, coupled, e.g., via linkers, to a CDP described herein. In an embodiment, the CDP-docetaxel conjugate comprises docetaxel, coupled via a linker shown in FIG. 2 to a CDP described herein. In an embodiment, the CDP-docetaxel conjugate is a CDP-docetaxel conjugate shown in FIG. 2.

In one embodiment, the CDP-docetaxel conjugate is administered at a dose and/or dosing schedule described herein.

In one embodiment, the CDP-taxane conjugate is a CDP-paclitaxel conjugate, e.g., a CDP-paclitaxel conjugate described herein, e.g., a CDP-paclitaxel conjugate comprising paclitaxel, coupled, e.g., via linkers, to a CDP described herein. In an embodiment, the CDP-paclitaxel conjugate comprises paclitaxel, coupled via a linker shown in FIG. 2 to a CDP described herein. In an embodiment, the CDP-paclitaxel conjugate is a CDP-paclitaxel conjugate shown in FIG. 2.

In one embodiment, the CDP-paclitaxel conjugate is administered at a dose and/or dosing schedule described herein.

In one embodiment, the CDP-taxane conjugate is a CDP-larotaxel conjugate, e.g., a CDP-larotaxel conjugate described herein, e.g., a CDP-larotaxel conjugate comprising larotaxel, coupled, e.g., via linkers, to a CDP described herein. In an embodiment, the CDP-larotaxel conjugate comprises larotaxel, coupled via a linker shown in FIG. 2 to a CDP described herein. In an embodiment, the CDP-larotaxel conjugate is a CDP-larotaxel conjugate shown in FIG. 2.

In one embodiment, the CDP-larotaxel conjugate is administered at a dose and/or dosing schedule described herein.

In one embodiment, the CDP-taxane conjugate is a CDP-cabazitaxel conjugate, e.g., a CDP-cabazitaxel conjugate described herein, e.g., a CDP-cabazitaxel conjugate comprising cabazitaxel, coupled, e.g., via linkers, to a CDP described herein. In an embodiment, the CDP-cabazitaxel conjugate comprises cabazitaxel, coupled via a linker shown in FIG. 2 to a CDP described herein. In an embodiment, the CDP-cabazitaxel conjugate is a CDP-cabazitaxel conjugate shown in FIG. 2.

In one embodiment, the CDP-cabazitaxel conjugate is administered at a dose and/or dosing schedule described herein.

In an embodiment, the CDP-taxane conjugate complex forms a particle or nanoparticle having a conjugate number described herein. By way of example, a CDP-therapeutic agent conjugate, forms, or is provided in, a particle or nanoparticle having a conjugate number of: 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In an embodiment the conjugate number is 2 to 4 or 2 to 5.

In an embodiment the conjugate number is 1, 2, 3, 4, 5, 6, 7, 8, 9, or 10.

In an embodiment the nanoparticle forms, or is provided in, a preparation of nanoparticles, e.g., a pharmaceutical preparation, wherein at least 40, 50, 60, 70, 80, 90 or 95% of the particles in the preparation have a conjugate number provided herein. In an embodiment the nanoparticle forms, or is provided in, a preparation of nanoparticles, e.g., a pharmaceutical preparation, wherein at least 60% of the particles in the preparation have a conjugate number of 1-5 or 2-5.

In an embodiment, the CDP-taxane conjugate is administered as a nanoparticle or preparation of nanoparticles, e.g., a pharmaceutical preparation, wherein at least 60% of the particles in the preparation have a conjugate number of 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In one aspect, the disclosure features a method of treating pancreatic cancer (e.g., locally advanced or metastatic pancreatic cancer) in a subject, e.g., a human. The method comprises: administering a CDP-taxane conjugate, e.g., a CDP-docetaxel conjugate, a CDP-paclitaxel conjugate, a CDP-larotaxel conjugate and/or a CDP-cabazitaxel conjugate described herein, to a subject in an amount effective to treat the cancer, to thereby treat the cancer.

In one embodiment, the cancer is refractory to, resistant to, and/or relapsed with treatment with one or more of: a taxane (e.g., paclitaxel and docetaxel).

In an embodiment, the CDP-taxane conjugate comprises taxane molecules (e.g., docetaxel, paclitaxel, larotaxel and/or cabazitaxel molecules), coupled, e.g., via a linker such as a linker described herein, to a CDP moiety, e.g., a CDP described herein. In an embodiment, the CDP-taxane conjugate comprises a taxane (e.g., docetaxel, paclitaxel, larotaxel and/or cabazitaxel), coupled via a linker shown in FIG. 2 to a CDP moiety, e.g., a CDP described herein. In an embodiment, the CDP-taxane conjugate is a CDP-taxane conjugate shown in FIG. 2.

In one embodiment, the CDP-taxane conjugate is administered after surgery or before and after surgery to remove the cancer.

In one embodiment, the CDP-taxane conjugate is administered in combination with one or more of an anti-metabolite, e.g., an antifolate, e.g., floxuridine, a pyrimidine analogue, e.g., SFU, capecitabine, and/or a nucleoside analog, e.g., gemcitabine. For example, in one embodiment, the CDP-taxane conjugate is administered in combination with a nucleoside analog, e.g., gemcitabine. In one embodiment, the CDP-taxane conjugate is further administered in combination with a platinum-based agent (e.g., cisplatin, carboplatin, oxaliplatin) and a pyrimidine analogue (e.g., 5FU and/or capecitabine). In one embodiment, the CDP-taxane conjugate is further administered in combination with an epidermal growth factor (EGF) pathway inhibitor, e.g., an EGF inhibitor or EGF receptor inhibitor. In one embodiment, the EGF receptor inhibitor is cetuximab, erlotinib, or gefitinib.

In some embodiments, the CDP-taxane conjugate is administered in combination with an anti-metabolite, e.g., 5FU, and leucovorin. In one embodiment, the CDP-taxane conjugate is administered in combination with radiation.

In some embodiments, the CDP-taxane conjugate is administered in combination with a vascular endothelial growth factor (VEGF) pathway inhibitor, e.g., a VEGF inhibitor (e.g., bevacizumab) or VEGF receptor inhibitor (e.g., CP-547632 and AZD2171). In one embodiment, the CDP-taxane conjugate is administered in combination with bevacizumab.

In some embodiments, the CDP-taxane conjugate is administered in combination with an mTOR inhibitor. Non-limiting examples of mTOR inhibitors include rapamycin, everolimus, AP23573, CCI-779 and SDZ-RAD.

In one embodiment, the CDP-taxane conjugate is a CDP-docetaxel conjugate, e.g., a CDP-docetaxel conjugate described herein, e.g., a CDP-docetaxel conjugate comprising docetaxel, coupled, e.g., via linkers, to a CDP described herein. In an embodiment, the CDP-docetaxel conjugate comprises docetaxel, coupled via a linker shown in FIG. 2 to a CDP described herein. In an embodiment, the CDP-docetaxel conjugate is a CDP-docetaxel conjugate shown in FIG. 2.

In one embodiment, the CDP-docetaxel conjugate is administered at a dose and/or dosing schedule described herein.

In one embodiment, the CDP-taxane conjugate is a CDP-paclitaxel conjugate, e.g., a CDP-paclitaxel conjugate described herein, e.g., a CDP-paclitaxel conjugate comprising paclitaxel, coupled, e.g., via linkers, to a CDP described herein. In an embodiment, the CDP-paclitaxel conjugate comprises paclitaxel, coupled via a linker shown in FIG. 2 to a CDP described herein. In an embodiment, the CDP-paclitaxel conjugate is a CDP-paclitaxel conjugate shown in FIG. 2.

In one embodiment, the CDP-paclitaxel conjugate is administered at a dose and/or dosing schedule described herein.

In one embodiment, the CDP-taxane conjugate is a CDP-larotaxel conjugate, e.g., a CDP-larotaxel conjugate described herein, e.g., a CDP-larotaxel conjugate comprising larotaxel, coupled, e.g., via linkers, to a CDP described herein. In an embodiment, the CDP-larotaxel conjugate comprises larotaxel, coupled via a linker shown in FIG. 2 to a CDP described herein. In an embodiment, the CDP-larotaxel conjugate is a CDP-larotaxel conjugate shown in FIG. 2.

In one embodiment, the CDP-larotaxel conjugate is administered at a dose and/or dosing schedule described herein.

In one embodiment, the CDP-taxane conjugate is a CDP-cabazitaxel conjugate, e.g., a CDP-cabazitaxel conjugate described herein, e.g., a CDP-cabazitaxel conjugate comprising cabazitaxel, coupled, e.g., via linkers, to a CDP described herein. In an embodiment, the CDP-cabazitaxel conjugate comprises cabazitaxel, coupled via a linker shown in FIG. 2 to a CDP described herein. In an embodiment, the CDP-cabazitaxel conjugate is a CDP-cabazitaxel conjugate shown in FIG. 2.

In one embodiment, the CDP-cabazitaxel conjugate is administered at a dose and/or dosing schedule described herein.

In an embodiment, the CDP-taxane conjugate complex forms a particle or nanoparticle having a conjugate number described herein. By way of example, a CDP-therapeutic agent conjugate, forms, or is provided in, a particle or nanoparticle having a conjugate number of: 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In an embodiment the conjugate number is 2 to 4 or 2 to 5.

In an embodiment the conjugate number is 1, 2, 3, 4, 5, 6, 7, 8, 9, or 10.

In an embodiment the nanoparticle forms, or is provided in, a preparation of nanoparticles, e.g., a pharmaceutical preparation, wherein at least 40, 50, 60, 70, 80, 90 or 95% of the particles in the preparation have a conjugate number provided herein. In an embodiment the nanoparticle forms, or is provided in, a preparation of nanoparticles, e.g., a pharmaceutical preparation, wherein at least 60% of the particles in the preparation have a conjugate number of 1-5 or 2-5.

In an embodiment, the CDP-taxane conjugate is administered as a nanoparticle or preparation of nanoparticles, e.g., a pharmaceutical preparation, wherein at least 60% of the particles in the preparation have a conjugate number of 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In one aspect, the disclosure features a method of treating pancreatic cancer, e.g. locally advanced or metastatic pancreatic cancer, in a subject, e.g., a human. The method comprises:

providing a subject who has pancreatic cancer and has been treated with a chemotherapeutic agent which did not effectively treat the cancer (e.g., the subject has a non-resectable cancer, a chemotherapeutic refractory, a chemotherapeutic resistant and/or a relapsed cancer) or which had an unacceptable side effect (e.g., the subject has a chemotherapeutic sensitive cancer), and

administering a CDP-taxane conjugate, e.g., a CDP-docetaxel conjugate, a CDP-paclitaxel conjugate, a CDP-larotaxel conjugate and/or a CDP-cabazitaxel conjugate described herein, to a subject in an amount effective to treat the cancer, to thereby treat the cancer.

In an embodiment, the CDP-taxane conjugate comprises taxane molecules (e.g., docetaxel, paclitaxel, larotaxel and/or cabazitaxel molecules), coupled, e.g., via a linker such as a linker described herein, to a CDP moiety, e.g., a CDP described herein. In an embodiment, the CDP-taxane conjugate comprises a taxane (e.g., docetaxel, paclitaxel, larotaxel and/or cabazitaxel), coupled via a linker shown in FIG. 2 to a CDP moiety, e.g., a CDP described herein. In an embodiment, the CDP-taxane conjugate is a CDP-taxane conjugate shown in FIG. 2.

In one embodiment, the cancer is refractory to, resistant to, and/or relapsed with treatment with one or more of: a taxane (e.g., paclitaxel, docetaxel, larotaxel, cabazitaxel), an anthracycline (e.g., daunorubicin, doxorubicin, epirubicin, valrubicin and idarubicin), an anti-metabolite, e.g., an antifolate (e.g., floxuridine, pemetrexed) or pyrimidine analogue (e.g., capecitabine, 5FU)), and a platinum-based agent (e.g., cisplatin, carboplatin, oxaliplatin).

In one embodiment, the cancer is a multidrug resistant cancer.

In one embodiment, the CDP-taxane conjugate is administered in combination with a pyrimidine analogue, e.g., a pyrimidine analogue described herein (e.g., capecitabine and/or 5FU). In one embodiment, the CDP-taxane conjugate is administered in combination with a pyrimidine analogue, e.g., SFU, and leucovorin. In one embodiment, the CDP-taxane conjugate is further administered in combination with a platinum-based agent (e.g., cisplatin, carboplatin, oxaliplatin).

In one embodiment, the CDP-taxane conjugate is a CDP-docetaxel conjugate, e.g., a CDP-docetaxel conjugate described herein, e.g., a CDP-docetaxel conjugate comprising docetaxel, coupled, e.g., via linkers, to a CDP described herein. In an embodiment, the CDP-docetaxel conjugate comprises docetaxel, coupled via a linker shown in FIG. 2 to a CDP described herein. In an embodiment, the CDP-docetaxel conjugate is a CDP-docetaxel conjugate shown in FIG. 2.

In one embodiment, the CDP-docetaxel conjugate is administered at a dose and/or dosing schedule described herein.

In one embodiment, the CDP-taxane conjugate is a CDP-paclitaxel conjugate, e.g., a CDP-paclitaxel conjugate described herein, e.g., a CDP-paclitaxel conjugate comprising paclitaxel, coupled, e.g., via linkers, to a CDP described herein. In an embodiment, the CDP-paclitaxel conjugate comprises paclitaxel, coupled via a linker shown in FIG. 2 to a CDP described herein. In an embodiment, the CDP-paclitaxel conjugate is a CDP-paclitaxel conjugate shown in FIG. 2.

In one embodiment, the CDP-paclitaxel conjugate is administered at a dose and/or dosing schedule described herein.

In one embodiment, the CDP-taxane conjugate is a CDP-larotaxel conjugate, e.g., a CDP-larotaxel conjugate described herein, e.g., a CDP-larotaxel conjugate comprising larotaxel, coupled, e.g., via linkers, to a CDP described herein. In an embodiment, the CDP-larotaxel conjugate comprises larotaxel, coupled via a linker shown in FIG. 2 to a CDP described herein. In an embodiment, the CDP-larotaxel conjugate is a CDP-larotaxel conjugate shown in FIG. 2.

In one embodiment, the CDP-larotaxel conjugate is administered at a dose and/or dosing schedule described herein.

In one embodiment, the CDP-taxane conjugate is a CDP-cabazitaxel conjugate, e.g., a CDP-cabazitaxel conjugate described herein, e.g., a CDP-cabazitaxel conjugate comprising cabazitaxel, coupled, e.g., via linkers, to a CDP described herein. In an embodiment, the CDP-cabazitaxel conjugate comprises cabazitaxel, coupled via a linker shown in FIG. 2 to a CDP described herein. In an embodiment, the CDP-cabazitaxel conjugate is a CDP-cabazitaxel conjugate shown in FIG. 2.

In one embodiment, the CDP-cabazitaxel conjugate is administered at a dose and/or dosing schedule described herein.

In an embodiment, the CDP-taxane conjugate complex forms a particle or nanoparticle having a conjugate number described herein. By way of example, a CDP-therapeutic agent conjugate, forms, or is provided in, a particle or nanoparticle having a conjugate number of: 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In an embodiment the conjugate number is 2 to 4 or 2 to 5.

In an embodiment the conjugate number is 1, 2, 3, 4, 5, 6, 7, 8, 9, or 10.

In an embodiment the nanoparticle forms, or is provided in, a preparation of nanoparticles, e.g., a pharmaceutical preparation, wherein at least 40, 50, 60, 70, 80, 90 or 95% of the particles in the preparation have a conjugate number provided herein. In an embodiment the nanoparticle forms, or is provided in, a preparation of nanoparticles, e.g., a pharmaceutical preparation, wherein at least 60% of the particles in the preparation have a conjugate number of 1-5 or 2-5.

In an embodiment, the CDP-taxane conjugate is administered as a nanoparticle or preparation of nanoparticles, e.g., a pharmaceutical preparation, wherein at least 60% of the particles in the preparation have a conjugate number of 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In yet another aspect, the invention features a method of treating advanced or metastatic colorectal cancer in a subject, e.g., a human. The method comprises: administering a composition comprising a CDP-taxane conjugate, e.g., a CDP-docetaxel conjugate, a CDP-paclitaxel conjugate, a CDP-larotaxel conjugate and/or a CDP-cabazitaxel conjugate described herein, to a subject in an amount effective to treat the cancer, to thereby treat the cancer.

In an embodiment, the CDP-taxane conjugate comprises taxane molecules (e.g., docetaxel, paclitaxel, larotaxel and/or cabazitaxel molecules), coupled, e.g., via a linker such as a linker described herein, to a CDP moiety, e.g., a CDP described herein. In an embodiment, the CDP-taxane conjugate comprises a taxane (e.g., docetaxel, paclitaxel, larotaxel and/or cabazitaxel), coupled via a linker shown in FIG. 2 to a CDP moiety, e.g., a CDP described herein. In an embodiment, the CDP-taxane conjugate is a CDP-taxane conjugate shown in FIG. 2.

In one embodiment, the cancer is refractory to, resistant to, and/or relapsed with treatment with one or more of: a taxane (e.g., paclitaxel and docetaxel).

In one embodiment, the CDP-taxane conjugate is administered in combination with an antimetabolite, e.g., an antifolate (e.g., pemetrexed, raltitrexed). In one embodiment, the CDP-taxane conjugate is administered in combination with an antimetabolite, e.g., 5FU, and leucovorin. In one embodiment, the CDP-taxane conjugate is further administered in combination with a platinum-based agent (e.g., cisplatin, carboplatin, oxaliplatin). For example, in one embodiment, the CDP-taxane conjugate is administered in combination with an antimetabolite, e.g., 5FU, leucovorin, and a platinum-based agent, e.g., oxaliplatin. In another embodiment, the antimetabolite is a pyrimidine analog, e.g., capecitabine.

In one embodiment, the CDP-taxane conjugate is administered in combination with a platinum-based agent (e.g., cisplatin, carboplatin, oxaliplatin).

In one embodiment, the CDP-taxane conjugate is administered in combination with a vascular endothelial growth factor (VEGF) pathway inhibitor, e.g., a VEGF inhibitor or VEGF receptor inhibitor. In one embodiment, the VEGF inhibitor is bevacizumab. In one embodiment, the VEGF receptor inhibitor is selected from CP-547632 and AZD2171. In one embodiment, the CDP-taxane conjugate is administered in combination with a VEGF pathway inhibitor, e.g., bevacizumab, and an antimetabolite, e.g., an antifolate (e.g., pemetrexed, raltitrexed) or pyrimidine analogue (e.g., 5FU). In one embodiment, the CDP-taxane conjugate is administered with a VEGF pathway inhibitor, e.g., bevacizumab, an antimetabolite, e.g., a pyrimidine analogue (e.g., 5FU), and leucovorin. In another embodiment, the CDP-taxane conjugate is administered with a VEGF pathway inhibitor, e.g., bevacizumab, an antimetabolite, e.g., a pyrimidine analogue (e.g., 5FU), leucovorin, a platinum-based agent (e.g., cisplatin, carboplatin, oxaliplatin) and/or a topoisomerase inhibitor (e.g., irinotecan, topotecan, etoposide, teniposide, lamellarin D, SN-38, camptothecin (e.g., CRLX101)). For example, in one embodiment, the CDP-taxane conjugate is administered with the following combination: a VEGF pathway inhibitor, e.g., bevacizumab, an antimetabolite (e.g., 5FU), leucovorin and a platinum-based agent (e.g., oxaliplatin); a VEGF pathway inhibitor, e.g., bevacizumab, an antimetabolite (e.g., 5FU), leucovorin, a platinum-based agent (e.g., oxaliplatin) and a topoisomerase inhibitor (e.g., irinotecan); or a VEGF pathway inhibitor, e.g., bevacizumab, an antimetabolite (e.g., 5FU), leucovorin and a topoisomerase inhibitor (e.g., irinotecan).

In another embodiment, the CDP-taxane conjugate is administered in combination with a VEGF pathway inhibitor, e.g., bevacizumab, and an antimetabolite wherein the antimetabolite is a pyrimidine analog, e.g., capecitabine. In one embodiment, the CDP-taxane conjugate is further administered in combination with a platinum-based agent (e.g., cisplatin, carboplatin, oxaliplatin) or a topoisomerase inhibitor (e.g., irinotecan, topotecan, etoposide, teniposide, lamellarin D, SN-38, camptothecin (e.g., CRLX101)). For example, in one embodiment, the CDP-taxane conjugate is administered with the following combination: a VEGF pathway inhibitor, e.g., bevacizumab, a pyrimidine analog, e.g., capecitabine, and a platinum-based agent (e.g., oxaliplatin); or a VEGF pathway inhibitor, e.g., bevacizumab, a pyrimidine analog, e.g., capecitabine, and a topoisomerase inhibitor (e.g., irinotecan).

In one embodiment, the CDP-taxane conjugate is administered in combination with an epidermal growth factor (EGF) pathway inhibitor, e.g., an EGF inhibitor or EGF receptor inhibitor. The EGF receptor inhibitor can be, e.g., cetuximab, erlotinib, gefitinib, panitumumab. In one embodiment, the CDP-taxane conjugate is administered in combination with an EGF pathway inhibitor, e.g., cetuximab or panitumumab, and a VEGF pathway inhibitor, e.g., bevacizumab.

In one embodiment, the CDP-taxane conjugate is administered in combination with a topoisomerase inhibitor (e.g., irinotecan, topotecan, etoposide, teniposide, lamellarin D, SN-38, camptothecin (e.g., CRLX101)). In one embodiment, the CDP-taxane conjugate is administered in combination with a topoisomerase inhibitor (e.g., irinotecan) and a VEGF pathway inhibitor, e.g., bevacizumab.

In one embodiment, the CDP-taxane conjugate is a CDP-docetaxel conjugate, e.g., a CDP-docetaxel conjugate described herein, e.g., a CDP-docetaxel conjugate comprising docetaxel, coupled, e.g., via linkers, to a CDP described herein. In an embodiment, the CDP-docetaxel conjugate comprises docetaxel, coupled via a linker shown in FIG. 2 to a CDP described herein. In an embodiment, the CDP-docetaxel conjugate is a CDP-docetaxel conjugate shown in FIG. 2.

In one embodiment, the CDP-docetaxel conjugate is administered at a dose and/or dosing schedule described herein.

In one embodiment, the CDP-taxane conjugate is a CDP-paclitaxel conjugate, e.g., a CDP-paclitaxel conjugate described herein, e.g., a CDP-paclitaxel conjugate comprising paclitaxel, coupled, e.g., via linkers, to a CDP described herein. In an embodiment, the CDP-paclitaxel conjugate comprises paclitaxel, coupled via a linker shown in FIG. 2 to a CDP described herein. In an embodiment, the CDP-paclitaxel conjugate is a CDP-paclitaxel conjugate shown in FIG. 2.

In one embodiment, the CDP-paclitaxel conjugate is administered at a dose and/or dosing schedule described herein.

In one embodiment, the CDP-taxane conjugate is a CDP-larotaxel conjugate, e.g., a CDP-larotaxel conjugate described herein, e.g., a CDP-larotaxel conjugate comprising larotaxel, coupled, e.g., via linkers, to a CDP described herein. In an embodiment, the CDP-larotaxel conjugate comprises larotaxel, coupled via a linker shown in FIG. 2 to a CDP described herein. In an embodiment, the CDP-larotaxel conjugate is a CDP-larotaxel conjugate shown in FIG. 2.

In one embodiment, the CDP-larotaxel conjugate is administered at a dose and/or dosing schedule described herein.

In one embodiment, the CDP-taxane conjugate is a CDP-cabazitaxel conjugate, e.g., a CDP-cabazitaxel conjugate described herein, e.g., a CDP-cabazitaxel conjugate comprising cabazitaxel, coupled, e.g., via linkers, to a CDP described herein. In an embodiment, the CDP-cabazitaxel conjugate comprises cabazitaxel, coupled via a linker shown in FIG. 2 to a CDP described herein. In an embodiment, the CDP-cabazitaxel conjugate is a CDP-cabazitaxel conjugate shown in FIG. 2.

In one embodiment, the CDP-cabazitaxel conjugate is administered at a dose and/or dosing schedule described herein.

In an embodiment, the CDP-taxane conjugate complex forms a particle or nanoparticle having a conjugate number described herein. By way of example, a CDP-therapeutic agent conjugate, forms, or is provided in, a particle or nanoparticle having a conjugate number of: 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In an embodiment the conjugate number is 2 to 4 or 2 to 5.

In an embodiment the conjugate number is 1, 2, 3, 4, 5, 6, 7, 8, 9, or 10.

In an embodiment the nanoparticle forms, or is provided in, a preparation of nanoparticles, e.g., a pharmaceutical preparation, wherein at least 40, 50, 60, 70, 80, 90 or 95% of the particles in the preparation have a conjugate number provided herein. In an embodiment the nanoparticle forms, or is provided in, a preparation of nanoparticles, e.g., a pharmaceutical preparation, wherein at least 60% of the particles in the preparation have a conjugate number of 1-5 or 2-5.

In an embodiment, the CDP-taxane conjugate is administered as a nanoparticle or preparation of nanoparticles, e.g., a pharmaceutical preparation, wherein at least 60% of the particles in the preparation have a conjugate number of 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In yet another aspect, the invention features a method of treating advanced or metastatic colorectal cancer in a subject, e.g., a human, the method comprising:

providing a subject who has advanced or metastatic colorectal cancer and has been treated with a chemotherapeutic agent that did not effectively treat the cancer (e.g., the subject has a chemotherapeutic refractory cancer, a chemotherapeutic resistant cancer and/or a relapsed cancer) or who had an unacceptable side effect (e.g., the subject has a chemotherapeutic sensitive cancer), and

administering a CDP-taxane conjugate, e.g., a CDP-docetaxel conjugate, a CDP-paclitaxel conjugate, a CDP-larotaxel conjugate and/or a CDP-cabazitaxel conjugate described herein, to a subject in an amount effective to treat the cancer, to thereby treat the cancer.

In one embodiment, the cancer is refractory to, resistant to, and/or relapsed with treatment with one or more of: a taxane (e.g., paclitaxel and docetaxel).

In an embodiment, the CDP-taxane conjugate comprises taxane molecules (e.g., docetaxel, paclitaxel, larotaxel and/or cabazitaxel molecules), coupled, e.g., via a linker such as a linker described herein, to a CDP moiety, e.g., a CDP described herein. In an embodiment, the CDP-taxane conjugate comprises a taxane (e.g., docetaxel, paclitaxel, larotaxel and/or cabazitaxel), coupled via a linker shown in FIG. 2 to a CDP moiety, e.g., a CDP described herein. In an embodiment, the CDP-taxane conjugate is a CDP-taxane conjugate shown in FIG. 2.

In one embodiment, the subject has been treated with an anti-metabolite, e.g., a pyrimidine analogue which did not effectively treat the cancer (e.g., the subject has a capecitabine and/or 5FU refractory, a capecitabine and/or 5FU resistant and/or relapsed cancer).

In one embodiment, the subject has been treated with a pyrimidine analog which did not effectively treat the cancer (e.g., the subject has a capecitabine refractory, a capecitabine resistant and/or a relapsed cancer).

In one embodiment, the CDP-taxane conjugate is administered in combination with a vascular endothelial growth factor (VEGF) pathway inhibitor, e.g., a VEGF inhibitor or VEGF receptor inhibitor. In one embodiment, the VEGF inhibitor is bevacizumab. In one embodiment, the VEGF receptor inhibitor is selected from CP-547632 and AZD2171. In one embodiment, the CDP-taxane conjugate is administered in combination with a VEGF pathway inhibitor, e.g., bevacizumab, and an antimetabolite, e.g., an antifolate (e.g., pemetrexed, raltitrexed) or pyrimidine analogue (e.g., 5FU). In one embodiment, the CDP-taxane conjugate is administered with a VEGF pathway inhibitor, e.g., bevacizumab, an antimetabolite (e.g., 5FU) and leucovorin. In another embodiment, the CDP-taxane conjugate is administered with a VEGF pathway inhibitor, e.g., bevacizumab, an antimetabolite (e.g., 5FU), leucovorin, a platinum-based agent (e.g., cisplatin, carboplatin, oxaliplatin) and/or a topoisomerase inhibitor (e.g., irinotecan, topotecan, etoposide, teniposide, lamellarin D, SN-38, camptothecin (e.g., CRLX101)). For example, in one embodiment, the CDP-taxane conjugate is administered with the following combination: a VEGF pathway inhibitor, e.g., bevacizumab, an antimetabolite (e.g., 5FU), leucovorin and a platinum-based agent (e.g., oxaliplatin); a VEGF pathway inhibitor, e.g., bevacizumab, an antimetabolite (e.g., 5FU), leucovorin, a platinum-based agent (e.g., oxaliplatin) and a topoisomerase inhibitor (e.g., irinotecan); or a VEGF pathway inhibitor, e.g., bevacizumab, an antimetabolite (e.g., 5FU), leucovorin and a topoisomerase inhibitor (e.g., irinotecan).

In another embodiment, the CDP-taxane conjugate is administered in combination with a VEGF pathway inhibitor, e.g., bevacizumab, and an antimetabolite wherein the antimetabolite is a pyrimidine analog, e.g., capecitabine. In one embodiment, the CDP-taxane conjugate is further administered in combination with a platinum-based agent (e.g., cisplatin, carboplatin, oxaliplatin) or a topoisomerase inhibitor (e.g., irinotecan, topotecan, etoposide, teniposide, lamellarin D, SN-38, camptothecin (e.g., CRLX101)). For example, in one embodiment, the CDP-taxane conjugate is administered with the following combination: a VEGF pathway inhibitor, e.g., bevacizumab, a pyrimidine analog, e.g., capecitabine, and a platinum-based agent (e.g., oxaliplatin); or a VEGF pathway inhibitor, e.g., bevacizumab, a pyrimidine analog, e.g., capecitabine, and a topoisomerase inhibitor (e.g., irinotecan).

In one embodiment, the CDP-taxane conjugate is administered in combination with an epidermal growth factor (EGF) pathway inhibitor, e.g., an EGF inhibitor or EGF receptor inhibitor. The EGF receptor inhibitor can be, e.g., cetuximab, erlotinib, gefitinib, panitumumab. In one embodiment, the CDP-taxane conjugate is administered in combination with an EGF pathway inhibitor, e.g., cetuximab or panitumumab, and a VEGF pathway inhibitor, e.g., bevacizumab.

In one embodiment, the CDP-taxane conjugate is administered in combination with a topoisomerase inhibitor (e.g., irinotecan, topotecan, etoposide, teniposide, lamellarin D, SN-38, camptothecin (e.g., CRLX101)). In one embodiment, the CDP-taxane conjugate is administered in combination with a topoisomerase inhibitor (e.g., irinotecan) and a VEGF pathway inhibitor, e.g., bevacizumab.

In one embodiment, the CDP-taxane conjugate is a CDP-docetaxel conjugate, e.g., a CDP-docetaxel conjugate described herein, e.g., a CDP-docetaxel conjugate comprising docetaxel, coupled, e.g., via linkers, to a CDP described herein. In an embodiment, the CDP-docetaxel conjugate comprises docetaxel, coupled via a linker shown in FIG. 2 to a CDP described herein. In an embodiment, the CDP-docetaxel conjugate is a CDP-docetaxel conjugate shown in FIG. 2.

In one embodiment, the CDP-docetaxel conjugate is administered at a dose and/or dosing schedule described herein.

In one embodiment, the CDP-taxane conjugate is a CDP-paclitaxel conjugate, e.g., a CDP-paclitaxel conjugate described herein, e.g., a CDP-paclitaxel conjugate comprising paclitaxel, coupled, e.g., via linkers, to a CDP described herein. In an embodiment, the CDP-paclitaxel conjugate comprises paclitaxel, coupled via a linker shown in FIG. 2 to a CDP described herein. In an embodiment, the CDP-paclitaxel conjugate is a CDP-docetaxel conjugate shown in FIG. 2.

In one embodiment, the CDP-paclitaxel conjugate is administered at a dose and/or dosing schedule described herein.

In one embodiment, the CDP-taxane conjugate is a CDP-larotaxel conjugate, e.g., a CDP-larotaxel conjugate described herein, e.g., a CDP-larotaxel conjugate comprising larotaxel, coupled, e.g., via linkers, to a CDP described herein. In an embodiment, the CDP-larotaxel conjugate comprises larotaxel, coupled via a linker shown in FIG. 2 to a CDP described herein. In an embodiment, the CDP-larotaxel conjugate is a CDP-larotaxel conjugate shown in FIG. 2.

In one embodiment, the CDP-larotaxel conjugate is administered at a dose and/or dosing schedule described herein.

In one embodiment, the CDP-taxane conjugate is a CDP-cabazitaxel conjugate, e.g., a CDP-cabazitaxel conjugate described herein, e.g., a CDP-cabazitaxel conjugate comprising cabazitaxel, coupled, e.g., via linkers, to a CDP described herein. In an embodiment, the CDP-cabazitaxel conjugate comprises cabazitaxel, coupled via a linker shown in FIG. 2 to a CDP described herein. In an embodiment, the CDP-cabazitaxel conjugate is a CDP-cabazitaxel conjugate shown in FIG. 2.

In one embodiment, the CDP-cabazitaxel conjugate is administered at a dose and/or dosing schedule described herein.

In an embodiment, the CDP-taxane conjugate complex forms a particle or nanoparticle having a conjugate number described herein. By way of example, a CDP-therapeutic agent conjugate, forms, or is provided in, a particle or nanoparticle having a conjugate number of: 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In an embodiment the conjugate number is 2 to 4 or 2 to 5.

In an embodiment the conjugate number is 1, 2, 3, 4, 5, 6, 7, 8, 9, or 10.

In an embodiment the nanoparticle forms, or is provided in, a preparation of nanoparticles, e.g., a pharmaceutical preparation, wherein at least 40, 50, 60, 70, 80, 90 or 95% of the particles in the preparation have a conjugate number provided herein. In an embodiment the nanoparticle forms, or is provided in, a preparation of nanoparticles, e.g., a pharmaceutical preparation, wherein at least 60% of the particles in the preparation have a conjugate number of 1-5 or 2-5.

In an embodiment, the CDP-taxane conjugate is administered as a nanoparticle or preparation of nanoparticles, e.g., a pharmaceutical preparation, wherein at least 60% of the particles in the preparation have a conjugate number of 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In yet another aspect, the invention features a method of identifying a subject, e.g., a human, having a proliferative disorder, e.g., cancer, for treatment with a CDP-taxane conjugate, e.g., a CDP-taxane conjugate described herein, the method comprising

identifying a subject having a proliferative disorder who has received an anticancer agent (e.g., a taxane) and has a neutrophil count less than a standard; and

identifying the subject as suitable for treatment with a CDP-taxane conjugate, e.g., a CDP-docetaxel conjugate, a CDP-paclitaxel conjugate, a CDP-larotaxel conjugate and/or a CDP-cabazitaxel conjugate described herein.

In one embodiment, the method further comprising administering a CDP-taxane conjugate, e.g., a CDP-taxane conjugate described herein in an amount effective to treat the disorder.

In an embodiment, the CDP-taxane conjugate comprises taxane molecules (e.g., docetaxel, paclitaxel, larotaxel and/or cabazitaxel molecules), coupled, e.g., via a linker such as a linker described herein, to a CDP moiety, e.g., a CDP described herein. In an embodiment, the CDP-taxane conjugate comprises a taxane (e.g., docetaxel, paclitaxel, larotaxel and/or cabazitaxel), coupled via a linker shown in FIG. 2 to a CDP moiety, e.g., a CDP described herein. In an embodiment, the CDP-taxane conjugate is a CDP-taxane conjugate shown in FIG. 2.

In one embodiment, the CDP-taxane conjugate is a CDP-docetaxel conjugate, e.g., a CDP-docetaxel conjugate described herein, e.g., a CDP-docetaxel conjugate comprising docetaxel, coupled, e.g., via linkers, to a CDP described herein. In an embodiment, the CDP-docetaxel conjugate comprises docetaxel, coupled via a linker shown in FIG. 2 to a CDP described herein. In an embodiment, the CDP-docetaxel conjugate is a CDP-docetaxel conjugate shown in FIG. 2.

In one embodiment, the CDP-docetaxel conjugate is administered at a dose and/or dosing schedule described herein.

In one embodiment, the CDP-taxane conjugate is a CDP-paclitaxel conjugate, e.g., a CDP-paclitaxel conjugate described herein, e.g., a CDP-paclitaxel conjugate comprising paclitaxel, coupled, e.g., via linkers, to a CDP described herein. In an embodiment, the CDP-paclitaxel conjugate comprises paclitaxel, coupled via a linker shown in FIG. 2 to a CDP described herein. In an embodiment, the CDP-paclitaxel conjugate is a CDP-docetaxel conjugate shown in FIG. 2.

In one embodiment, the CDP-paclitaxel conjugate is administered at a dose and/or dosing schedule described herein.

In one embodiment, the CDP-taxane conjugate is a CDP-larotaxel conjugate, e.g., a CDP-larotaxel conjugate described herein, e.g., a CDP-larotaxel conjugate comprising larotaxel, coupled, e.g., via linkers, to a CDP described herein. In an embodiment, the CDP-larotaxel conjugate comprises larotaxel, coupled via a linker shown in FIG. 2 to a CDP described herein. In an embodiment, the CDP-larotaxel conjugate is a CDP-larotaxel conjugate shown in FIG. 2.

In one embodiment, the CDP-larotaxel conjugate is administered at a dose and/or dosing schedule described herein.

In one embodiment, the CDP-taxane conjugate is a CDP-cabazitaxel conjugate, e.g., a CDP-cabazitaxel conjugate described herein, e.g., a CDP-cabazitaxel conjugate comprising cabazitaxel, coupled, e.g., via linkers, to a CDP described herein. In an embodiment, the CDP-cabazitaxel conjugate comprises cabazitaxel, coupled via a linker shown in FIG. 2 to a CDP described herein. In an embodiment, the CDP-cabazitaxel conjugate is a CDP-cabazitaxel conjugate shown in FIG. 2.

In one embodiment, the CDP-cabazitaxel conjugate is administered at a dose and/or dosing schedule described herein.

In one embodiment, the cancer is a cancer described herein. In one embodiment, the CDP-taxane conjugate is administered in combination with one or more additional chemotherapeutic agent, e.g., a chemotherapeutic agent or combination of chemotherapeutic agents described herein. In one embodiment, the CDP-taxane conjugate is administered in combination with a granulocyte colony stimulating factor, e.g., GCSF or GMCSF.

In one embodiment, the standard is a neutrophil count below or equal to 1500 cells/mm³. In some embodiments, the standard is based on a neutrophil count prior to receiving an anticancer agent, e.g., mean neutrophil count decreased from the mean neutrophil count prior to treatment with the anticancer agent, e.g., by at least 20%, 30%, 40% or 50% after administration of the anticancer agent.

In an embodiment, the CDP-taxane conjugate complex forms a particle or nanoparticle having a conjugate number described herein. By way of example, a CDP-therapeutic agent conjugate, forms, or is provided in, a particle or nanoparticle having a conjugate number of: 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In an embodiment the conjugate number is 2 to 4 or 2 to 5.

In an embodiment the conjugate number is 1, 2, 3, 4, 5, 6, 7, 8, 9, or 10.

In an embodiment the nanoparticle forms, or is provided in, a preparation of nanoparticles, e.g., a pharmaceutical preparation, wherein at least 40, 50, 60, 70, 80, 90 or 95% of the particles in the preparation have a conjugate number provided herein. In an embodiment the nanoparticle forms, or is provided in, a preparation of nanoparticles, e.g., a pharmaceutical preparation, wherein at least 60% of the particles in the preparation have a conjugate number of 1-5 or 2-5.

In an embodiment, the CDP-taxane conjugate is administered as a nanoparticle or preparation of nanoparticles, e.g., a pharmaceutical preparation, wherein at least 60% of the particles in the preparation have a conjugate number of 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30;

or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In another aspect, the invention features a method of treating a subject, e.g., a human, with a proliferative disorder, e.g., cancer, the method comprising

selecting a subject having a proliferative disease who has received an anticancer agent (e.g., a taxane) and has a neutrophil count less than a standard; and

administering a CDP-taxane conjugate, e.g., a CDP-docetaxel conjugate, a CDP-paclitaxel conjugate, a CDP-larotaxel conjugate and/or a CDP-cabazitaxel conjugate described herein, to the subject in an amount effective to treat the proliferative disorder, to thereby treat the disorder.

In an embodiment, the CDP-taxane conjugate comprises taxane molecules (e.g., docetaxel, paclitaxel, larotaxel and/or cabazitaxel molecules), coupled, e.g., via a linker such as a linker described herein, to a CDP moiety, e.g., a CDP described herein. In an embodiment, the CDP-taxane conjugate comprises a taxane (e.g., docetaxel, paclitaxel, larotaxel and/or cabazitaxel), coupled via a linker shown in FIG. 2 to a CDP moiety, e.g., a CDP described herein. In an embodiment, the CDP-taxane conjugate is a CDP-taxane conjugate shown in FIG. 2.

In one embodiment, the CDP-taxane conjugate is a CDP-docetaxel conjugate, e.g., a CDP-docetaxel conjugate described herein, e.g., a CDP-docetaxel conjugate comprising docetaxel, coupled, e.g., via linkers, to a CDP described herein. In an embodiment, the CDP-docetaxel conjugate comprises docetaxel, coupled via a linker shown in FIG. 2 to a CDP described herein. In an embodiment, the CDP-docetaxel conjugate is a CDP-docetaxel conjugate shown in FIG. 2.

In one embodiment, the CDP-docetaxel conjugate is administered at a dose and/or dosing schedule described herein.

In one embodiment, the CDP-taxane conjugate is a CDP-paclitaxel conjugate, e.g., a CDP-paclitaxel conjugate described herein, e.g., a CDP-paclitaxel conjugate comprising paclitaxel, coupled, e.g., via linkers, to a CDP described herein. In an embodiment, the CDP-paclitaxel conjugate comprises paclitaxel, coupled via a linker shown in FIG. 2 to a CDP described herein. In an embodiment, the CDP-paclitaxel conjugate is a CDP-docetaxel conjugate shown in FIG. 2.

In one embodiment, the CDP-paclitaxel conjugate is administered at a dose and/or dosing schedule described herein.

In one embodiment, the CDP-taxane conjugate is a CDP-larotaxel conjugate, e.g., a CDP-larotaxel conjugate described herein, e.g., a CDP-larotaxel conjugate comprising larotaxel, coupled, e.g., via linkers, to a CDP described herein. In an embodiment, the

CDP-larotaxel conjugate comprises larotaxel, coupled via a linker shown in FIG. 2 to a CDP described herein. In an embodiment, the CDP-larotaxel conjugate is a CDP-larotaxel conjugate shown in FIG. 2.

In one embodiment, the CDP-larotaxel conjugate is administered at a dose and/or dosing schedule described herein.

In one embodiment, the CDP-taxane conjugate is a CDP-cabazitaxel conjugate, e.g., a CDP-cabazitaxel conjugate described herein, e.g., a CDP-cabazitaxel conjugate comprising cabazitaxel, coupled, e.g., via linkers, to a CDP described herein. In an embodiment, the CDP-cabazitaxel conjugate comprises cabazitaxel, coupled via a linker shown in FIG. 2 to a CDP described herein. In an embodiment, the CDP-cabazitaxel conjugate is a CDP-cabazitaxel conjugate shown in FIG. 2.

In one embodiment, the CDP-cabazitaxel conjugate is administered at a dose and/or dosing schedule described herein.

In one embodiment, the cancer is a cancer described herein. In one embodiment, the CDP-taxane conjugate is administered in combination with one or more additional chemotherapeutic agent, e.g., a chemotherapeutic agent or combination of chemotherapeutic agents described herein. In one embodiment, the CDP-taxane conjugate is administered in combination with a granulocyte colony stimulating factor, e.g., GCSF or GMCSF.

In one embodiment, the standard is a neutrophil count below or equal to 1500 cells/mm³. In some embodiments, the standard is based on a neutrophil count prior to receiving an anticancer agent, e.g., mean neutrophil count decreased from the mean neutrophil count prior to treatment with the anticancer agent, e.g., by at least 20%, 30%, 40% or 50% after administration of the anticancer agent.

In an embodiment, the CDP-taxane conjugate complex forms a particle or nanoparticle having a conjugate number described herein. By way of example, a CDP-therapeutic agent conjugate, forms, or is provided in, a particle or nanoparticle having a conjugate number of: 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In an embodiment the conjugate number is 2 to 4 or 2 to 5.

In an embodiment the conjugate number is 1, 2, 3, 4, 5, 6, 7, 8, 9, or 10.

In an embodiment the nanoparticle forms, or is provided in, a preparation of nanoparticles, e.g., a pharmaceutical preparation, wherein at least 40, 50, 60, 70, 80, 90 or 95% of the particles in the preparation have a conjugate number provided herein. In an embodiment the nanoparticle forms, or is provided in, a preparation of nanoparticles, e.g., a pharmaceutical preparation, wherein at least 60% of the particles in the preparation have a conjugate number of 1-5 or 2-5.

In an embodiment, the CDP-taxane conjugate is administered as a nanoparticle or preparation of nanoparticles, e.g., a pharmaceutical preparation, wherein at least 60% of the particles in the preparation have a conjugate number of 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In yet another aspect, the invention features a method for selecting a subject, e.g., a human, with a proliferative disorder, e.g., cancer, for treatment with a CDP-taxane conjugate, e.g., a CDP-docetaxel conjugate, a CDP-paclitaxel conjugate, a CDP-larotaxel conjugate and/or a CDP-cabazitaxel conjugate described herein, comprising:

determining whether a subject with a proliferative disorder has moderate to severe neutropenia; and

selecting a subject for treatment with a CDP-taxane conjugate on the basis that the subject has moderate to severe neutropenia.

In an embodiment, the CDP-taxane conjugate comprises taxane molecules (e.g., docetaxel, paclitaxel, larotaxel and/or cabazitaxel molecules), coupled, e.g., via a linker such as a linker described herein, to a CDP moiety, e.g., a CDP described herein. In an embodiment, the CDP-taxane conjugate comprises a taxane (e.g., docetaxel, paclitaxel, larotaxel and/or cabazitaxel), coupled via a linker shown in FIG. 2 to a CDP moiety, e.g., a CDP described herein. In an embodiment, the CDP-taxane conjugate is a CDP-taxane conjugate shown in FIG. 2.

In one embodiment, the CDP-taxane conjugate is a CDP-docetaxel conjugate, e.g., a CDP-docetaxel conjugate described herein, e.g., a CDP-docetaxel conjugate comprising docetaxel, coupled, e.g., via linkers, to a CDP described herein. In an embodiment, the CDP-docetaxel conjugate comprises docetaxel, coupled via a linker shown in FIG. 2 to a CDP described herein. In an embodiment, the CDP-docetaxel conjugate is a CDP-docetaxel conjugate shown in FIG. 2.

In one embodiment, the CDP-docetaxel conjugate is administered at a dose and/or dosing schedule described herein.

In one embodiment, the CDP-docetaxel conjugate is administered at a dose and/or dosing schedule described herein. In one embodiment, the dosing schedule is not changed between doses. For example, when the dosing schedule is every three weeks, an additional dose is administered in three weeks. In one embodiment, the dose does not change or is increased for an additional dose (or doses). For example, when a dose of the CDP-docetaxel conjugate is administered in an amount such that the conjugate includes 60 mg/m² of docetaxel, an additional dose is administered in an amount such that the conjugate includes 60 mg/m² or greater of docetaxel.

In one embodiment, the CDP-taxane conjugate is a CDP-paclitaxel conjugate, e.g., a CDP-paclitaxel conjugate described herein, e.g., a CDP-paclitaxel conjugate comprising paclitaxel, coupled, e.g., via linkers, to a CDP described herein. In an embodiment, the CDP-paclitaxel conjugate comprises paclitaxel, coupled via a linker shown in FIG. 2 to a CDP described herein. In an embodiment, the CDP-paclitaxel conjugate is a CDP-docetaxel conjugate shown in FIG. 2.

In one embodiment, the CDP-paclitaxel conjugate is administered at a dose and/or dosing schedule described herein. In one embodiment, the dosing schedule is not changed between doses. For example, when the dosing schedule is every three weeks, an additional dose is administered in three weeks. In one embodiment, the dose does not change or is increased for an additional dose (or doses). For example, when a dose of the CDP-paclitaxel conjugate is administered in an amount such that the conjugate includes 135 mg/m² or greater of paclitaxel, an additional dose is administered in an amount such that the conjugate includes 135 mg/m² or greater of paclitaxel.

In one embodiment, the CDP-taxane conjugate is a CDP-larotaxel conjugate, e.g., a CDP-larotaxel conjugate described herein, e.g., a CDP-larotaxel conjugate comprising larotaxel, coupled, e.g., via linkers, to a CDP described herein. In an embodiment, the CDP-larotaxel conjugate comprises larotaxel, coupled via a linker shown in FIG. 2 to a CDP described herein. In an embodiment, the CDP-larotaxel conjugate is a CDP-larotaxel conjugate shown in FIG. 2.

In one embodiment, the CDP-larotaxel conjugate is administered at a dose and/or dosing schedule described herein. In one embodiment, the dosing schedule is not changed between doses. For example, when the dosing schedule is every three weeks, an additional dose is administered in three weeks. In one embodiment, the dose does not change or is increased for an additional dose (or doses).

In one embodiment, the CDP-taxane conjugate is a CDP-cabazitaxel conjugate, e.g., a CDP-cabazitaxel conjugate described herein, e.g., a CDP-cabazitaxel conjugate comprising cabazitaxel, coupled, e.g., via linkers, to a CDP described herein. In an embodiment, the CDP-cabazitaxel conjugate comprises cabazitaxel, coupled via a linker shown in FIG. 2 to a CDP described herein. In an embodiment, the CDP-cabazitaxel conjugate is a CDP-cabazitaxel conjugate shown in FIG. 2.

In one embodiment, the CDP-cabazitaxel conjugate is administered at a dose and/or dosing schedule described herein. In one embodiment, the dosing schedule is not changed between doses. For example, when the dosing schedule is every three weeks, an additional dose is administered in three weeks. In one embodiment, the dose does not change or is increased for an additional dose (or doses).

In one embodiment, the method further comprises administering a CDP-taxane conjugate, e.g., a CDP-taxane conjugate described herein, to the subject.

In one embodiment, the subject experienced moderate to severe neutropenia from treatment with an anticancer agent (e.g., a taxane). In one embodiment, the subject has one or more symptom of febrile neutropenia.

In one embodiment, the cancer is a cancer described herein. In one embodiment, the CDP-taxane conjugate is administered in combination with one or more additional chemotherapeutic agent, e.g., a chemotherapeutic agent or combination of chemotherapeutic agents described herein. In one embodiment, the CDP-taxane conjugate is administered in combination with a granulocyte colony stimulating factor, e.g., GCSF or GMCSF.

In one embodiment, the standard for moderate neutropenia is a neutrophil count of 1000 to 500 cells/mm³. In one embodiment, the standard for severe neutropenia is a neutrophil count of less than 500 cells/mm³.

In an embodiment, the CDP-taxane conjugate complex forms a particle or nanoparticle having a conjugate number described herein. By way of example, a CDP-therapeutic agent conjugate, forms, or is provided in, a particle or nanoparticle having a conjugate number of: 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In an embodiment the conjugate number is 2 to 4 or 2 to 5.

In an embodiment the conjugate number is 1, 2, 3, 4, 5, 6, 7, 8, 9, or 10.

In an embodiment the nanoparticle forms, or is provided in, a preparation of nanoparticles, e.g., a pharmaceutical preparation, wherein at least 40, 50, 60, 70, 80, 90 or 95% of the particles in the preparation have a conjugate number provided herein. In an embodiment the nanoparticle forms, or is provided in, a preparation of nanoparticles, e.g., a pharmaceutical preparation, wherein at least 60% of the particles in the preparation have a conjugate number of 1-5 or 2-5.

In an embodiment, the CDP-taxane conjugate is administered as a nanoparticle or preparation of nanoparticles, e.g., a pharmaceutical preparation, wherein at least 60% of the particles in the preparation have a conjugate number of 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In yet another aspect, the invention features a method for treating a subject, e.g., a human, with a proliferative disorder, e.g., cancer, comprising:

selecting a subject with a proliferative disorder, e.g., cancer, who has moderate to severe neutropenia; and

administering a CDP-taxane conjugate, e.g., a CDP-docetaxel conjugate, a CDP-paclitaxel conjugate, a CDP-larotaxel conjugate and/or a CDP-cabazitaxel conjugate described herein, to the subject in an amount effective to treat the disorder, to thereby treat the proliferative disorder.

In an embodiment, the CDP-taxane conjugate comprises taxane molecules (e.g., docetaxel, paclitaxel, larotaxel and/or cabazitaxel molecules), coupled, e.g., via a linker such as a linker described herein, to a CDP moiety, e.g., a CDP described herein. In an embodiment, the CDP-taxane conjugate comprises a taxane (e.g., docetaxel, paclitaxel, larotaxel and/or cabazitaxel), coupled via a linker shown in FIG. 2 to a CDP moiety, e.g., a CDP described herein. In an embodiment, the CDP-taxane conjugate is a CDP-taxane conjugate shown in FIG. 2.

In one embodiment, the CDP-taxane conjugate is a CDP-docetaxel conjugate, e.g., a CDP-docetaxel conjugate described herein, e.g., a CDP-docetaxel conjugate comprising docetaxel, coupled, e.g., via linkers, to a CDP described herein. In an embodiment, the CDP-docetaxel conjugate comprises docetaxel, coupled via a linker shown in FIG. 2 to a CDP described herein. In an embodiment, the CDP-docetaxel conjugate is a CDP-docetaxel conjugate shown in FIG. 2.

In one embodiment, the CDP-docetaxel conjugate is administered at a dose and/or dosing schedule described herein. In one embodiment, the dosing schedule is not changed between doses. For example, when the dosing schedule is every three weeks, an additional dose is administered in three weeks. In one embodiment, the dose does not change or is increased for an additional dose (or doses). For example, when a dose of the CDP-docetaxel conjugate is administered in an amount such that the conjugate includes 60 mg/m² of docetaxel, an additional dose is administered in an amount such that the conjugate includes 60 mg/m² or greater of docetaxel.

In one embodiment, the CDP-taxane conjugate is a CDP-paclitaxel conjugate, e.g., a CDP-paclitaxel conjugate described herein, e.g., a CDP-paclitaxel conjugate comprising paclitaxel, coupled, e.g., via linkers, to a CDP described herein. In an embodiment, the CDP-paclitaxel conjugate comprises paclitaxel, coupled via a linker shown in FIG. 2 to a CDP described herein. In an embodiment, the CDP-paclitaxel conjugate is a CDP-docetaxel conjugate shown in FIG. 2.

In one embodiment, the CDP-paclitaxel conjugate is administered at a dose and/or dosing schedule described herein. In one embodiment, the dosing schedule is not changed between doses. For example, when the dosing schedule is every three weeks, an additional dose is administered in three weeks. In one embodiment, the dose does not change or is increased for an additional dose (or doses). For example, when a dose of the CDP-paclitaxel conjugate is administered in an amount such that the conjugate includes 135 mg/m² or greater of paclitaxel, an additional dose is administered in an amount such that the conjugate includes 135 mg/m² or greater of paclitaxel.

In one embodiment, the CDP-taxane conjugate is a CDP-larotaxel conjugate, e.g., a CDP-larotaxel conjugate described herein, e.g., a CDP-larotaxel conjugate comprising larotaxel, coupled, e.g., via linkers, to a CDP described herein. In an embodiment, the CDP-larotaxel conjugate comprises larotaxel, coupled via a linker shown in FIG. 2 to a CDP described herein. In an embodiment, the CDP-larotaxel conjugate is a CDP-larotaxel conjugate shown in FIG. 2.

In one embodiment, the CDP-larotaxel conjugate is administered at a dose and/or dosing schedule described herein. In one embodiment, the dosing schedule is not changed between doses. For example, when the dosing schedule is every three weeks, an additional dose is administered in three weeks. In one embodiment, the dose does not change or is increased for an additional dose (or doses).

In one embodiment, the CDP-taxane conjugate is a CDP-cabazitaxel conjugate, e.g., a CDP-cabazitaxel conjugate described herein, e.g., a CDP-cabazitaxel conjugate comprising cabazitaxel, coupled, e.g., via linkers, to a CDP described herein. In an embodiment, the CDP-cabazitaxel conjugate comprises cabazitaxel, coupled via a linker shown in FIG. 2 to a CDP described herein. In an embodiment, the CDP-cabazitaxel conjugate is a CDP-cabazitaxel conjugate shown in FIG. 2.

In one embodiment, the CDP-cabazitaxel conjugate is administered at a dose and/or dosing schedule described herein. In one embodiment, the dosing schedule is not changed between doses. For example, when the dosing schedule is every three weeks, an additional dose is administered in three weeks. In one embodiment, the dose does not change or is increased for an additional dose (or doses).

In one embodiment, the method further comprises administering a CDP-taxane conjugate, e.g., a CDP-taxane conjugate described herein, to the subject.

In one embodiment, the subject experienced moderate to severe neutropenia from treatment with an anticancer agent (e.g., a taxane). In one embodiment, the subject has one or more symptom of febrile neutropenia.

In one embodiment, the cancer is a cancer described herein. In one embodiment, the CDP-taxane conjugate is administered in combination with one or more additional chemotherapeutic agent, e.g., a chemotherapeutic agent or combination of chemotherapeutic agents described herein. In one embodiment, the CDP-taxane conjugate is administered in combination with a granulocyte colony stimulating factor, e.g., GCSF or GMCSF.

In one embodiment, the standard for moderate neutropenia is a neutrophil count of 1000 to 500 cells/mm³. In one embodiment, the standard for severe neutropenia is a neutrophil count of less than 500 cells/mm³.

In an embodiment, the CDP-taxane conjugate complex forms a particle or nanoparticle having a conjugate number described herein. By way of example, a CDP-therapeutic agent conjugate, forms, or is provided in, a particle or nanoparticle having a conjugate number of: 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In an embodiment the conjugate number is 2 to 4 or 2 to 5.

In an embodiment the conjugate number is 1, 2, 3, 4, 5, 6, 7, 8, 9, or 10.

In an embodiment the nanoparticle forms, or is provided in, a preparation of nanoparticles, e.g., a pharmaceutical preparation, wherein at least 40, 50, 60, 70, 80, 90 or 95% of the particles in the preparation have a conjugate number provided herein. In an embodiment the nanoparticle forms, or is provided in, a preparation of nanoparticles, e.g., a pharmaceutical preparation, wherein at least 60% of the particles in the preparation have a conjugate number of 1-5 or 2-5.

In an embodiment, the CDP-taxane conjugate is administered as a nanoparticle or preparation of nanoparticles, e.g., a pharmaceutical preparation, wherein at least 60% of the particles in the preparation have a conjugate number of 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In yet another aspect, the invention features a method for selecting a subject, e.g., a human, with a proliferative disorder, e.g., cancer, for treatment with a CDP-taxane conjugate, e.g., a CDP-docetaxel conjugate, a CDP-paclitaxel conjugate, a CDP-larotaxel conjugate and/or a CDP-cabazitaxel conjugate described herein, comprising:

determining whether a subject with a proliferative disorder, e.g., cancer, has experienced neuropathy from treatment with an anticancer agent, e.g., a taxane, a vinca alkaloid, an alkylating agent, a platinum-based agent, a proteosome inhibitor or an epothilone; and

selecting a subject for treatment with a CDP-taxane conjugate, e.g., a CDP-taxane conjugate described herein, on the basis that the subject has experienced neuropathy from treatment with a chemotherapeutic agent, e.g., a taxane, a vinca alkaloid, an alkylating agent, a platinum-based agent, a proteosome inhibitor or an epothilone.

In an embodiment, the CDP-taxane conjugate comprises taxane molecules (e.g., docetaxel, paclitaxel, larotaxel and/or cabazitaxel molecules), coupled, e.g., via a linker such as a linker described herein, to a CDP moiety, e.g., a CDP described herein. In an embodiment, the CDP-taxane conjugate comprises a taxane (e.g., docetaxel, paclitaxel, larotaxel and/or cabazitaxel), coupled via a linker shown in FIG. 2 to a CDP moiety, e.g., a CDP described herein. In an embodiment, the CDP-taxane conjugate is a CDP-taxane conjugate shown in FIG. 2.

In one embodiment, the CDP-taxane conjugate is a CDP-docetaxel conjugate, e.g., a CDP-docetaxel conjugate described herein, e.g., a CDP-docetaxel conjugate comprising docetaxel, coupled, e.g., via linkers, to a CDP described herein. In an embodiment, the CDP-docetaxel conjugate comprises docetaxel, coupled via a linker shown in FIG. 2 to a CDP described herein. In an embodiment, the CDP-docetaxel conjugate is a CDP-docetaxel conjugate shown in FIG. 2.

In one embodiment, the CDP-docetaxel conjugate is administered at a dose and/or dosing schedule described herein.

In one embodiment, the dosing schedule is not changed between doses. For example, when the dosing schedule is every three weeks, an additional dose is administered in three weeks. In one embodiment, the dose does not change or is increased for an additional dose (or doses). For example, when a dose of the CDP-docetaxel conjugate is administered in an amount such that the conjugate includes 60 mg/m² of docetaxel, an additional dose is administered in an amount such that the conjugate includes 60 mg/m² or greater of docetaxel.

In one embodiment, the CDP-taxane conjugate is a CDP-paclitaxel conjugate, e.g., a CDP-paclitaxel conjugate described herein, e.g., a CDP-paclitaxel conjugate comprising paclitaxel, coupled, e.g., via linkers, to a CDP described herein. In an embodiment, the CDP-paclitaxel conjugate comprises paclitaxel, coupled via a linker shown in FIG. 2 to a CDP described herein. In an embodiment, the CDP-paclitaxel conjugate is a CDP-docetaxel conjugate shown in FIG. 2.

In one embodiment, the CDP-paclitaxel conjugate is administered at a dose and/or dosing schedule described herein. In one embodiment, the dosing schedule is not changed between doses. For example, when the dosing schedule is every three weeks, an additional dose is administered in three weeks. In one embodiment, the dose does not change or is increased for an additional dose (or doses). For example, when a dose of the CDP-paclitaxel conjugate is administered in an amount such that the conjugate includes 135 mg/m² or greater of paclitaxel, an additional dose is administered in an amount such that the conjugate includes 135 mg/m² or greater of paclitaxel.

In one embodiment, the CDP-taxane conjugate is a CDP-larotaxel conjugate, e.g., a CDP-larotaxel conjugate described herein, e.g., a CDP-larotaxel conjugate comprising larotaxel, coupled, e.g., via linkers, to a CDP described herein. In an embodiment, the CDP-larotaxel conjugate comprises larotaxel, coupled via a linker shown in FIG. 2 to a CDP described herein. In an embodiment, the CDP-larotaxel conjugate is a CDP-larotaxel conjugate shown in FIG. 2.

In one embodiment, the CDP-larotaxel conjugate is administered at a dose and/or dosing schedule described herein. In one embodiment, the dosing schedule is not changed between doses. For example, when the dosing schedule is every three weeks, an additional dose is administered in three weeks. In one embodiment, the dose does not change or is increased for an additional dose (or doses).

In one embodiment, the CDP-taxane conjugate is a CDP-cabazitaxel conjugate, e.g., a CDP-cabazitaxel conjugate described herein, e.g., a CDP-cabazitaxel conjugate comprising cabazitaxel, coupled, e.g., via linkers, to a CDP described herein. In an embodiment, the CDP-cabazitaxel conjugate comprises cabazitaxel, coupled via a linker shown in FIG. 2 to a CDP described herein. In an embodiment, the CDP-cabazitaxel conjugate is a CDP-cabazitaxel conjugate shown in FIG. 2.

In one embodiment, the CDP-cabazitaxel conjugate is administered at a dose and/or dosing schedule described herein. In one embodiment, the dosing schedule is not changed between doses. For example, when the dosing schedule is every three weeks, an additional dose is administered in three weeks. In one embodiment, the dose does not change or is increased for an additional dose (or doses).

In one embodiment, the neuropathy is peripheral neuropathy. In one embodiment, the neuropathy is sensory neuropathy, motor neuropathy or both.

In one embodiment, the cancer is a cancer described herein. In one embodiment, the subject is selected for treatment with the CDP-taxane conjugate in combination with one or more additional chemotherapeutic agent, e.g., a chemotherapeutic agent or combination of chemotherapeutic agents described herein. In one embodiment, the CDP-taxane conjugate is administered in combination with a granulocyte colony stimulating factor, e.g., GCSF or GMCSF.

In an embodiment, the CDP-taxane conjugate complex forms a particle or nanoparticle having a conjugate number described herein. By way of example, a CDP-therapeutic agent conjugate, forms, or is provided in, a particle or nanoparticle having a conjugate number of: 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In an embodiment the conjugate number is 2 to 4 or 2 to 5.

In an embodiment the conjugate number is 1, 2, 3, 4, 5, 6, 7, 8, 9, or 10.

In an embodiment the nanoparticle forms, or is provided in, a preparation of nanoparticles, e.g., a pharmaceutical preparation, wherein at least 40, 50, 60, 70, 80, 90 or 95% of the particles in the preparation have a conjugate number provided herein. In an embodiment the nanoparticle forms, or is provided in, a preparation of nanoparticles, e.g., a pharmaceutical preparation, wherein at least 60% of the particles in the preparation have a conjugate number of 1-5 or 2-5.

In an embodiment, the CDP-taxane conjugate is administered as a nanoparticle or preparation of nanoparticles, e.g., a pharmaceutical preparation, wherein at least 60% of the particles in the preparation have a conjugate number of 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In yet another aspect, the invention features a method for treating a subject, e.g., a human, with a proliferative disorder, e.g., cancer, comprising:

selecting a subject with a proliferative disorder, e.g., cancer, who has experienced one or more symptom of neuropathy from treatment with a chemotherapeutic agent, e.g., a taxane (e.g., docetaxel or paclitaxel), a vinca alkaloid, an alkylating agent, a platinum-based agent, a proteosome inhibitor or an epothilone; and

administering a CDP-taxane conjugate, e.g., a CDP-docetaxel conjugate, a CDP-paclitaxel conjugate, a CDP-larotaxel conjugate and/or a CDP-cabazitaxel conjugate described herein, to the subject in an amount effective to treat the disorder, to thereby treat the proliferative disorder.

In an embodiment, the CDP-taxane conjugate comprises taxane molecules (e.g., docetaxel, paclitaxel, larotaxel and/or cabazitaxel molecules), coupled, e.g., via a linker such as a linker described herein, to a CDP moiety, e.g., a CDP described herein. In an embodiment, the CDP-taxane conjugate comprises a taxane (e.g., docetaxel, paclitaxel, larotaxel and/or cabazitaxel), coupled via a linker shown in FIG. 2 to a CDP moiety, e.g., a CDP described herein. In an embodiment, the CDP-taxane conjugate is a CDP-taxane conjugate shown in FIG. 2.

In one embodiment, the CDP-taxane conjugate is a CDP-docetaxel conjugate, e.g., a CDP-docetaxel conjugate described herein, e.g., a CDP-docetaxel conjugate comprising docetaxel, coupled, e.g., via linkers, to a CDP described herein. In an embodiment, the CDP-docetaxel conjugate comprises docetaxel, coupled via a linker shown in FIG. 2 to a CDP described herein. In an embodiment, the CDP-docetaxel conjugate is a CDP-docetaxel conjugate shown in FIG. 2.

In one embodiment, the CDP-docetaxel conjugate is administered at a dose and/or dosing schedule described herein. In one embodiment, the dosing schedule is not changed between doses. For example, when the dosing schedule is every three weeks, an additional dose is administered in three weeks. In one embodiment, the dose does not change or is increased for an additional dose (or doses). For example, when a dose of the CDP-docetaxel conjugate is administered in an amount such that the conjugate includes 60 mg/m² of docetaxel, an additional dose is administered in an amount such that the conjugate includes 60 mg/m² or greater of docetaxel.

In one embodiment, the CDP-taxane conjugate is a CDP-paclitaxel conjugate, e.g., a CDP-paclitaxel conjugate described herein, e.g., a CDP-paclitaxel conjugate comprising paclitaxel, coupled, e.g., via linkers, to a CDP described herein. In an embodiment, the CDP-paclitaxel conjugate comprises paclitaxel, coupled via a linker shown in FIG. 2 to a CDP described herein. In an embodiment, the CDP-paclitaxel conjugate is a CDP-docetaxel conjugate shown in FIG. 2.

In one embodiment, the CDP-paclitaxel conjugate is administered at a dose and/or dosing schedule described herein. In one embodiment, the dosing schedule is not changed between doses. For example, when the dosing schedule is every three weeks, an additional dose is administered in three weeks. In one embodiment, the dose does not change or is increased for an additional dose (or doses). For example, when a dose of the CDP-paclitaxel conjugate is administered in an amount such that the conjugate includes 135 mg/m² or greater of paclitaxel, an additional dose is administered in an amount such that the conjugate includes 135 mg/m² or greater of paclitaxel.

In one embodiment, the CDP-taxane conjugate is a CDP-larotaxel conjugate, e.g., a CDP-larotaxel conjugate described herein, e.g., a CDP-larotaxel conjugate comprising larotaxel, coupled, e.g., via linkers, to a CDP described herein. In an embodiment, the CDP-larotaxel conjugate comprises larotaxel, coupled via a linker shown in FIG. 2 to a CDP described herein. In an embodiment, the CDP-larotaxel conjugate is a CDP-larotaxel conjugate shown in FIG. 2.

In one embodiment, the CDP-larotaxel conjugate is administered at a dose and/or dosing schedule described herein. In one embodiment, the dosing schedule is not changed between doses. For example, when the dosing schedule is every three weeks, an additional dose is administered in three weeks. In one embodiment, the dose does not change or is increased for an additional dose (or doses).

In one embodiment, the CDP-taxane conjugate is a CDP-cabazitaxel conjugate, e.g., a CDP-cabazitaxel conjugate described herein, e.g., a CDP-cabazitaxel conjugate comprising cabazitaxel, coupled, e.g., via linkers, to a CDP described herein. In an embodiment, the CDP-cabazitaxel conjugate comprises cabazitaxel, coupled via a linker shown in FIG. 2 to a CDP described herein. In an embodiment, the CDP-cabazitaxel conjugate is a CDP-cabazitaxel conjugate shown in FIG. 2.

In one embodiment, the CDP-cabazitaxel conjugate is administered at a dose and/or dosing schedule described herein. In one embodiment, the dosing schedule is not changed between doses. For example, when the dosing schedule is every three weeks, an additional dose is administered in three weeks. In one embodiment, the dose does not change or is increased for an additional dose (or doses).

In one embodiment, the subject experienced moderate to severe neuropathy from treatment with a chemotherapeutic agent. In one embodiment, the neuropathy is peripheral neuropathy. In one embodiment, the neuropathy is sensory neuropathy, motor neuropathy or both.

In one embodiment, the subject has experienced neuropathy after two, three, four, five cycles of treatment with an anticancer agent.

In one embodiment, the cancer is a cancer described herein. In one embodiment, the CDP-taxane conjugate is administered in combination with one or more additional chemotherapeutic agent, e.g., a chemotherapeutic agent or combination of chemotherapeutic agents described herein.

In an embodiment, the CDP-taxane conjugate complex forms a particle or nanoparticle having a conjugate number described herein. By way of example, a CDP-therapeutic agent conjugate, forms, or is provided in, a particle or nanoparticle having a conjugate number of: 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In an embodiment the conjugate number is 2 to 4 or 2 to 5.

In an embodiment the conjugate number is 1, 2, 3, 4, 5, 6, 7, 8, 9, or 10.

In an embodiment the nanoparticle forms, or is provided in, a preparation of nanoparticles, e.g., a pharmaceutical preparation, wherein at least 40, 50, 60, 70, 80, 90 or 95% of the particles in the preparation have a conjugate number provided herein. In an embodiment the nanoparticle forms, or is provided in, a preparation of nanoparticles, e.g., a pharmaceutical preparation, wherein at least 60% of the particles in the preparation have a conjugate number of 1-5 or 2-5.

In an embodiment, the CDP-taxane conjugate is administered as a nanoparticle or preparation of nanoparticles, e.g., a pharmaceutical preparation, wherein at least 60% of the particles in the preparation have a conjugate number of 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In another aspect, the invention features a method for selecting a subject, e.g., a human, with a proliferative disorder, e.g., cancer, for treatment with a CDP-taxane conjugate, e.g., a CDP-docetaxel conjugate, a CDP-paclitaxel conjugate, a CDP-larotaxel conjugate and/or a CDP-cabazitaxel conjugate described herein, comprising:

determining whether a subject with a proliferative disorder, e.g., cancer, has experienced an infusion site reaction (e.g., during or within 12 hours of infusion of an anticancer agent (e.g., a taxane, e.g., docetaxel or paclitaxel)) or has or is at risk for having hypersensitivity to treatment with an anticancer agent (e.g., a taxane, e.g., docetaxel or paclitaxel),

selecting a subject for treatment with a CDP-taxane conjugate on the basis that the subject is in need of a reduced infusion site reaction (e.g., reduced as compared to the reaction associated with or caused by the treatment with an anticancer agent (e.g., taxane)) or the subject has or is at risk for having hypersensitivity to treatment with an anticancer agent (e.g., a taxane, e.g., paclitaxel or docetaxel).

In an embodiment, the CDP-taxane conjugate comprises taxane molecules (e.g., docetaxel, paclitaxel, larotaxel and/or cabazitaxel molecules), coupled, e.g., via a linker such as a linker described herein, to a CDP moiety, e.g., a CDP described herein. In an embodiment, the CDP-taxane conjugate comprises a taxane (e.g., docetaxel, paclitaxel, larotaxel and/or cabazitaxel), coupled via a linker shown in FIG. 2 to a CDP moiety, e.g., a CDP described herein. In an embodiment, the CDP-taxane conjugate is a CDP-taxane conjugate shown in FIG. 2.

In one embodiment, the CDP-taxane conjugate is a CDP-docetaxel conjugate, e.g., a CDP-docetaxel conjugate described herein, e.g., a CDP-docetaxel conjugate comprising docetaxel, coupled, e.g., via linkers, to a CDP described herein. In an embodiment, the CDP-docetaxel conjugate comprises docetaxel, coupled via a linker shown in FIG. 2 to a CDP described herein. In an embodiment, the CDP-docetaxel conjugate is a CDP-docetaxel conjugate shown in FIG. 2.

In one embodiment, the CDP-docetaxel conjugate is administered at a dose and/or dosing schedule described herein.

In one embodiment, the CDP-taxane conjugate is a CDP-paclitaxel conjugate, e.g., a CDP-paclitaxel conjugate described herein, e.g., a CDP-paclitaxel conjugate comprising paclitaxel, coupled, e.g., via linkers, to a CDP described herein. In an embodiment, the CDP-paclitaxel conjugate comprises paclitaxel, coupled via a linker shown in FIG. 2 to a CDP described herein. In an embodiment, the CDP-paclitaxel conjugate is a CDP-docetaxel conjugate shown in FIG. 2.

In one embodiment, the CDP-paclitaxel conjugate is administered at a dose and/or dosing schedule described herein. In one embodiment, the dosing schedule is not changed between doses. For example, when the dosing schedule is every three weeks, an additional dose is administered in three weeks. In one embodiment, the dose does not change or is increased for an additional dose (or doses). For example, when a dose of the CDP-paclitaxel conjugate is administered in an amount such that the conjugate includes 135 mg/m² or greater of paclitaxel, an additional dose is administered in an amount such that the conjugate includes 135 mg/m² or greater of paclitaxel.

In one embodiment, the CDP-taxane conjugate is a CDP-larotaxel conjugate, e.g., a CDP-larotaxel conjugate described herein, e.g., a CDP-larotaxel conjugate comprising larotaxel, coupled, e.g., via linkers, to a CDP described herein. In an embodiment, the CDP-larotaxel conjugate comprises larotaxel, coupled via a linker shown in FIG. 2 to a CDP described herein. In an embodiment, the CDP-larotaxel conjugate is a CDP-larotaxel conjugate shown in FIG. 2.

In one embodiment, the CDP-larotaxel conjugate is administered at a dose and/or dosing schedule described herein.

In one embodiment, the CDP-taxane conjugate is a CDP-cabazitaxel conjugate, e.g., a CDP-cabazitaxel conjugate described herein, e.g., a CDP-cabazitaxel conjugate comprising cabazitaxel, coupled, e.g., via linkers, to a CDP described herein. In an embodiment, the CDP-cabazitaxel conjugate comprises cabazitaxel, coupled via a linker shown in FIG. 2 to a CDP described herein. In an embodiment, the CDP-cabazitaxel conjugate is a CDP-cabazitaxel conjugate shown in FIG. 2.

In one embodiment, the CDP-cabazitaxel conjugate is administered at a dose and/or dosing schedule described herein.

In one embodiment, the subject has exhibited one or more symptom of infusion site reaction to a previous treatment with the anticancer agent (e.g., taxane). Symptoms of infusion site reaction include: phlebitis, cellulitis, induration, skin exfoliation, necrosis, fibrosis, hyperpigmentation, inflammation and extravasation.

In one embodiment, the subject has exhibited one or more symptom of hypersensitivity to a previous treatment with the anticancer agent (e.g., the taxane, e.g., a docetaxel or paclitaxel) or to a treatment formulated with Cremaphor and/or polysorbate.

Symptoms hypersensitivity include: dyspnea, hypotension, angioedema, urticaria, bronchospasm and erythema.

In one embodiment, the cancer is a cancer described herein. In one embodiment, the CDP-taxane is selected for administration in combination with one or more additional chemotherapeutic agent, e.g., a chemotherapeutic agent or combination of chemotherapeutic agents described herein.

In one embodiment, the subject is further administered, e.g., prior to administration of the CDP-taxane conjugate, one or more of: an antihistamine (e.g., dexchloropheniramine and diphenhydramine), a steroid (e.g., a corticosteroid (e.g., dexamethasone), and an H₂ antagonist (e.g., ranitidine). In one embodiment, the subject is further administered one or more antiemetic (e.g., a 5HT3 receptor antagonist (dolasetron, granisetron, ondansetron, tropisetron, palonosetron, and mirtazapine), a dopamine antagonist (e.g., domperidone, droperidol, haloperidol, chloropromazine, promethazine, prochlorperazine, metoclopramide, alizapride and prochlorperazine), a NK1 receptor antagonist (e.g., aprepitant and casopitant), a cannabinoid (e.g., cannabis, dronabinol, nabilone and sativex), benzodiazepine (e.g., midazolam and lorazepam), an anticholinergics (e.g., hyoscone) and other antiemetics (e.g., trimethobenzomide, emetrol, propofol and muscimol).

In an embodiment, the CDP-taxane conjugate complex forms a particle or nanoparticle having a conjugate number described herein. By way of example, a CDP-therapeutic agent conjugate, forms, or is provided in, a particle or nanoparticle having a conjugate number of: 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In an embodiment the conjugate number is 2 to 4 or 2 to 5.

In an embodiment the conjugate number is 1, 2, 3, 4, 5, 6, 7, 8, 9, or 10.

In an embodiment the nanoparticle forms, or is provided in, a preparation of nanoparticles, e.g., a pharmaceutical preparation, wherein at least 40, 50, 60, 70, 80, 90 or 95% of the particles in the preparation have a conjugate number provided herein. In an embodiment the nanoparticle forms, or is provided in, a preparation of nanoparticles, e.g., a pharmaceutical preparation, wherein at least 60% of the particles in the preparation have a conjugate number of 1-5 or 2-5.

In an embodiment, the CDP-taxane conjugate is administered as a nanoparticle or preparation of nanoparticles, e.g., a pharmaceutical preparation, wherein at least 60% of the particles in the preparation have a conjugate number of 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In yet another aspect, the invention features a method of treating a subject, e.g., a human, with a proliferative disorder, e.g., cancer, comprising:

selecting a subject with a proliferative disorder, e.g., cancer, who has experienced an infusion site reaction to treatment with an anticancer agent (e.g., a taxane, e.g., a docetaxel or paclitaxel) or has or is at risk for having hypersensitivity to an anticancer agent (e.g., a taxane, e.g., a docetaxel or paclitaxel)); and

administering a CDP-taxane conjugate, e.g., a CDP-docetaxel conjugate, a CDP-paclitaxel conjugate, a CDP-larotaxel conjugate and/or a CDP-cabazitaxel conjugate described herein, to the subject in an amount effective to treat the disorder, to thereby treat the proliferative disorder.

In an embodiment, the CDP-taxane conjugate comprises taxane molecules (e.g., docetaxel, paclitaxel, larotaxel and/or cabazitaxel molecules), coupled, e.g., via a linker such as a linker described herein, to a CDP moiety, e.g., a CDP described herein. In an embodiment, the CDP-taxane conjugate comprises a taxane (e.g., docetaxel, paclitaxel, larotaxel and/or cabazitaxel), coupled via a linker shown in FIG. 2 to a CDP moiety, e.g., a CDP described herein. In an embodiment, the CDP-taxane conjugate is a CDP-taxane conjugate shown in FIG. 2.

In one embodiment, the CDP-taxane conjugate is a CDP-docetaxel conjugate, e.g., a CDP-docetaxel conjugate described herein, e.g., a CDP-docetaxel conjugate comprising docetaxel, coupled, e.g., via linkers, to a CDP described herein. In an embodiment, the CDP-docetaxel conjugate comprises docetaxel, coupled via a linker shown in FIG. 2 to a CDP described herein. In an embodiment, the CDP-docetaxel conjugate is a CDP-docetaxel conjugate shown in FIG. 2.

In one embodiment, the CDP-docetaxel conjugate is administered at a dose and/or dosing schedule described herein.

In one embodiment, the CDP-taxane conjugate is a CDP-paclitaxel conjugate, e.g., a CDP-paclitaxel conjugate described herein, e.g., a CDP-paclitaxel conjugate comprising paclitaxel, coupled, e.g., via linkers, to a CDP described herein. In an embodiment, the CDP-paclitaxel conjugate comprises paclitaxel, coupled via a linker shown in FIG. 2 to a CDP described herein. In an embodiment, the CDP-paclitaxel conjugate is a CDP-paclitaxel conjugate shown in FIG. 2.

In one embodiment, the CDP-paclitaxel conjugate is administered at a dose and/or dosing schedule described herein.

In one embodiment, the CDP-taxane conjugate is a CDP-larotaxel conjugate, e.g., a CDP-larotaxel conjugate described herein, e.g., a CDP-larotaxel conjugate comprising larotaxel, coupled, e.g., via linkers, to a CDP described herein. In an embodiment, the CDP-larotaxel conjugate comprises larotaxel, coupled via a linker shown in FIG. 2 to a CDP described herein. In an embodiment, the CDP-larotaxel conjugate is a CDP-larotaxel conjugate shown in FIG. 2.

In one embodiment, the CDP-larotaxel conjugate is administered at a dose and/or dosing schedule described herein.

In one embodiment, the CDP-taxane conjugate is a CDP-cabazitaxel conjugate, e.g., a CDP-cabazitaxel conjugate described herein, e.g., a CDP-cabazitaxel conjugate comprising cabazitaxel, coupled, e.g., via linkers, to a CDP described herein. In an embodiment, the CDP-cabazitaxel conjugate comprises cabazitaxel, coupled via a linker shown in FIG. 2 to a CDP described herein. In an embodiment, the CDP-cabazitaxel conjugate is a CDP-cabazitaxel conjugate shown in FIG. 2.

In one embodiment, the CDP-cabazitaxel conjugate is administered at a dose and/or dosing schedule described herein.

In one embodiment, the subject has exhibited one or more symptom of infusion site reaction to a previous treatment with the anticancer agent (e.g., taxane, e.g., a docetaxel or paclitaxel). Symptoms of infusion site reaction include: phlebitis, cellulitis, induration, skin exfoliation, necrosis, fibrosis, hyperpigmentation, inflammation and extravasation.

In one embodiment, the subject has exhibited one or more symptom of hypersensitivity to a previous treatment with the anticancer agent (e.g., the taxane) or a treatment formulated with Cremaphor and/or polysorbate. Symptoms hypersensitivity include: dyspnea, hypotension, angioedema, urticaria, bronchospasm and erythema.

In one embodiment, the cancer is a cancer described herein. In one embodiment, the CDP-taxane conjugate is administered in combination with one or more additional chemotherapeutic agent, e.g., a chemotherapeutic agent or combination of chemotherapeutic agents described herein.

In one embodiment, the subject is further administered, e.g., prior to administration of the CDP-taxane conjugate, one or more of: an antihistamine (e.g., dexchloropheniramine and diphenhydramine), a steroid (e.g., a corticosteroid (e.g., dexamethasone), and an H₂ antagonist (e.g., ranitidine). In one embodiment, the subject is further administered one or more antiemetic (e.g., a 5HT3 receptor antagonist (dolasetron, granisetron, ondansetron, tropisetron, palonosetron, and mirtazapine), a dopamine antagonist (e.g., domperidone, droperidol, haloperidol, chloropromazine, promethazine, prochlorperazine, metoclopramide, alizapride and prochlorperazine), a NK1 receptor antagonist (e.g., aprepitant and casopitant), a cannabinoid (e.g., cannabis, dronabinol, nabilone and sativex), benzodiazepine (e.g., midazolam and lorazepam), an anticholinergics (e.g., hyoscone) and other antiemetics (e.g., trimethobenzomide, emetrol, propofol and muscimol).

In an embodiment, the CDP-taxane conjugate complex forms a particle or nanoparticle having a conjugate number described herein. By way of example, a CDP-therapeutic agent conjugate, forms, or is provided in, a particle or nanoparticle having a conjugate number of: 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In an embodiment the conjugate number is 2 to 4 or 2 to 5.

In an embodiment the conjugate number is 1, 2, 3, 4, 5, 6, 7, 8, 9, or 10.

In an embodiment the nanoparticle forms, or is provided in, a preparation of nanoparticles, e.g., a pharmaceutical preparation, wherein at least 40, 50, 60, 70, 80, 90 or 95% of the particles in the preparation have a conjugate number provided herein. In an embodiment the nanoparticle forms, or is provided in, a preparation of nanoparticles, e.g., a pharmaceutical preparation, wherein at least 60% of the particles in the preparation have a conjugate number of 1-5 or 2-5.

In an embodiment, the CDP-taxane conjugate is administered as a nanoparticle or preparation of nanoparticles, e.g., a pharmaceutical preparation, wherein at least 60% of the particles in the preparation have a conjugate number of 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In yet another aspect, the invention features a method of treating a subject, e.g., a human, with a proliferative disorder, e.g., cancer, comprising:

administering a CDP-taxane conjugate, e.g., a CDP-docetaxel conjugate, a CDP-paclitaxel conjugate, a CDP-larotaxel conjugate and/or a CDP-cabazitaxel conjugate described herein, to a subject with a proliferative disorder, e.g., cancer, in an amount effective to treat the disorder and in the absence of administration of one or more of a corticosteroid, an antihistamine, an H1 antagonist, an H2 antagonist and an antiemetic, to thereby treat the proliferative disorder.

In an embodiment, the CDP-taxane conjugate comprises taxane molecules (e.g., docetaxel, paclitaxel, larotaxel and/or cabazitaxel molecules), coupled, e.g., via a linker such as a linker described herein, to a CDP moiety, e.g., a CDP described herein. In an embodiment, the CDP-taxane conjugate comprises a taxane (e.g., docetaxel, paclitaxel, larotaxel and/or cabazitaxel), coupled via a linker shown in FIG. 2 to a CDP moiety, e.g., a CDP described herein. In an embodiment, the CDP-taxane conjugate is a CDP-taxane conjugate shown in FIG. 2.

In one embodiment, the CDP-taxane conjugate is a CDP-docetaxel conjugate, e.g., a CDP-docetaxel conjugate described herein, e.g., a CDP-docetaxel conjugate comprising docetaxel, coupled, e.g., via linkers, to a CDP described herein. In an embodiment, the CDP-docetaxel conjugate comprises docetaxel, coupled via a linker shown in FIG. 2 to a CDP described herein. In an embodiment, the CDP-docetaxel conjugate is a CDP-docetaxel conjugate shown in FIG. 2.

In one embodiment, the CDP-docetaxel conjugate is administered at a dose and/or dosing schedule described herein.

In one embodiment, the CDP-taxane conjugate is a CDP-paclitaxel conjugate, e.g., a CDP-paclitaxel conjugate described herein, e.g., a CDP-paclitaxel conjugate comprising paclitaxel, coupled, e.g., via linkers, to a CDP described herein. In an embodiment, the CDP-paclitaxel conjugate comprises paclitaxel, coupled via a linker shown in FIG. 2 to a CDP described herein. In an embodiment, the CDP-paclitaxel conjugate is a CDP-paclitaxel conjugate shown in FIG. 2.

In one embodiment, the CDP-paclitaxel conjugate is administered at a dose and/or dosing schedule described herein.

In one embodiment, the CDP-taxane conjugate is a CDP-larotaxel conjugate, e.g., a CDP-larotaxel conjugate described herein, e.g., a CDP-larotaxel conjugate comprising larotaxel, coupled, e.g., via linkers, to a CDP described herein. In an embodiment, the CDP-larotaxel conjugate comprises larotaxel, coupled via a linker shown in FIG. 2 to a CDP described herein. In an embodiment, the CDP-larotaxel conjugate is a CDP-larotaxel conjugate shown in FIG. 2.

In one embodiment, the CDP-larotaxel conjugate is administered at a dose and/or dosing schedule described herein.

In one embodiment, the CDP-taxane conjugate is a CDP-cabazitaxel conjugate, e.g., a CDP-cabazitaxel conjugate described herein, e.g., a CDP-cabazitaxel conjugate comprising cabazitaxel, coupled, e.g., via linkers, to a CDP described herein. In an embodiment, the CDP-cabazitaxel conjugate comprises cabazitaxel, coupled via a linker shown in FIG. 2 to a CDP described herein. In an embodiment, the CDP-cabazitaxel conjugate is a CDP-cabazitaxel conjugate shown in FIG. 2.

In one embodiment, the CDP-cabazitaxel conjugate is administered at a dose and/or dosing schedule described herein.

In one embodiment, the CDP-taxane conjugate is administered in the absence of administration of a corticosteroid (e.g., dexamethasone). In one embodiment, the CDP-taxane conjugate is administered in the absence of administration of diphenhydramine and/or dexchloropheniramine. In one embodiment, the CDP-taxane conjugate is administered in the absence of administration of cimetidine and/or ranitidine. In one embodiment, the CDP-taxane conjugate is administered in the absence of an H₂ antagonist (e.g., ranitidine). In one embodiment, the subject is further administered a CSP-taxane conjugate in the absence of an antiemetic (e.g., a 5HT3 receptor antagonist (dolasetron, granisetron, ondansetron, tropisetron, palonosetron, and mirtazapine), a dopamine antagonist (e.g., domperidone, droperidol, haloperidol, chloropromazine, promethazine, prochlorperazine, metoclopramide, alizapride and prochlorperazine), a NK1 receptor antagonist (e.g., aprepitant and casopitant), a cannabinoid (e.g., cannabis, dronabinol, nabilone and sativex), benzodiazepine (e.g., midazolam and lorazepam), an anticholinergics (e.g., hyoscone) or other antiemetics (e.g., trimethobenzomide, emetrol, propofol and muscimol).

In one embodiment, the cancer is a cancer described herein. In one embodiment, the CDP-taxane conjugate is administered in combination with one or more additional chemotherapeutic agent, e.g., a chemotherapeutic agent or combination of chemotherapeutic agents described herein.

In an embodiment, the CDP-taxane conjugate complex forms a particle or nanoparticle having a conjugate number described herein. By way of example, a CDP-therapeutic agent conjugate, forms, or is provided in, a particle or nanoparticle having a conjugate number of: 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In an embodiment the conjugate number is 2 to 4 or 2 to 5.

In an embodiment the conjugate number is 1, 2, 3, 4, 5, 6, 7, 8, 9, or 10.

In an embodiment the nanoparticle forms, or is provided in, a preparation of nanoparticles, e.g., a pharmaceutical preparation, wherein at least 40, 50, 60, 70, 80, 90 or 95% of the particles in the preparation have a conjugate number provided herein. In an embodiment the nanoparticle forms, or is provided in, a preparation of nanoparticles, e.g., a pharmaceutical preparation, wherein at least 60% of the particles in the preparation have a conjugate number of 1-5 or 2-5.

In an embodiment, the CDP-taxane conjugate is administered as a nanoparticle or preparation of nanoparticles, e.g., a pharmaceutical preparation, wherein at least 60% of the particles in the preparation have a conjugate number of 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In yet another aspect, the invention features a method of treating a subject, e.g., a human, with a proliferative disorder, e.g., cancer, comprising:

administering a CDP-taxane conjugate, e.g., a CDP-docetaxel conjugate, a CDP-paclitaxel conjugate, a CDP-larotaxel conjugate and/or a CDP-cabazitaxel conjugate described herein, to a subject with a proliferative disorder, e.g., cancer, in an amount effective to treat the disorder and in combination with a corticosteroid (e.g., dexamethasone), e.g., wherein the corticosteroid (e.g., dexamethasone) is administered at a dose less than 60 mg, 55 mg, 50 mg, 45 mg, 40 mg, 35 mg, 30 mg or the corticosteroid is administered at a dose less than 10 mg, 8 mg, 6 mg or 4 mg, to thereby treat the disorder.

In an embodiment, the CDP-taxane conjugate comprises taxane molecules (e.g., docetaxel, paclitaxel, larotaxel and/or cabazitaxel molecules), coupled, e.g., via a linker such as a linker described herein, to a CDP moiety, e.g., a CDP described herein. In an embodiment, the CDP-taxane conjugate comprises a taxane (e.g., docetaxel, paclitaxel, larotaxel and/or cabazitaxel), coupled via a linker shown in FIG. 2 to a CDP moiety, e.g., a CDP described herein. In an embodiment, the CDP-taxane conjugate is a CDP-taxane conjugate shown in FIG. 2.

In one embodiment, the CDP-taxane conjugate is a CDP-docetaxel conjugate, e.g., a CDP-docetaxel conjugate described herein, e.g., a CDP-docetaxel conjugate comprising docetaxel, coupled, e.g., via linkers, to a CDP described herein. In an embodiment, the CDP-docetaxel conjugate comprises docetaxel, coupled via a linker shown in FIG. 2 to a CDP described herein. In an embodiment, the CDP-docetaxel conjugate is a CDP-docetaxel conjugate shown in FIG. 2.

In one embodiment, the CDP-docetaxel conjugate is administered at a dose and/or dosing schedule described herein.

In one embodiment, the CDP-taxane conjugate is a CDP-paclitaxel conjugate, e.g., a CDP-paclitaxel conjugate described herein, e.g., a CDP-paclitaxel conjugate comprising paclitaxel, coupled, e.g., via linkers, to a CDP described herein. In an embodiment, the CDP-paclitaxel conjugate comprises paclitaxel, coupled via a linker shown in FIG. 2 to a CDP described herein. In an embodiment, the CDP-paclitaxel conjugate is a CDP-docetaxel conjugate shown in FIG. 2.

In one embodiment, the CDP-paclitaxel conjugate is administered at a dose and/or dosing schedule described herein.

In one embodiment, the CDP-taxane conjugate is a CDP-larotaxel conjugate, e.g., a CDP-larotaxel conjugate described herein, e.g., a CDP-larotaxel conjugate comprising larotaxel, coupled, e.g., via linkers, to a CDP described herein. In an embodiment, the CDP-larotaxel conjugate comprises larotaxel, coupled via a linker shown in FIG. 2 to a CDP described herein. In an embodiment, the CDP-larotaxel conjugate is a CDP-larotaxel conjugate shown in FIG. 2.

In one embodiment, the CDP-larotaxel conjugate is administered at a dose and/or dosing schedule described herein.

In one embodiment, the CDP-taxane conjugate is a CDP-cabazitaxel conjugate, e.g., a CDP-cabazitaxel conjugate described herein, e.g., a CDP-cabazitaxel conjugate comprising cabazitaxel, coupled, e.g., via linkers, to a CDP described herein. In an embodiment, the CDP-cabazitaxel conjugate comprises cabazitaxel, coupled via a linker shown in FIG. 2 to a CDP described herein. In an embodiment, the CDP-cabazitaxel conjugate is a CDP-cabazitaxel conjugate shown in FIG. 2.

In one embodiment, the CDP-cabazitaxel conjugate is administered at a dose and/or dosing schedule described herein.

In one embodiment, the cancer is a cancer described herein. In one embodiment, the CDP-taxane is administered in combination with one or more additional chemotherapeutic agent, e.g., a chemotherapeutic agent or combination of chemotherapeutic agents described herein.

In an embodiment, the CDP-taxane conjugate complex forms a particle or nanoparticle having a conjugate number described herein. By way of example, a CDP-therapeutic agent conjugate, forms, or is provided in, a particle or nanoparticle having a conjugate number of: 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In an embodiment the conjugate number is 2 to 4 or 2 to 5.

In an embodiment the conjugate number is 1, 2, 3, 4, 5, 6, 7, 8, 9, or 10.

In an embodiment the nanoparticle forms, or is provided in, a preparation of nanoparticles, e.g., a pharmaceutical preparation, wherein at least 40, 50, 60, 70, 80, 90 or 95% of the particles in the preparation have a conjugate number provided herein. In an embodiment the nanoparticle forms, or is provided in, a preparation of nanoparticles, e.g., a pharmaceutical preparation, wherein at least 60% of the particles in the preparation have a conjugate number of 1-5 or 2-5.

In an embodiment, the CDP-taxane conjugate is administered as a nanoparticle or preparation of nanoparticles, e.g., a pharmaceutical preparation, wherein at least 60% of the particles in the preparation have a conjugate number of 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In yet another aspect, the invention features a method of treating a subject, e.g., a human, with a proliferative disorder, e.g., cancer, comprising:

administering a CDP-taxane conjugate, e.g., a CDP-docetaxel conjugate, a CDP-paclitaxel conjugate, a CDP-larotaxel conjugate and/or a CDP-cabazitaxel conjugate described herein, to a subject with a proliferative disorder, e.g., cancer, in an amount effective to treat the disorder and in combination with an antihistamine, a corticosteroid (e.g., dexamethasone), an antiemetic, an H1 antagonist (e.g., dexachlorapheniramine and/or diphenyhydramine) and/or an H2 antagonist (e.g., cimetidine and/or ranitidine),

wherein the corticosteroid (e.g., dexamethasone) is administered at a dose less than 20 mg, 15 mg, 10 mg, 8 mg, or 5 mg; the H1 antagonist (e.g., diphenyhydramine) is administered at a dose of less than 50 mg, 45 mg, 30 mg, 20 mg, 15 mg, 10 mg, or 5 mg and/or the H1 antagonist (dexachlorapheniramine) is administered at a dose less than 10 mg, 8 mg, 5 mg, or 3 mg; and/or the H2 antagonist (e.g., cimetidine) is administered at a dose of less than 300 mg, 275 mg, 250 mg, 225 mg, 200 mg, 175 mg, 150 mg, 125 mg, 100 mg and/or the H2 antagonist (e.g., ranitidime) is administered at a dose less than 50 mg, 45 mg, 40 mg, 35 mg, 30 mg, 25 mg, 20 mg, to thereby treat the proliferative disorder.

In an embodiment, the CDP-taxane conjugate comprises taxane molecules (e.g., docetaxel, paclitaxel, larotaxel and/or cabazitaxel molecules), coupled, e.g., via a linker such as a linker described herein, to a CDP moiety, e.g., a CDP described herein. In an embodiment, the CDP-taxane conjugate comprises a taxane (e.g., docetaxel, paclitaxel, larotaxel and/or cabazitaxel), coupled via a linker shown in FIG. 2 to a CDP moiety, e.g., a CDP described herein. In an embodiment, the CDP-taxane conjugate is a CDP-taxane conjugate shown in FIG. 2.

In one embodiment, the CDP-taxane conjugate is a CDP-docetaxel conjugate, e.g., a CDP-docetaxel conjugate described herein, e.g., a CDP-docetaxel conjugate comprising docetaxel, coupled, e.g., via linkers, to a CDP described herein. In an embodiment, the CDP-docetaxel conjugate comprises docetaxel, coupled via a linker shown in FIG. 2 to a CDP described herein. In an embodiment, the CDP-docetaxel conjugate is a CDP-docetaxel conjugate shown in FIG. 2.

In one embodiment, the CDP-docetaxel conjugate is administered at a dose and/or dosing schedule described herein.

In one embodiment, the CDP-taxane conjugate is a CDP-paclitaxel conjugate, e.g., a CDP-paclitaxel conjugate described herein, e.g., a CDP-paclitaxel conjugate comprising paclitaxel, coupled, e.g., via linkers, to a CDP described herein. In an embodiment, the CDP-paclitaxel conjugate comprises paclitaxel, coupled via a linker shown in FIG. 2 to a CDP described herein. In an embodiment, the CDP-paclitaxel conjugate is a CDP-paclitaxel conjugate shown in FIG. 2.

In one embodiment, the CDP-paclitaxel conjugate is administered at a dose and/or dosing schedule described herein.

In one embodiment, the CDP-taxane conjugate is a CDP-larotaxel conjugate, e.g., a CDP-larotaxel conjugate described herein, e.g., a CDP-larotaxel conjugate comprising larotaxel, coupled, e.g., via linkers, to a CDP described herein. In an embodiment, the CDP-larotaxel conjugate comprises larotaxel, coupled via a linker shown in FIG. 2 to a CDP described herein. In an embodiment, the CDP-larotaxel conjugate is a CDP-larotaxel conjugate shown in FIG. 2.

In one embodiment, the CDP-larotaxel conjugate is administered at a dose and/or dosing schedule described herein.

In one embodiment, the CDP-taxane conjugate is a CDP-cabazitaxel conjugate, e.g., a CDP-cabazitaxel conjugate described herein, e.g., a CDP-cabazitaxel conjugate comprising cabazitaxel, coupled, e.g., via linkers, to a CDP described herein. In an embodiment, the CDP-cabazitaxel conjugate comprises cabazitaxel, coupled via a linker shown in FIG. 2 to a CDP described herein. In an embodiment, the CDP-cabazitaxel conjugate is a CDP-cabazitaxel conjugate shown in FIG. 2.

In one embodiment, the CDP-cabazitaxel conjugate is administered at a dose and/or dosing schedule described herein.

In one embodiment, the cancer is a cancer described herein. In one embodiment, the CDP-taxane conjugate is administered in combination with one or more additional chemotherapeutic agent, e.g., a chemotherapeutic agent or combination of chemotherapeutic agents described herein.

In an embodiment, the CDP-taxane conjugate complex forms a particle or nanoparticle having a conjugate number described herein. By way of example, a CDP-therapeutic agent conjugate, forms, or is provided in, a particle or nanoparticle having a conjugate number of: 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In an embodiment the conjugate number is 2 to 4 or 2 to 5.

In an embodiment the conjugate number is 1, 2, 3, 4, 5, 6, 7, 8, 9, or 10.

In an embodiment the nanoparticle forms, or is provided in, a preparation of nanoparticles, e.g., a pharmaceutical preparation, wherein at least 40, 50, 60, 70, 80, 90 or 95% of the particles in the preparation have a conjugate number provided herein. In an embodiment the nanoparticle forms, or is provided in, a preparation of nanoparticles, e.g., a pharmaceutical preparation, wherein at least 60% of the particles in the preparation have a conjugate number of 1-5 or 2-5.

In an embodiment, the CDP-taxane conjugate is administered as a nanoparticle or preparation of nanoparticles, e.g., a pharmaceutical preparation, wherein at least 60% of the particles in the preparation have a conjugate number of 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30;

or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In yet another aspect, the invention features a method of selecting a subject, e.g., a human, with a proliferative disorder, e.g., cancer, for treatment with a CDP-taxane conjugate, e.g., a CDP-docetaxel conjugate, a CDP-paclitaxel conjugate, a CDP-larotaxel conjugate and/or a CDP-cabazitaxel conjugate described herein, comprising:

determining if a subject has hepatic impairment, e.g., if the subject has alanine aminotransferase (ALT), aspartate aminotransferase (AST) and/or bilirubin levels in a subject having a proliferative disorder; and selecting a subject having hepatic impairment, e.g., a subject having ALT and/or

AST levels greater than 1.5 times the upper limit of normal (ULN) (e.g., 2.5 times greater than the ULN) and/or bilirubin levels greater than 1.5 or 2 times the ULN for treatment with a CDP-taxane conjugate, e.g., a CDP-taxane conjugate described herein.

In an embodiment, the CDP-taxane conjugate comprises taxane molecules (e.g., docetaxel, paclitaxel, larotaxel and/or cabazitaxel molecules), coupled, e.g., via a linker such as a linker described herein, to a CDP moiety, e.g., a CDP described herein. In an embodiment, the CDP-taxane conjugate comprises a taxane (e.g., docetaxel, paclitaxel, larotaxel and/or cabazitaxel), coupled via a linker shown in FIG. 2 to a CDP moiety, e.g., a CDP described herein. In an embodiment, the CDP-taxane conjugate is a CDP-taxane conjugate shown in FIG. 2.

In one embodiment, the CDP-taxane conjugate is a CDP-docetaxel conjugate, e.g., a CDP-docetaxel conjugate described herein, e.g., a CDP-docetaxel conjugate comprising docetaxel, coupled, e.g., via linkers, to a CDP described herein. In an embodiment, the CDP-docetaxel conjugate comprises docetaxel, coupled via a linker shown in FIG. 2 to a CDP described herein. In an embodiment, the CDP-docetaxel conjugate is a CDP-docetaxel conjugate shown in FIG. 2.

In one embodiment, the CDP-docetaxel conjugate is administered at a dose and/or dosing schedule described herein.

In one embodiment, the CDP-taxane conjugate is a CDP-paclitaxel conjugate, e.g., a CDP-paclitaxel conjugate described herein, e.g., a CDP-paclitaxel conjugate comprising paclitaxel, coupled, e.g., via linkers, to a CDP described herein. In an embodiment, the CDP-paclitaxel conjugate comprises paclitaxel, coupled via a linker shown in FIG. 2 to a CDP described herein. In an embodiment, the CDP-paclitaxel conjugate is a CDP-paclitaxel conjugate shown in FIG. 2.

In one embodiment, the CDP-paclitaxel conjugate is administered at a dose and/or dosing schedule described herein.

In one embodiment, the CDP-taxane conjugate is a CDP-larotaxel conjugate, e.g., a CDP-larotaxel conjugate described herein, e.g., a CDP-larotaxel conjugate comprising larotaxel, coupled, e.g., via linkers, to a CDP described herein. In an embodiment, the CDP-larotaxel conjugate comprises larotaxel, coupled via a linker shown in FIG. 2 to a CDP described herein. In an embodiment, the CDP-larotaxel conjugate is a CDP-larotaxel conjugate shown in FIG. 2.

In one embodiment, the CDP-larotaxel conjugate is administered at a dose and/or dosing schedule described herein.

In one embodiment, the CDP-taxane conjugate is a CDP-cabazitaxel conjugate, e.g., a CDP-cabazitaxel conjugate described herein, e.g., a CDP-cabazitaxel conjugate comprising cabazitaxel, coupled, e.g., via linkers, to a CDP described herein. In an embodiment, the CDP-cabazitaxel conjugate comprises cabazitaxel, coupled via a linker shown in FIG. 2 to a CDP described herein. In an embodiment, the CDP-cabazitaxel conjugate is a CDP-cabazitaxel conjugate shown in FIG. 2.

In one embodiment, the CDP-cabazitaxel conjugate is administered at a dose and/or dosing schedule described herein.

In one embodiment, the cancer is a cancer described herein. In one embodiment, the subject is selected for treatment with the CDP-taxane conjugate in combination with one or more additional chemotherapeutic agent, e.g., a chemotherapeutic agent or combination of chemotherapeutic agents described herein.

In an embodiment, the CDP-taxane conjugate complex forms a particle or nanoparticle having a conjugate number described herein. By way of example, a CDP-therapeutic agent conjugate, forms, or is provided in, a particle or nanoparticle having a conjugate number of: 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In an embodiment the conjugate number is 2 to 4 or 2 to 5.

In an embodiment the conjugate number is 1, 2, 3, 4, 5, 6, 7, 8, 9, or 10.

In an embodiment the nanoparticle forms, or is provided in, a preparation of nanoparticles, e.g., a pharmaceutical preparation, wherein at least 40, 50, 60, 70, 80, 90 or 95% of the particles in the preparation have a conjugate number provided herein. In an embodiment the nanoparticle forms, or is provided in, a preparation of nanoparticles, e.g., a pharmaceutical preparation, wherein at least 60% of the particles in the preparation have a conjugate number of 1-5 or 2-5.

In an embodiment, the CDP-taxane conjugate is administered as a nanoparticle or preparation of nanoparticles, e.g., a pharmaceutical preparation, wherein at least 60% of the particles in the preparation have a conjugate number of 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In yet another aspect, the invention features a method of treating a subject, e.g., a human, having a proliferative disorder, e.g., cancer, comprising:

selecting a subject with a proliferative disorder who has hepatic impairment, e.g., a subject who has alanine aminotransferase (ALT) and/or aspartate aminotransferase (AST) levels greater than 1.5 times the upper limit of normal (ULN) (e.g., 2.5 times the ULN) and/or bilirubin levels greater than 1.5 or 2 times the ULN; and

administering a CDP-taxane conjugate, e.g., a CDP-docetaxel conjugate, a CDP-paclitaxel conjugate, a CDP-larotaxel conjugate and/or a CDP-cabazitaxel conjugate described herein, to the subject in an amount effective to treat the disorder, to thereby treat the proliferative disorder.

In an embodiment, the CDP-taxane conjugate comprises taxane molecules (e.g., docetaxel, paclitaxel, larotaxel and/or cabazitaxel molecules), coupled, e.g., via a linker such as a linker described herein, to a CDP moiety, e.g., a CDP described herein. In an embodiment, the CDP-taxane conjugate comprises a taxane (e.g., docetaxel, paclitaxel, larotaxel and/or cabazitaxel), coupled via a linker shown in FIG. 2 to a CDP moiety, e.g., a CDP described herein. In an embodiment, the CDP-taxane conjugate is a CDP-taxane conjugate shown in FIG. 2.

In one embodiment, the CDP-taxane conjugate is a CDP-docetaxel conjugate, e.g., a CDP-docetaxel conjugate described herein, e.g., a CDP-docetaxel conjugate comprising docetaxel, coupled, e.g., via linkers, to a CDP described herein. In an embodiment, the CDP-docetaxel conjugate comprises docetaxel, coupled via a linker shown in FIG. 2 to a CDP described herein. In an embodiment, the CDP-docetaxel conjugate is a CDP-docetaxel conjugate shown in FIG. 2.

In one embodiment, the CDP-docetaxel conjugate is administered at a dose and/or dosing schedule described herein.

In one embodiment, the CDP-taxane conjugate is a CDP-paclitaxel conjugate, e.g., a CDP-paclitaxel conjugate described herein, e.g., a CDP-paclitaxel conjugate comprising paclitaxel, coupled, e.g., via linkers, to a CDP described herein. In an embodiment, the CDP-paclitaxel conjugate comprises paclitaxel, coupled via a linker shown in FIG. 2 to a CDP described herein. In an embodiment, the CDP-paclitaxel conjugate is a CDP-paclitaxel conjugate shown in FIG. 2.

In one embodiment, the CDP-paclitaxel conjugate is administered at a dose and/or dosing schedule described herein.

In one embodiment, the CDP-taxane conjugate is a CDP-larotaxel conjugate, e.g., a CDP-larotaxel conjugate described herein, e.g., a CDP-larotaxel conjugate comprising larotaxel, coupled, e.g., via linkers, to a CDP described herein. In an embodiment, the CDP-larotaxel conjugate comprises larotaxel, coupled via a linker shown in FIG. 2 to a CDP described herein. In an embodiment, the CDP-larotaxel conjugate is a CDP-larotaxel conjugate shown in FIG. 2.

In one embodiment, the CDP-larotaxel conjugate is administered at a dose and/or dosing schedule described herein.

In one embodiment, the CDP-taxane conjugate is a CDP-cabazitaxel conjugate, e.g., a CDP-cabazitaxel conjugate described herein, e.g., a CDP-cabazitaxel conjugate comprising cabazitaxel, coupled, e.g., via linkers, to a CDP described herein. In an embodiment, the CDP-cabazitaxel conjugate comprises cabazitaxel, coupled via a linker shown in FIG. 2 to a CDP described herein. In an embodiment, the CDP-cabazitaxel conjugate is a CDP-cabazitaxel conjugate shown in FIG. 2.

In one embodiment, the CDP-cabazitaxel conjugate is administered at a dose and/or dosing schedule described herein.

In one embodiment, the cancer is a cancer described herein. In one embodiment, the subject is selected for treatment with the CDP-taxane conjugate in combination with one or more additional chemotherapeutic agent, e.g., a chemotherapeutic agent or combination of chemotherapeutic agents described herein.

In an embodiment, the CDP-taxane conjugate complex forms a particle or nanoparticle having a conjugate number described herein. By way of example, a CDP-therapeutic agent conjugate, forms, or is provided in, a particle or nanoparticle having a conjugate number of: 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In an embodiment the conjugate number is 2 to 4 or 2 to 5.

In an embodiment the conjugate number is 1, 2, 3, 4, 5, 6, 7, 8, 9, or 10.

In an embodiment the nanoparticle forms, or is provided in, a preparation of nanoparticles, e.g., a pharmaceutical preparation, wherein at least 40, 50, 60, 70, 80, 90 or 95% of the particles in the preparation have a conjugate number provided herein. In an embodiment the nanoparticle forms, or is provided in, a preparation of nanoparticles, e.g., a pharmaceutical preparation, wherein at least 60% of the particles in the preparation have a conjugate number of 1-5 or 2-5.

In an embodiment, the CDP-taxane conjugate is administered as a nanoparticle or preparation of nanoparticles, e.g., a pharmaceutical preparation, wherein at least 60% of the particles in the preparation have a conjugate number of 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In yet another aspect, the invention features a method of selecting a subject, e.g., a human, with a proliferative disorder, e.g., cancer, for treatment with a CDP-taxane conjugate, e.g., a CDP-docetaxel conjugate, a CDP-paclitaxel conjugate, a CDP-larotaxel conjugate, and/or a CDP-cabazitaxel conjugate described herein, comprising:

determining if a subject has hepatic impairment, e.g., the subject has alkaline phosphatase (ALP), serum glutamate oxaloacetate transaminase (SGOT), serum glutamate pyruvate transaminase (SGPT) and/or bilirubin levels in a subject having a proliferative disorder; and

selecting a subject having hepatic impairment, e.g., a subject having ALP levels greater than 2.5 times the upper limit of normal (ULN), SGOT and/or SGPT levels greater than 1.5 times the upper limit of normal (ULN) and/or bilirubin levels greater than the ULN for treatment with a CDP-taxane conjugate, e.g., a CDP-taxane conjugate described herein.

In an embodiment, the CDP-taxane conjugate comprises taxane molecules (e.g., docetaxel, paclitaxel, larotaxel and/or cabazitaxel molecules), coupled, e.g., via a linker such as a linker described herein, to a CDP moiety, e.g., a CDP described herein. In an embodiment, the CDP-taxane conjugate comprises a taxane (e.g., docetaxel, paclitaxel, larotaxel and/or cabazitaxel), coupled via a linker shown in FIG. 2 to a CDP moiety, e.g., a CDP described herein. In an embodiment, the CDP-taxane conjugate is a CDP-taxane conjugate shown in FIG. 2.

In one embodiment, the CDP-taxane conjugate is a CDP-docetaxel conjugate, e.g., a CDP-docetaxel conjugate described herein, e.g., a CDP-docetaxel conjugate comprising docetaxel, coupled, e.g., via linkers, to a CDP described herein. In an embodiment, the CDP-docetaxel conjugate comprises docetaxel, coupled via a linker shown in FIG. 2 to a CDP described herein. In an embodiment, the CDP-docetaxel conjugate is a CDP-docetaxel conjugate shown in FIG. 2.

In one embodiment, the CDP-docetaxel conjugate is administered at a dose and/or dosing schedule described herein.

In one embodiment, the CDP-taxane conjugate is a CDP-paclitaxel conjugate, e.g., a CDP-paclitaxel conjugate described herein, e.g., a CDP-paclitaxel conjugate comprising paclitaxel, coupled, e.g., via linkers, to a CDP described herein. In an embodiment, the CDP-paclitaxel conjugate comprises paclitaxel, coupled via a linker shown in FIG. 2 to a CDP described herein. In an embodiment, the CDP-paclitaxel conjugate is a CDP-paclitaxel conjugate shown in FIG. 2.

In one embodiment, the CDP-paclitaxel conjugate is administered at a dose and/or dosing schedule described herein.

In one embodiment, the CDP-taxane conjugate is a CDP-larotaxel conjugate, e.g., a CDP-larotaxel conjugate described herein, e.g., a CDP-larotaxel conjugate comprising larotaxel, coupled, e.g., via linkers, to a CDP described herein. In an embodiment, the CDP-larotaxel conjugate comprises larotaxel, coupled via a linker shown in FIG. 2 to a CDP described herein. In an embodiment, the CDP-larotaxel conjugate is a CDP-larotaxel conjugate shown in FIG. 2.

In one embodiment, the CDP-larotaxel conjugate is administered at a dose and/or dosing schedule described herein.

In one embodiment, the CDP-taxane conjugate is a CDP-cabazitaxel conjugate, e.g., a CDP-cabazitaxel conjugate described herein, e.g., a CDP-cabazitaxel conjugate comprising cabazitaxel, coupled, e.g., via linkers, to a CDP described herein. In an embodiment, the CDP-cabazitaxel conjugate comprises cabazitaxel, coupled via a linker shown in FIG. 2 to a CDP described herein. In an embodiment, the CDP-cabazitaxel conjugate is a CDP-cabazitaxel conjugate shown in FIG. 2.

In one embodiment, the CDP-cabazitaxel conjugate is administered at a dose and/or dosing schedule described herein.

In one embodiment, the cancer is a cancer described herein. In one embodiment, the subject is selected for treatment with the CDP-taxane conjugate in combination with one or more additional chemotherapeutic agent, e.g., a chemotherapeutic agent or combination of chemotherapeutic agents described herein.

In an embodiment, the CDP-taxane conjugate complex forms a particle or nanoparticle having a conjugate number described herein. By way of example, a CDP-therapeutic agent conjugate, forms, or is provided in, a particle or nanoparticle having a conjugate number of: 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In an embodiment the conjugate number is 2 to 4 or 2 to 5.

In an embodiment the conjugate number is 1, 2, 3, 4, 5, 6, 7, 8, 9, or 10.

In an embodiment the nanoparticle forms, or is provided in, a preparation of nanoparticles, e.g., a pharmaceutical preparation, wherein at least 40, 50, 60, 70, 80, 90 or 95% of the particles in the preparation have a conjugate number provided herein. In an embodiment the nanoparticle forms, or is provided in, a preparation of nanoparticles, e.g., a pharmaceutical preparation, wherein at least 60% of the particles in the preparation have a conjugate number of 1-5 or 2-5.

In an embodiment, the CDP-taxane conjugate is administered as a nanoparticle or preparation of nanoparticles, e.g., a pharmaceutical preparation, wherein at least 60% of the particles in the preparation have a conjugate number of 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In yet another aspect, the invention features a method of treating a subject, e.g., a human, having a proliferative disorder, e.g., cancer, comprising:

selecting a subject with a proliferative disorder who has hepatic impairment, e.g., a subject who has alkaline phosphatase (ALP) levels greater than 2.5 times the upper limit of normal (ULN), serum glutamate oxaloacetate transaminase (SGOT) and/or serum glutamate pyruvate transaminase (SGPT) levels greater than 1.5 times the ULN and/or bilirubin levels greater than the ULN; and

administering a CDP-taxane conjugate, e.g., a CDP-docetaxel conjugate, a CDP-paclitaxel conjugate, a CDP-larotaxel conjugate and/or a CDP-cabazitaxel conjugate described herein, to the subject in an amount effective to treat the disorder, to thereby treat the proliferative disorder.

In an embodiment, the CDP-taxane conjugate comprises taxane molecules (e.g., docetaxel, paclitaxel, larotaxel and/or cabazitaxel molecules), coupled, e.g., via a linker such as a linker described herein, to a CDP moiety, e.g., a CDP described herein. In an embodiment, the CDP-taxane conjugate comprises a taxane (e.g., docetaxel, paclitaxel, larotaxel and/or cabazitaxel), coupled via a linker shown in FIG. 2 to a CDP moiety, e.g., a CDP described herein. In an embodiment, the CDP-taxane conjugate is a CDP-taxane conjugate shown in FIG. 2.

In one embodiment, the CDP-taxane conjugate is a CDP-docetaxel conjugate, e.g., a CDP-docetaxel conjugate described herein, e.g., a CDP-docetaxel conjugate comprising docetaxel, coupled, e.g., via linkers, to a CDP described herein. In an embodiment, the CDP-docetaxel conjugate comprises docetaxel, coupled via a linker shown in FIG. 2 to a CDP described herein. In an embodiment, the CDP-docetaxel conjugate is a CDP-docetaxel conjugate shown in FIG. 2.

In one embodiment, the CDP-docetaxel conjugate is administered at a dose and/or dosing schedule described herein.

In one embodiment, the CDP-taxane conjugate is a CDP-paclitaxel conjugate, e.g., a CDP-paclitaxel conjugate described herein, e.g., a CDP-paclitaxel conjugate comprising paclitaxel, coupled, e.g., via linkers, to a CDP described herein. In an embodiment, the CDP-paclitaxel conjugate comprises paclitaxel, coupled via a linker shown in FIG. 2 to a CDP described herein. In an embodiment, the CDP-paclitaxel conjugate is a CDP-paclitaxel conjugate shown in FIG. 2.

In one embodiment, the CDP-paclitaxel conjugate is administered at a dose and/or dosing schedule described herein.

In one embodiment, the CDP-taxane conjugate is a CDP-larotaxel conjugate, e.g., a CDP-larotaxel conjugate described herein, e.g., a CDP-larotaxel conjugate comprising larotaxel, coupled, e.g., via linkers, to a CDP described herein. In an embodiment, the CDP-larotaxel conjugate comprises larotaxel, coupled via a linker shown in FIG. 2 to a CDP described herein. In an embodiment, the CDP-larotaxel conjugate is a CDP-larotaxel conjugate shown in FIG. 2.

In one embodiment, the CDP-larotaxel conjugate is administered at a dose and/or dosing schedule described herein.

In one embodiment, the CDP-taxane conjugate is a CDP-cabazitaxel conjugate, e.g., a CDP-cabazitaxel conjugate described herein, e.g., a CDP-cabazitaxel conjugate comprising cabazitaxel, coupled, e.g., via linkers, to a CDP described herein. In an embodiment, the CDP-cabazitaxel conjugate comprises cabazitaxel, coupled via a linker shown in FIG. 2 to a CDP described herein. In an embodiment, the CDP-cabazitaxel conjugate is a CDP-cabazitaxel conjugate shown in FIG. 2.

In one embodiment, the CDP-cabazitaxel conjugate is administered at a dose and/or dosing schedule described herein.

In one embodiment, the cancer is a cancer described herein. In one embodiment, the subject is selected for treatment with the CDP-taxane conjugate in combination with one or more additional chemotherapeutic agent, e.g., a chemotherapeutic agent or combination of chemotherapeutic agents described herein.

In an embodiment, the CDP-taxane conjugate complex forms a particle or nanoparticle having a conjugate number described herein. By way of example, a CDP-therapeutic agent conjugate, forms, or is provided in, a particle or nanoparticle having a conjugate number of: 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In an embodiment the conjugate number is 2 to 4 or 2 to 5.

In an embodiment the conjugate number is 1, 2, 3, 4, 5, 6, 7, 8, 9, or 10.

In an embodiment the nanoparticle forms, or is provided in, a preparation of nanoparticles, e.g., a pharmaceutical preparation, wherein at least 40, 50, 60, 70, 80, 90 or 95% of the particles in the preparation have a conjugate number provided herein. In an embodiment the nanoparticle forms, or is provided in, a preparation of nanoparticles, e.g., a pharmaceutical preparation, wherein at least 60% of the particles in the preparation have a conjugate number of 1-5 or 2-5.

In an embodiment, the CDP-taxane conjugate is administered as a nanoparticle or preparation of nanoparticles, e.g., a pharmaceutical preparation, wherein at least 60% of the particles in the preparation have a conjugate number of 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In yet another aspect, the invention features a method of selecting a subject, e.g., a human, with a proliferative disorder, e.g., cancer, for treatment with a CDP-taxane conjugate, e.g., a CDP-docetaxel conjugate, a CDP-paclitaxel conjugate, a CDP-larotaxel conjugate and/or a CDP-cabazitaxel conjugate described herein, comprising:

determining if a subject having a proliferative disorder is currently being administered (e.g., the subject has been administered a cytochrome P450 isoenzyme inhibitor, e.g., a CYP3A4 inhibitor or a CYP2C8 inhibitor, the same day as chemotherapy treatment or within 1, 2, 3, 4, 5, 6, or 7 days before chemotherapy treatment) or will be administered (e.g., will be administered on the same day as the chemotherapy treatment or within 1, 2, 3, 4, 5, 6, or 7 days after chemotherapy treatment) a cytochrome P450 isoenzyme inhibitor, e.g., CYP3A4 inhibitor (e.g., ketoconazole, itraconazole, clarithromycin, atazanavir, nefazodone, saquinavir, telithromycin, ritonavir, amprenavir, indinavir, nelfinavir, delavirdine or voriconazole) and/or a CYP2C8 inhibitor (e.g., quercetin); and

selecting a subject with a proliferative disorder, e.g., cancer, who is currently being administered or will be administered a cytochrome P450 isoenzyme, e.g., a CYP3A4 inhibitor and/or a CYP2C8 inhibitor, for treatment with a CDP-taxane conjugate, e.g., a CDP-taxane conjugate described herein, at a dose described herein.

In an embodiment, the CDP-taxane conjugate comprises taxane molecules (e.g., docetaxel, paclitaxel, larotaxel and/or cabazitaxel molecules), coupled, e.g., via a linker such as a linker described herein, to a CDP moiety, e.g., a CDP described herein. In an embodiment, the CDP-taxane conjugate comprises a taxane (e.g., docetaxel, paclitaxel, larotaxel and/or cabazitaxel), coupled via a linker shown in FIG. 2 to a CDP moiety, e.g., a CDP described herein. In an embodiment, the CDP-taxane conjugate is a CDP-taxane conjugate shown in FIG. 2.

In one embodiment, the CDP-taxane conjugate is a CDP-docetaxel conjugate, e.g., a CDP-docetaxel conjugate described herein, e.g., a CDP-docetaxel conjugate comprising docetaxel, coupled, e.g., via linkers, to a CDP described herein. In an embodiment, the CDP-docetaxel conjugate comprises docetaxel, coupled via a linker shown in FIG. 2 to a CDP described herein. In an embodiment, the CDP-docetaxel conjugate is a CDP-docetaxel conjugate shown in FIG. 2.

In one embodiment, the CDP-docetaxel conjugate is administered at a dose and/or dosing schedule described herein.

In one embodiment, the CDP-taxane conjugate is a CDP-larotaxel conjugate, e.g., a CDP-larotaxel conjugate described herein, e.g., a CDP-larotaxel conjugate comprising larotaxel, coupled, e.g., via linkers, to a CDP described herein. In an embodiment, the CDP-larotaxel conjugate comprises larotaxel, coupled via a linker shown in FIG. 2 to a CDP described herein. In an embodiment, the CDP-larotaxel conjugate is a CDP-larotaxel conjugate shown in FIG. 2.

In one embodiment, the CDP-larotaxel conjugate is administered at a dose and/or dosing schedule described herein.

In one embodiment, the CDP-taxane conjugate is a CDP-cabazitaxel conjugate, e.g., a CDP-cabazitaxel conjugate described herein, e.g., a CDP-cabazitaxel conjugate comprising cabazitaxel, coupled, e.g., via linkers, to a CDP described herein. In an embodiment, the CDP-cabazitaxel conjugate comprises cabazitaxel, coupled via a linker shown in FIG. 2 to a CDP described herein. In an embodiment, the CDP-cabazitaxel conjugate is a CDP-cabazitaxel conjugate shown in FIG. 2.

In one embodiment, the CDP-cabazitaxel conjugate is administered at a dose and/or dosing schedule described herein.

In one embodiment, the CDP-taxane conjugate is a CDP-paclitaxel conjugate, e.g., a CDP-paclitaxel conjugate described herein, e.g., a CDP-paclitaxel conjugate comprising paclitaxel, coupled, e.g., via linkers, to a CDP described herein. In an embodiment, the CDP-paclitaxel conjugate comprises paclitaxel, coupled via a linker shown in FIG. 2 to a CDP described herein. In an embodiment, the CDP-paclitaxel conjugate is a CDP-paclitaxel conjugate shown in FIG. 2.

In one embodiment, the CDP-paclitaxel conjugate is administered at a dose and/or dosing schedule described herein.

In one embodiment, the cancer is a cancer described herein. In one embodiment, the CDP-taxane conjugate is administered in combination with one or more additional chemotherapeutic agent, e.g., a chemotherapeutic agent or combination of chemotherapeutic agents described herein.

In an embodiment, the CDP-taxane conjugate complex forms a particle or nanoparticle having a conjugate number described herein. By way of example, a CDP-therapeutic agent conjugate, forms, or is provided in, a particle or nanoparticle having a conjugate number of: 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In an embodiment the conjugate number is 2 to 4 or 2 to 5.

In an embodiment the conjugate number is 1, 2, 3, 4, 5, 6, 7, 8, 9, or 10.

In an embodiment the nanoparticle forms, or is provided in, a preparation of nanoparticles, e.g., a pharmaceutical preparation, wherein at least 40, 50, 60, 70, 80, 90 or 95% of the particles in the preparation have a conjugate number provided herein. In an embodiment the nanoparticle forms, or is provided in, a preparation of nanoparticles, e.g., a pharmaceutical preparation, wherein at least 60% of the particles in the preparation have a conjugate number of 1-5 or 2-5.

In an embodiment, the CDP-taxane conjugate is administered as a nanoparticle or preparation of nanoparticles, e.g., a pharmaceutical preparation, wherein at least 60% of the particles in the preparation have a conjugate number of 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In another aspect, the invention features a method of treating a subject, e.g., a human, having a proliferative disorder, e.g., cancer, comprising:

selecting a subject with a proliferative disorder, e.g., cancer, who is currently being administered or will be, administered a cytochrome P450 isoenzyme, e.g., a CYP3A4 inhibitor and/or a CYP2C8 inhibitor;

administering a CDP-taxane conjugate, e.g., a CDP-docetaxel conjugate, a CDP-paclitaxel conjugate, a CDP-larotaxel conjugate and/or a CDP-cabazitaxel conjugate, described herein, to the subject at a dose described herein, to thereby treat the disorder.

In an embodiment, the CDP-taxane conjugate comprises taxane molecules (e.g., docetaxel, paclitaxel, larotaxel and/or cabazitaxel molecules), coupled, e.g., via a linker such as a linker described herein, to a CDP moiety, e.g., a CDP described herein. In an embodiment, the CDP-taxane conjugate comprises a taxane (e.g., docetaxel, paclitaxel, larotaxel and/or cabazitaxel), coupled via a linker shown in FIG. 2 to a CDP moiety, e.g., a CDP described herein. In an embodiment, the CDP-taxane conjugate is a CDP-taxane conjugate shown in FIG. 2.

In one embodiment, the CDP-taxane conjugate is a CDP-docetaxel conjugate, e.g., a CDP-docetaxel conjugate described herein, e.g., a CDP-docetaxel conjugate comprising docetaxel, coupled, e.g., via linkers, to a CDP described herein. In an embodiment, the CDP-docetaxel conjugate comprises docetaxel, coupled via a linker shown in FIG. 2 to a CDP described herein. In an embodiment, the CDP-docetaxel conjugate is a CDP-docetaxel conjugate shown in FIG. 2.

In one embodiment, the CDP-docetaxel conjugate is administered at a dose and/or dosing schedule described herein.

In one embodiment, the CDP-taxane conjugate is a CDP-larotaxel conjugate, e.g., a CDP-larotaxel conjugate described herein, e.g., a CDP-larotaxel conjugate comprising larotaxel, coupled, e.g., via linkers, to a CDP described herein. In an embodiment, the CDP-larotaxel conjugate comprises larotaxel, coupled via a linker shown in FIG. 2 to a CDP described herein. In an embodiment, the CDP-larotaxel conjugate is a CDP-larotaxel conjugate shown in FIG. 2.

In one embodiment, the CDP-larotaxel conjugate is administered at a dose and/or dosing schedule described herein.

In one embodiment, the CDP-taxane conjugate is a CDP-cabazitaxel conjugate, e.g., a CDP-cabazitaxel conjugate described herein, e.g., a CDP-cabazitaxel conjugate comprising cabazitaxel, coupled, e.g., via linkers, to a CDP described herein. In an embodiment, the CDP-cabazitaxel conjugate comprises cabazitaxel, coupled via a linker shown in FIG. 2 to a CDP described herein. In an embodiment, the CDP-cabazitaxel conjugate is a CDP-cabazitaxel conjugate shown in FIG. 2.

In one embodiment, the CDP-cabazitaxel conjugate is administered at a dose and/or dosing schedule described herein.

In one embodiment, the CDP-taxane conjugate is a CDP-paclitaxel conjugate, e.g., a CDP-paclitaxel conjugate, e.g., a CDP-paclitaxel conjugate comprising paclitaxel, coupled, e.g., via linkers, to a CDP described herein. In an embodiment, the CDP-paclitaxel conjugate comprises paclitaxel, coupled via a linker shown in FIG. 2 to a CDP described herein. In an embodiment, the CDP-paclitaxel conjugate is a CDP-paclitaxel conjugate shown in FIG. 2.

In one embodiment, the CDP-paclitaxel conjugate is administered at a dose and/or dosing schedule described herein.

In one embodiment, the cancer is a cancer described herein. In one embodiment, the CDP-taxane conjugate is administered in combination with one or more additional chemotherapeutic agent, e.g., a chemotherapeutic agent or combination of chemotherapeutic agents described herein.

In an embodiment, the CDP-taxane conjugate complex forms a particle or nanoparticle having a conjugate number described herein. By way of example, a CDP-therapeutic agent conjugate, forms, or is provided in, a particle or nanoparticle having a conjugate number of: 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In an embodiment the conjugate number is 2 to 4 or 2 to 5.

In an embodiment the conjugate number is 1, 2, 3, 4, 5, 6, 7, 8, 9, or 10.

In an embodiment the nanoparticle forms, or is provided in, a preparation of nanoparticles, e.g., a pharmaceutical preparation, wherein at least 40, 50, 60, 70, 80, 90 or 95% of the particles in the preparation have a conjugate number provided herein. In an embodiment the nanoparticle forms, or is provided in, a preparation of nanoparticles, e.g., a pharmaceutical preparation, wherein at least 60% of the particles in the preparation have a conjugate number of 1-5 or 2-5.

In an embodiment, the CDP-taxane conjugate is administered as a nanoparticle or preparation of nanoparticles, e.g., a pharmaceutical preparation, wherein at least 60% of the particles in the preparation have a conjugate number of 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In yet another aspect, the invention features a method of selecting a subject, e.g., a human, with a proliferative disorder, e.g., cancer, for treatment with a CDP-taxane conjugate, e.g., a CDP-docetaxel conjugate, a CDP-paclitaxel conjugate, a CDP-larotaxel conjugate and/or a CDP-cabazitaxel conjugate described herein, comprising:

determining if a subject having a proliferative disorder has or is at risk for having fluid retention and/or effusion and

selecting a subject with a proliferative disorder, e.g., cancer, who has or is at risk for having fluid retention, for treatment with a CDP-taxane conjugate, e.g., a CDP-taxane conjugate described herein, at a dose described herein.

In an embodiment, the CDP-taxane conjugate comprises taxane molecules (e.g., docetaxel, paclitaxel, larotaxel and/or cabazitaxel molecules), coupled, e.g., via a linker such as a linker described herein, to a CDP moiety, e.g., a CDP described herein. In an embodiment, the CDP-taxane conjugate comprises a taxane (e.g., docetaxel, paclitaxel, larotaxel and/or cabazitaxel), coupled via a linker shown in FIG. 2 to a CDP moiety, e.g., a CDP described herein. In an embodiment, the CDP-taxane conjugate is a CDP-taxane conjugate shown in FIG. 2.

In one embodiment, the CDP-taxane conjugate is a CDP-docetaxel conjugate, e.g., a CDP-docetaxel conjugate described herein, e.g., a CDP-docetaxel conjugate comprising docetaxel, coupled, e.g., via linkers, to a CDP described herein. In an embodiment, the CDP-docetaxel conjugate comprises docetaxel, coupled via a linker shown in FIG. 2 to a CDP described herein. In an embodiment, the CDP-docetaxel conjugate is a CDP-docetaxel conjugate shown in FIG. 2.

In one embodiment, the CDP-docetaxel conjugate is administered at a dose and/or dosing schedule described herein.

In one embodiment, the CDP-taxane conjugate is a CDP-larotaxel conjugate, e.g., a CDP-larotaxel conjugate described herein, e.g., a CDP-larotaxel conjugate comprising larotaxel, coupled, e.g., via linkers, to a CDP described herein. In an embodiment, the CDP-larotaxel conjugate comprises larotaxel, coupled via a linker shown in FIG. 2 to a CDP described herein. In an embodiment, the CDP-larotaxel conjugate is a CDP-larotaxel conjugate shown in FIG. 2.

In one embodiment, the CDP-larotaxel conjugate is administered at a dose and/or dosing schedule described herein.

In one embodiment, the CDP-taxane conjugate is a CDP-cabazitaxel conjugate, e.g., a CDP-cabazitaxel conjugate described herein, e.g., a CDP-cabazitaxel conjugate comprising cabazitaxel, coupled, e.g., via linkers, to a CDP described herein. In an embodiment, the CDP-cabazitaxel conjugate comprises cabazitaxel, coupled via a linker shown in FIG. 2 to a CDP described herein. In an embodiment, the CDP-cabazitaxel conjugate is a CDP-cabazitaxel conjugate shown in FIG. 2.

In one embodiment, the CDP-cabazitaxel conjugate is administered at a dose and/or dosing schedule described herein.

In one embodiment, the CDP-taxane conjugate is a CDP-paclitaxel conjugate, e.g., a CDP-paclitaxel conjugate described herein, e.g., a CDP-paclitaxel conjugate comprising paclitaxel, coupled, e.g., via linkers, to a CDP described herein. In an embodiment, the CDP-paclitaxel conjugate comprises paclitaxel, coupled via a linker shown in FIG. 2 to a CDP described herein. In an embodiment, the CDP-paclitaxel conjugate is a CDP-docetaxel conjugate shown in FIG. 2.

In one embodiment, the CDP-paclitaxel conjugate is administered at a dose and/or dosing schedule described herein.

In one embodiment, the subject has one or more of the following symptoms of fluid retention: edema (e.g., peripheral, localized, generalized, lymphedema, pulmonary edema, or unspecified edema) and effusion (e.g., pleural, pericardial and ascites).

In one embodiment, the cancer is a cancer described herein. In one embodiment, the CDP-taxane conjugate is administered in combination with one or more additional chemotherapeutic agent, e.g., a chemotherapeutic agent or combination of chemotherapeutic agents described herein.

In an embodiment, the CDP-taxane conjugate complex forms a particle or nanoparticle having a conjugate number described herein. By way of example, a CDP-therapeutic agent conjugate, forms, or is provided in, a particle or nanoparticle having a conjugate number of: 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In an embodiment the conjugate number is 2 to 4 or 2 to 5.

In an embodiment the conjugate number is 1, 2, 3, 4, 5, 6, 7, 8, 9, or 10.

In an embodiment the nanoparticle forms, or is provided in, a preparation of nanoparticles, e.g., a pharmaceutical preparation, wherein at least 40, 50, 60, 70, 80, 90 or 95% of the particles in the preparation have a conjugate number provided herein. In an embodiment the nanoparticle forms, or is provided in, a preparation of nanoparticles, e.g., a pharmaceutical preparation, wherein at least 60% of the particles in the preparation have a conjugate number of 1-5 or 2-5.

In an embodiment, the CDP-taxane conjugate is administered as a nanoparticle or preparation of nanoparticles, e.g., a pharmaceutical preparation, wherein at least 60% of the particles in the preparation have a conjugate number of 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In another aspect, the invention features a method of treating a subject, e.g., a human, having a proliferative disorder, e.g., cancer, comprising:

selecting a subject with a proliferative disorder, e.g., cancer, who has or is at risk for having fluid retention;

administering a CDP-taxane conjugate, e.g., a CDP-docetaxel conjugate, a CDP-paclitaxel conjugate, a CDP-larotaxel conjugate and/or a CDP-cabazitaxel conjugate, described herein, to the subject at a dose described herein, to thereby treat the disorder.

In an embodiment, the CDP-taxane conjugate comprises taxane molecules (e.g., docetaxel, paclitaxel, larotaxel and/or cabazitaxel molecules), coupled, e.g., via a linker such as a linker described herein, to a CDP moiety, e.g., a CDP described herein. In an embodiment, the CDP-taxane conjugate comprises a taxane (e.g., docetaxel, paclitaxel, larotaxel and/or cabazitaxel), coupled via a linker shown in FIG. 2 to a CDP moiety, e.g., a CDP described herein. In an embodiment, the CDP-taxane conjugate is a CDP-taxane conjugate shown in FIG. 2.

In one embodiment, the CDP-taxane conjugate is a CDP-docetaxel conjugate, e.g., a CDP-docetaxel conjugate described herein, e.g., a CDP-docetaxel conjugate comprising docetaxel, coupled, e.g., via linkers, to a CDP described herein. In an embodiment, the CDP-docetaxel conjugate comprises docetaxel, coupled via a linker shown in FIG. 2 to a CDP described herein. In an embodiment, the CDP-docetaxel conjugate is a CDP-docetaxel conjugate shown in FIG. 2.

In one embodiment, the CDP-docetaxel conjugate is administered at a dose and/or dosing schedule described herein.

In one embodiment, the CDP-taxane conjugate is a CDP-larotaxel conjugate, e.g., a CDP-larotaxel conjugate described herein, e.g., a CDP-larotaxel conjugate comprising larotaxel, coupled, e.g., via linkers, to a CDP described herein. In an embodiment, the CDP-larotaxel conjugate comprises larotaxel, coupled via a linker shown in FIG. 2 to a CDP described herein. In an embodiment, the CDP-larotaxel conjugate is a CDP-larotaxel conjugate shown in FIG. 2.

In one embodiment, the CDP-larotaxel conjugate is administered at a dose and/or dosing schedule described herein.

In one embodiment, the CDP-taxane conjugate is a CDP-cabazitaxel conjugate, e.g., a CDP-cabazitaxel conjugate described herein, e.g., a CDP-cabazitaxel conjugate comprising cabazitaxel, coupled, e.g., via linkers, to a CDP described herein. In an embodiment, the CDP-cabazitaxel conjugate comprises cabazitaxel, coupled via a linker shown in FIG. 2 to a CDP described herein. In an embodiment, the CDP-cabazitaxel conjugate is a CDP-cabazitaxel conjugate shown in FIG. 2.

In one embodiment, the CDP-cabazitaxel conjugate is administered at a dose and/or dosing schedule described herein.

In one embodiment, the CDP-taxane conjugate is a CDP-paclitaxel conjugate, e.g., a CDP-paclitaxel conjugate described herein, e.g., a CDP-paclitaxel conjugate comprising paclitaxel, coupled, e.g., via linkers, to a CDP described herein. In an embodiment, the CDP-paclitaxel conjugate comprises paclitaxel, coupled via a linker shown in FIG. 2 to a CDP described herein. In an embodiment, the CDP-paclitaxel conjugate is a CDP-docetaxel conjugate shown in FIG. 2.

In one embodiment, the CDP-paclitaxel conjugate is administered at a dose and/or dosing schedule described herein.

In one embodiment, the subject has one or more of the following symptoms of fluid retention: edema (e.g., peripheral, localized, generalized, lymphedema, pulmonary edema, or unspecified edema) and effusion (e.g., pleural, pericardial and ascites).

In one embodiment, the cancer is a cancer described herein. In one embodiment, the CDP-taxane conjugate is administered in combination with one or more additional chemotherapeutic agent, e.g., a chemotherapeutic agent or combination of chemotherapeutic agents described herein.

In an embodiment, the CDP-taxane conjugate complex forms a particle or nanoparticle having a conjugate number described herein. By way of example, a CDP-therapeutic agent conjugate, forms, or is provided in, a particle or nanoparticle having a conjugate number of: 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In an embodiment the conjugate number is 2 to 4 or 2 to 5.

In an embodiment the conjugate number is 1, 2, 3, 4, 5, 6, 7, 8, 9, or 10.

In an embodiment the nanoparticle forms, or is provided in, a preparation of nanoparticles, e.g., a pharmaceutical preparation, wherein at least 40, 50, 60, 70, 80, 90 or 95% of the particles in the preparation have a conjugate number provided herein. In an embodiment the nanoparticle forms, or is provided in, a preparation of nanoparticles, e.g., a pharmaceutical preparation, wherein at least 60% of the particles in the preparation have a conjugate number of 1-5 or 2-5.

In an embodiment, the CDP-taxane conjugate is administered as a nanoparticle or preparation of nanoparticles, e.g., a pharmaceutical preparation, wherein at least 60% of the particles in the preparation have a conjugate number of 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In another aspect, the disclosure features a method of selecting a subject, e.g., a human, with a proliferative disorder, e.g., cancer, for treatment treating the subject with a CDP-taxane conjugate, e.g., a CDP-docetaxel conjugate, a CDP-paclitaxel conjugate, a CDP-larotaxel conjugate and/or a CDP-cabazitaxel conjugate, described herein, comprising:

determining if a subject with a proliferative disorder, e.g., a cancer, is at risk for or has a gastrointestinal disorder, e.g., diarrhea, nausea and/or vomiting, or has experienced a gastrointestinal disorder (e.g., diarrhea, nausea and/or vomiting) from treatment with an anticancer agent, e.g., cabazitaxel, and

selecting a subject who is at risk for or has a gastrointestinal disorder (e.g., diarrhea, nausea and/or vomiting) or has experienced a gastrointestinal disorder (e.g., diarrhea, nausea and/or vomiting) from treatment with an anticancer agent (e.g., cabazitaxel) for treatment with a CDP-taxane conjugate, e.g., a CDP-docetaxel conjugate, a CDP-paclitaxel conjugate, a CDP-larotaxel conjugate and/or a CDP-cabazitaxel conjugate, described herein.

In one embodiment, the method further comprises administering a CDP-taxane conjugate to the subject.

In one embodiment, the polymer-anticancer agent conjugate, particle or composition is a CDP-cabazitaxel conjugate, e.g., a CDP-cabazitaxel conjugate described herein, e.g., a CDP-cabazitaxel conjugate comprising cabazitaxel, coupled, e.g., directly or via linkers, to a CDP described herein. In one embodiment, the CDP-cabazitaxel conjugate is administered at a dose and/or dosing schedule described herein.

In one embodiment, the CDP-taxane conjugate described herein e.g., a CDP-docetaxel conjugate, a CDP-paclitaxel conjugate, a CDP-larotaxel conjugate and/or a CDP-cabazitaxel conjugate, described herein, is administered in combination with one or more of: an anti-diarrheal agent and an antiemetic. The anti-diarrheal agent can be, e.g., an opioid (e.g., codeine, oxicodeine, Percocet, paregoric, tincture of opium, diphenoxylate, or diflenoxin), loperamide, bismuth subsalicylate, lanreotide, vapreotide, motilin antagonists, COX2 inhibitors (e.g., celecoxib), glutamine, thalidomide, a kaolin agent, a pectin agent, a berberine agent, a muscarinic agent, octreotide or a DPP-IV inhibitor. The antiemetic can be, e.g., a 5HT3 receptor antagonist (dolasetron, granisetron, ondansetron, tropisetron, palonosetron, and mirtazapine), a dopamine antagonist (e.g., domperidone, droperidol, haloperidol, chloropromazine, promethazine, prochlorperazine, metoclopramide, alizapride and prochlorperazine), a NK1 receptor antagonist (e.g., aprepitant and casopitant), a cannabinoid (e.g., cannabis, dronabinol, nabilone and sativex), benzodiazepine (e.g., midazolam and lorazepam), an anticholinergics (e.g., hyoscone) and other antiemetics (e.g., trimethobenzomide, emetrol, propofol and muscimol).

In an embodiment, the CDP-taxane conjugate complex forms a particle or nanoparticle having a conjugate number described herein. By way of example, a CDP-therapeutic agent conjugate, forms, or is provided in, a particle or nanoparticle having a conjugate number of: 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In an embodiment the conjugate number is 2 to 4 or 2 to 5.

In an embodiment the conjugate number is 1, 2, 3, 4, 5, 6, 7, 8, 9, or 10.

In an embodiment the nanoparticle forms, or is provided in, a preparation of nanoparticles, e.g., a pharmaceutical preparation, wherein at least 40, 50, 60, 70, 80, 90 or 95% of the particles in the preparation have a conjugate number provided herein. In an embodiment the nanoparticle forms, or is provided in, a preparation of nanoparticles, e.g., a pharmaceutical preparation, wherein at least 60% of the particles in the preparation have a conjugate number of 1-5 or 2-5.

In an embodiment, the CDP-taxane conjugate is administered as a nanoparticle or preparation of nanoparticles, e.g., a pharmaceutical preparation, wherein at least 60% of the particles in the preparation have a conjugate number of 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In another aspect, the disclosure features a method of selecting a subject, e.g., a human, with a proliferative disorder, e.g., cancer, for treatment treating the subject with a CDP-taxane conjugate, e.g., a CDP-docetaxel conjugate, a CDP-paclitaxel conjugate, a CDP-larotaxel conjugate and/or a CDP-cabazitaxel conjugate, described herein, comprising:

determining if a subject with a proliferative disorder, e.g., a cancer, is at risk for or has experienced renal failure, e.g., has one or more of sepsis, dehydration and obstructive uropathy, and

selecting a subject who is at risk for or has experienced renal failure for treatment with a CDP-taxane conjugate, e.g., a CDP-docetaxel conjugate, a CDP-paclitaxel conjugate, a CDP-larotaxel conjugate and/or a CDP-cabazitaxel conjugate, described herein.

In one embodiment, the method further comprises administering a CDP-taxane conjugate to the subject.

In one embodiment, the CDP-taxane conjugate is a CDP-cabazitaxel conjugate, e.g., a CDP-cabazitaxel conjugate described herein, e.g., a CDP-cabazitaxel conjugate comprising cabazitaxel, coupled, e.g., directly or via linkers, to a CDP described herein. In one embodiment, the CDP-cabazitaxel conjugate is administered at a dose and/or dosing schedule described herein.

In one embodiment, the CDP-taxane conjugate described herein e.g., a CDP-docetaxel conjugate, a CDP-paclitaxel conjugate, a CDP-larotaxel conjugate and/or a CDP-cabazitaxel conjugate, described herein, is administered in combination with one or more of: an anti-diarrheal agent and an antiemetic. The anti-diarrheal agent can be, e.g., an opioid (e.g., codeine, oxicodeine, Percocet, paregoric, tincture of opium, diphenoxylate, or diflenoxin), loperamide, bismuth subsalicylate, lanreotide, vapreotide, motilin antagonists, COX2 inhibitors (e.g., celecoxib), glutamine, thalidomide, a kaolin agent, a pectin agent, a berberine agent, a muscarinic agent, octreotide or a DPP-IV inhibitor. The antiemetic can be, e.g., one or more of a 5HT3 receptor antagonist (dolasetron, granisetron, ondansetron, tropisetron, palonosetron, and mirtazapine), a dopamine antagonist (e.g., domperidone, droperidol, haloperidol, chloropromazine, promethazine, prochlorperazine, metoclopramide, alizapride and prochlorperazine), a NK1 receptor antagonist (e.g., aprepitant and casopitant), a cannabinoid (e.g., cannabis, dronabinol, nabilone and sativex), benzodiazepine (e.g., midazolam and lorazepam), an anticholinergics (e.g., hyoscone) and other antiemetics (e.g., trimethobenzomide, emetrol, propofol and muscimol).

In an embodiment, the CDP-taxane conjugate complex forms a particle or nanoparticle having a conjugate number described herein. By way of example, a CDP-therapeutic agent conjugate, forms, or is provided in, a particle or nanoparticle having a conjugate number of: 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In an embodiment the conjugate number is 2 to 4 or 2 to 5.

In an embodiment the conjugate number is 1, 2, 3, 4, 5, 6, 7, 8, 9, or 10.

In an embodiment the nanoparticle forms, or is provided in, a preparation of nanoparticles, e.g., a pharmaceutical preparation, wherein at least 40, 50, 60, 70, 80, 90 or 95% of the particles in the preparation have a conjugate number provided herein. In an embodiment the nanoparticle forms, or is provided in, a preparation of nanoparticles, e.g., a pharmaceutical preparation, wherein at least 60% of the particles in the preparation have a conjugate number of 1-5 or 2-5.

In an embodiment, the CDP-taxane conjugate is administered as a nanoparticle or preparation of nanoparticles, e.g., a pharmaceutical preparation, wherein at least 60% of the particles in the preparation have a conjugate number of 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In another aspect, the invention features, a method of evaluating a particle or a preparation of particles, wherein said particles, comprise one or a plurality of CDP therapeutic agent conjugate molecules, e.g., CDP-taxane conjugates, e.g., CDP-docetaxel conjugates, CDP-larotaxel conjugate, or CDP-cabazitaxel conjugates. The method comprises:

providing a sample comprising one or a plurality of said particles;

determining a value for the number of CDP-conjugate molecules in a particle in said sample (the conjugate number),

thereby evaluating a preparation of particles.

In an embodiment the method comprises one or both of:

a) comparing said determined value with a reference value, e.g., a range of values, or

b) responsive to said determination, classifying said particles,

In an embodiment the particle is a nanoparticle.

In an embodiment the method further comprises comparing said determined value with a reference standard. In an embodiment the reference value can be selected from a value, e.g., a range, provided herein, e.g., 1 or 2 to 8, 1 or 2 to 7, 1 or 2 to 6, 1 or 2 to 5, or 2-4.

In an embodiment the conjugate number is from In an embodiment the reference value can be selected from a value, e.g., a range, provided herein, e.g., 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In an embodiment, responsive to said comparison, a decision or step is taken, e.g., a production parameter in a process for making a particle is altered, the sample is classified, selected, accepted or discarded, released or withheld, processed into a drug product, shipped, moved to a different location, formulated, e.g., formulated with another substance, e.g., an excipient, labeled, packaged, released into commerce, or sold or offered for sale.

In an embodiment said CDP-therapeutic agent (e.g., taxane) conjugate is selected from those disclosed in herein.

In an embodiment said therapeutic agent (e.g., taxane) is selected from those disclosed herein.

In an embodiment said particle is selected from those disclosed in herein.

In an embodiment, the determined value for conjugate number is compared with a reference, and responsive to said comparison said particle or preparation of particles is classified, e.g., as suitable for use in human subjects, not suitable for use in human subjects, suitable for sale, meeting a release specification, or not meeting a release specification.

In another aspect, the invention features, a particle, e.g., a nanoparticle, comprising one or more CDP-therapeutic agent (e.g., taxane) conjugates described herein, having a conjuagate number of: 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

The details of one or more embodiments of the invention are set forth in the description below. Other features, objects, and advantages of the invention will be apparent from the description and the drawings, and from the claims.

### BRIEF DESCRIPTION OF THE FIGURES

FIG. 1 depicts a cyclodextrin containing polymer (CDP).

FIG. 2 depicts a table which shows exemplary CDP-taxane conjugates.

FIG. 3 is a schematic representation of a cyclodextrin, using (β)-cyclodextrin as an example.

FIG. 4 depicts a general strategy for synthesizing linear, branched or grafted cyclodextrin-containing polymers (CDPs) for loading a taxane, and an optional targeting ligand.

FIG. 5 depicts a general scheme for graft polymers.

FIG. 6 depicts a general scheme of preparing linear CDPs.

FIG. 7 shows CRLX101 particle size dependence on conjugate number.

FIG. 8 depicts an exemplary synthetic scheme for covalently bonding a derivatized CD to a taxane.

### DETAILED DESCRIPTION OF THE INVENTION

The present invention relates to novel compositions of therapeutic cyclodextrin-containing polymers conjugated to a taxane, particles containing therapeutic cyclodextrin-containing polymers conjugated to a taxane, compositions and mixtures comprising cyclodextran-containing polymers, and methods of use thereof. In certain embodiments, these cyclodextrin-containing polymers improve taxane stability and/or taxane solubility, and/or reduce taxane toxicity, and/or improve efficacy of the taxane when used in vivo.

By selecting from a variety of linker groups used to link a taxane to a CDP, the rate of taxane release from the CDP can be attenuated for controlled delivery. The invention also relates to methods of treating subjects, e.g., humans, with a CDP-taxane conjugate described herein. The invention further relates to methods for conducting a pharmaceutical business comprising manufacturing, licensing, or distributing kits containing or relating to the CDP-taxane conjugates described herein.

More generally, the present invention provides water-soluble, biocompatible polymer conjugates comprising a water-soluble, biocompatible cyclodextrin containing polymer covalently attached to a taxane through attachments that are cleaved under biological conditions to release the taxane.

Polymeric conjugates featured in the present invention may be useful to improve solubility and/or stability of a bioactive/therapeutic agent, such as taxane, reduce drug-drug interactions, reduce interactions with blood elements including plasma proteins, reduce or eliminate immunogenicity, protect the agent from metabolism, modulate drug-release kinetics, improve circulation time, improve drug half-life (e.g., in the serum, or in selected tissues, such as tumors), attenuate toxicity, improve efficacy, normalize drug metabolism across subjects of different species, ethnicities, and/or races, and/or provide for targeted delivery into specific cells or tissues. Poorly soluble and/or toxic compounds may benefit particularly from incorporation into polymeric compounds of the invention.

An “effective amount” or “an amount effective” refers to an amount of the CDP-taxane conjugate which is effective, upon single or multiple dose administrations to a subject, in treating a cell, or curing, alleviating, relieving or improving a symptom of a disorder. An effective amount of the composition may vary according to factors such as the disease state, age, sex, and weight of the individual, and the ability of the compound to elicit a desired response in the individual. An effective amount is also one in which any toxic or detrimental effects of the composition is outweighed by the therapeutically beneficial effects.

“Pharmaceutically acceptable carrier or adjuvant,” as used herein, refers to a carrier or adjuvant that may be administered to a patient, together with a CDP-taxane conjugate described herein, and which does not destroy the pharmacological activity thereof and is nontoxic when administered in doses sufficient to deliver a therapeutic amount of the particle. Some examples of materials which can serve as pharmaceutically acceptable carriers include: (1) sugars, such as lactose, glucose, mannitol and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical compositions.

As used herein the term “low aqueous solubility” refers to water insoluble compounds having poor solubility in water, that is <5 mg/ml at physiological pH (6.5-7.4). Preferably, their water solubility is <1 mg/ml, more preferably <0.1 mg/ml. It is desirable that the drug is stable in water as a dispersion; otherwise a lyophilized or spray-dried solid form may be desirable.

As used herein, the term “prevent” or “preventing” as used in the context of the administration of an agent to a subject, refers to subjecting the subject to a regimen, e.g., the administration of a CDP-taxane conjugate such that the onset of at least one symptom of the disorder is delayed as compared to what would be seen in the absence of the regimen.

As used herein, the term “subject” is intended to include human and non-human animals. Exemplary human subjects include a human patient having a disorder, e.g., a disorder described herein, or a normal subject. The term “non-human animals” includes all vertebrates, e.g., non-mammals (such as chickens, amphibians, reptiles) and mammals, such as non-human primates, domesticated and/or agriculturally useful animals, e.g., sheep, dog, cat, cow, pig, etc.

As used herein, the term “treat” or “treating” a subject having a disorder refers to subjecting the subject to a regimen, e.g., the administration of a CDP-taxane conjugate such that at least one symptom of the disorder is cured, healed, alleviated, relieved, altered, remedied, ameliorated, or improved. Treating includes administering an amount effective to alleviate, relieve, alter, remedy, ameliorate, improve or affect the disorder or the symptoms of the disorder. The treatment may inhibit deterioration or worsening of a symptom of a disorder.

The term “alkenyl” refers to an aliphatic group containing at least one double bond.

The terms “alkoxyl” or “alkoxy” refers to an alkyl group, as defined below, having an oxygen radical attached thereto. Representative alkoxyl groups include methoxy, ethoxy, propyloxy, tert-butoxy and the like. An “ether” is two hydrocarbons covalently linked by an oxygen.

The term “alkyl” refers to the radical of saturated aliphatic groups, including straight-chain alkyl groups, branched-chain alkyl groups, cycloalkyl (alicyclic) groups, alkyl-substituted cycloalkyl groups, and cycloalkyl-substituted alkyl groups. In preferred embodiments, a straight chain or branched chain alkyl has 30 or fewer carbon atoms in its backbone (e.g., C₁-C₃₀ for straight chains, C₃-C₃₀ for branched chains), and more preferably 20 or fewer, and most preferably 10 or fewer Likewise, preferred cycloalkyls have from 3-10 carbon atoms in their ring structure, and more preferably have 5, 6 or 7 carbons in the ring structure.

The term “alkynyl” refers to an aliphatic group containing at least one triple bond.

The term “aralkyl” or “arylalkyl” refers to an alkyl group substituted with an aryl group (e.g., a phenyl or naphthyl).

The term “aryl” includes 5-14 membered single-ring or bicyclic aromatic groups, for example, benzene, naphthalene, and the like. The aromatic ring can be substituted at one or more ring positions with such substituents as described above, for example, halogen, azide, alkyl, aralkyl, alkenyl, alkynyl, cycloalkyl, polycyclyl, hydroxyl, alkoxyl, amino, nitro, sulfhydryl, imino, amido, phosphate, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN, or the like. The term “aryl” also includes polycyclic ring systems having two or more cyclic rings in which two or more carbons are common to two adjoining rings (the rings are “fused rings”) wherein at least one of the rings is aromatic, e.g., the other cyclic rings can be cycloalkyls, cycloalkenyls, cycloalkynyls, aryls and/or heterocyclyls. Each ring can contain, e.g., 5-7 members. The term “arylene” refers to a divalent aryl, as defined herein.

The term “arylalkenyl” refers to an alkenyl group substituted with an aryl group.

The terms “halo” and “halogen” means halogen and includes chloro, fluoro, bromo, and iodo.

The terms “hetaralkyl”, “heteroaralkyl” or “heteroarylalkyl” refers to an alkyl group substituted with a heteroaryl group.

The term “heteroaryl” refers to an aromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively), wherein 0, 1, 2, 3, or 4 atoms of each ring may be substituted by a substituent. Examples of heteroaryl groups include pyridyl, furyl or furanyl, imidazolyl, benzimidazolyl, pyrimidinyl, thiophenyl or thienyl, quinolinyl, indolyl, thiazolyl, and the like. The term “heteroarylene” refers to a divalent heteroaryl, as defined herein.

The term “heteroarylalkenyl” refers to an alkenyl group substituted with a heteroaryl group.

CDP-Taxane Conjugates

Described herein are cyclodextrin containing polymer (“CDP”)-taxane conjugates, wherein one or more taxane are covalently attached to the CDP (e.g., either directly or through a linker). The CDP-taxane conjugates include linear or branched cyclodextrin-containing polymers and polymers grafted with cyclodextrin. Exemplary cyclodextrin-containing polymers that may be modified as described herein are taught in U.S. Pat. Nos. 7,270,808, 6,509,323, 7,091,192, 6,884,789, U.S. Publication Nos. 20040087024, 20040109888 and 20070025952.

Accordingly, in one embodiment the CDP-taxane conjugate is represented by Formula I:

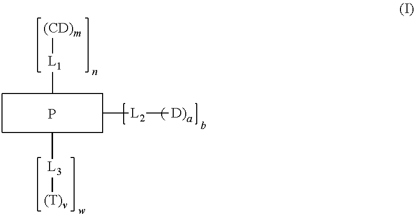

wherein

P represents a linear or branched polymer chain;

CD represents a cyclic moiety such as a cyclodextrin moiety;

L₁, L₂ and L₃, independently for each occurrence, may be absent or represent a linker group;

D, independently for each occurrence, represents a taxane or a prodrug thereof;

T, independently for each occurrence, represents a targeting ligand or precursor thereof;

a, m, and v, independently for each occurrence, represent integers in the range of 1 to 10 (preferably 1 to 8, 1 to 5, or even 1 to 3);

n and w, independently for each occurrence, represent an integer in the range of 0 to about 30,000 (preferably <25,000, <20,000, <15,000, <10,000, <5,000, <1,000, <500, <100, <50, <25, <10, or even <5); and

b represents an integer in the range of 1 to about 30,000 (preferably <25,000, <20,000, <15,000, <10,000, <5,000, <1,000, <500, <100, <50, <25, <10, or even <5),

wherein either P comprises cyclodextrin moieties or n is at least 1.

In some embodiments, one or more of the taxane moieties in the CDP-taxane conjugate can be replaced with another therapeutic agent, e.g., another anticancer agent or anti-inflammatory agent. Examples of other anticancer agents are described herein. Examples of anti-inflammatory agents include a steroid, e.g., prednisone, and a NSAID.

In certain embodiments, P contains a plurality of cyclodextrin moieties within the polymer chain as opposed to the cyclodextrin moieties being grafted on to pendant groups off of the polymeric chain. Thus in certain embodiments, the polymer chain of formula I further comprises n′ units of U, wherein n′ represents an integer in the range of 1 to about 30,000, e.g., from 4-100, 4-50, 4-25, 4-15, 6-100, 6-50, 6-25, and 6-15 (preferably <25,000, <20,000, <15,000, <10,000, <5,000, <1,000, <500, <100, <50, <25, <20, <15, <10, or even <5); and U is represented by one of the general formulae below:

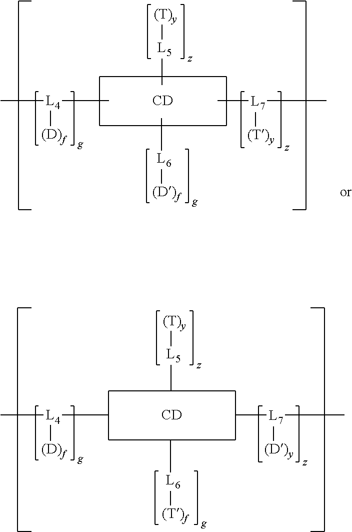

wherein

CD represents a cyclic moiety, such as a cyclodextrin moiety, or derivative thereof;

L₄, L₅, L₆, and L₇, independently for each occurrence, may be absent or represent a linker group;

D and D′, independently for each occurrence, represent the same or different taxane or prodrug forms thereof;

T and T′, independently for each occurrence, represent the same or different targeting ligand or precursor thereof;

f and y, independently for each occurrence, represent an integer in the range of 1 and 10; and

g and z, independently for each occurrence, represent an integer in the range of 0 and 10.

Preferably the polymer has a plurality of D or D′ moieties. In some embodiments, at least 50% of the U units have at least one D or D′. In some embodiments, one or more of the taxane moieties in the CDP-taxane conjugate can be replaced with another therapeutic agent, e.g., another anticancer agent or anti-inflammatory agent.

In preferred embodiments, L₄ and L₇ represent linker groups.

The CDP may include a polycation, polyanion, or non-ionic polymer. A polycationic or polyanionic polymer has at least one site that bears a positive or negative charge, respectively. In certain such embodiments, at least one of the linker moiety and the cyclic moiety comprises such a charged site, so that every occurrence of that moiety includes a charged site. In some embodiments, the CDP is biocompatible.

In certain embodiments, the CDP may include polysaccharides, and other non-protein biocompatible polymers, and combinations thereof, that contain at least one terminal hydroxyl group, such as polyvinylpyrrollidone, poly(oxyethylene)glycol (PEG), polysuccinic anhydride, polysebacic acid, PEG-phosphate, polyglutamate, polyethylenimine, maleic anhydride divinylether (DIVMA), cellulose, pullulans, inulin, polyvinyl alcohol (PVA), N-(2-hydroxypropyl)methacrylamide (HPMA), dextran and hydroxyethyl starch (HES), and have optional pendant groups for grafting therapeutic agents, targeting ligands and/or cyclodextrin moieties. In certain embodiments, the polymer may be biodegradable such as poly(lactic acid), poly(glycolic acid), poly(alkyl 2-cyanoacrylates), polyanhydrides, and polyorthoesters, or bioerodible such as polylactide-glycolide copolymers, and derivatives thereof, non-peptide polyaminoacids, polyiminocarbonates, poly alpha-amino acids, polyalkyl-cyano-acrylate, polyphosphazenes or acyloxymethyl poly aspartate and polyglutamate copolymers and mixtures thereof.

In another embodiment the CDP-taxane conjugate is represented by Formula II:

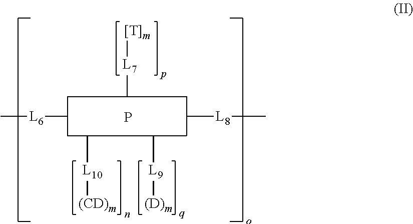

wherein

P represents a monomer unit of a polymer that comprises cyclodextrin moieties; T, independently for each occurrence, represents a targeting ligand or a precursor thereof;

L₆, L₇, L₈, L₉, and L₁₀, independently for each occurrence, may be absent or represent a linker group;

CD, independently for each occurrence, represents a cyclodextrin moiety or a derivative thereof;

D, independently for each occurrence, represents a taxane or a prodrug form thereof;

m, independently for each occurrence, represents an integer in the range of 1 to 10 (preferably 1 to 8, 1 to 5, or even 1 to 3);

o represents an integer in the range of 1 to about 30,000 (preferably <25,000, <20,000, <15,000, <10,000, <5,000, <1,000, <500, <100, <50, <25, <10, or even <5); and

p, n, and q, independently for each occurrence, represent an integer in the range of 0 to 10 (preferably 0 to 8, 0 to 5, 0 to 3, or even 0 to about 2),

wherein CD and D are preferably each present at least 1 location (preferably at least 5, 10, 25, or even 50 or 100 locations) in the compound.

In some embodiments, one or more of the taxane moieties in the CDP-taxane conjugate can be replaced with another therapeutic agent, e.g., another anticancer agent or anti-inflammatory agent. Examples of an anticancer agent are described herein. Examples of anti-inflammatory agents include a steroid, e.g., prednisone, or a NSAID.

In another embodiment the CDP-taxane conjugate is represented either of the formulae below:

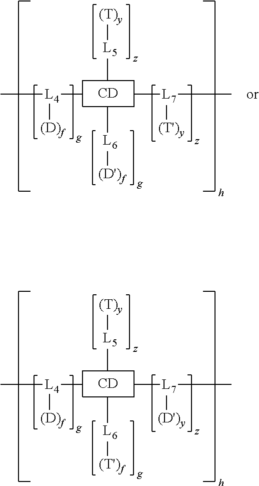

wherein

CD represents a cyclic moiety, such as a cyclodextrin moiety, or derivative thereof;

L₄, L₅, L₆, and L₇, independently for each occurrence, may be absent or represent a linker group;

D and D′, independently for each occurrence, represent the same or different taxane or prodrug thereof;

T and T′, independently for each occurrence, represent the same or different targeting ligand or precursor thereof;

f and y, independently for each occurrence, represent an integer in the range of 1 and 10 (preferably 1 to 8, 1 to 5, or even 1 to 3);

g and z, independently for each occurrence, represent an integer in the range of 0 and 10 (preferably 0 to 8, 0 to 5, 0 to 3, or even 0 to about 2); and

h represents an integer in the range of 1 and 30,000, e.g., from 4-100, 4-50, 4-25, 4-15, 6-100, 6-50, 6-25, and 6-15 (preferably <25,000, <20,000, <15,000, <10,000, <5,000, <1,000, <500, <100, <50, <25, <20, <15, <10, or even <5),

wherein at least one occurrence (and preferably at least 5, 10, or even at least 20, 50, or 100 occurrences) of g represents an integer greater than 0.

Preferably the polymer has a plurality of D or D′ moieties. In some embodiments, at least 50% of the polymer repeating units have at least one D or D′. In some embodiments, one or more of the taxane moieties in the CDP-taxane conjugate can be replaced with another therapeutic agent, e.g., another anticancer agent or anti-inflammatory agent.

In preferred embodiments, L4 and L7 represent linker groups.

In certain such embodiments, the CDP comprises cyclic moieties alternating with linker moieties that connect the cyclic structures, e.g., into linear or branched polymers, preferably linear polymers. The cyclic moieties may be any suitable cyclic structures, such as cyclodextrins, crown ethers (e.g., 18-crown-6,15-crown-5,12-crown-4, etc.), cyclic oligopeptides (e.g., comprising from 5 to 10 amino acid residues), cryptands or cryptates (e.g., cryptand [2.2.2], cryptand-2,1,1, and complexes thereof), calixarenes, or cavitands, or any combination thereof. Preferably, the cyclic structure is (or is modified to be) water-soluble. In certain embodiments, e.g., for the preparation of a linear polymer, the cyclic structure is selected such that under polymerization conditions, exactly two moieties of each cyclic structure are reactive with the linker moieties, such that the resulting polymer comprises (or consists essentially of) an alternating series of cyclic moieties and linker moieties, such as at least four of each type of moiety. Suitable difunctionalized cyclic moieties include many that are commercially available and/or amenable to preparation using published protocols. In certain embodiments, conjugates are soluble in water to a concentration of at least 0.1 g/mL, preferably at least 0.25 g/mL

Thus, in certain embodiments, the invention relates to novel compositions of therapeutic cyclodextrin-containing polymeric compounds designed for drug delivery of a taxane. In certain embodiments, these CDPs improve drug stability and/or solubility, and/or reduce toxicity, and/or improve efficacy of the taxane when used in vivo. Furthermore, by selecting from a variety of linker groups, and/or targeting ligands, the rate of taxane release from the CDP can be attenuated for controlled delivery.

In certain embodiments, the CDP comprises a linear cyclodextrin-containing polymer, e.g., the polymer backbone includes cyclodextrin moieties. For example, the polymer may be a water-soluble, linear cyclodextrin polymer produced by providing at least one cyclodextrin derivative modified to bear one reactive site at each of exactly two positions, and reacting the cyclodextrin derivative with a linker having exactly two reactive moieties capable of forming a covalent bond with the reactive sites under polymerization conditions that promote reaction of the reactive sites with the reactive moieties to form covalent bonds between the linker and the cyclodextrin derivative, whereby a linear polymer comprising alternating units of cyclodextrin derivatives and linkers is produced. Alternatively the polymer may be a water-soluble, linear cyclodextrin polymer having a linear polymer backbone, which polymer comprises a plurality of substituted or unsubstituted cyclodextrin moieties and linker moieties in the linear polymer backbone, wherein each of the cyclodextrin moieties, other than a cyclodextrin moiety at the terminus of a polymer chain, is attached to two of said linker moieties, each linker moiety covalently linking two cyclodextrin moieties. In yet another embodiment, the polymer is a water-soluble, linear cyclodextrin polymer comprising a plurality of cyclodextrin moieties covalently linked together by a plurality of linker moieties, wherein each cyclodextrin moiety, other than a cyclodextrin moiety at the terminus of a polymer chain, is attached to two linker moieties to form a linear cyclodextrin polymer.

Described herein are CDP-taxane conjugates, wherein one or more taxane is covalently attached to the CDP. The CDP can include linear or branched cyclodextrin-containing polymers and/or polymers grafted with cyclodextrin. Exemplary cyclodextrin-containing polymers that may be modified as described herein are taught in U.S. Pat. Nos. 7,270,808, 6,509,323, 7,091,192, 6,884,789, U.S. Publication Nos. 20040087024, 20040109888 and 20070025952, which are incorporated herein by reference in their entirety.

In some embodiments, the CDP-taxane conjugate comprises a water soluble linear polymer conjugate comprising: cyclodextrin moieties; comonomers which do not contain cyclodextrin moieties (comonomers); and a plurality of taxanes; wherein the CDP-taxane conjugate comprises at least four, five six, seven, eight, etc., cyclodextrin moieties and at least four, five six, seven, eight, or more, comonomers. In some embodiments, the taxane is a taxane described herein, for example, the taxane is docetaxel, paclitaxel, larotaxel and/or cabazitaxel. The taxane can be attached to the CDP via a functional group such as a hydroxyl group, or where appropriate, an amino group.

In some embodiments, one or more of the taxane moieties in the CDP-taxane conjugate can be replaced with another therapeutic agent, e.g., another anticancer agent or anti-inflammatory agent.

In some embodiments, the least four cyclodextrin moieties and at least four comonomers alternate in the CDP-taxane conjugate. In some embodiments, said taxanes are cleaved from said CDP-taxane conjugate under biological conditions to release taxane. In some embodiments, the cyclodextrin moieties comprise linkers to which taxanes are linked. In some embodiments, the taxanes are attached via linkers.

In some embodiments, the comonomer comprises residues of at least two functional groups through which reaction and linkage of the cyclodextrin monomers was achieved. In some embodiments, the functional groups, which may be the same or different, terminal or internal, of each comonomer comprise an amino, acid, imidazole, hydroxyl, thio, acyl halide, —HC═CH—, —C≡C— group, or derivative thereof. In some embodiments, the two functional groups are the same and are located at termini of the comonomer precursor. In some embodiments, a comonomer contains one or more pendant groups with at least one functional group through which reaction and thus linkage of a taxane was achieved. In some embodiments, the functional groups, which may be the same or different, terminal or internal, of each comonomer pendant group comprise an amino, acid, imidazole, hydroxyl, thiol, acyl halide, ethylene, ethyne group, or derivative thereof. In some embodiments, the pendant group is a substituted or unsubstituted branched, cyclic or straight chain C₁-C₁₀ alkyl, or arylalkyl optionally containing one or more heteroatoms within the chain or ring. In some embodiments, the cyclodextrin moiety comprises an alpha, beta, or gamma cyclodextrin moiety. In some embodiments, at least about 50% of available positions on the CDP are reacted with a taxane and/or a linker taxane (e.g., at least about 55%, 60%, 65%, 70%, 75%, 80%, 85%, 90%, or 95%). In some embodiments, the taxane is at least 5%, 10%, 15%, 20%, 25%, 30%, or 35% by weight of CDP-taxane conjugate.

In some embodiments, the comonomer comprises polyethylene glycol of molecular weight 3,400 Da, the cyclodextrin moiety comprises beta-cyclodextrin, the theoretical maximum loading of the taxane on the CDP-taxane conjugate is about 25% by weight, and the taxane is about 17-21% by weight of CDP-taxane conjugate. In some embodiments, the taxane is poorly soluble in water. In some embodiments, the solubility of the taxane is <5 mg/ml at physiological pH. In some embodiments, the taxane is a hydrophobic compound with a log P>0.4, >0.6, >0.8, >1, >2, >3, >4, or >5.

In some embodiments, the taxane is attached to the CDP via a second compound.

In some embodiments, administration of the CDP-taxane conjugate to a subject results in release of the taxane over a period of at least 6 hours. In some embodiments, administration of the CDP-taxane conjugate to a subject results in release of the taxane over a period of 2 hours, 3 hours, 5 hours, 6 hours, 8 hours, 10 hours, 15 hours, 20 hours, 1 day, 2 days, 3 days, 4 days, 7 days, 10 days, 14 days, 17 days, 20 days, 24 days, 27 days up to a month. In some embodiments, upon administration of the CDP-taxane conjugate to a subject the rate of taxane release is dependent primarily upon the rate of hydrolysis as opposed to enzymatic cleavage.

In some embodiments, the CDP-taxane conjugate has a molecular weight of 10,000-500,000. In some embodiments, the cyclodextrin moieties make up at least about 2%, 5%, 10%, 20%, 30%, 50% or 80% of the CDP-taxane conjugate by weight.

In some embodiments, the CDP-taxane conjugate is made by a method comprising providing cyclodextrin moiety precursors modified to bear one reactive site at each of exactly two positions, and reacting the cyclodextrin moiety precursors with comonomer precursors having exactly two reactive moieties capable of forming a covalent bond with the reactive sites under polymerization conditions that promote reaction of the reactive sites with the reactive moieties to form covalent bonds between the comonomers and the cyclodextrin moieties, whereby a CDP comprising alternating units of a cyclodextrin moiety and a comonomer is produced. In some embodiments, the cyclodextrin moiety precursors are in a composition, the composition being substantially free of cyclodextrin moieties having other than two positions modified to bear a reactive site (e.g., cyclodextrin moieties having 1, 3, 4, 5, 6, or 7 positions modified to bear a reactive site).

In some embodiments, a comonomer of the CDP-taxane conjugate comprises a moiety selected from the group consisting of: an alkylene chain, polysuccinic anhydride, poly-L-glutamic acid, poly(ethyleneimine), an oligosaccharide, and an amino acid chain. In some embodiments, a CDP-taxane conjugate comonomer comprises a polyethylene glycol chain. In some embodiments, a comonomer comprises a moiety selected from: polyglycolic acid and polylactic acid chain. In some embodiments, a comonomer comprises a hydrocarbylene group wherein one or more methylene groups is optionally replaced by a group Y (provided that none of the Y groups are adjacent to each other), wherein each Y, independently for each occurrence, is selected from, substituted or unsubstituted aryl, heteroaryl, cycloalkyl, heterocycloalkyl, or —O—, C(═X) (wherein X is NR₁, O or S), —OC(O)—, —C(═O)O, —NR₁—, —NR₁CO—, —C(O)NR₁—, —S(O)ₙ— (wherein n is 0, 1, or 2), —OC(O)—NR₁, —NR₁—C(O)—NR₁—, —NR₁1-C(NR₁)—NR₁—, and —B(OR₁)—; and R₁, independently for each occurrence, represents H or a lower alkyl.

In some embodiments, the CDP-taxane conjugate is a polymer having attached thereto a plurality of D moieties of the following formula:

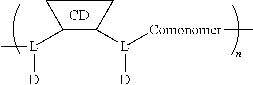

wherein each L is independently a linker, and each D is independently a taxane, a prodrug derivative thereof, or absent; and each comonomer is independently a comonomer described herein, and n is at least 4, 5, 6, 7, 8, 9, 10, 11, 12, 13, 14, 15, 16, 17, 18, 19 or 20, provided that the polymer comprises at least one taxane and in some embodiments, at least two taxane moieties. In some embodiments, the molecular weight of the comonomer is from about 2000 to about 5000 Da (e.g., from about 2000 to about 4500, from about 3000 to about 4000 Da, or less than about 4000, (e.g., about 3400 Da)).

In some embodiments, the taxane is a taxane described herein, for example, the taxane is docetaxel, paclitaxel, larotaxel or cabazitaxel. The taxane can be attached to the CDP via a functional group such as a hydroxyl group, or where appropriate, an amino group. In some embodiments, one or more of the taxane moieties in the CDP-taxane conjugate can be replaced with another therapeutic agent, e.g., another anticancer agent or anti-inflammatory agent.

In some embodiments, the CDP-taxane conjugate is a polymer having attached thereto a plurality of D moieties of the following formula:

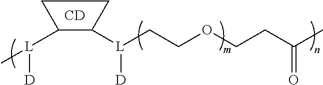

wherein each L is independently a linker, and each D is independently a taxane, a prodrug derivative thereof, or absent, provided that the polymer comprises at least one taxane and in some embodiments, at least two taxane moieties (e.g., at least 3, 4, 5, 6, 7, 8, 9, 10, 11, 12, 13, 14, 15, 16, 17, 18, 19, 20 or more); and

wherein the group

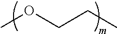

has a Mw of 4.0 kDa or less, e.g., 3.2 to 3.8 kDa, e.g., 3.4 kDa and n is at least 4, 5, 6, 7, 8, 9, 10, 11, 12, 13, 14, 15, 16, 17, 18, 19 or 20.

In some embodiments, the taxane is a taxane described herein, for example, the taxane is docetaxel, paclitaxel, larotaxel or cabazitaxel. The taxane can be attached to the CDP via a functional group such as a hydroxyl group, or where appropriate, an amino group. In some embodiments, one or more of the taxane moieties in the CDP-taxane conjugate can be replaced with another therapeutic agent, e.g., another anticancer agent or anti-inflammatory agent.

In some embodiments, less than all of the L moieties are attached to D moieties, meaning in some embodiments, at least one D is absent. In some embodiments, the loading of the D moieties on the CDP-taxane conjugate is from about 1 to about 50% (e.g., from about 1 to about 25%, from about 5 to about 20% or from about 5 to about 15%). In some embodiments, each L independently comprises an amino acid or a derivative thereof. In some embodiments, each L independently comprises a plurality of amino acids or derivatives thereof. In some embodiments, each L is independently a dipeptide or derivative thereof.

In some embodiments, the CDP-taxane conjugate is a polymer having attached thereto a plurality of L-D moieties of the following formula:

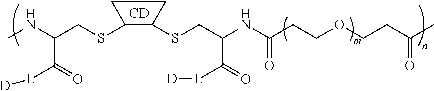

wherein each L is independently a linker or absent and each D is independently a taxane, a prodrug derivative thereof, or absent and wherein the group

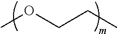

has a Mw of 4.0 kDa or less, e.g., 3.2 to 3.8 kDa, e.g., 3.4 kDa and n is at least 4, 5, 6, 7, 8, 9, 10, 11, 12, 13, 14, 15, 16, 17, 18, 19 or 20, provided that the polymer comprises at least one taxane and in some embodiments, at least two taxane moieties (e.g., at least 3, 4, 5, 6, 7, 8, 9, 10, 11, 12, 13, 14, 15, 16, 17, 18, 19, 20 or more).

In some embodiments, the taxane is a taxane described herein, for example, the taxane is docetaxel, paclitaxel, larotaxel or cabazitaxel.

In some embodiments, less than all of the C(═O) moieties are attached to L-D moieties, meaning in some embodiments, at least one L and/or D is absent. In some embodiments, the loading of the L, D and/or L-D moieties on the CDP-taxane conjugate is from about 1 to about 50% (e.g., from about 1 to about 25%, from about 5 to about 20% or from about 5 to about 15%). In some embodiments, each L is independently an amino acid or derivative thereof. In some embodiments, each L is glycine or a derivative thereof.

In some embodiments, one or more of the taxane moieties in the CDP-taxane conjugate can be replaced with another therapeutic agent, e.g., another anticancer agent or anti-inflammatory agent.

In some embodiments, the CDP-taxane conjugate is a polymer having the following formula:

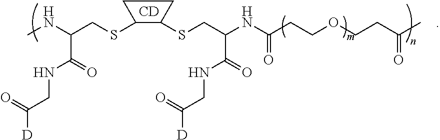

In some embodiments, less than all of the C(═O) moieties are attached to

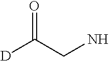

moieties, meaning in some embodiments,

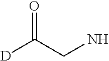

is absent, provided that the polymer comprises at least one taxane and in some embodiments, at least two taxane moieties (e.g., at least 3, 4, 5, 6, 7, 8, 9, 10, 11, 12, 13, 14, 15, 16, 17, 18, 19, 20 or more). In some embodiments, the loading of the

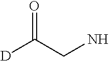

moieties on the CDP-taxane conjugate is from about 1 to about 50% (e.g., from about 1 to about 25%, from about 5 to about 25% or from about 15 to about 15%).

In some embodiments, the taxane is a taxane described herein, for example, the taxane is docetaxel, paclitaxel, larotaxel or cabazitaxel.

In some embodiments, one or more of the taxane moieties in the CDP-taxane conjugate can be replaced with another therapeutic agent, e.g., another anticancer agent or anti-inflammatory agent.

In some embodiments, the CDP-taxane conjugate will contain a taxane and at least one additional therapeutic agent. For instance, a taxane and one more different cancer drugs, an immunosuppressant, an antibiotic or an anti-inflammatory agent may be grafted on to the polymer via optional linkers. By selecting different linkers for different drugs, the release of each drug may be attenuated to achieve maximal dosage and efficacy.

Cyclodextrins

In certain embodiments, the cyclodextrin moieties make up at least about 2%, 5% or 10% by weight, up to 20%, 30%, 50% or even 80% of the CDP by weight. In certain embodiments, the taxanes, or targeting ligands make up at least about 1%, 5%, 10% or 15%, 20%, 25%, 30% or even 35% of the CDP by weight. Number-average molecular weight (Mₙ) may also vary widely, but generally fall in the range of about 1,000 to about 500,000 daltons, preferably from about 5000 to about 200,000 daltons and, even more preferably, from about 10,000 to about 100,000. Most preferably, M. varies between about 12,000 and 65,000 daltons. In certain other embodiments, M. varies between about 3000 and 150,000 daltons. Within a given sample of a subject polymer, a wide range of molecular weights may be present. For example, molecules within the sample may have molecular weights that differ by a factor of 2, 5, 10, 20, 50, 100, or more, or that differ from the average molecular weight by a factor of 2, 5, 10, 20, 50, 100, or more. Exemplary cyclodextrin moieties include cyclic structures consisting essentially of from 7 to 9 saccharide moieties, such as cyclodextrin and oxidized cyclodextrin. A cyclodextrin moiety optionally comprises a linker moiety that forms a covalent linkage between the cyclic structure and the polymer backbone, preferably having from 1 to 20 atoms in the chain, such as alkyl chains, including dicarboxylic acid derivatives (such as glutaric acid derivatives, succinic acid derivatives, and the like), and heteroalkyl chains, such as oligoethylene glycol chains.

Cyclodextrins are cyclic polysaccharides containing naturally occurring D-(+)-glucopyranose units in an α-(1,4) linkage. The most common cyclodextrins are alpha ((α)-cyclodextrins, beta (β)-cyclodextrins and gamma (γ)-cyclodextrins which contain, respectively six, seven, or eight glucopyranose units. Structurally, the cyclic nature of a cyclodextrin forms a torus or donut-like shape having an inner apolar or hydrophobic cavity, the secondary hydroxyl groups situated on one side of the cyclodextrin torus and the primary hydroxyl groups situated on the other. Thus, using (β)-cyclodextrin as an example, a cyclodextrin is often represented schematically as follows.

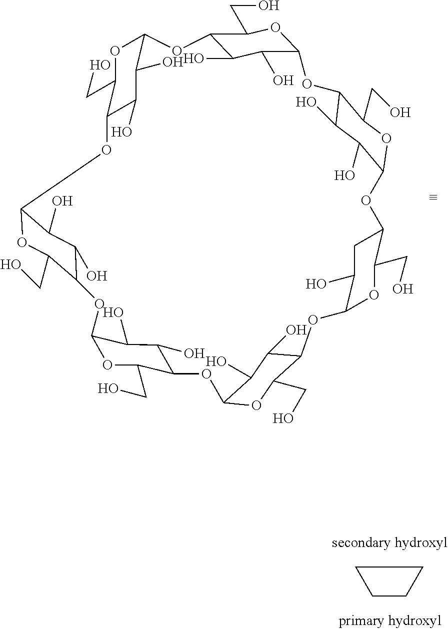

The side on which the secondary hydroxyl groups are located has a wider diameter than the side on which the primary hydroxyl groups are located. The present invention contemplates covalent linkages to cyclodextrin moieties on the primary and/or secondary hydroxyl groups. The hydrophobic nature of the cyclodextrin inner cavity allows for host-guest inclusion complexes of a variety of compounds, e.g., adamantane. (Comprehensive Supramolecular Chemistry, Volume 3, J. L. Atwood et al., eds., Pergamon Press (1996); T. Cserhati, Analytical Biochemistry, 225:328-332 (1995); Husain et al., Applied Spectroscopy, 46:652-658 (1992); FR 2 665 169). Additional methods for modifying polymers are disclosed in Suh, J. and Noh, Y., Bioorg. Med. Chem. Lett. 1998, 8, 1327-1330.

In certain embodiments, the compounds comprise cyclodextrin moieties and wherein at least one or a plurality of the cyclodextrin moieties of the CDP-taxane conjugate is oxidized. In certain embodiments, the cyclodextrin moieties of P alternate with linker moieties in the polymer chain.

### Comonomers

In addition to a cyclodextrin moiety, the CDP can also include a comonomer, for example, a comonomer described herein. In some embodiments, a comonomer of the CDP-taxane conjugate comprises a moiety selected from the group consisting of: an alkylene chain, polysuccinic anhydride, poly-L-glutamic acid, poly(ethyleneimine), an oligosaccharide, and an amino acid chain. In some embodiments, a CDP-taxane conjugate comonomer comprises a polyethylene glycol chain. In some embodiments, a comonomer comprises a moiety selected from: polyglycolic acid and polylactic acid chain. In some embodiments, a comonomer comprises a hydrocarbylene group wherein one or more methylene groups is optionally replaced by a group Y (provided that none of the Y groups are adjacent to each other), wherein each Y, independently for each occurrence, is selected from, substituted or unsubstituted aryl, heteroaryl, cycloalkyl, heterocycloalkyl, or —O—, C(═X) (wherein X is NR₁, O or S), —OC(O)—, —C(═O)O, —NR₁—, —NR₁CO—, —C(O)NR₁—, —S(O)ₙ— (wherein n is 0, 1, or 2), —OC(O)—NR₁, —NR₁—C(O)—NR₁—, —NR₁1-C(NR₁)—NR₁—, and —B(OR₁)—; and R₁, independently for each occurrence, represents H or a lower alkyl.

In some embodiments, a comonomer can be and/or can comprise a linker such as a linker described herein.

### Linkers/Tethers

The CDPs described herein can include one or more linkers. In some embodiments, a linker, such as a linker described herein, can link a cyclodextrin moiety to a comonomer. In some embodiments, a linker can link a taxane to a CDP. In some embodiments, for example, when referring to a linker that links a taxane to the CDP, the linker can be referred to as a tether.

In certain embodiments, a plurality of the linker moieties are attached to a taxane or prodrug thereof and are cleaved under biological conditions.

Described herein are CDP-taxane conjugates that comprise a CDP covalently attached to taxanes through attachments that are cleaved under biological conditions to release the taxane. In certain embodiments, a CDP-taxane conjugate comprises a taxane covalently attached to a polymer, preferably a biocompatible polymer, through a tether, e.g., a linker, wherein the tether comprises a selectivity-determining moiety and a self-cyclizing moiety which are covalently attached to one another in the tether, e.g., between the polymer and the taxane.

In some embodiments, such taxanes are covalently attached to CDPs through functional groups comprising one or more heteroatoms, for example, hydroxy, thiol, carboxy, amino, and amide groups. Such groups may be covalently attached to the subject polymers through linker groups as described herein, for example, biocleavable linker groups, and/or through tethers, such as a tether comprising a selectivity-determining moiety and a self-cyclizing moiety which are covalently attached to one another.

In certain embodiments, the CDP-taxane conjugate comprises a taxane covalently attached to the CDP through a tether, wherein the tether comprises a self-cyclizing moiety. In some embodiments, the tether further comprises a selectivity-determining moiety. Thus, one aspect of the invention relates to a polymer conjugate comprising a therapeutic agent covalently attached to a polymer, preferably a biocompatible polymer, through a tether, wherein the tether comprises a selectivity-determining moiety and a self-cyclizing moiety which are covalently attached to one another.

In some embodiments, the selectivity-determining moiety is bonded to the self-cyclizing moiety between the self-cyclizing moiety and the CDP.

In certain embodiments, the selectivity-determining moiety is a moiety that promotes selectivity in the cleavage of the bond between the selectivity-determining moiety and the self-cyclizing moiety. Such a moiety may, for example, promote enzymatic cleavage between the selectivity-determining moiety and the self-cyclizing moiety. Alternatively, such a moiety may promote cleavage between the selectivity-determining moiety and the self-cyclizing moiety under acidic conditions or basic conditions.

In certain embodiments, the invention contemplates any combination of the foregoing. Those skilled in the art will recognize that, for example, any CDP of the invention in combination with any linker (e.g., a linker described herein such as a self-cyclizing moiety, any selectivity-determining moiety, and/or any taxane) are within the scope of the invention.

In certain embodiments, the selectivity-determining moiety is selected such that the bond is cleaved under acidic conditions.

In certain embodiments where the selectivity-determining moiety is selected such that the bond is cleaved under basic conditions, the selectivity-determining moiety is an aminoalkylcarbonyloxyalkyl moiety. In certain embodiments, the selectivity-determining moiety has a structure

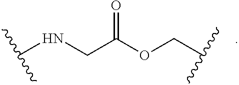 [SMILES: *COC(=O)CN*]

In certain embodiments where the selectivity-determining moiety is selected such that the bond is cleaved enzymatically, it may be selected such that a particular enzyme or class of enzymes cleaves the bond. In certain preferred such embodiments, the selectivity-determining moiety may be selected such that the bond is cleaved by a cathepsin, preferably cathepsin B.

In certain embodiments the selectivity-determining moiety comprises a peptide, preferably a dipeptide, tripeptide, or tetrapeptide. In certain such embodiments, the peptide is a dipeptide is selected from KF and FK, In certain embodiments, the peptide is a tripeptide is selected from GFA, GLA, AVA, GVA, GIA, GVL, GVF, and AVF. In certain embodiments, the peptide is a tetrapeptide selected from GFYA and GFLG, preferably GFLG.

In certain such embodiments, a peptide, such as GFLG, is selected such that the bond between the selectivity-determining moiety and the self-cyclizing moiety is cleaved by a cathepsin, preferably cathepsin B.

In certain embodiments, the selectivity-determining moiety is represented by Formula A:

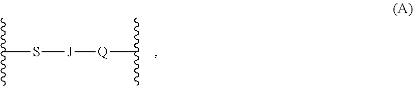

wherein
S a sulfur atom that is part of a disulfide bond;
J is optionally substituted hydrocarbyl; and
Q is O or NR¹³, wherein R¹³ is hydrogen or alkyl.

In certain embodiments, J may be polyethylene glycol, polyethylene, polyester, alkenyl, or alkyl. In certain embodiments, J may represent a hydrocarbylene group comprising one or more methylene groups, wherein one or more methylene groups is optionally replaced by a group Y (provided that none of the Y groups are adjacent to each other), wherein each Y, independently for each occurrence, is selected from, substituted or unsubstituted aryl, heteroaryl, cycloalkyl, heterocycloalkyl, or —O—, C(═X) (wherein X is NR³⁰, O or S), —OC(O)—, —C(═O)O, —NR³⁰—, —NR₁CO—, —C(O)NR³⁰—, —S(O)ₙ— (wherein n is 0, 1, or 2), —OC(O)—NR³⁰, —NR³⁰—C(O)—NR³⁰—, —NR³⁰—C(NR³⁰)—NR³⁰—, and —B(OR³⁰)—; and R³⁰, independently for each occurrence, represents H or a lower alkyl. In certain embodiments, J may be substituted or unsubstituted lower alkylene, such as ethylene. For example, the selectivity-determining moiety may be

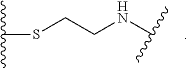 [SMILES: *NCCS*]

In certain embodiments, the selectivity-determining moiety is represented by Formula B:

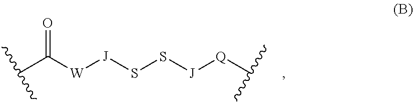

wherein
W is either a direct bond or selected from lower alkyl, NR¹⁴, S, O;
S is sulfur;
J, independently and for each occurrence, is hydrocarbyl or polyethylene glycol;
Q is O or NR¹³, wherein R¹³ is hydrogen or alkyl; and
R¹⁴ is selected from hydrogen and alkyl.

In certain such embodiments, J may be substituted or unsubstituted lower alkyl, such as methylene. In certain such embodiments, J may be an aryl ring. In certain embodiments, the aryl ring is a benzo ring. In certain embodiments W and S are in a 1,2-relationship on the aryl ring. In certain embodiments, the aryl ring may be optionally substituted with alkyl, alkenyl, alkoxy, aralkyl, aryl, heteroaryl, halogen, —CN, azido, —NR^{x}R^{x}, —CO₂OR^{x}, —C(O)—NR^{x}R^{x}, —C(O)—R^{x}, —NR^{x}—C(O)—R^{x}, —NR^{x}SO₂R^{x}, —SR^{X}, —S(O)R^{x}, —SO₂R^{x}, —SO₂NR^{x}R^{x}, —(C(R^{x})₂)ₙOR^{x}, —(C(R^{x})₂)ₙ—NR^{x}R^{x}, and —(C(R^{x})₂)ₙ—SO₂R^{x}; wherein R^{x} is, independently for each occurrence, H or lower alkyl; and n is, independently for each occurrence, an integer from 0 to 2.

In certain embodiments, the aryl ring is optionally substituted with alkyl, alkenyl, alkoxy, aralkyl, aryl, heteroaryl, halogen, —CN, azido, —NR^{x}R^{x}, —CO₂OR^{x}, —C(O)—NR^{x}R^{x}, —C(O)—R^{x}, —NR^{x}—C(O)—R^{x}, —NR^{x}SO₂R^{x}, —SR^{X}, —S(O)R^{X}, —SO₂R^{x}, —SO₂NR^{x}R^{x}, —(C(R^{x})₂)ₙ—OR^{x}, —(C(R^{x})₂)ₙ—NR^{x}R^{x}, and —(C(R^{x})₂)ₙ—SO₂R^{x}; wherein R^{x} is, independently for each occurrence, H or lower alkyl; and n is, independently for each occurrence, an integer from 0 to 2.

In certain embodiments, J, independently and for each occurrence, is polyethylene glycol, polyethylene, polyester, alkenyl, or alkyl.

In certain embodiments, independently and for each occurrence, the linker comprises a hydrocarbylene group comprising one or more methylene groups, wherein one or more methylene groups is optionally replaced by a group Y (provided that none of the Y groups are adjacent to each other), wherein each Y, independently for each occurrence, is selected from, substituted or unsubstituted aryl, heteroaryl, cycloalkyl, heterocycloalkyl, or —O—, C(═X) (wherein X is NR³⁰, O or S), —OC(O)—, —C(═O)O, —NR³⁰—, —NR₁CO—, —C(O)NR³⁰—, —S(O)ₙ— (wherein n is 0, 1, or 2), —OC(O)—NR³⁰ , —NR³⁰—C(O)—NR³⁰—, —NR³⁰—C—NR³⁰—, and —B(OR³⁰)—; and R³⁰, independently for each occurrence, represents H or a lower alkyl.

In certain embodiments, J, independently and for each occurrence, is substituted or unsubstituted lower alkylene. In certain embodiments, J, independently and for each occurrence, is substituted or unsubstituted ethylene.

In certain embodiments, the selectivity-determining moiety is selected from

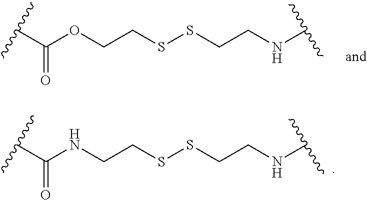 [SMILES: *NCCSSCCOC(*)=O ; *NCCSSCCNC(*)=O]

The selectivity-determining moiety may include groups with bonds that are cleavable under certain conditions, such as disulfide groups. In certain embodiments, the selectivity-determining moiety comprises a disulfide-containing moiety, for example, comprising aryl and/or alkyl group(s) bonded to a disulfide group. In certain embodiments, the selectivity-determining moiety has a structure

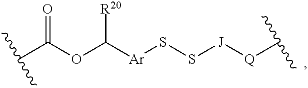

wherein
Ar is a substituted or unsubstituted benzo ring;
J is optionally substituted hydrocarbyl; and

### Q is O or NR¹³,

wherein R¹³ is hydrogen or alkyl.

In certain embodiments, Ar is unsubstituted. In certain embodiments, Ar is a 1,2-benzo ring. For example, suitable moieties within Formula B include

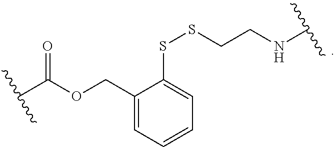 [SMILES: *NCCSSc1ccccc1COC(*)=O]

In certain embodiments, the self-cyclizing moiety is selected such that upon cleavage of the bond between the selectivity-determining moiety and the self-cyclizing moiety, cyclization occurs thereby releasing the therapeutic agent. Such a cleavage-cyclization-release cascade may occur sequentially in discrete steps or substantially simultaneously. Thus, in certain embodiments, there may be a temporal and/or spatial difference between the cleavage and the self-cyclization. The rate of the self-cyclization cascade may depend on pH, e.g., a basic pH may increase the rate of self-cyclization after cleavage. Self-cyclization may have a half-life after introduction in vivo of 24 hours, 18 hours, 14 hours, 10 hours, 6 hours, 3 hours, 2 hours, 1 hour, 30 minutes, 10 minutes, 5 minutes, or 1 minute.

In certain such embodiments, the self-cyclizing moiety may be selected such that, upon cyclization, a five- or six-membered ring is formed, preferably a five-membered ring. In certain such embodiments, the five- or six-membered ring comprises at least one heteroatom selected from oxygen, nitrogen, or sulfur, preferably at least two, wherein the heteroatoms may be the same or different. In certain such embodiments, the heterocyclic ring contains at least one nitrogen, preferably two. In certain such embodiments, the self-cyclizing moiety cyclizes to form an imidazolidone.

In certain embodiments, the self-cyclizing moiety has a structure

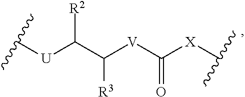

wherein
U is selected from NR¹ and S;
X is selected from O, NR⁵, and S, preferably O or S;
V is selected from O, S and NR⁴, preferably O or NR⁴;
R² and R³ are independently selected from hydrogen, alkyl, and alkoxy; or R² and R³ together with the carbon atoms to which they are attached form a ring; and
R¹, R⁴, and R⁵ are independently selected from hydrogen and alkyl.

In certain embodiments, U is NR¹ and/or V is NR⁴, and R¹ and R⁴ are independently selected from methyl, ethyl, propyl, and isopropyl. In certain embodiments, both R¹ and R⁴ are methyl. On certain embodiments, both R² and R³ are hydrogen. In certain embodiments R² and R³ are independently alkyl, preferably lower alkyl. In certain embodiments, R² and R³ together are —(CH₂)ₙ— wherein n is 3 or 4, thereby forming a cyclopentyl or cyclohexyl ring. In certain embodiments, the nature of R² and R³ may affect the rate of cyclization of the self-cyclizing moiety. In certain such embodiments, it would be expected that the rate of cyclization would be greater when R² and R³ together with the carbon atoms to which they are attached form a ring than the rate when R² and R³ are independently selected from hydrogen, alkyl, and alkoxy. In certain embodiments, U is bonded to the self-cyclizing moiety.

In certain embodiments, the self-cyclizing moiety is selected from

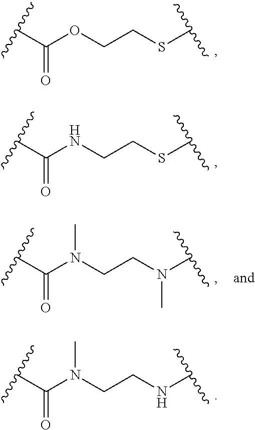

In certain embodiments, the selectivity-determining moiety may connect to the self-cyclizing moiety through carbonyl-heteroatom bonds, e.g., amide, carbamate, carbonate, ester, thioester, and urea bonds.

In certain embodiments, a taxane is covalently attached to a polymer through a tether, wherein the tether comprises a selectivity-determining moiety and a self-cyclizing moiety which are covalently attached to one another. In certain embodiments, the self-cyclizing moiety is selected such that after cleavage of the bond between the selectivity-determining moiety and the self-cyclizing moiety, cyclization of the self-cyclizing moiety occurs, thereby releasing the therapeutic agent. As an illustration, ABC may be a selectivity-determining moiety, and DEFGH maybe be a self-cyclizing moiety, and ABC may be selected such that enzyme Y cleaves between C and D. Once cleavage of the bond between C and D progresses to a certain point, D will cyclize onto H, thereby releasing therapeutic agent X, or a prodrug thereof.

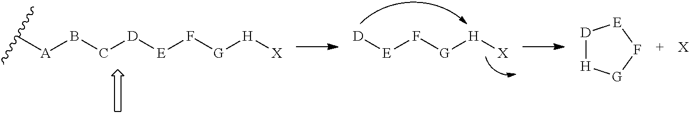

In certain embodiments, taxane X may further comprise additional intervening components, including, but not limited to another self-cyclizing moiety or a leaving group linker, such as CO₂ or methoxymethyl, that spontaneously dissociates from the remainder of the molecule after cleavage occurs.

In some embodiments, a linker may be and/or comprise an alkylene chain, a polyethylene glycol (PEG) chain, polysuccinic anhydride, poly-L-glutamic acid, poly(ethyleneimine), an oligosaccharide, an amino acid (e.g., glycine or cysteine), an amino acid chain, or any other suitable linkage. In certain embodiments, the linker group itself can be stable under physiological conditions, such as an alkylene chain, or it can be cleavable under physiological conditions, such as by an enzyme (e.g., the linkage contains a peptide sequence that is a substrate for a peptidase), or by hydrolysis (e.g., the linkage contains a hydrolyzable group, such as an ester or thioester). The linker groups can be biologically inactive, such as a PEG, polyglycolic acid, or polylactic acid chain, or can be biologically active, such as an oligo- or polypeptide that, when cleaved from the moieties, binds a receptor, deactivates an enzyme, etc. Various oligomeric linker groups that are biologically compatible and/or bioerodible are known in the art, and the selection of the linkage may influence the ultimate properties of the material, such as whether it is durable when implanted, whether it gradually deforms or shrinks after implantation, or whether it gradually degrades and is absorbed by the body. The linker group may be attached to the moieties by any suitable bond or functional group, including carbon-carbon bonds, esters, ethers, amides, amines, carbonates, carbamates, sulfonamides, etc.

In certain embodiments the linker group(s) of the present invention represent a hydrocarbylene group wherein one or more methylene groups is optionally replaced by a group Y (provided that none of the Y groups are adjacent to each other), wherein each Y, independently for each occurrence, is selected from, substituted or unsubstituted aryl, heteroaryl, cycloalkyl, heterocycloalkyl, or —O—, C(═X) (wherein X is NR₁, O or S), —OC(O)—, —C(═O)O, —NR₁—, —NR₁CO—, —C(O)NR₁—, —S(O)ₙ— (wherein n is 0, 1, or 2), —OC(O)—NR₁, —NR₁—C(O)—NR₁—, —NR₁—C(NR₁)—NR₁—, and —B(OR₁)—; and R₁, independently for each occurrence, represents H or a lower alkyl.

In certain embodiments, the linker group represents a derivatized or non-derivatized amino acid (e.g., glycine or cysteine). In certain embodiments, linker groups with one or more terminal carboxyl groups may be conjugated to the polymer. In certain embodiments, one or more of these terminal carboxyl groups may be capped by covalently attaching them to a therapeutic agent, a targeting moiety, or a cyclodextrin moiety via an (thio)ester or amide bond. In still other embodiments, linker groups with one or more terminal hydroxyl, thiol, or amino groups may be incorporated into the polymer. In preferred embodiments, one or more of these terminal hydroxyl groups may be capped by covalently attaching them to a therapeutic agent, a targeting moiety, or a cyclodextrin moiety via an (thio)ester, amide, carbonate, carbamate, thiocarbonate, or thiocarbamate bond. In certain embodiments, these (thio)ester, amide, (thio)carbonate or (thio)carbamates bonds may be biohydrolyzable, i.e., capable of being hydrolyzed under biological conditions.

In certain embodiments, a linker group represents a hydrocarbylene group wherein one or more methylene groups is optionally replaced by a group Y (provided that none of the Y groups are adjacent to each other), wherein each Y, independently for each occurrence, is selected from, substituted or unsubstituted aryl, heteroaryl, cycloalkyl, heterocycloalkyl, or —O—, C(═X) (wherein X is NR₁, O or S), —OC(O)—, —C(═O)O, —NR₁—, —NR₁CO—, —C(O)NR₁, —S(O)ₙ— (wherein n is 0, 1, or 2), —OC(O)—NR₁, —NR₁—C(O)—NR₁—, —NR₁—C(NR₁)—NR₁—, and —B(OR₁)—; and R₁, independently for each occurrence, represents H or a lower alkyl.

In certain embodiments, a linker group, e.g., between a taxane and the CDP, comprises a self-cyclizing moiety. In certain embodiments, a linker group, e.g., between a taxane and the CDP, comprises a selectivity-determining moiety.

In certain embodiments as disclosed herein, a linker group, e.g., between a taxane and the CDP, comprises a self-cyclizing moiety and a selectivity-determining moiety.

In certain embodiments as disclosed herein, the taxane or targeting ligand is covalently bonded to the linker group via a biohydrolyzable bond (e.g., an ester, amide, carbonate, carbamate, or a phosphate).

In certain embodiments as disclosed herein, the CDP comprises cyclodextrin moieties that alternate with linker moieties in the polymer chain.

In certain embodiments, the linker moieties are attached to taxanes or prodrugs thereof that are cleaved under biological conditions.

In certain embodiments, at least one linker that connects the taxane or prodrug thereof to the polymer comprises a group represented by the formula

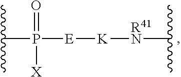

wherein
P is phosphorus;
O is oxygen;
E represents oxygen or N⁴⁰;
K represents hydrocarbyl;
X is selected from OR⁴² or NR⁴³R⁴⁴; and
R⁴⁰, R⁴¹, R⁴², R⁴³, and R⁴⁴ independently represent hydrogen or optionally substituted alkyl.

In certain embodiments, E is N⁴⁰ and R⁴⁰ is hydrogen.

In certain embodiments, K is lower alkylene (e.g., ethylene).

In certain embodiments, at least one linker comprises a group selected from

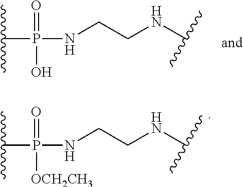

In certain embodiments, X is OR⁴².

In certain embodiments, the linker group comprises an amino acid or peptide, or derivative thereof (e.g., a glycine or cysteine).

In certain embodiments as disclosed herein, the linker is connected to the taxane through a hydroxyl group (e.g., forming an ester bond). In certain embodiments as disclosed herein, the linker is connected to the taxane through an amino group (e.g., forming an amide bond).

In certain embodiments, the linker group that connects to the taxane may comprise a self-cyclizing moiety, or a selectivity-determining moiety, or both. In certain embodiments, the selectivity-determining moiety is a moiety that promotes selectivity in the cleavage of the bond between the selectivity-determining moiety and the self-cyclizing moiety. Such a moiety may, for example, promote enzymatic cleavage between the selectivity-determining moiety and the self-cyclizing moiety. Alternatively, such a moiety may promote cleavage between the selectivity-determining moiety and the self-cyclizing moiety under acidic conditions or basic conditions.

In certain embodiments, any of the linker groups may comprise a self-cyclizing moiety or a selectivity-determining moiety, or both. In certain embodiments, the selectivity-determining moiety may be bonded to the self-cyclizing moiety between the self-cyclizing moiety and the polymer.

In certain embodiments, any of the linker groups may independently be or include an alkyl chain, a polyethylene glycol (PEG) chain, polysuccinic anhydride, poly-L-glutamic acid, poly(ethyleneimine), an oligosaccharide, an amino acid chain, or any other suitable linkage. In certain embodiments, the linker group itself can be stable under physiological conditions, such as an alkyl chain, or it can be cleavable under physiological conditions, such as by an enzyme (e.g., the linkage contains a peptide sequence that is a substrate for a peptidase), or by hydrolysis (e.g., the linkage contains a hydrolyzable group, such as an ester or thioester). The linker groups can be biologically inactive, such as a PEG, polyglycolic acid, or polylactic acid chain, or can be biologically active, such as an oligo- or polypeptide that, when cleaved from the moieties, binds a receptor, deactivates an enzyme, etc. Various oligomeric linker groups that are biologically compatible and/or bioerodible are known in the art, and the selection of the linkage may influence the ultimate properties of the material, such as whether it is durable when implanted, whether it gradually deforms or shrinks after implantation, or whether it gradually degrades and is absorbed by the body. The linker group may be attached to the moieties by any suitable bond or functional group, including carbon-carbon bonds, esters, ethers, amides, amines, carbonates, carbamates, sulfonamides, etc.

In certain embodiments, any of the linker groups may independently be an alkyl group wherein one or more methylene groups is optionally replaced by a group Y (provided that none of the Y groups are adjacent to each other), wherein each Y, independently for each occurrence, is selected from aryl, heteroaryl, carbocyclyl, heterocyclyl, or —O—, C(═X) (wherein X is NR¹, O or S), —OC(O)—, —C(═O)O—, —NR¹—, —NR¹CO—, —C(O)NR¹—, —S(O)ₙ— (wherein n is 0, 1, or 2), —OC(O)—NR¹—, —NR¹—C(O)—NR¹—, —NR¹—C(NR¹)—NR¹—, and —B(OR¹)—; and R¹, independently for each occurrence, is H or lower alkyl.

In one embodiment, the linker used to link taxane to a CDP controls the rate of taxane release from the CDP. For example, the linker can be a linker which in the PBS protocol described herein, releases within 24 hours as free taxane, e.g., docetaxel, paclitaxel and/or cabazitaxel, 70%, 75%, 80%, 85%, 86%, 87%, 88%, 89%, 90%, 91%, 92%, 93%, 94%, 95%, 96%, 97%, 98%, 99% or all of the taxane in the CDP-conjugated taxane initially present in the assay. In some embodiments, in the PBS protocol described herein, the linker releases 71±10% of the taxane, e.g., docetaxel, paclitaxel and/or cabazitaxel from the CDP-conjugated taxane, e.g., docetaxel, paclitaxel and/or cabazitaxel within 24 hours, wherein 71 is the % of taxane, e.g., docetaxel, paclitaxel and/or cabazitaxel released from the CDP-conjugate taxane, e.g., docetaxel, paclitaxel and/or cabazitaxel at 24 hours by a reference structure, e.g., a taxane such as docetaxel paclitaxel and/or cabazitaxel coupled via 2-(2-(2-aminoethoxy)ethoxy)acetic acetate (i.e., aminoethoxyethoxy) to the same CDP in the PBS protocol described herein. In other embodiments, the linker releases 88±10% of the taxane from the CDP-conjugated taxane, e.g., docetaxel, paclitaxel and/or cabazitaxel, within 24 hours, wherein 88 is the % of taxane, e.g., docetaxel, paclitaxel and/or cabazitaxel, released from the CDP-conjugate taxane, e.g., docetaxel, paclitaxel and/or cabazitaxel, at 24 hours by a reference structure, e.g., taxane, e.g., docetaxel, paclitaxel and/or cabazitaxel, coupled via glycine to the same CDP in the PBS protocol described herein or the linker releases 95±5% of the taxane, e.g., docetaxel, paclitaxel and/or cabazitaxel, from the CDP-conjugated taxane, e.g., docetaxel, paclitaxel and/or cabazitaxel, within 24 hours, wherein 95 is the % of taxane, e.g., docetaxel, paclitaxel and/or cabazitaxel, released from the CDP-conjugate taxane, e.g., docetaxel, paclitaxel and/or cabazitaxel, at 24 hours by a reference structure, e.g., taxane, e.g., docetaxel, paclitaxel and/or cabazitaxel coupled via alanine glycolate to the same CDP in the PBS protocol described herein. Such linkers include linkers which are released by hydrolysis of an ester bond, which hydrolysis releases taxane, e.g., docetaxel, paclitaxel and/or cabazitaxel conjugated to CDP from CDP. In one embodiment, the linker is selected from glycine, alanine glycolate and 2-(2-(2-aminoethoxy)ethoxy)acetic acetate (i.e., aminoethoxyethoxy). In one embodiment, the linker used to link taxane to a CDP attaches to the taxane via an ester linkage and the CDP via an amide linkage. In some preferred embodiments, the linker includes a heteroatom attached to the carbon positioned alpha to the carbonyl carbon that forms the ester linkage with the taxane.

In one embodiment, the linker used to link taxane to a CDP has the following formula

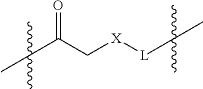

wherein

X is O, NH, or Nalkyl; and

L is an alkylenyl or heteroalkylenyl chain, wherein one or more of the carbons of the alkylenyl or heteroalkylenyl are optionally substituted (e.g., with an oxo moiety), or wherein L is absent;

wherein the carbonyl portion of the linker attaches to the taxane to form an ester linkage; and

wherein the X-L portion of the linker attaches to the CDP to form an amide bond.

In one embodiment, X is NH. In one embodiment, X is NH and L is absent.

In one embodiment, X is O. In one embodiment, X is O and L is an alkylenyl or heteroalkylenyl chain, wherein one or more of the carbons of the alkylenyl or heteroalkylenyl are optionally substituted (e.g., with an oxo moiety). In one embodiment, L is —C(O)CH₂CH₂NH—.

In some embodiments, the linker can be a linker which in the B16.F10 cell assay described herein, releases free taxane, e.g., docetaxel, paclitaxel and/or cabazitaxel, of the taxane, e.g., docetaxel, paclitaxel and/or cabazitaxel, in the CDP-conjugated taxane, e.g., docetaxel, paclitaxel and/or cabazitaxel, such that the IC₅₀ of the taxane, e.g., docetaxel, paclitaxel and/or cabazitaxel, is less than 25 nM, 20 nM, 15 nM, 10 nM, 5 nM, 4 nM, 3 nM, 2 nM, 1 nM, 0.5 nM or 0.1 nM. In some embodiments, in the B16.F10 assay described herein, the linker releases taxane, e.g., docetaxel, paclitaxel and/or cabazitaxel, from the CDP-conjugated taxane, e.g., docetaxel, paclitaxel and/or cabazitaxel such that the IC₅₀ of the taxane, e.g., docetaxel, paclitaxel and/or cabazitaxel is less than 5 nM, 4 nM, 3 nM, 2 nM, 1 nM, 0.5 nM. Such linkers include linkers which are released by hydrolysis of an ester bond, which hydrolysis releases docetaxel conjugated to CDP from CDP and linkers which are released by chemical or enzymatic cleavage of a disulfide bond, whereby enzymatic cleavage releases taxane, e.g., docetaxel, paclitaxel and/or cabazitaxel conjugated to CDP from CDP. In one embodiment, the linker is selected from glycine, alanine glycolate and dithiolethyloxy-carbonate.

In certain embodiments, the present invention contemplates a CDP, wherein a plurality of taxanes are covalently attached to the polymer through attachments that are cleaved under biological conditions to release the therapeutic agents as discussed above, wherein administration of the polymer to a subject results in release of the therapeutic agent over a period of at least 2 hours, 3 hours, 5 hours, 6 hours, 8 hours, 10 hours, 15 hours, 20 hours, 1 day, 2 days, 3 days, 4 days, 7 days, 10 days, 14 days, 17 days, 20 days, 24 days, 27 days up to a month.

In some embodiments, the conjugation of the taxane to the CDP improves the aqueous solubility of the taxane and hence the bioavailability. Accordingly, in one embodiment of the invention, the taxane has a log P>0.4, >0.6, >0.8, >1, >2, >3, >4, or even >5.

The CDP-taxane of the present invention preferably has a molecular weight in the range of 10,000 to 500,000; 30,000 to 200,000; or even 70,000 to 150,000 amu. In certain embodiments, the present invention contemplates attenuating the rate of release of the taxane by introducing various tether and/or linking groups between the therapeutic agent and the polymer. Thus, in certain embodiments, the CDP-taxane conjugates of the present invention are compositions for controlled delivery of the taxane.

### Taxanes

The term “taxane,” as used herein, refers to any naturally occurring, synthetic, or semi-synthetic taxane structure, for example, known in the art. Exemplary taxanes include those compounds shown below, including, for example, formula (X), (XIIa), and (XIIb).

In one embodiment, the taxane is a compound of the following formula (X):

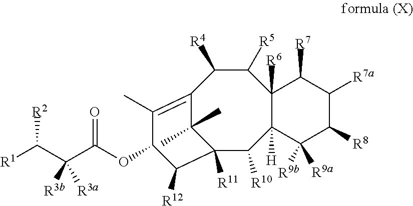

wherein;

R¹ is aryl (e.g., phenyl), heteroaryl (e.g., furanyl, thiophenyl, or pyridyl), alkyl (e.g., butyl such as isobutyl or tert-butyl), cycloalyl (e.g., cyclopropyl), heterocycloalkyl (epoxyl), or R¹, when taken together with one of R^{3b}, R^{9b}, or R¹⁰ and the carbons to which they are attached, forms a mono- or bi-cyclic ring system; wherein R¹ is optionally substituted with 1-3 R^{1a};

R² is NR^{2a}R^{2b} or OR^{2c};

R^{3a} is H, OH, Opolymer, OC(O)alkyl, or OC(O)alkenyl;

R^{3b} is H or OH; or together with R¹ and the carbon to which it is attached, forms a mono- or bi-cyclic ring system;

R⁴ is OH, alkoxy (e.g., methoxy), OC(O)alkyl (e.g., Oacyl), OC(O)cycloalkyl, heterocycloalkylalkyl; or R⁴ together with R⁵ and the carbons to which they are attached, form an optionally substituted ring; or R⁴, together with the carbon to which it is attached, forms a ring (forming a spirocyclic ring) or an oxo;

R⁵ is OH, OC(O)alkyl (e.g., Oacyl); or R⁵ together with R⁴ or R⁷ and the carbons to which they are attached, form an optionally substituted ring; or R⁵, together with the carbon to which it is attached, forms a ring (forming a spirocyclic ring) or an oxo;

R⁶ is alkyl (e.g., methyl); or R⁶ together with R⁷ and the carbons to which they are attached, form an optionally substituted ring (e.g., a cyclopropyl ring);

R⁷ is H, OH, alkoxy (e.g., methoxy), OC(O)Oalkyl, OalkylSalkyl (e.g., OCH₂SMe), or OalkylOalkyl (e.g., OCH₂OMe), thioalkyl, SalkylOalkyl (e.g., SCH₂OMe); or R⁷ together with R⁵ or R⁶ and the carbons to which they are attached, form an optionally substituted ring (e.g., a cyclopropyl ring);

R^{7a}H or OH;

R⁸ is OH or a leaving group (e.g., a mesylate, or halo); or R⁸ taken together with R^{9a} and the carbons to which they are attached form a ring;

R^{9a} is an activated alkyl (e.g. CH₂I); or R^{9a} taken together with R⁸ and the carbons to which they are attached form a ring; or R^{9a}, together with R^{9b} and the carbon to which it is attached, forms a ring (forming a spirocyclic ring);

R^{9b} is OH, OC(O)alkyl (e.g., Oacyl), OC(O)Oalkyl (e.g., OC(O)OMe), or OC(O)cycloalkyl; or R^{9b}, taken together with R and the carbons to which they are attached, form a ring; or R^{9b}, together with R^{9a} and the carbon to which it is attached, forms a ring (forming a spirocyclic ring);

R¹⁰ is OH, OC(O)aryl (e.g., wherein aryl is optionally substituted for example with halo, alkoxy, or N₃) or OC(O)alkyl; or R¹⁶ taken together with R¹ or R¹¹ and the carbons to which they are attached, forms a ring;

R¹¹H or OH; or R¹¹ taken together with R¹⁰ or R¹² and the carbons to which they are attached, forms a ring;

R¹² is H, or OH; or R¹² taken together with R¹¹ and the carbons to which they are attached, forms a ring;

each R^{1a} is independently halo (e.g., fluoro), alkyl (e.g., methyl)

each R^{2a} and R^{2b} is independently H, C(O)aryl (e.g., C(O)phenyl), C(O)alkyl (e.g., acyl), C(O)H, C(O)Oalkyl; wherein C(O)aryl (e.g., C(O)phenyl), C(O)alkyl (e.g., acyl), and C(O)Oalkyl is each optionally further substituted, for example, with a substituent as described in R^{1a}; and

R^{2c} is H or C(O)NHalkyl.

In some embodiments, R¹ is phenyl (e.g., optionally substituted for example with halo such as fluoro). In some embodiments, R¹ is heteroaryl, for example, furanyl, thiophenyl, or pyridyl (e.g., an optionally substituted pyridyl).

In some embodiments, R¹ is alkyl, e.g., butyl such as isobutyl or tert-butyl.

In some embodiments, R¹ is heterocycloalkyl (e.g., epoxyl optionally substituted, for example, with one or more alkyl groups such as methyl).

In some embodiments, R¹, taken together with R^{3b} and the carbons to which they are attached form a bicyclic ring system (e.g.,

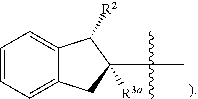

In some embodiments, R¹, taken together with R¹⁰ and the carbons to which they are attached, form a ring, e.g., a mono- or bi-cyclic ring system).

In some embodiments, R¹, taken together with R^{9b} and the carbons to which they are attached, form a ring, e.g., a mono- or bi-cyclic ring system).

In some embodiments, R² is NR^{2a}R^{2b}. In some embodiments, at least one of R^{2a} or R^{2b} is H. In some embodiments, R^{2a} is H and R^{2b} is C(O)aryl (e.g., C(O)phenyl), C(O)alkyl (e.g., acyl), C(O)H, or C(O)Oalkyl. In some embodiments, R² is NHC(O)aryl or NHC(O)Oalkyl.

In some embodiments, R^{3a} is OH. In some embodiments, R^{3a} is Opolymer. In some embodiments, polymer is polyglutamic acid. In some embodiments, R^{3a} is OC(O)C₂₁alkenyl.

In some embodiments, one of R^{3a} or R^{3b} is H and the other of R^{3a} or R^{3b} is OH.

In some embodiments, R⁴ is OAcyl. In some embodiments, R⁴ is OH. In some embodiments, R⁴ is methoxy. In some embodiments, R⁴ together with R⁵ and the carbons to which they are attached forms

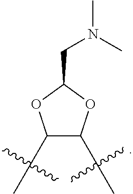

In some embodiments, R⁴, together with the carbon to which it is attached, forms

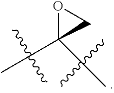

In some embodiments, R⁴, together with the carbon to which it is attached, forms an oxo. In some embodiments, R⁴ is heterocycloalkylalkyl (e.g.,

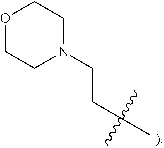 [SMILES: *CCN1CCOCC1]

In some embodiments, R⁵, together with the carbon to which it is attached, forms an oxo. In some embodiments, R⁵ together with R⁷ and the carbons to which they are attached forms

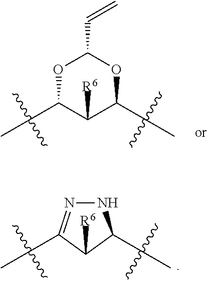

In some embodiments, R⁶ is methyl. In some embodiments, R⁶ together with R⁷ and the carbons to which they are attached form a ring (e.g., cyclopropyl).

In some embodiments, R⁷ is OH. In some embodiments, R⁷ is H. In some embodiments, when R⁷ is H, R^{7a} is OH.

In some embodiments, R^{7a} is H. In some embodiments, R^{7a} is OH.

In some embodiments, R⁸ together with R^{9a} and the carbons to which they are attached form

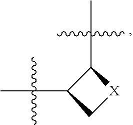

wherein X is O, S, Se, or NR^{8a} (e.g., O), wherein R^{8a} is H, alkyl, arylalkyl (e.g., benzyl), C(O)alkyl, or C(O)H. In some embodiments, R⁸ together with R^{9a} and the carbons to which they are attached form a cyclopropyl ring.

In some embodiments, R^{9b} is OAc.

In some embodiments, R¹⁰ is OC(O)phenyl. In some embodiments, R¹⁰ taken together with R¹¹ and the carbon to which it is attached, forms a ring such as

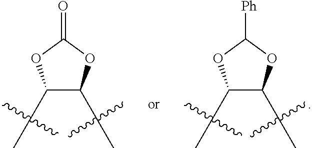 [SMILES: *[C@@H]1OC(=O)O[C@H]1* ; *[C@@H]1OC(c2ccccc2)O[C@H]1*]

In some embodiments, R¹¹ is OH. In some embodiments, R¹¹ taken together with R¹² and the carbon to which it is attached, forms a ring such as

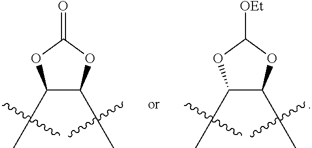 [SMILES: *[C@@H]1OC(=O)O[C@@H]1* ; *[C@@H]1OC(OCC)O[C@H]1*]

In some embodiments, R¹² is H.

In some embodiments, the variables defined above are chosen so as to form docetaxel, paclitaxel, larotaxel, or cabazitaxel or a structural analogue thereof.

In some embodiments, the taxane is a compound of formula (Xa)

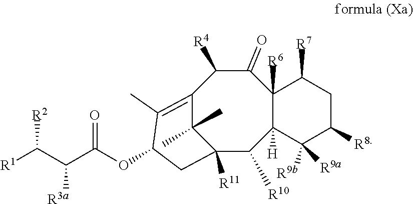

In some embodiments, the taxane is a compound of formula (Xb)

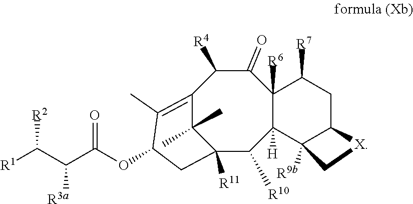

In some embodiments, the compound is a compound of formula Xc

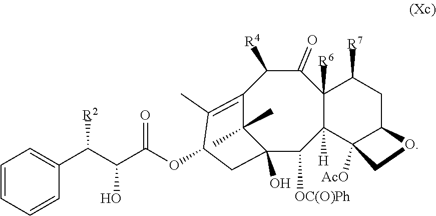

In some embodiments, R² is NHC(O)aryl or NHC(O)Oalkyl.

In some embodiments, R⁴ is OH or OAc.

In some embodiments, R⁶ is methyl.

In some embodiments, R⁷ is OH or OMe.

In some embodiments, R⁶ and R⁷, together with the carbons to which they are attached, form a ring.

In some embodiments, the variables defined above are chosen so as to form docetaxel, paclitaxel, larotaxel, or cabazitaxel or a structural analogue thereof.

In one embodiment, the taxane is a compound of formula (XI)

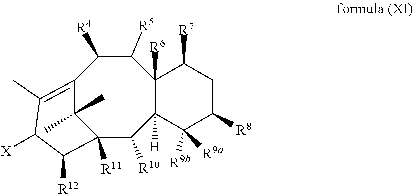

wherein

X is OH, oxo (i.e., when forming a double bond with the carbon to which it is attached), alkoxy, OC(O)alkyl (e.g., Oacyl), or OPg;

R⁴ is OH, alkoxy (e.g., methoxy), OC(O)alkyl (e.g., Oacyl), OC(O)cycloalkyl, OPg, heterocycloalkylalkyl; or R⁴ together with R⁵ and the carbons to which they are attached, form an optionally substituted ring; or R⁴, together with the carbon to which it is attached, forms a ring (forming a spirocyclic ring) or an oxo;

R⁵ is OH, OC(O)alkyl (e.g., Oacyl), or OPg; or R⁵ together with R⁴ and the carbons to which they are attached, form an optionally substituted ring; or R⁵, together with the carbon to which it is attached, forms an oxo;

R⁶ is alkyl (e.g., methyl);

R⁷ is H, OH, alkoxy (e.g., methoxy), OC(O)alkyl (e.g., OAc); OPg (e.g., OTES or OTroc), or OC(O)alkenyl (wherein alkenyl is substituted, e.g., with aryl (e.g., napthyl) (e.g., OC(O)CHCHnapthyl), or R⁷, together with the carbon to which it is attached, forms an oxo;

R⁸ is OH, optionally substituted OC(O)arylalkyl (e.g., OC(O)CHCHphenyl), OC(O)(CH₂)₁₋₃aryl (e.g., OC(O)CH₂CH₂-phenyl), or a leaving group (e.g., a mesylate, or halo); or R⁸ taken together with R^{9a} and the carbons to which they are attached form a ring;

R^{9a} is an activated alkyl (e.g. CH₂I); or R^{9a} taken together with R⁸ and the carbons to which they are attached form a ring; or R^{9a}, together with R^{9b} and the carbon to which it is attached, forms a ring (forming a spirocyclic ring)or R^{9a} taken together with R^{9b} and the carbon to which they are attached form an alylenyl;

R^{9b} is OH, alkoxy, OC(O)alkyl (e.g., Oacyl), OC(O)Oalkyl (e.g., OC(O)OMe), OC(O)cycloalkyl, or OPg; or R^{9b}, together with R^{9a} and the carbon to which it is attached, forms a ring (forming a spirocyclic ring); or R^{9b} taken together with R^{9a} and the carbon to which they are attached form an alylenyl;

R¹⁰ is OH, OC(O)aryl (e.g., wherein aryl is optionally substituted for example with halo, alkoxy, or N₃) or OC(O)alkyl; or R¹⁰ taken together with R¹¹ and the carbons to which they are attached, forms a ring;

R¹¹H, OH; or R¹¹ taken together with R¹⁶ or R¹² and the carbons to which they are attached, forms a ring;

R¹² is H, OH, or OC(O)alkyl, wherein alkyl is substituted with 1-4 substituents; or R¹² taken together with R¹¹ and the carbons to which they are attached, forms a ring;

Pg is a protecting group for a heteroatom such as O or N (e.g., Bn, Bz, TES, TMS, DMS, Troc, or Ac); and [symbol] is a single or double bond

In some embodiments, X is OH. In some embodiments, X is oxo. In some embodiments, X is OAc.

In some embodiments, [symbol] is a single bond.

In some embodiments, R⁴ is OAcyl. In some embodiments, R⁴ is OH. In some embodiments, R⁴ is methoxy. In some embodiments, R⁴ is OPg (e.g., OTroc or OAc). In some embodiments, R⁴ together with R⁵ and the carbons to which they are attached forms a ring.

In some embodiments, R⁵, together with the carbon to which it is attached, forms an oxo. In some embodiments, R⁵ is OH or OPg.

In some embodiments, R⁶ is methyl.

In some embodiments, R⁷ is H. In some embodiments, R⁷ is OH or OPg. In some embodiments, R⁷, together with the carbon to which it is attached, forms an oxo.

In some embodiments, R⁸ is

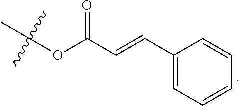 [SMILES: *OC(=O)/C=C/c1ccccc1]

In some embodiments, R⁸ together with R^{9a} and the carbons to which they are attached form

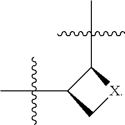

wherein X is O, S, Se, or NR^{8a} (e.g., 0), wherein R^{8a} is H, alkyl, arylalkyl (e.g., benzyl), C(O)alkyl, Pg, or C(O)H. In some embodiments, R⁸ together with R^{9a} and the carbons to which they are attached form a cyclopropyl ring. In some embodiments,

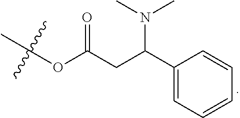 [SMILES: *OC(=O)CC(c1ccccc1)N(C)C]

In some embodiments, R^{9a} and R^{9b}, together with the carbon to which they are attached form

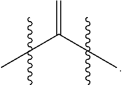 [SMILES: *C(*)=C]

In some embodiments, R^{9b} is OAc.

In some embodiments, R¹⁰ is OC(O)phenyl. In some embodiments, R¹⁰ taken together with R¹¹ and the carbon to which it is attached, forms a ring such as

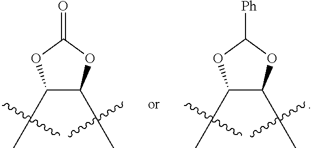 [SMILES: *[C@@H]1OC(=O)O[C@H]1* ; *[C@@H]1OC(c2ccccc2)O[C@H]1*]

In some embodiments, R¹¹ is H. In some embodiments, R¹¹ is OH.

In some embodiments, R¹² is H. In some embodiments, R¹² is OH. In some embodiments, R¹² is

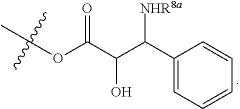

In one embodiment, the taxane is a compound of formula (XIIa)

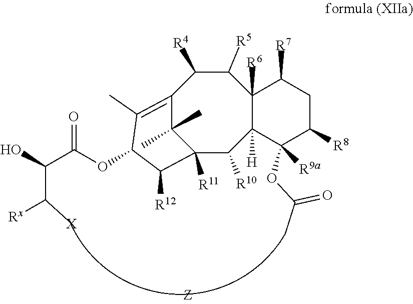

wherein

Z forms a ring by linking O with the atom X attached to —CHR^{x};

R⁴ is OH, alkoxy (e.g., methoxy), OC(O)alkyl (e.g., Oacyl), OC(O)cycloalkyl, heterocycloalkylalkyl; or R⁴ together with R⁵ and the carbons to which they are attached, form an optionally substituted ring; or R⁴, together with the carbon to which it is attached, forms a ring (forming a spirocyclic ring) or an oxo;

R⁵ is OH, OC(O)alkyl (e.g., Oacyl); or R⁵ together with R⁴ or R⁷ and the carbons to which they are attached, form an optionally substituted ring; or R⁵, together with the carbon to which it is attached, forms a ring (forming a spirocyclic ring) or an oxo;

R⁶ is alkyl (e.g., methyl); or R⁶ together with R⁷ and the carbons to which they are attached, form an optionally substituted ring (e.g., a cyclopropyl ring);

R⁷ is H, OH, alkoxy (e.g., methoxy), OC(O)Oalkyl, OalkylSalkyl (e.g., OCH₂SMe), or OalkylOalkyl (e.g., OCH₂OMe), thioalkyl, SalkylOalkyl (e.g., SCH₂OMe); or R⁷ together with R⁵ or R⁶ and the carbons to which they are attached, form an optionally substituted ring (e.g., a cyclopropyl ring);

R^{7a}H or OH;

R⁸ is OH or a leaving group (e.g., a mesylate, or halo); or R⁸ taken together with R^{9a} and the carbons to which they are attached form a ring;

R^{9a} is an activated alkyl (e.g. CH₂I); or R^{9a} taken together with R⁸ and the carbons to which they are attached form a ring;

R¹⁰ is OH, OC(O)aryl (e.g., wherein aryl is optionally substituted for example with halo, alkoxy, or N₃) or OC(O)alkyl; or R¹⁰ taken together with R¹ or R¹¹ and the carbons to which they are attached, forms a ring;

R¹¹H or OH; or R¹¹ taken together with R¹⁰ or R¹² and the carbons to which they are attached, forms a ring;

R¹² is H, or OH; or R¹² taken together with R¹¹ and the carbons to which they are attached, forms a ring;

R^{x} is NHPg or aryl;

X is C or N; and

Pg is a protecting group for a heteroatom such as O or N (e.g., Bn, Bz, TES, TMS, DMS, Troc, Boc or Ac).

In some embodiments, Z includes one or more phenyl rings.

In some embodiments, Z includes one or more double bonds.

In some embodiments, Z includes one or more heteroatoms.

In some embodiments, Z is

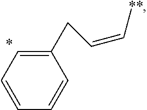

wherein * indicates the atom X attached to CHR^{X} and ** indicates the carbon attached to C(O). In some embodiments, Z is

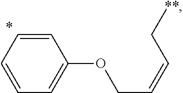

wherein * indicates the atom X attached to CHR^{X} and ** indicates the carbon attached to C(O). In some embodiments, Z is

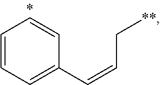

wherein * indicates the atom X attached to CHR^{x} and ** indicates the carbon attached to C(O).

In some embodiments, the taxane is a compound of formula (XIIb)

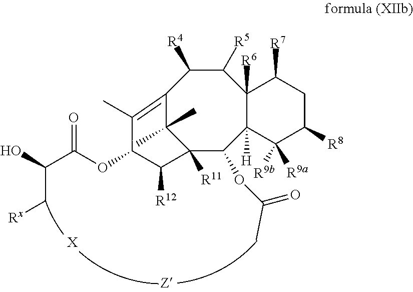

wherein

Z′ forms a ring by linking 0 with the atom X, which is attached to —CHR^{x};

R⁴ is OH, alkoxy (e.g., methoxy), OC(O)alkyl (e.g., Oacyl), OC(O)cycloalkyl, heterocycloalkylalkyl; or R⁴ together with R⁵ and the carbons to which they are attached, form an optionally substituted ring; or R⁴, together with the carbon to which it is attached, forms a ring (forming a spirocyclic ring) or an oxo;

R⁵ is OH, OC(O)alkyl (e.g., Oacyl); or R⁵ together with R⁴ or R⁷ and the carbons to which they are attached, form an optionally substituted ring; or R⁵, together with the carbon to which it is attached, forms a ring (forming a spirocyclic ring) or an oxo;

R⁶ is alkyl (e.g., methyl); or R⁶ together with R⁷ and the carbons to which they are attached, form an optionally substituted ring (e.g., a cyclopropyl ring);

R⁷ is H, OH, alkoxy (e.g., methoxy), OC(O)Oalkyl, OalkylSalkyl (e.g., OCH₂SMe), or OalkylOalkyl (e.g., OCH₂OMe), thioalkyl, SalkylOalkyl (e.g., SCH₂OMe); or R⁷ together with R⁵ or R⁶ and the carbons to which they are attached, form an optionally substituted ring (e.g., a cyclopropyl ring);

R^{7a}H or OH;

R⁸ is OH or a leaving group (e.g., a mesylate, or halo); or R⁸ taken together with R^{9a} and the carbons to which they are attached form a ring;

R^{9a} is an activated alkyl (e.g. CH₂I); or R^{9a} taken together with R⁸ and the carbons to which they are attached form a ring; or R^{9a}, together with R^{9b} and the carbon to which it is attached, forms a ring (forming a spirocyclic ring);

R^{9b} is OH, OC(O)alkyl (e.g., Oacyl), OC(O)Oalkyl (e.g., OC(O)OMe), or OC(O)cycloalkyl; or R^{9b}, together with R^{9a} and the carbon to which it is attached, forms a ring (forming a spirocyclic ring);

R¹¹H or OH; or R¹¹ taken together with R¹⁰ or R¹² and the carbons to which they are attached, forms a ring;

R¹² is H, or OH; or R¹² taken together with R¹¹ and the carbons to which they are attached, forms a ring;

R^{x} is NHPg or aryl;

X is C or N; and

Pg is a protecting group for a heteroatom such as O or N (e.g., Bn, Bz, TES, TMS, DMS, Troc, Boc or Ac).

In some embodiments, Z′ includes one or more phenyl rings.

In some embodiments, Z′ includes one or more double bonds.

In some embodiments, Z′ includes one or more heteroatoms.

In some embodiments, Z′ is

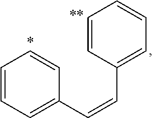 [SMILES: C(=C/c1ccccc1)/c1ccccc1]

wherein * indicates the atom X attached to CHR^{x} and ** indicates the carbon attached to C(O). In some embodiments, Z′ is

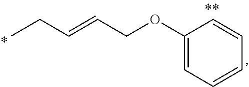

wherein * indicates the atom X attached to CHR^{x} and ** indicates the carbon attached to C(O). In some embodiments, Z′ is

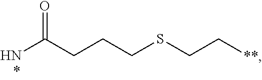

wherein * indicates the atom X attached to CHR^{x} and ** indicates the carbon attached to C(O).

In some embodiments, the taxane is a compound of formula (XIII)

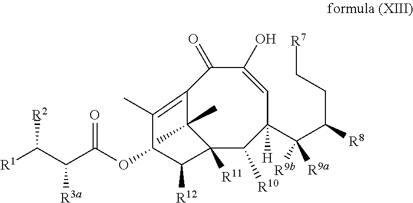

wherein;

R¹ is aryl (e.g., phenyl), heteroaryl (e.g., furanyl, thiophenyl, or pyridyl), alkyl (e.g., butyl such as isobutyl or tert-butyl), cycloalyl (e.g., cyclopropyl), heterocycloalkyl (epoxyl), or R¹, when taken together with one of R^{3b}, R^{9b}, or R¹⁰ and the carbons to which they are attached, forms a mono- or bi-cyclic ring system; wherein R¹ is optionally substituted with 1-3 R^{1a};

R² is NR^{2a}R^{2b} or OR^{2c};

R^{3a} is H, OH, Opolymer, OC(O)alkyl, or OC(O)alkenyl;

R⁷ is OH, alkoxy (e.g., methoxy), OC(O)Oalkyl;

R⁸ is OH or a leaving group (e.g., a mesylate, or halo); or R⁸ taken together with R^{9a} and the carbons to which they are attached form a ring;

R^{9a} is an activated alkyl (e.g. CH₂I); or R^{9a} taken together with R⁸ and the carbons to which they are attached form a ring; or R^{9a}, together with R^{9b} and the carbon to which it is attached, forms a ring (forming a spirocyclic ring)

R^{9b} is OH, OC(O)alkyl (e.g., Oacyl), OC(O)Oalkyl (e.g., OC(O)OMe), or OC(O)cycloalkyl; or R^{9b}, taken together with R¹ and the carbons to which they are attached, form a ring; or R^{9b}, together with R^{9a} and the carbon to which it is attached, forms a ring (forming a spirocyclic ring);

R¹⁰ is OH, OC(O)aryl (e.g., wherein aryl is optionally substituted for example with halo, alkoxy, or N₃) or OC(O)alkyl; or R¹⁰ taken together with R¹ or R¹¹ and the carbons to which they are attached, forms a ring;

R¹¹H or OH; or R¹¹ taken together with R¹⁰ or R¹² and the carbons to which they are attached, forms a ring;

R¹² is H, or OH; or R¹² taken together with R¹¹ and the carbons to which they are attached, forms a ring;

each R^{1a} is independently halo (e.g., fluoro), alkyl (e.g., methyl)

each R^{2a} and R^{2b} is independently H, C(O)aryl (e.g., C(O)phenyl), C(O)alkyl (e.g., acyl), C(O)H, C(O)Oalkyl; wherein C(O)aryl (e.g., C(O)phenyl), C(O)alkyl (e.g., acyl), and C(O)Oalkyl is each optionally further substituted, for example, with a substituent as described in R^{1a};

R^{2c} is H or C(O)NHalkyl; and

R^{8a} is H, alkyl, arylalkyl (e.g., benzyl), C(O)alkyl, or C(O)H.

In some embodiments, R⁷ is OH.

In some preferred embodiments, the taxane is docetaxel, larotaxel, milataxel, TPI-287, TL-310, BMS-275183, BMS-184476, BMS-188797, ortataxel, tesetaxel, or cabazitaxel. Additional taxanes are provided in Fan, Mini-Reviews in Medicinal Chemistry, 2005, 5, 1-12; Gueritte, Current Pharmaceutical Design, 2001, 7, 1229-1249; Kingston, J. Nat. Prod., 2009, 72, 507-515; and Ferlini, Exper Opin. Invest. Drugs, 2008, 17, 3, 335-347; the contents of each of which is incorporated herein by reference in its entirety.

Exemplary CDP-Taxane Conjugates

CDP-taxane conjugates can be made using many different combinations of components described herein. For example, various combinations of cyclodextrins (e.g., beta-cyclodextrin), comonomers (e.g., PEG containing comonomers), linkers linking the cyclodextrins and comonomers, and/or linkers tethering the taxane to the CDP are described herein.

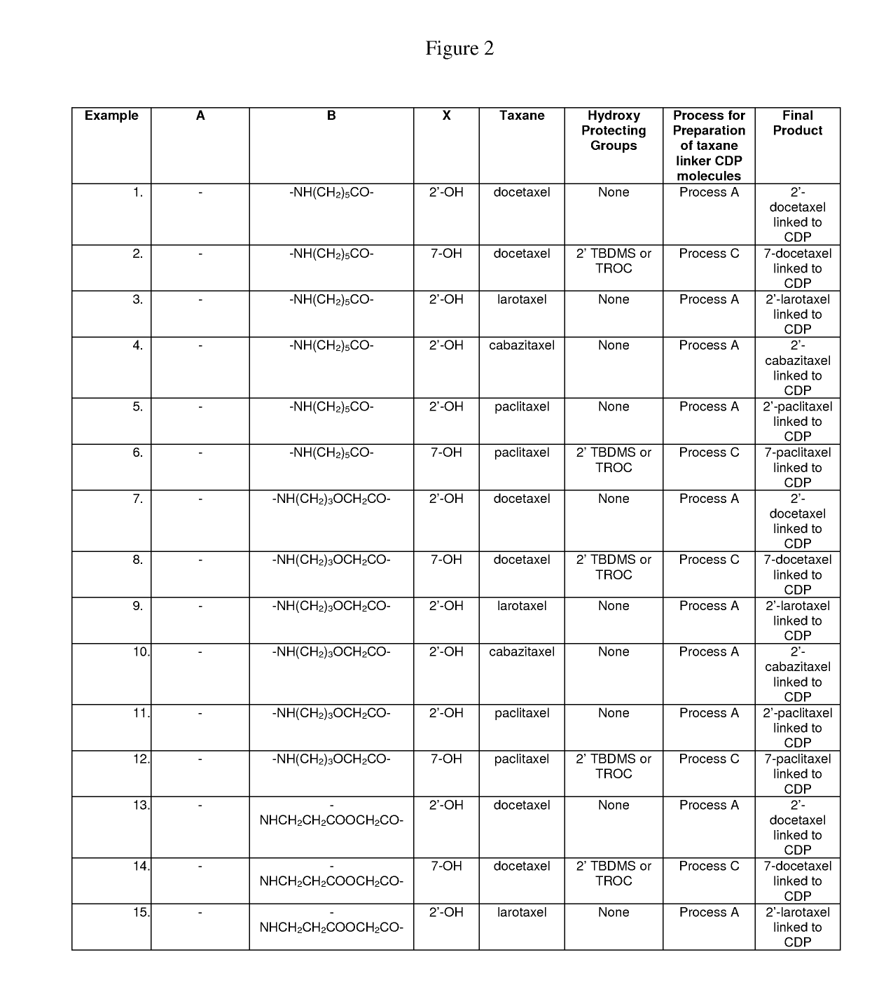

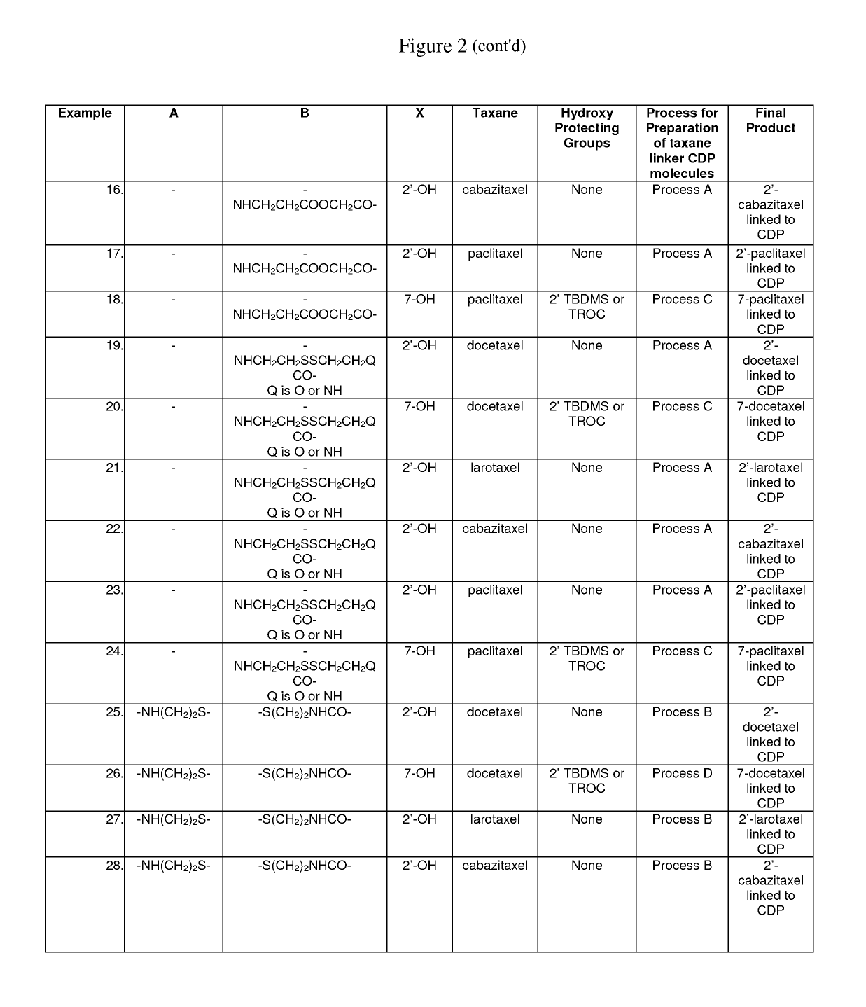

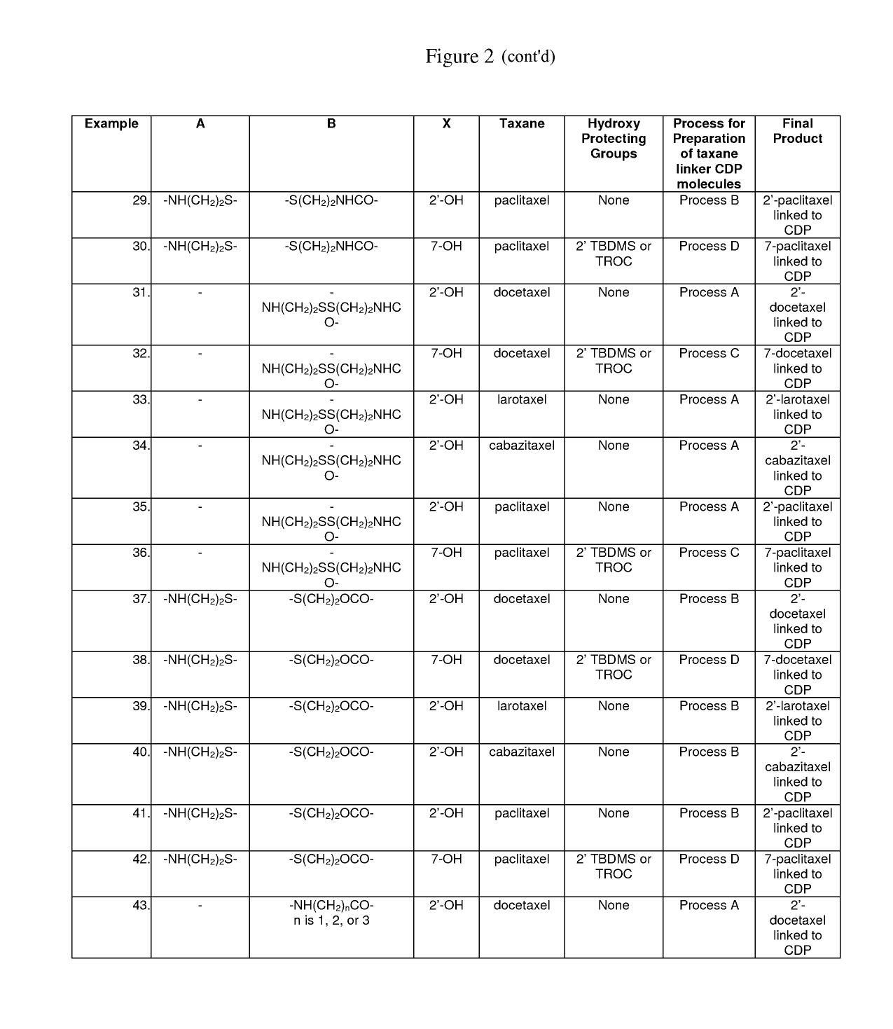

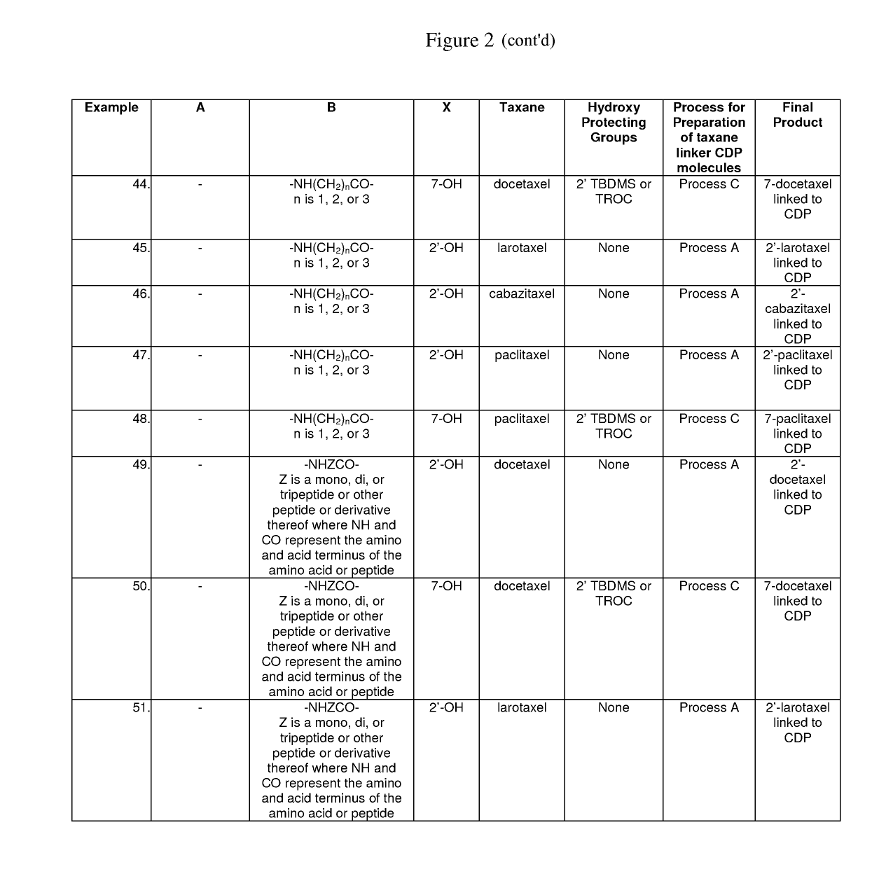

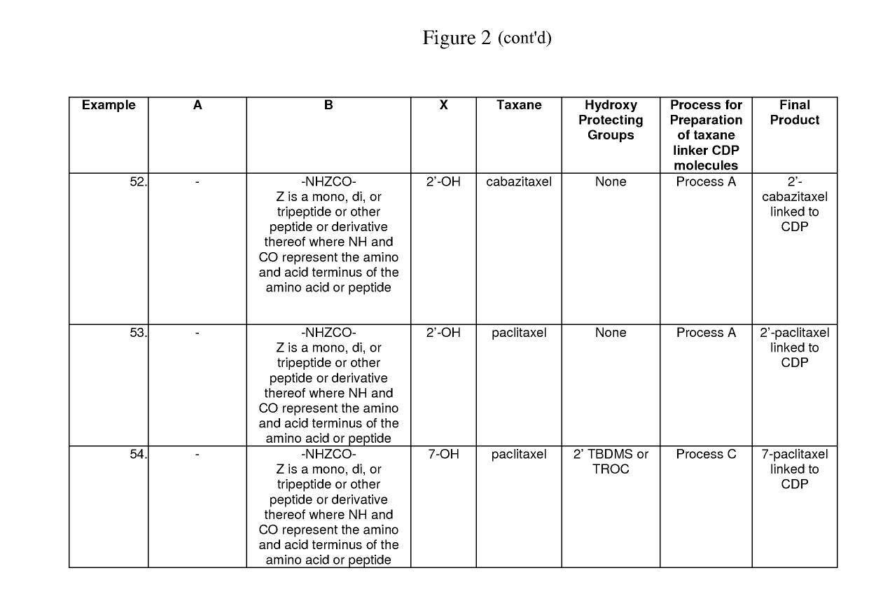

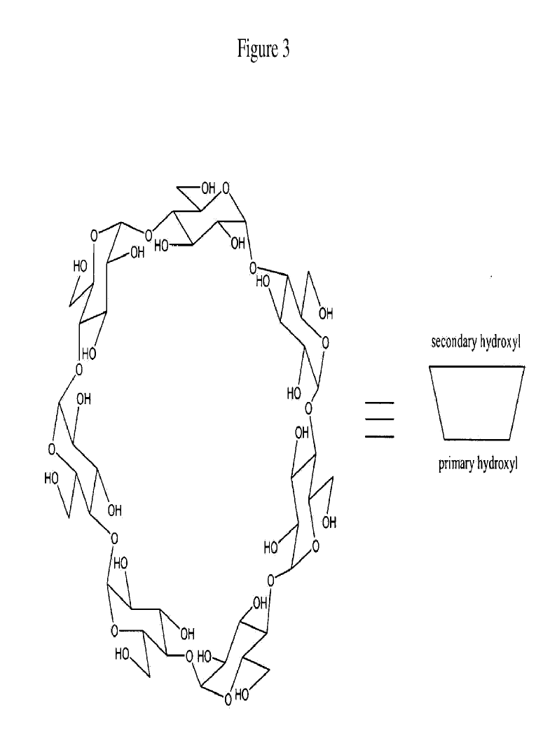

FIG. 2 is a table depicting examples of different CDP-taxane conjugates. The CDP-taxane conjugates in FIG. 2 are represented by the following formula:

CDP-CO-ABX-Taxane

In this formula,

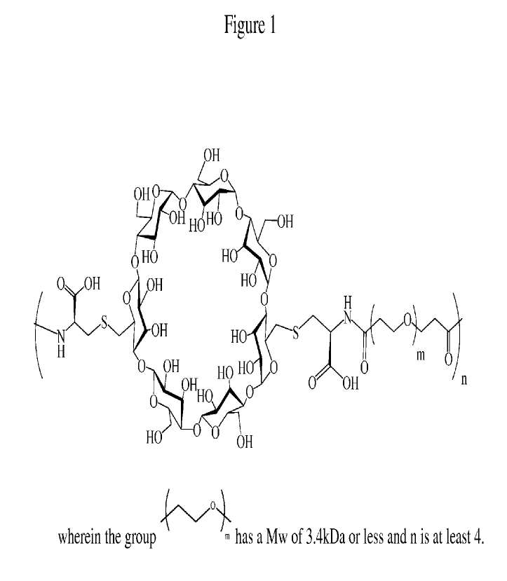

CDP is the cyclodextrin-containing polymer shown below (as well as in FIG. 1):

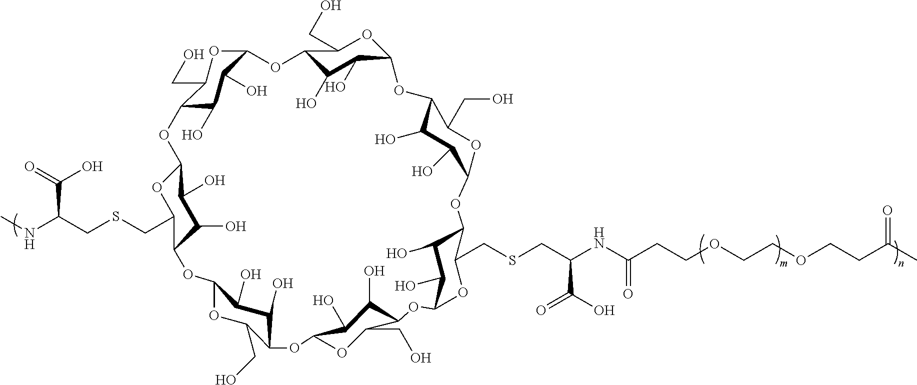

wherein the group

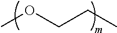

has a Mw of 3.4 kDa or less and n is at least 4, 5, 6, 7, 8, 9, 10, 11, 12, 13, 14, 15, 16, 17, 18, 19 or 20. Note that the taxane is conjugated to the CDP through the carboxylic acid moieties of the polymer as provided above. Full loading of the taxane onto the CDP is not required. In some embodiments, at least one, e.g., at least 2, 3, 4, 5, 6 or 7, of the carboxylic acid moieties remains unreacted with the taxane after conjugation (e.g., a plurality of the carboxylic acid moieties remain unreacted).

CO represents the carbonyl group of the cysteine residue of the CDP;

A and B represent the link between the CDP and the taxane. Position A is either a bond between linker B and the cysteine acid carbonyl of CDP (represented as a “-” in FIG. 2), a bond between the taxane and the cysteine acid carbonyl of CDP (represented as a “-” in FIG. 2) or depicts a portion of the linker that is attached via a bond to the cysteine acid carbonyl of the CDP. Position B is either not occupied (represented by “-” in FIG. 2) or represents the linker or the portion of the linker that is attached via a bond to the taxane; and

X represents the heteroatom to which the linker is coupled on the taxane.

As provided in FIG. 2, the column with the heading “Taxane” indicates which taxane is included in the CDP-taxane conjugate.

The three columns on the right of the table in FIG. 2 indicate respectively, what, if any, protecting groups are used to protect the indicated position of the taxane, the process for producing the CDP-taxane conjugate, and the final product of the process for producing the CDP-taxane conjugate.

The processes referred to in FIG. 2 are given a letter representation, e.g., Process A, Process B, etc. as seen in the second column from the right. The steps for each these processes respectively are provided below.
Process A: Couple the protected linker of position B to the taxane, deprotect the linker and couple to CDP via the carboxylic acid group of the CDP to afford the 2′-taxane linked to CDP.
Process B: Couple the activated linker of position B to the 2′-hydroxyl of taxane, and couple to CDP containing linker of position A via the linker of A to afford the 2′-taxane linked to CDP.
Process C: Protect the C2′ hydroxy group of the taxane, couple the protected linker of position B to the taxane, deprotect the linker and the C2′ hydroxy group, and couple to CDP via the carboxylic acid group of the CDP to afford the 7-taxane linked to CDP.
Process D: Protect the C2′ hydroxy group of the taxane, couple the activated linker of position B to the 7-hydroxyl of the taxane, deprotect the C2′ hydroxy group and couple to CDP containing linker of position A via the linker of A to afford afford the 7-taxane linked to CDP.

As shown specifically in FIG. 2, the CDP-taxane conjugates can be prepared using a variety of methods known in the art, including those described herein. In some embodiments, the CDP-taxane conjugates can be prepared using no protecting groups on the taxane (see, e.g., examples 1, 3 and 4). For taxanes having hydroxyl groups at both the 2′ and the 7-positions, one of skill in the art will understand that the 2′-position is more reactive, and therefore when using no protecting groups, the major product of the reaction(s) will be that which is linked via the 2′ position.

One or more protecting groups can be used in the processes described above to make the CDP-taxane conjugates described herein. A protecting group can be used to control the point of attachment of the taxane and/or taxane linker to position A. In some embodiments, the protecting group is removed and, in other embodiments, the protecting group is not removed. If a protecting group is not removed, then it can be selected so that it is removed in vivo (e.g., acting as a prodrug). An example is hexanoic acid which has been shown to be removed by lipases in vivo if used to protect a hydroxyl group in doxorubicin. Protecting groups are generally selected for both the reactive groups of the taxane and the reactive groups of the linker that are not targeted to be part of the coupling reaction. The protecting group should be removable under conditions which will not degrade the taxane and/or linker material. Examples include t-butyldimethylsilyl (“TBDMS”) and TROC (derived from 2,2,2-trichloroethoxy chloroformate). Carboxybenzyl (“CBz”) can also be used in place of TROC if there is selectivity seen for removal over olefin reduction. This can be addressed by using a group which is more readily removed by hydrogenation such as -methoxybenzyl OCO—. Other protecting groups may also be acceptable. One of skill in the art can select suitable protecting groups for the products and methods described herein.

CDP-Taxane Conjugate Characteristics

In some embodiments, the CDP and/or CDP-taxane conjugates as described herein have polydispersities less than about 3, or even less than about 2.

One embodiment of the present invention provides an improved delivery of certain taxanes by covalently conjugating them to a CDP. Such conjugation improves the aqueous solubility and hence the bioavailability of the taxane. Accordingly, in one embodiment of the invention, the taxane is a hydrophobic compound with a log P>0.4, >0.6, >0.8, >1, >2, >3, >4, or even >5. In other embodiments, a taxane may be attached to another compound, such as an amino acid, prior to covalently attaching the conjugate onto the CDP.

The CDP-taxane conjugates described herein preferably have molecular weights in the range of 10,000 to 500,000; 30,000 to 200,000; or even 70,000 to 150,000 amu. In certain embodiments as disclosed herein, the compound has a number average (Mₙ) molecular weight between 1,000 to 500,000 amu, or between 5,000 to 200,000 amu, or between 10,000 to 100,000 amu. One method to determine molecular weight is by gel permeation chromatography (“GPC”), e.g., mixed bed columns, CH₂Cl₂ solvent, light scattering detector, and off-line do/dc. Other methods are known in the art.

In certain embodiments as disclosed herein, the CDP-taxane conjugate is biodegradable or bioerodable.

In certain embodiments as disclosed herein, the taxane or prodrug thereof makes up at least 3% (e.g., at least about 5%, 10%, 15%, or 20%) by weight of the compound. In certain embodiments, the taxane or prodrug thereof makes up at least 15% or 20% by weight of the compound (e.g., from 17-21% by weight).

In other embodiments, the CDP-taxane conjugate may be a flexible or flowable material. When the CDP used is itself flowable, the CDP composition of the invention, even when viscous, need not include a biocompatible solvent to be flowable, although trace or residual amounts of biocompatible solvents may still be present.

When a solvent is used to facilitate mixing or to maintain the flowability of the CDP-taxane conjugate, it should be non-toxic, otherwise biocompatible, and should be used in relatively small amounts. Examples of suitable biocompatible solvents, when used, include N-methyl-2-pyrrolidone, 2-pyrrolidone, ethanol, propylene glycol, acetone, methyl acetate, ethyl acetate, methyl ethyl ketone, dimethylformamide, dimethylsulfoxide, tetrahydrofuran, caprolactam, oleic acid, or 1-dodecylazacylcoheptanone. Preferred solvents include N-methylpyrrolidone, 2-pyrrolidone, dimethylsulfoxide, and acetone because of their solvating ability and their biocompatibility.

In certain embodiments, the CDP-taxane conjugates are soluble in one or more common organic solvents for ease of fabrication and processing. Common organic solvents include such solvents as chloroform, dichloromethane, dichloroethane, 2-butanone, butyl acetate, ethyl butyrate, acetone, ethyl acetate, dimethylacetamide, N-methylpyrrolidone, dimethylformamide, and dimethylsulfoxide.

In certain embodiments, the CDP-taxane conjugates described herein, upon contact with body fluids, undergo gradual degradation. The life of a biodegradable polymer in vivo depends upon, among other things, its molecular weight, crystallinity, biostability, and the degree of crosslinking. In general, the greater the molecular weight, the higher the degree of crystallinity, and the greater the biostability, the slower biodegradation will be.

If a subject composition is formulated with a taxane or other material, release of the taxane or other material for a sustained or extended period as compared to the release from an isotonic saline solution generally results. Such release profile may result in prolonged delivery (over, say 1 to about 2,000 hours, or alternatively about 2 to about 800 hours) of effective amounts (e.g., about 0.0001 mg/kg/hour to about 10 mg/kg/hour, e.g., 0.001 mg/kg/hour, 0.01 mg/kg/hour, 0.1 mg/kg/hour, 1.0 mg/kg/hour) of the taxane or any other material associated with the polymer.

A variety of factors may affect the desired rate of hydrolysis of CDP-taxane conjugates, the desired softness and flexibility of the resulting solid matrix, rate and extent of bioactive material release. Some of such factors include the selection/identity of the various subunits, the enantiomeric or diastereomeric purity of the monomeric subunits, homogeneity of subunits found in the polymer, and the length of the polymer. For instance, the present invention contemplates heteropolymers with varying linkages, and/or the inclusion of other monomeric elements in the polymer, in order to control, for example, the rate of biodegradation of the matrix.

To illustrate further, a wide range of degradation rates may be obtained by adjusting the hydrophobicities of the backbones or side chains of the polymers while still maintaining sufficient biodegradability for the use intended for any such polymer. Such a result may be achieved by varying the various functional groups of the polymer. For example, the combination of a hydrophobic backbone and a hydrophilic linkage produces heterogeneous degradation because cleavage is encouraged whereas water penetration is resisted.

One protocol generally accepted in the field that may be used to determine the release rate of a therapeutic agent such as a taxane or other material loaded in the CDP-taxane conjugates of the present invention involves degradation of any such matrix in a 0.1 M PBS solution (pH 7.4) at 37° C., an assay known in the art. For purposes of the present invention, the term “PBS protocol” is used herein to refer to such protocol.

In certain instances, the release rates of different CDP-taxane conjugates of the present invention may be compared by subjecting them to such a protocol. In certain instances, it may be necessary to process polymeric systems in the same fashion to allow direct and relatively accurate comparisons of different systems to be made. For example, the present invention teaches several different methods of formulating the CDP-taxane conjugates. Such comparisons may indicate that any one CDP-taxane conjugate releases incorporated material at a rate from about 2 or less to about 1000 or more times faster than another polymeric system.

Alternatively, a comparison may reveal a rate difference of about 3, 5, 7, 10, 25, 50, 100, 250, 500 or 750 times. Even higher rate differences are contemplated by the present invention and release rate protocols.

In certain embodiments, when formulated in a certain manner, the release rate for CDP-taxane conjugates of the present invention may present as mono- or bi-phasic.

Release of any material incorporated into the polymer matrix, which is often provided as a microsphere, may be characterized in certain instances by an initial increased release rate, which may release from about 5 to about 50% or more of any incorporated material, or alternatively about 10, 15, 20, 25, 30 or 40%, followed by a release rate of lesser magnitude.

The release rate of any incorporated material may also be characterized by the amount of such material released per day per mg of polymer matrix. For example, in certain embodiments, the release rate may vary from about 1 ng or less of any incorporated material per day per mg of polymeric system to about 500 or more ng/day/mg. Alternatively, the release rate may be about 0.05, 0.5, 5, 10, 25, 50, 75, 100, 125, 150, 175, 200, 250, 300, 350, 400, 450, or 500 ng/day/mg. In still other embodiments, the release rate of any incorporated material may be 10,000 ng/day/mg, or even higher. In certain instances, materials incorporated and characterized by such release rate protocols may include therapeutic agents, fillers, and other substances.

In another aspect, the rate of release of any material from any CDP-taxane conjugate of the present invention may be presented as the half-life of such material in the matrix.

In addition to the embodiment involving protocols for in vitro determination of release rates, in vivo protocols, whereby in certain instances release rates for polymeric systems may be determined in vivo, are also contemplated by the present invention. Other assays useful for determining the release of any material from the polymers of the present system are known in the art.

Physical Structures of the CDP-Taxane Conjugates

The CDP-taxane conjugates may be formed in a variety of shapes. For example, in certain embodiments, the CDP-taxane conjugates may be presented in the form of a nanoparticle. In one embodiment, the CDP-taxane conjugate self assembles into a nanoparticle. In one embodiment, the CDP-taxane conjugate self assembles into a nanoparticle in an aqueous solution, e.g., water.

In addition to intracellular delivery of a taxane, it also possible that nanoparticles of the CDP-taxane conjugates may undergo endocytosis, thereby obtaining access to the cell. The frequency of such an endocytosis process will likely depend on the size of any nanoparticle.

In one embodiment, the surface charge of the molecule is neutral, or slightly negative. In some embodiments, the zeta potential of the particle surface is from about -80 mV to about 50 mV.

Particles: Conjugate Number

Conjugate number, as used herein, is the number of cyclodextrin containing polymer (“CDP”) therapeutic agent conjugate molecules, present in a particle or nanoparticle. For purposes of determining conjugate number, a particle or nanoparticle is an entity having one, or typically, more than one CDP therapeutic agent conjugate molecules, which, at the concentration suitable for administration to humans, behaves as a single unit in any of water, e.g., water at neutral pH, PBS, e.g., PBS at pH 7.4, or in a formulation in which it will be administered to patients. For purposes of calculating conjugate number, a CDP therapeutic agent (e.g., taxane) conjugate molecule is a single CDP polymer with its covalently linked therapeutic agent (e.g., taxane).

Methods disclosed herein provide for evaluating a particle, e.g., a nanoparticle, or preparation of particles, e.g., nanoparticles, wherein said particles, e.g., nanoparticles, comprise a CDP therapeutic agent (e.g., taxane) conjugate. Generally, the method comprises providing a sample comprising a plurality of said particles, e.g., nanoparticles, determining a value for the number of CDP therapeutic agent (e.g., taxane) conjugates in a particle, e.g., nanoparticle, in the sample, to thereby evaluate a preparation of particles, e.g., nanoparticles.

Typically the value for a particle will be a function of the values obtained for a plurality of particles, e.g., the value will be the average of values determined for a plurality of particles.

In embodiments the method further comprises comparing the determined value with a reference value. The comparison can be used in a number of ways. By way of example, in response to a comparison or determination made in the method, a decision or step is taken, e.g., a production parameter in a process for making a particle is altered, the sample is classified, selected, accepted or discarded, released or withheld, processed into a drug product, shipped, moved to a different location, formulated, e.g., formulated with another substance, e.g., an excipient, labeled, packaged, released into commerce, or sold or offered for sale. E.g., based on the result of the determination, or upon comparison to a reference standard, the batch from which the sample is taken can be processed, e.g., as just described.

As discussed above, conjugate number is defined as the number of CDP-therapeutic agent (e.g., taxane) conjugate molecules that self-assemble into a particle or nanoparticle, thus

C_{J}=[CDP-therapeutic agent (e.g., taxane) conjugate]/P (or NP)

where Cj is conjugate number, [CDP-therapeutic agent (e.g., taxane) conjugate]/is the number of CDP-therapeutic agent (e.g., taxane) conjugate molecules, and P (or NP) is a single particle (or nanoparticle).

In order to arrive and conjugate number one determines the size of a particle, e.g., by dynamic light scattering. The size should be viscosity-adjusted size. The hydrodynamic volume of a CDP-therapeutic agent (e.g., taxane) conjugate, or a molecule of similar molecular weight, is determined, to provide an expected hydrodynamic volume. Comparison of the expected hydrodynamic volume for the CDP-therapeutic agent conjugate with the volume for a particle of determined size provides conjugate number.

The determination of conjugate number is demonstrated with CRLX101, in which camptothecin is coupled to the CDP backbone. In the case of CRLX101, a number of fundamental assumptions are made in postulating nanoparticle characteristics. First, macromolecular volume estimates are based on work done with bovine serum albumin (BSA), a biological macromolecule of similar size to CRLX101 (BSA MS=67 kDa, 101 MW=66.5 kDa). It has been demonstrated that a single strand of BSA has a hydrodynamic diameter of 9.5 nm Simple volume calculations yield a volume of 3589 nm³. Extending this to CRLX 101 with an average 30 nm particle, gives a volume of 33,485 nm³. With a particle size of 5-40 nm the conjugate number is 1-30. FIG. 1 shows a calculated strand dependence on particle size.

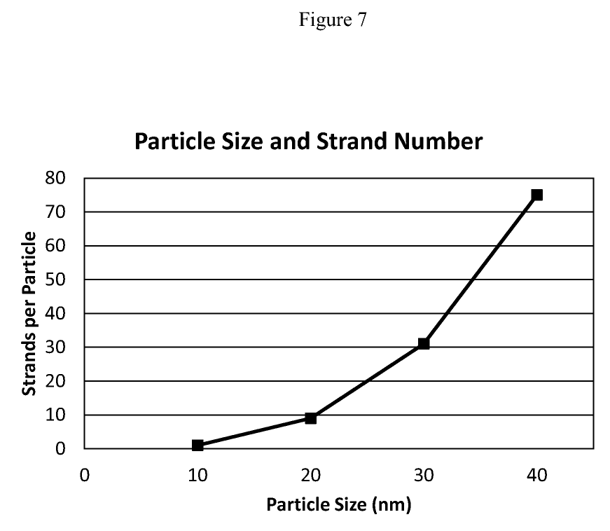

Given the particle size distribution of CRLX101, the conjugate number can range from 30-75, as shown in FIG. 7.

Polymer Polydispersity. CRLX101 molecules fall within a range of molecular weights, with molecules of varying weight providing varying contributions to the particle diameter and conjugate number. Particles could form which are made up of strands which are larger and smaller than the average. Strands may also associate to a maximum size which could be shear-limited.

Particle Shape. Particle shape is assumed to be roughly spherical, and driven by either (or both) the hydrophobic region created by the CDP-therapeutic agent (e.g., taxane) conjugate , or by guest-host complexation with pendant therapeutic agent molecules making inclusion complexes with CDs from adjacent strands. One critical point of note is that as a drug product, the NPs are in a somewhat controlled environment as they are characterized. Upon administration, myriad possibilities exist for interaction with endogenous substances: inclusion complexes of circulating small molecules, metal ion complexation with the PEG subunits, etc. Any one of these are all of them in concert could dramatically alter the NP structure and function.

### CDPs, Methods of Making Same, and Methods of Conjugating CDPs to Taxanes

Generally, the CDP-taxane conjugates described herein can be prepared in one of two ways: monomers bearing taxanes, targeting ligands, and/or cyclodextrin moieties can be polymerized, or polymer backbones can be derivatized with taxanes, targeting ligands, and/or cyclodextrin moieties.

Thus, in one embodiment, the synthesis of the CDP-taxane conjugates can be accomplished by reacting monomers M-L-CD and M-L-D (and, optionally, M-L-T), wherein

CD represents a cyclic moiety, such as a cyclodextrin molecule, or derivative thereof;

L, independently for each occurrence, may be absent or represents a linker group;

D, independently for each occurrence, represents the same or different taxane or prodrug thereof;

T, independently for each occurrence, represents the same or different targeting ligand or precursor thereof; and

M represents a monomer subunit bearing one or more reactive moieties capable of undergoing a polymerization reaction with one or more other M in the monomers in the reaction mixture, under conditions that cause polymerization of the monomers to take place.

In some embodiments, one or more of the taxane moieties in the CDP-taxane conjugate can be replaced with another therapeutic agent, e.g., another anticancer agent or anti-inflammatory agent.

In certain embodiments, the reaction mixture may further comprise monomers that do not bear CD, T, or D moieties, e.g., to space the derivatized monomer units throughout the polymer.

In an alternative embodiment, the invention contemplates synthesizing a CDP-taxane conjugate by reacting a polymer P (the polymer bearing a plurality of reactive groups, such as carboxylic acids, alcohols, thiols, amines, epoxides, etc.) with grafting agents X-L-CD and/or Y-L-D (and, optionally, Z-L-T), wherein

CD represents a cyclic moiety, such as a cyclodextrin molecule, or derivative thereof;

L, independently for each occurrence, may be absent or represents a linker group;

D, independently for each occurrence, represents the same or different taxane or prodrug thereof;

T, independently for each occurrence, represents the same or different targeting ligand or precursor thereof;

X, independently for each occurrence, represents a reactive group, such as carboxylic acids, alcohols, thiols, amines, epoxides, etc., capable of forming a covalent bond with a reactive group of the polymer; and

Y and Z, independently for each occurrence, represent inclusion hosts or reactive groups, such as carboxylic acids, alcohols, thiols, amines, epoxides, etc., capable of forming a covalent bond with a reactive group of the polymer or inclusion complexes with CD moieties grafted to the polymer, under conditions that cause the grafting agents to form covalent bonds and/or inclusion complexes, as appropriate, with the polymer or moieties grafted to the polymer.

In some embodiments, one or more of the taxane moieties in the CDP-taxane conjugate can be replaced with another therapeutic agent, e.g., another anticancer agent or anti-inflammatory agent.

For example, if the CDP includes alcohols, thiols, or amines as reactive groups, the grafting agents may include reactive groups that react with them, such as isocyanates, isothiocyanates, acid chlorides, acid anhydrides, epoxides, ketenes, sulfonyl chlorides, activated carboxylic acids (e.g., carboxylic acids treated with an activating agent such as PyBrOP, carbonyldiimidazole, or another reagent that reacts with a carboxylic acid to form a moiety susceptible to nucleophilic attack), or other electrophilic moieties known to those of skill in the art. In certain embodiments, a catalyst may be needed to cause the reaction to take place (e.g., a Lewis acid, a transition metal catalyst, an amine base, etc.) as will be understood by those of skill in the art.

In certain embodiments, the different grafting agents are reacted with the polymer simultaneously or substantially simultaneously (e.g., in a one-pot reaction), or are reacted sequentially with the polymer (optionally with a purification and/or wash step between reactions).

Another aspect of the present invention is a method for manufacturing the linear or branched CDPs and CDP-taxane conjugates as described herein. While the discussion below focuses on the preparation of linear cyclodextrin molecules, one skilled in the art would readily recognize that the methods described can be adapted for producing branched polymers by choosing an appropriate comonomer precursor.

Accordingly, one embodiment of the invention is a method of preparing a linear CDP. According to the invention, a linear CDP may be prepared by copolymerizing a cyclodextrin monomer precursor disubstituted with one or more appropriate leaving groups with a comonomer precursor capable of displacing the leaving groups. The leaving group, which may be the same or different, may be any leaving group known in the art which may be displaced upon copolymerization with a comonomer precursor. In a preferred embodiment, a linear CDP may be prepared by iodinating a cyclodextrin monomer precursor to form a diiodinated cyclodextrin monomer precursor and copolymerizing the diiodinated cyclodextrin monomer precursor with a comonomer precursor to form a linear CDP having a repeating unit of formula I or II, provided in the section entitles “CDP-Taxane conjugates” or a combination thereof, each as described above. In some embodiments, the cyclodextrin moiety precursors are in a composition, the composition being substantially free of cyclodextrin moieties having other than two positions modified to bear a reactive site (e.g., 1, 3, 4, 5, 6, or 7). While examples presented below discuss iodinated cyclodextrin moieties, one skilled in the art would readily recognize that the present invention contemplates and encompasses cyclodextrin moieties wherein other leaving groups such as alkyl and aryl sulfonate may be present instead of iodo groups. In a preferred embodiment, a method of preparing a linear cyclodextrin copolymer of the invention by iodinating a cyclodextrin monomer precursor as described above to form a diiodinated cyclodextrin monomer precursor of formula IVa, IVb, IVc or a mixture thereof:

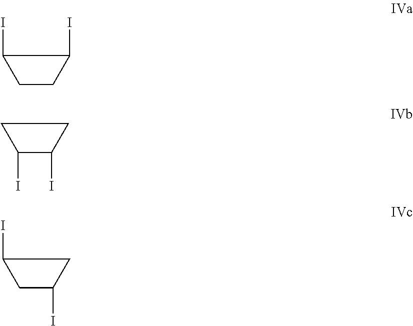

In some embodiments, the iodine moieties as shown on the cyclodextrin moieties are positioned such that the derivatization on the cyclodextrin is on the A and D glucopyranose moieties. In some embodiments, the iodine moieties as shown on the cyclodextrin moieties are positioned in such that the derivatization on the cyclodextrin is on the A and C glucopyranose moieties. In some embodiments, the iodine moieties as shown on the cyclodextrin moieties are positioned in such that the derivatization on the cyclodextrin is on the A and F glucopyranose moieties. In some embodiments, the iodine moieties as shown on the cyclodextrin moieties are positioned in such that the derivatization on the cyclodextrin is on the A and E glucopyranose moieties.

The diiodinated cyclodextrin may be prepared by any means known in the art. (Tabushi et al. J. Am. Chem. 106, 5267-5270 (1984); Tabushi et al. J. Am. Chem. 106, 4580-4584 (1984)). For example, β-cyclodextrin may be reacted with biphenyl-4,4′-disulfonyl chloride in the presence of anhydrous pyridine to form a biphenyl-4,4′-disulfonyl chloride capped β-cyclodextrin which may then be reacted with potassium iodide to produce diiodo-β-cyclodextrin. The cyclodextrin monomer precursor is iodinated at only two positions. By copolymerizing the diiodinated cyclodextrin monomer precursor with a comonomer precursor, as described above, a linear cyclodextrin polymer having a repeating unit of Formula Ia, Ib, or a combination thereof, also as described above, may be prepared. If appropriate, the iodine or iodo groups may be replaced with other known leaving groups.

Also according to the invention, the iodo groups or other appropriate leaving group may be displaced with a group that permits reaction with a comonomer precursor, as described above. For example, a diiodinated cyclodextrin monomer precursor of formula IVa, IVb, IVc or a mixture thereof may be aminated to form a diaminated cyclodextrin monomer precursor of formula Va, Vb, Vc or a mixture thereof:

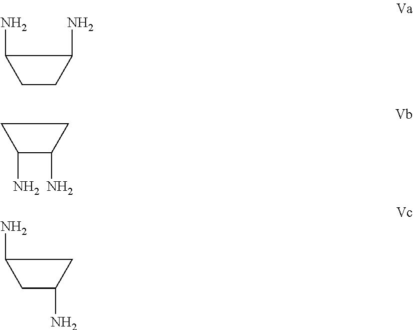

In some embodiments, the amino moieties as shown on the cyclodextrin moieties are positioned such that the derivatization on the cyclodextrin is on the A and D glucopyranose moieties. In some embodiments, the amino moieties as shown on the cyclodextrin moieties are positioned in such that the derivatization on the cyclodextrin is on the A and C glucopyranose moieties. In some embodiments, the amino moieties as shown on the cyclodextrin moieties are positioned in such that the derivatization on the cyclodextrin is on the A and F glucopyranose moieties. In some embodiments, the amino moieties as shown on the cyclodextrin moieties are positioned in such that the derivatization on the cyclodextrin is on the A and E glucopyranose moieties.

The diaminated cyclodextrin monomer precursor may be prepared by any means known in the art. (Tabushi et al. Tetrahedron Lett. 18:11527-1530 (1977); Mungall et al., J. Org. Chem. 16591662 (1975)). For example, a diiodo-β-cyclodextrin may be reacted with sodium azide and then reduced to form a diamino-⊖-cyclodextrin). The cyclodextrin monomer precursor is aminated at only two positions. The diaminated cyclodextrin monomer precursor may then be copolymerized with a comonomer precursor, as described above, to produce a linear cyclodextrin copolymer having a repeating unit of formula I-II provided in the section entitles “CDP-Taxane conjugates” or a combination thereof, also as described above. However, the amino functionality of a diaminated cyclodextrin monomer precursor need not be directly attached to the cyclodextrin moiety. Alternatively, the amino functionality or another nucleophilic functionality may be introduced by displacement of the iodo or other appropriate leaving groups of a cyclodextrin monomer precursor with amino group containing moieties such as, for example, HSCH₂CH₂NH₂ (or a di-nucleophilic molecule more generally represented by HW—(CR₁R₂)ₙ—WH wherein W, independently for each occurrence, represents O, S, or NR₁; R₁ and R₂, independently for each occurrence, represent H, (un)substituted alkyl, (un)substituted aryl, (un)substituted heteroalkyl, (un)substituted heteroaryl) with an appropriate base such as a metal hydride, alkali or alkaline carbonate, or tertiary amine to form a diaminated cyclodextrin monomer precursor of formula Vd, Ve, Vf or a mixture thereof:

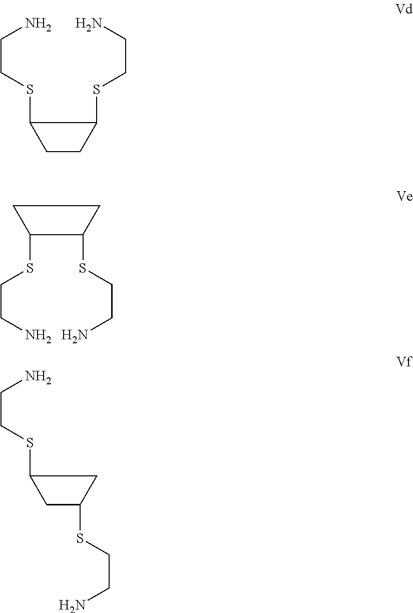

In some embodiments, the —SCH₂CH₂NH₂ moieties as shown on the cyclodextrin moieties are positioned such that the derivatization on the cyclodextrin is on the A and D glucopyranose moieties. In some embodiments, the —SCH₂CH₂NH₂ moieties as shown on the cyclodextrin moieties are positioned in such that the derivatization on the cyclodextrin is on the A and C glucopyranose moieties. In some embodiments, the —SCH₂CH₂NH₂ moieties as shown on the cyclodextrin moieties are positioned in such that the derivatization on the cyclodextrin is on the A and F glucopyranose moieties. In some embodiments, the —SCH₂CH₂NH₂ moieties as shown on the cyclodextrin moieties are positioned in such that the derivatization on the cyclodextrin is on the A and E glucopyranose moieties.

A linear oxidized CDP may also be prepared by oxidizing a reduced linear cyclodextrin-containing copolymer as described below. This method may be performed as long as the comonomer does not contain an oxidation sensitive moiety or group such as, for example, a thiol.

A linear CDP of the invention may be oxidized so as to introduce at least one oxidized cyclodextrin monomer into the copolymer such that the oxidized cyclodextrin monomer is an integral part of the polymer backbone. A linear CDP which contains at least one oxidized cyclodextrin monomer is defined as a linear oxidized cyclodextrin copolymer or a linear oxidized cyclodextrin-containing polymer. The cyclodextrin monomer may be oxidized on either the secondary or primary hydroxyl side of the cyclodextrin moiety. If more than one oxidized cyclodextrin monomer is present in a linear oxidized cyclodextrin copolymer of the invention, the same or different cyclodextrin monomers oxidized on either the primary hydroxyl side, the secondary hydroxyl side, or both may be present. For illustration purposes, a linear oxidized cyclodextrin copolymer with oxidized secondary hydroxyl groups has, for example, at least one unit of formula VIa or VIb:

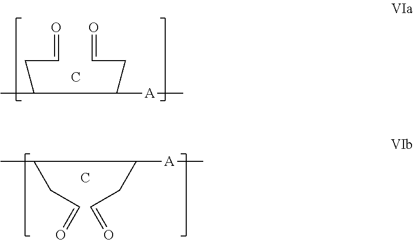

In formulae VIa and VIb, C is a substituted or unsubstituted oxidized cyclodextrin monomer and the comonomer (i.e., shown herein as A) is a comonomer bound, i.e., covalently bound, to the oxidized cyclodextrin C. Also in formulae VIa and VIb, oxidation of the secondary hydroxyl groups leads to ring opening of the cyclodextrin moiety and the formation of aldehyde groups.

A linear oxidized CDP copolymer may be prepared by oxidation of a linear cyclodextrin copolymer as discussed above. Oxidation of a linear cyclodextrin copolymer of the invention may be accomplished by oxidation techniques known in the art. (Hisamatsu et al., Starch 44:188-191 (1992)). Preferably, an oxidant such as, for example, sodium periodate is used. It would be understood by one of ordinary skill in the art that under standard oxidation conditions that the degree of oxidation may vary or be varied per copolymer. Thus in one embodiment of the invention, a CDP may contain one oxidized cyclodextrin monomer. In another embodiment, substantially all cyclodextrin monomers of the copolymer would be oxidized.

Another method of preparing a linear oxidized CDP involves the oxidation of a diiodinated or diaminated cyclodextrin monomer precursor, as described above, to form an oxidized diiodinated or diaminated cyclodextrin monomer precursor and copolymerization of the oxidized diiodinated or diaminated cyclodextrin monomer precursor with a comonomer precursor. In a preferred embodiment, an oxidized diiodinated cyclodextrin monomer precursor of formula VIIa, VIIb, VIIc, or a mixture thereof:

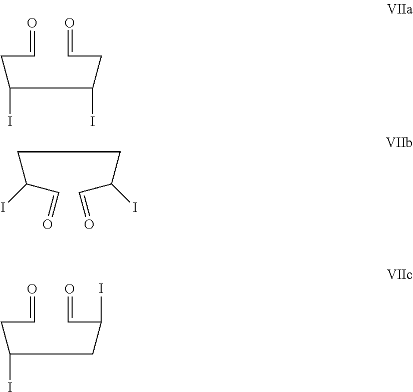

may be prepared by oxidation of a diiodinated cyclodextrin monomer precursor of formulae IVa, IVb, IVc, or a mixture thereof, as described above. In another preferred embodiment, an oxidized diaminated cyclodextrin monomer precursor of formula VIIIa, VIIIb, VIIIc or a mixture thereof:

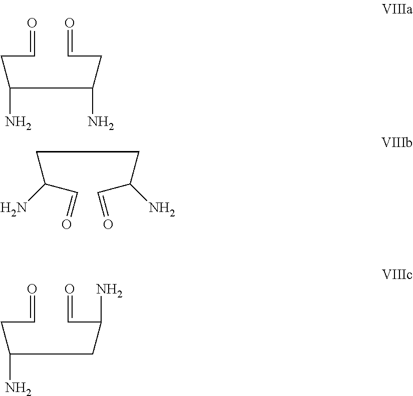

may be prepared by amination of an oxidized diiodinated cyclodextrin monomer precursor of formulae VIIa, VIIb, VIIc, or a mixture thereof, as described above. In still another preferred embodiment, an oxidized diaminated cyclodextrin monomer precursor of formula IXa, IXb, IXc or a mixture thereof:

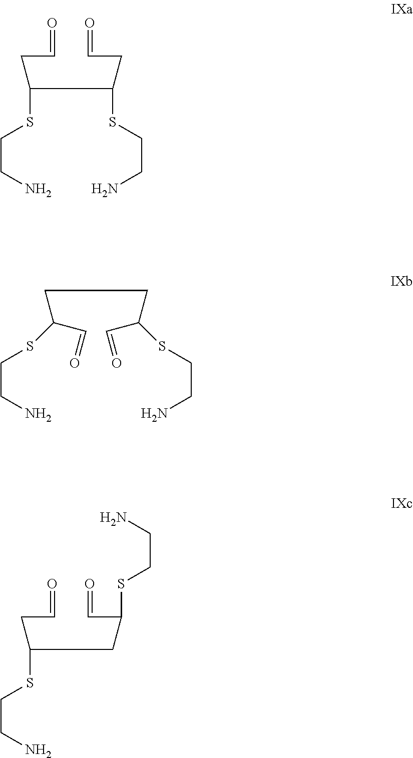 [SMILES: NCCSC(CC=O)C(CC=O)SCCN ; NCCSC(C=O)CCC(C=O)SCCN ; NCCSC(C=O)CC(CC=O)SCCN]

may be prepared by displacement of the iodo or other appropriate leaving groups of an oxidized cyclodextrin monomer precursor disubstituted with an iodo or other appropriate leaving group with the amino or other nucleophilic group containing moiety such as, e.g. HSCH₂CH₂NH₂ (or a di-nucleophilic molecule more generally represented by HW—(CR₁R₂)ₙ—WH wherein W, independently for each occurrence, represents O, S, or NRi; R₁ and R₂, independently for each occurrence, represent H, (un)substituted alkyl, (un)substituted aryl, (un)substituted heteroalkyl, (un)substituted heteroaryl) with an appropriate base such as a metal hydride, alkali or alkaline carbonate, or tertiary amine

Alternatively, an oxidized diiodinated or diaminated cyclodextrin monomer precursor, as described above, may be prepared by oxidizing a cyclodextrin monomer precursor to form an oxidized cyclodextrin monomer precursor and then diiodinating and/or diaminating the oxidized cyclodextrin monomer, as described above. As discussed above, the cyclodextrin moiety may be modified with other leaving groups other than iodo groups and other amino group containing functionalities. The oxidized diiodinated or diaminated cyclodextrin monomer precursor may then be copolymerized with a comonomer precursor, as described above, to form a linear oxidized cyclodextrin copolymer of the invention.

A linear oxidized CDP may also be further modified by attachment of at least one ligand to the copolymer. The ligand is as described above.

In some embodiments, a CDP comprises: cyclodextrin moieties, and comonomers which do not contain cyclodextrin moieties (comonomers), and wherein the CDP comprises at least four, five six, seven, eight, nine, ten, eleven, twelve, thirteen, fourteen, fifteen, sixteen, seventeen, eighteen, nineteen or twenty cyclodextrin moieties and at least four, five six, seven, eight, nine, ten, eleven, twelve, thirteen, fourteen, fifteen, sixteen, seventeen, eighteen, nineteen or twenty comonomers.

In some embodiments, the at least four, five six, seven, eight, etc., cyclodextrin moieties and at least four, five six, seven, eight, nine, ten, eleven, twelve, thirteen, fourteen, fifteen, sixteen, seventeen, eighteen, nineteen or twenty comonomers alternate in the water soluble linear polymer.

In some embodiments, the cyclodextrin moieties comprise linkers to which therapeutic agents may be further linked.

In some embodiments, the CDP has no taxanes attached. In some embodiments, the CDP has a plurality (i.e., more than one) of taxanes attached (e.g., through a linker). In some embodiments, the taxanes are attached via a second linker

In some embodiments, the comonomer is a compound containing residues of least two functional groups through which reaction and thus linkage of the cyclodextrin monomers is achieved. In some embodiments, the functional groups, which may be the same or different, terminal or internal, of each comonomer comprise an amino, acid, imidazole, hydroxyl, thio, acyl halide, —HC═CH—, —C≡C— group, or derivative thereof. In some embodiments, the residues of the two functional groups are the same and are located at termini of the comonomer. In some embodiments, a comonomer contains one or more pendant groups with at least one functional group through which reaction and thus linkage of a taxane can be achieved. In some embodiments, the functional groups, which may be the same or different, terminal or internal, of each comonomer pendant group comprise an amino, acid, imidazole, hydroxyl, thiol, acyl halide, ethylene, ethyne group, or derivative thereof. In some embodiments, the pendant group is a substituted or unsubstituted branched, cyclic or straight chain C₁-C₁₀ alkyl, or arylalkyl optionally containing one or more heteroatoms within the chain or ring.

In some embodiments, the cyclodextrin moiety comprises an alpha, beta, or gamma cyclodextrin moiety.

In some embodiments, the CDP is suitable for the attachment of sufficient taxane such that up to at least 5%, 10%, 15%, 20%, 25%, 30%, or even 35% by weight of the water soluble linear polymer, when conjugated, is taxane.

In some embodiments, the molecular weight of the CDP is 10,000-500,000 Da, e.g., about 30,000 to about 100,000 Da.

In some embodiments, the cyclodextrin moieties make up at least about 2%, 5%, 10%, 11%, 12%, 13%, 14%, 15%, 16%, 17%, 18%, 19%, 20%, 30%, 50% or 80% of the polymer by weight.

In some embodiments, the CDP is made by a method comprising providing cyclodextrin moiety precursors modified to bear one reactive site at each of exactly two positions, and reacting the cyclodextrin moiety with comonomer precursors having exactly two reactive moieties capable of forming a covalent bond with the reactive sites under polymerization conditions that promote reaction of the reactive sites with the reactive moieties to form covalent bonds between the comonomers and the cyclodextrin moieties, whereby a CDP comprising alternating units of a cyclodextrin moiety and comonomer is produced.

In some embodiments, the CDP comprises a comonomer selected from the group consisting of: an alkylene chain, polysuccinic anhydride, poly-L-glutamic acid, poly(ethyleneimine), an oligosaccharide, and an amino acid chain. In some embodiments, a comonomer comprises a polyethylene glycol chain. In some embodiments, the CDP comprises a comonomer selected from the group consisting of: polyglycolic acid and polylactic acid chain.

In some embodiments, a comonomer comprises a hydrocarbylene group wherein one or more methylene groups is optionally replaced by a group Y (provided that none of the Y groups are adjacent to each other), wherein each Y, independently for each occurrence, is selected from, substituted or unsubstituted aryl, heteroaryl, cycloalkyl, heterocycloalkyl, or —O—, C(═X) (wherein X is NR₁, O or S), —OC(O)—, —C(═O)O, —NR₁—, —NR₁CO—, —C(O)NR₁—, —S(O)ₙ— (wherein n is 0, 1, or 2), —OC(O)—NR₁, —NR₁—C(O)—NR₁—, —NR₁1-C(NR₁)—NR₁—, and —B(OR₁)—; and R₁, independently for each occurrence, represents H or a lower alkyl.

In some embodiments, the CDP is a polymer of the following formula:

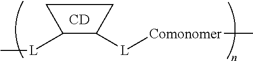

wherein each L is independently a linker, each comonomer is independently a comonomer described herein, and n is at least 4, 5, 6, 7, 8, 9, 10, 11, 12, 13, 14, 15, 16, 17, 18, 19 or 20. In some embodiments, the molecular weight of the comonomer is from about 2000 to about 5000 Da (e.g., from about 3000 to about 4000 Da (e.g., about 3400 Da).

In some embodiments, the CDP is a polymer of the following formula:

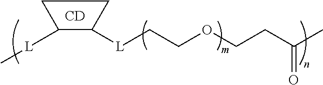

wherein each L is independently a linker,
wherein the group

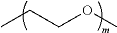

has a Mw of 3.4 kDa or less and n is at least 4, 5, 6, 7, 8, 9, 10, 11, 12, 13, 14, 15, 16, 17, 18, 19 or 20.

In some embodiments,

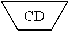

is alpha, beta or gamma cyclodextrin, e.g., beta cyclodextrin.

In some embodiments, each L independently comprises an amino acid or a derivative thereof. In some embodiments, at least one L comprises cysteine or a derivative thereof. In some embodiments, each L comprises cysteine. In some embodiments, each L is cysteine and the cysteine is connected to the CD by way of a thiol linkage.

In some embodiments, the CDP is a polymer of the following formula:

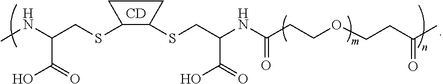

wherein the group

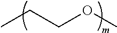

has a Mw of 3.4 kDa or less and n is at least 4, 5, 6, 7, 8, 9, 10, 11, 12, 13, 14, 15, 16, 17, 18, 19 or 20.

In some embodiments,

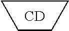

is alpha, beta or gamma cyclodextrin, e.g., beta cyclodextrin.

In some embodiments, the CDP is a polymer of the following formula:

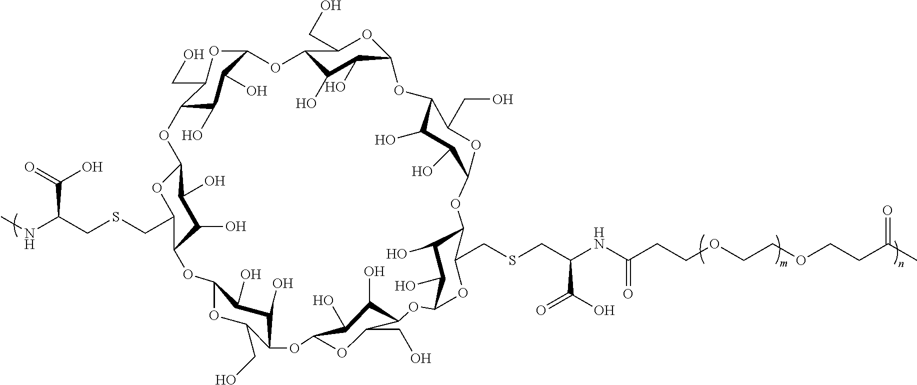

wherein the group

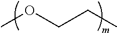

has a Mw of 3.4 kDa or less and n is at least 4, 5, 6, 7, 8, 9, 10, 11, 12, 13, 14, 15, 16, 17, 18, 19 or 20.

In some embodiments, the group

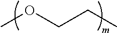

has a Mw of 3.4 kDa and the Mw of the compound as a whole is from 27 kDa to 99.6 kDa.

The CDPs described herein can be made using a variety of methods including those described herein. In some embodiments, a CDP can be made by: providing cyclodextrin moiety precursors; providing comonomer precursors which do not contain cyclodextrin moieties (comonomer precursors); and copolymerizing the said cyclodextrin moiety precursors and comonomer precursors to thereby make a CDP wherein CDP comprises at least four, five six, seven, eight, or more, cyclodextrin moieties and at least four, five six, seven, eight, or more, comonomers.

In some embodiments, the at least four, five, six, seven, eight, or more cyclodextrin moieties and at least four, five, six, seven, eight, or more comonomers alternate in the water soluble linear polymer. In some embodiments, the method includes providing cyclodextrin moiety precursors modified to bear one reactive site at each of exactly two positions, and reacting the cyclodextrin moiety precursors with comonomer precursors having exactly two reactive moieties capable of forming a covalent bond with the reactive sites under polymerization conditions that promote reaction of the reactive sites with the reactive moieties to form covalent bonds between the comonomers and the cyclodextrin moieties, whereby a CDP comprising alternating units of a cyclodextrin moiety and a comonomer is produced.

In some embodiments, the cyclodextrin comonomers comprise linkers to which taxanes may be further linked. In some embodiments, the taxanes are linked via second linkers.

In some embodiments, the comonomer precursor is a compound containing at least two functional groups through which reaction and thus linkage of the cyclodextrin moieties is achieved. In some embodiments, the functional groups, which may be the same or different, terminal or internal, of each comonomer precursor comprise an amino, acid, imidazole, hydroxyl, thio, acyl halide, —HC═CH—, —C≡C— group, or derivative thereof. In some embodiments, the two functional groups are the same and are located at termini of the comonomer precursor. In some embodiments, a comonomer contains one or more pendant groups with at least one functional group through which reaction and thus linkage of a therapeutic agent can be achieved. In some embodiments, the functional groups, which may be the same or different, terminal or internal, of each comonomer pendant group comprise an amino, acid, imidazole, hydroxyl, thiol, acyl halide, ethylene, ethyne group, or derivative thereof. In some embodiments, the pendant group is a substituted or unsubstituted branched, cyclic or straight chain C₁-C₁₀ alkyl, or arylalkyl optionally containing one or more heteroatoms within the chain or ring.

In some embodiments, the cyclodextrin moiety comprises an alpha, beta, or gamma cyclodextrin moiety.

In some embodiments, the CDP is suitable for the attachment of sufficient taxane such that up to at least 3%, 5%, 10%, 15%, 20%, 25%, 30%, or even 35% by weight of the CDP, when conjugated, is taxane.

In some embodiments, the CDP has a molecular weight of 10,000-500,000. In some embodiments, the cyclodextrin moieties make up at least about 2%, 5%, 10%, 20%, 30%, 50% or 80% of the CDP by weight.

In some embodiments, the CDP comprises a comonomer selected from the group consisting of: an alkylene chain, polysuccinic anhydride, poly-L-glutamic acid, poly(ethyleneimine), an oligosaccharide, and an amino acid chain. In some embodiments, a comonomer comprises a polyethylene glycol chain. In some embodiments, the CDP comprises a comonomer selected from the group consisting of: polyglycolic acid and polylactic acid chain. the CDP comprises a comonomer selected from the group consisting of a comonomer comprises a hydrocarbylene group wherein one or more methylene groups is optionally replaced by a group Y (provided that none of the Y groups are adjacent to each other), wherein each Y, independently for each occurrence, is selected from, substituted or unsubstituted aryl, heteroaryl, cycloalkyl, heterocycloalkyl, or —O—, C(═X) (wherein X is NR₁, O or S), —OC(O)—, —C(═O)O, —NR₁—, —NR₁CO—, —C(O)NR₁—, —S(O)ₙ— (wherein n is 0, 1, or 2), —OC(O)—NR₁, —NR₁—C(O)—NR₁—, —NR₁—C(NR₁)—NR₁—, and —B(OR₁)—; and R₁, independently for each occurrence, represents H or a lower alkyl.

In some embodiments, a CDP of the following formula can be made by the scheme below:

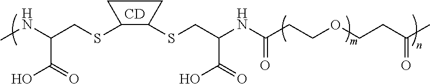

providing a compound of formula A and formula B:

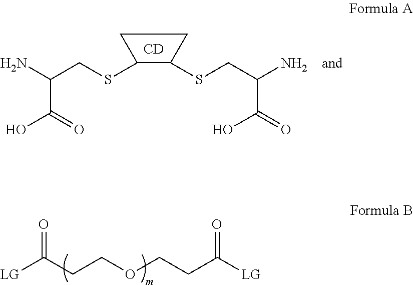

wherein LG is a leaving group;
and contacting the compounds under conditions that allow for the formation of a covalent bond between the compounds of formula A and B, to form a polymer of the following formula:

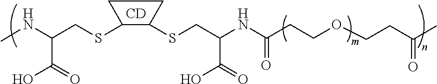

wherein the group

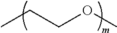

has a Mw of 3.4 kDa or less and n is at least four.

In some embodiments, Formula B is

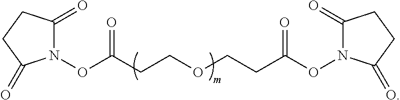

In some embodiments, the group

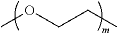

has a Mw of 3.4 kDa and the Mw of the compound is from 27 kDa to 99.6 kDa.

In some embodiments, the compounds of formula A and formula B are contacted in the presence of a base. In some embodiments, the base is an amine containing base. In some embodiments, the base is DEA.

In some embodiments, a CDP of the following formula can be made by the scheme below:

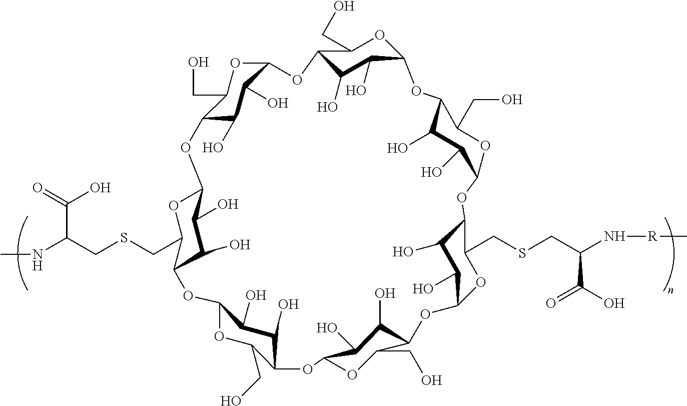

wherein R is of the form:

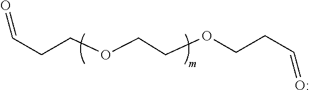

comprising the steps of:
reacting a compound of the formula below:

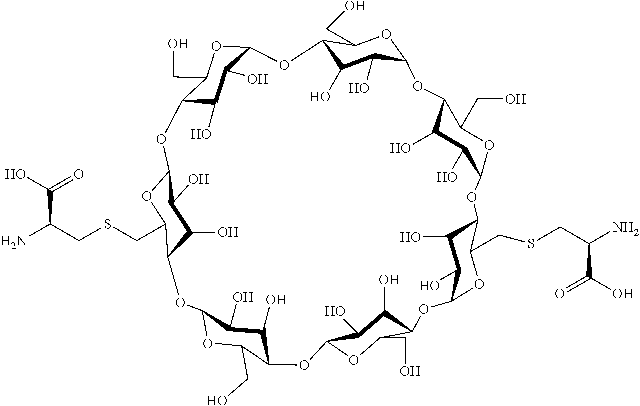 [SMILES: N[C@H](CSCC1O[C@@H]2O[C@@H]3C(CO)O[C@@H](O[C@@H]4C(CO)O[C@@H](O[C@@H]5C(CSC[C@@H](N)C(=O)O)O[C@@H](O[C@@H]6C(CO)O[C@H](O[C@@H]7C(CO)O[C@H](O[C@@H]8C(CO)O[C@H](O[C@H]1[C@H](O)C2O)C(O)[C@H]8O)C(O)[C@H]7O)C(O)[C@H]6O)C(O)[C@H]5O)C(O)[C@H]4O)C(O)[C@H]3O)C(=O)O]

with a compound of the formula below:

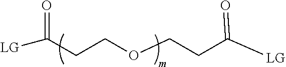

wherein the group

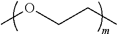

has a Mw of 3.4 kDa or less and n is at least four,

in the presence of a non-nucleophilic organic base in a solvent.

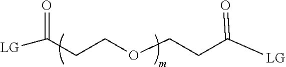

In some embodiments, is

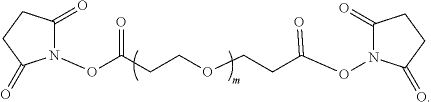

In some embodiments, the solvent is a polar aprotic solvent. In some embodiments, the solvent is DMSO.

In some embodiments, the method also includes the steps of dialysis; and lyophylization.

In some embodiments, a CDP provided below can be made by the following scheme:

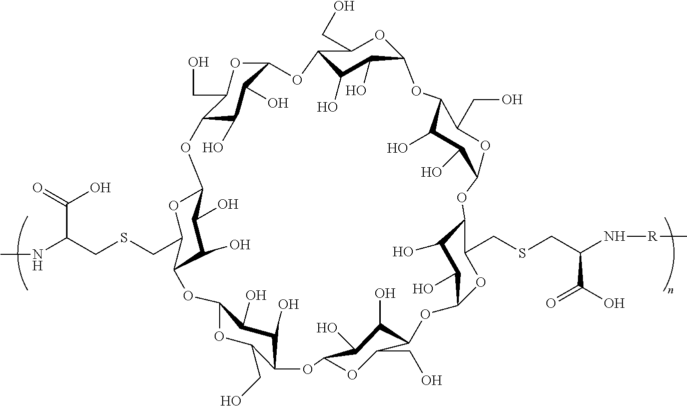

wherein R is of the form:

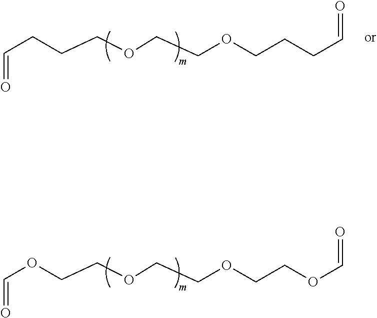

comprising the steps of:

reacting a compound of the formula below:

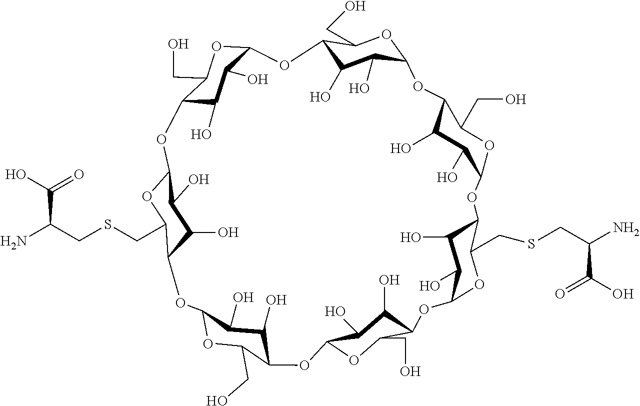 [SMILES: N[C@H](CSCC1O[C@@H]2O[C@@H]3C(CO)O[C@@H](O[C@@H]4C(CO)O[C@@H](O[C@@H]5C(CSC[C@@H](N)C(=O)O)O[C@@H](O[C@@H]6C(CO)O[C@H](O[C@@H]7C(CO)O[C@H](O[C@@H]8C(CO)O[C@H](O[C@H]1[C@H](O)C2O)C(O)[C@H]8O)C(O)[C@H]7O)C(O)[C@H]6O)C(O)[C@H]5O)C(O)[C@H]4O)C(O)[C@H]3O)C(=O)O]

with a compound of the formula below:

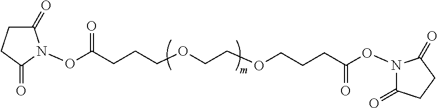

wherein the group

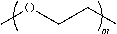

has a Mw of 3.4 kDa or less and n is at least four,

or with a compound provided below:

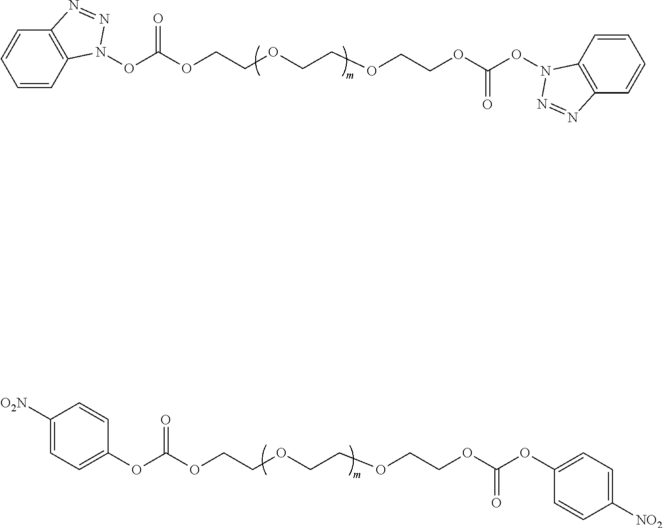

wherein the group

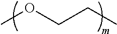

has a Mw of 3.4 kDa; in the presence of a non-nucleophilic organic base in DMSO;
and dialyzing and lyophilizing the following polymer

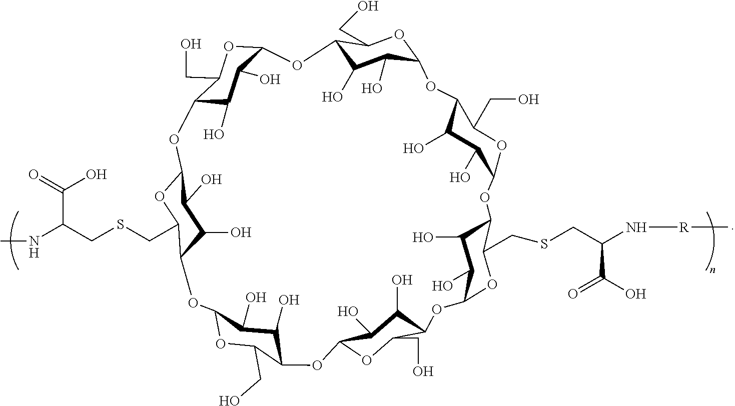

A CDP described herein may be attached to or grafted onto a substrate. The substrate may be any substrate as recognized by those of ordinary skill in the art. In another preferred embodiment of the invention, a CDP may be crosslinked to a polymer to form, respectively, a crosslinked cyclodextrin copolymer or a crosslinked oxidized cyclodextrin copolymer. The polymer may be any polymer capable of crosslinking with a CDP (e.g., polyethylene glycol (PEG) polymer, polyethylene polymer). The polymer may also be the same or different CDP. Thus; for example, a linear CDP may be crosslinked to any polymer including, but not limited to, itself, another linear CDP, and a linear oxidized CDP. A crosslinked linear CDP may be prepared by reacting a linear CDP with a polymer in the presence of a crosslinking agent. A crosslinked linear oxidized CDP may be prepared by reacting a linear oxidized CDP with a polymer in the presence of an appropriate crosslinking agent. The crosslinking agent may be any crosslinking agent known in the art. Examples of crosslinking agents include dihydrazides and disulfides. In a preferred embodiment, the crosslinking agent is a labile group such that a crosslinked copolymer may be uncrosslinked if desired.

A linear CDP and a linear oxidized CDP may be characterized by any means known in the art. Such characterization methods or techniques include, but are not limited to, gel permeation chromatography (GPC), matrix assisted laser desorption ionization-time of flight mass spectrometry (MALDI-TOF Mass spec), ¹H and ¹³C NMR, light scattering and titration.

The invention also provides a cyclodextrin composition containing at least one linear CDP and at least one linear oxidized CDP as described above. Accordingly, either or both of the linear CDP and linear oxidized CDP may be crosslinked to another polymer and/or bound to a ligand as described above. Therapeutic compositions according to the invention contain a taxane and a linear CDP or a linear oxidized CDP, including crosslinked copolymers. A linear CDP, a linear oxidized CDP and their crosslinked derivatives are as described above. The taxane may be any synthetic, semi-synthetic or naturally occurring biologically active taxane, including those known in the art.

One aspect of the present invention contemplates attaching a taxane to a CDP for delivery of a taxane. The present invention discloses various types of linear, branched, or grafted CDPs wherein a taxane is covalently bound to the polymer. In certain embodiments, the taxane is covalently linked via a biohydrolyzable bond, for example, an ester, amide, carbamates, or carbonate.

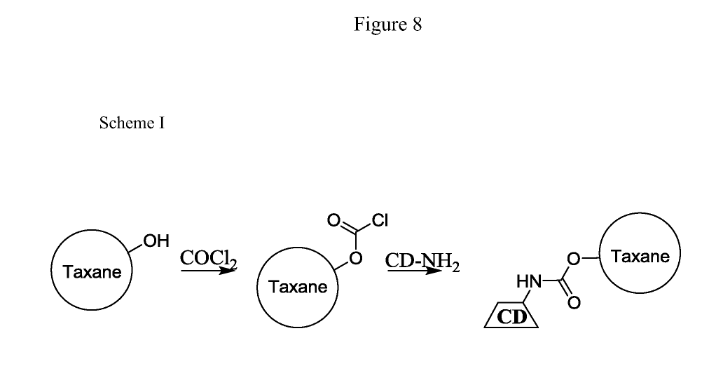

An exemplary synthetic scheme for covalently bonding a derivatized CD to a taxane is shown in FIG. 8.

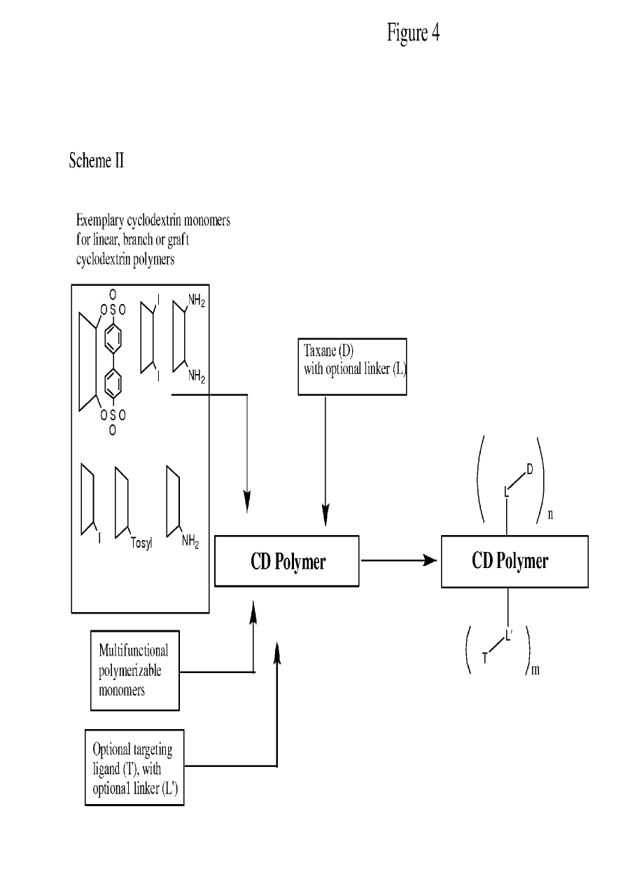

A general strategy for synthesizing linear, branched or grafted cyclodextrin-containing polymers (CDPs) for loading a taxane, and an optional targeting ligand is shown in FIG. 4.

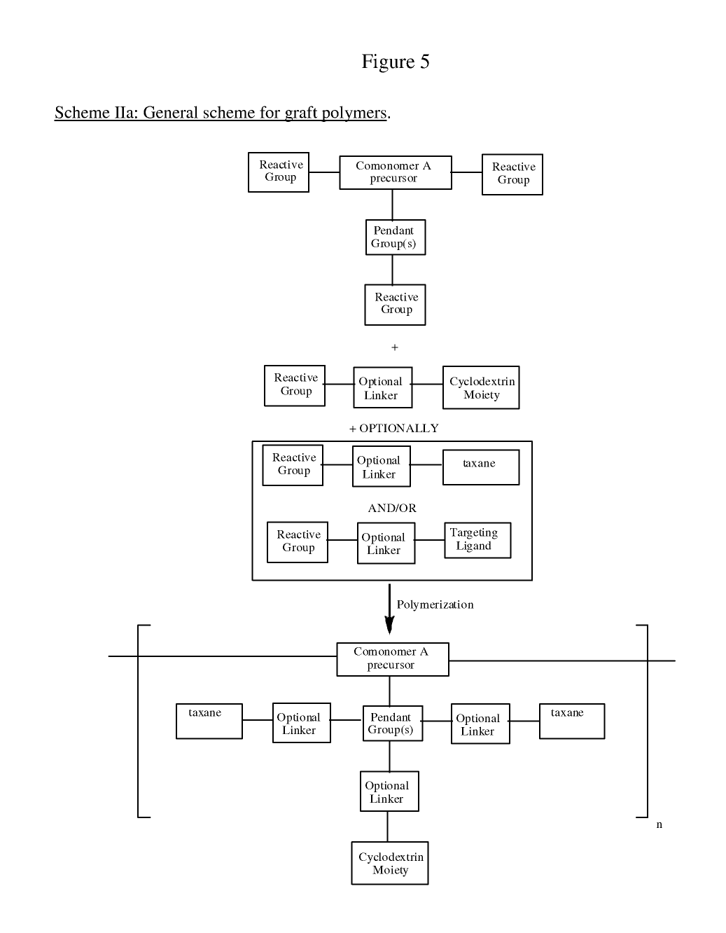

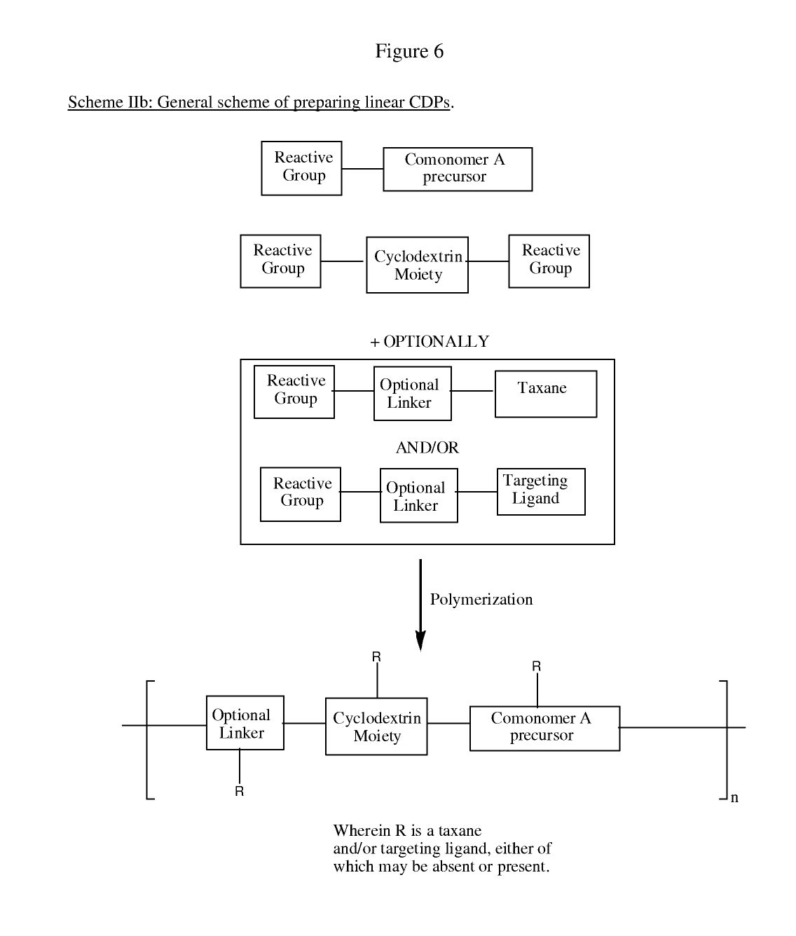

To illustrate further, comonomer precursors (shown in the scheme in FIG. 5), cyclodextrin moieties, taxanes, and/or targeting ligands may be assembled as shown in Schemes IIa-IIb (FIGS. 5 and 6). Note that in schemes IIa-IIb, in any given reaction there may be more than one comonomer precursor, cyclodextrin moiety, therapeutic agent or targeting ligand that is of the same type or different. Furthermore, prior to polymerization, one or more comonomer precursor, cyclodextrin moiety, therapeutic agent or targeting ligand may be covalently linked with each other in one or more separate step. The scheme as provided above includes embodiments, where not all available positions for attachment of the taxane are occupied on the CDP. For example, in some embodiments, less than all of the available points of attachments are reacted, leaving less than 100% yield of the taxane onto the polymer. Accordingly, the loading of the taxane onto the polymer can vary. This is also the case regarding a targeting agent when a targeting agent is included.

Scheme IIa: General Scheme for Graft Polymers.

The comonomer A precursor, cyclodextrin moiety, taxane and optional targeting ligand are as defined above. Furthermore, one skilled in the art may choose from a variety of reactive groups, e.g., hydroxyls, carboxyls, halides, amines, and activated ethenes, ethynes, or aromatic groups in order achieve polymerization. For further examples of reactive groups are disclosed in Advanced Organic Chemistry: Reactions, Mechanisms, and Structure, 5th Edition, 2000.

In some embodiments, one or more of the taxane moieties in the CDP-taxane conjugate can be replaced with another therapeutic agent, e.g., another anticancer agent or anti-inflammatory agent.

Scheme IIb: General Scheme of Preparing Linear CDPs.

One skilled in the art would recognize that by choosing a comonomer A precursor that has multiple reactive groups polymer branching can be achieved (see FIG. 6).

In some embodiments, one or more of the taxane moieties in the CDP-taxane conjugate can be replaced with another therapeutic agent, e.g., another anticancer agent or anti-inflammatory agent.

Examples of different ways of synthesizing CDP-taxane conjugates are shown in Schemes III-VIII below. In each of Schemes III-VIII, one or more of the taxane moieties in the CDP-taxane conjugate can be replaced with another therapeutic agent, e.g., another anticancer agent or anti-inflammatory agent.

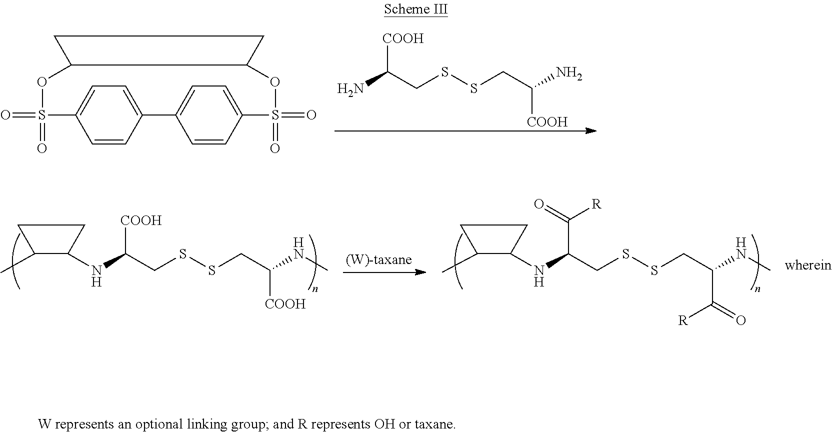

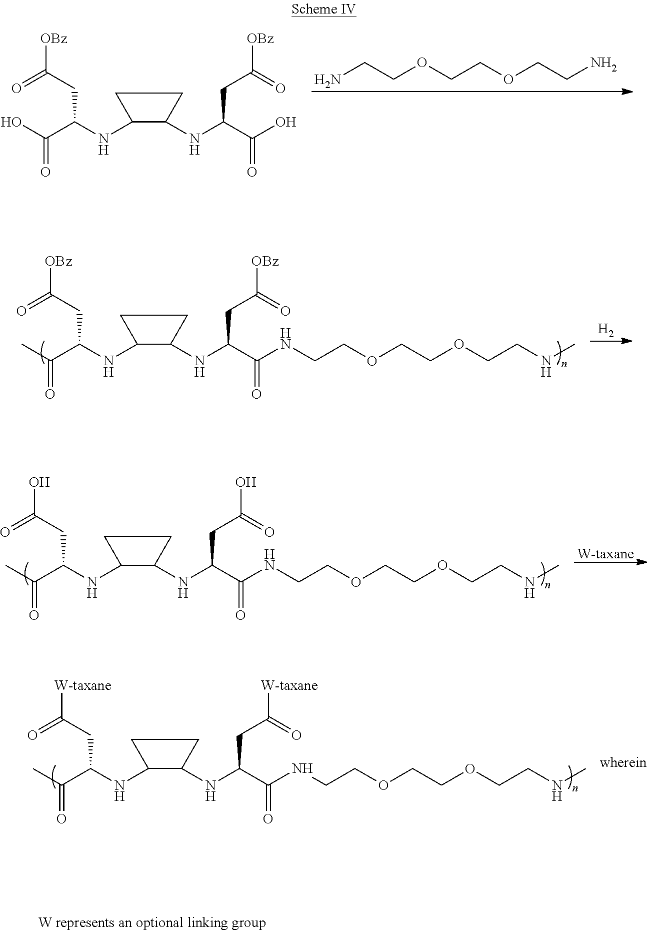

Scheme IV, as provided above, includes embodiments where W-taxane is absent in one or more positions as provided above. This can be achieved, for example, when less than 100% yield is achieved when coupling the taxane to the polymer and/or when less than an equivalent amount of taxane is used in the reaction. Accordingly, the loading of the taxane, by weight of the polymer, can vary.

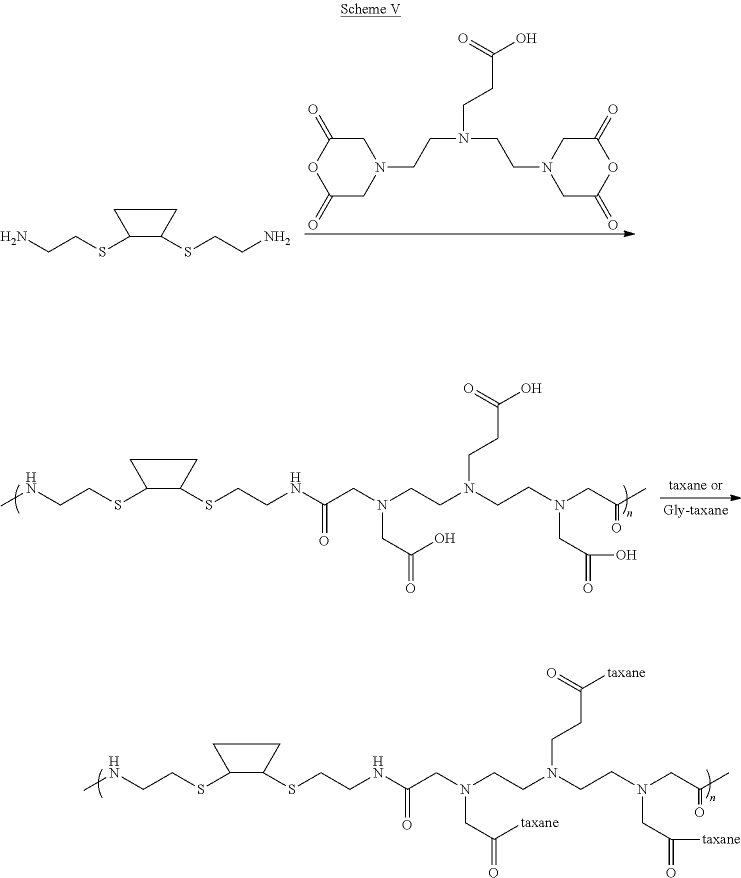

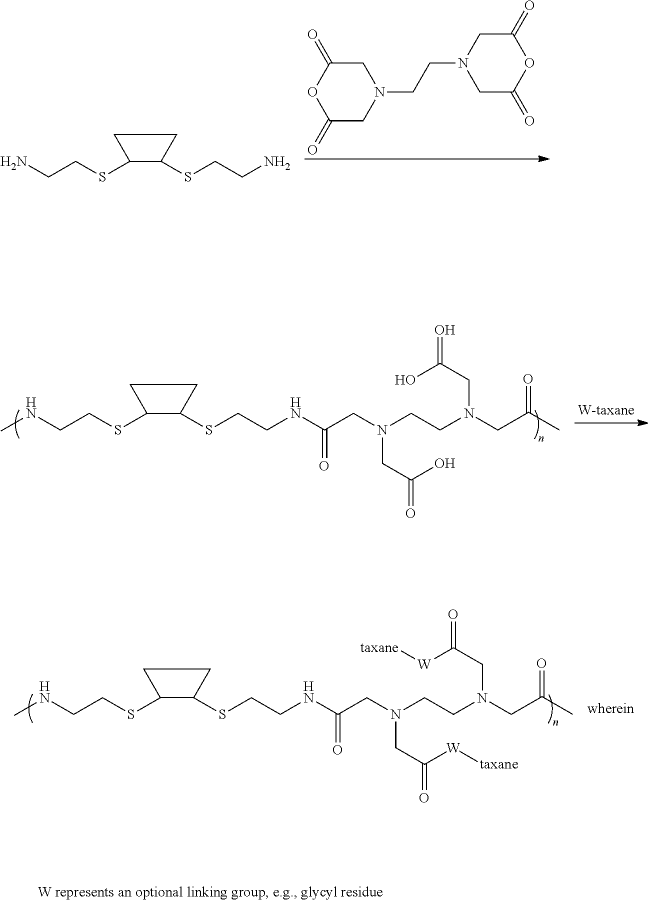

Scheme V, as provided above, includes embodiments where W-taxane is absent in one or more positions as provided above. This can be achieved, for example, when less than 100% yield is achieved when coupling the taxane to the polymer and/or when less than an equivalent amount of taxane is used in the reaction. Accordingly, the loading of the taxane, by weight of the polymer, can vary.

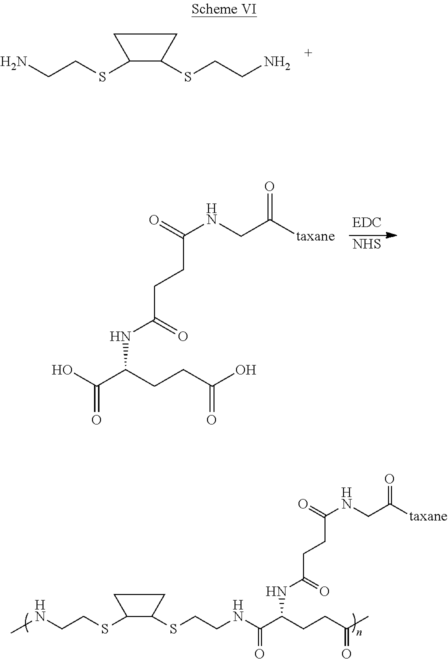

Scheme VI, as provided above, includes embodiments where taxane is absent in one or more positions as provided above. This can be achieved, for example, when less than 100% yield is achieved when coupling the taxane to the polymer and/or when less than an equivalent amount of taxane is used in the reaction. Accordingly, the loading of the taxane, by weight of the polymer, can vary.

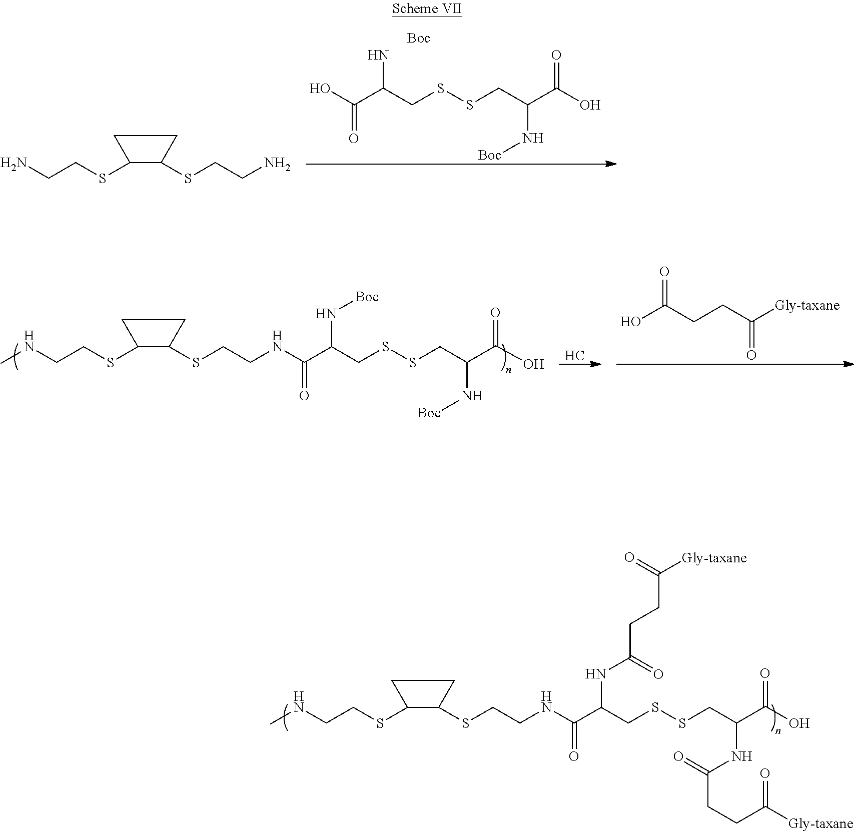

Scheme VII, as provided above, includes embodiments where gly-taxane is absent in one or more positions as provided above. This can be achieved, for example, when less than 100% yield is achieved when coupling the taxane to the polymer and/or when less than an equivalent amount of taxane is used in the reaction. Accordingly, the loading of the taxane, by weight of the polymer, can vary.

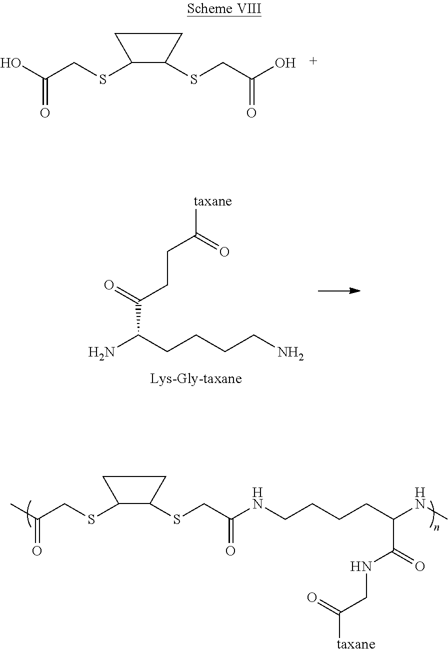

Scheme VIII, as provided above, includes embodiments where taxane is absent in one or more positions as provided above. This can be achieved, for example, when less than 100% yield is achieved when coupling the taxane to the polymer and/or when less than an equivalent amount of taxane is used in the reaction. Accordingly, the loading of the taxane, by weight of the polymer, can vary.

Additional examples of methods of synthesizing CDP-taxane conjugates are shown in Schemes IX-XIV below. In each of Schemes IX-XIV, one or more of the taxane moieties in the CDP-taxane conjugate can be replaced with another therapeutic agent, e.g., another anticancer agent or anti-inflammatory agent.

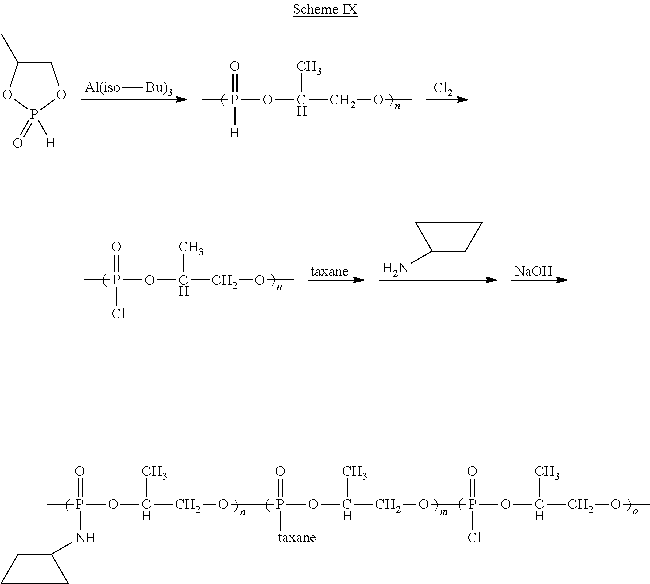

Scheme IX, as provided above, includes embodiments where taxane is absent in one or more positions as provided above. This can be achieved, for example, when less than 100% yield is achieved when coupling the taxane to the polymer and/or when less than an equivalent amount of taxane is used in the reaction. Accordingly, the loading of the taxane, by weight of the polymer, can vary.

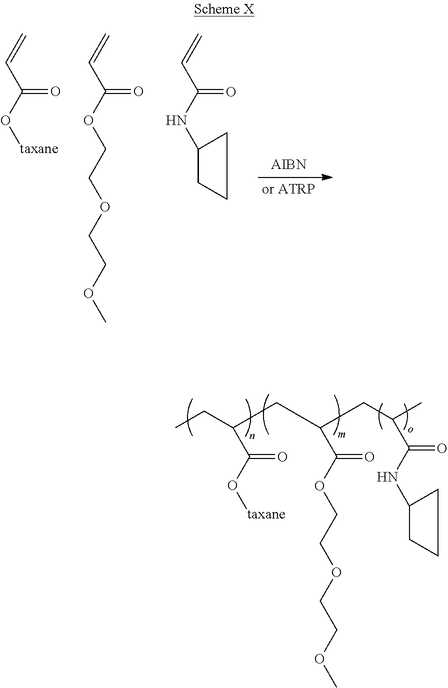

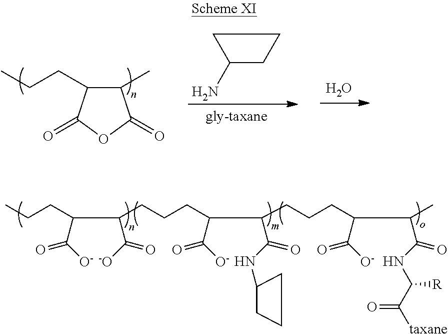

Scheme XI, as provided above, includes embodiments where gly-taxane is absent in one or more positions as provided above. This can be achieved, for example, when less than 100% yield is achieved when coupling the taxane to the polymer and/or when less than an equivalent amount of taxane is used in the reaction. Accordingly, the loading of the taxane, by weight of the polymer, can vary.

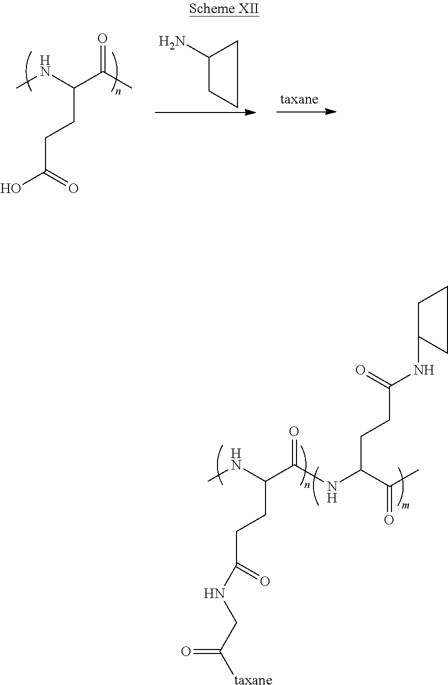

Scheme XII, as provided above, includes embodiments where taxane is absent in one or more positions as provided above. This can be achieved, for example, when less than 100% yield is achieved when coupling the taxane to the polymer and/or when less than an equivalent amount of taxane is used in the reaction. Accordingly, the loading of the taxane, by weight of the polymer, can vary.

The present invention further contemplates CDPs and CDP-conjugates synthesized using CD-biscysteine monomer and a di-NHS ester such as PEG-DiSPA or PEG-BTC as shown in Schemes XIII-XIV below.

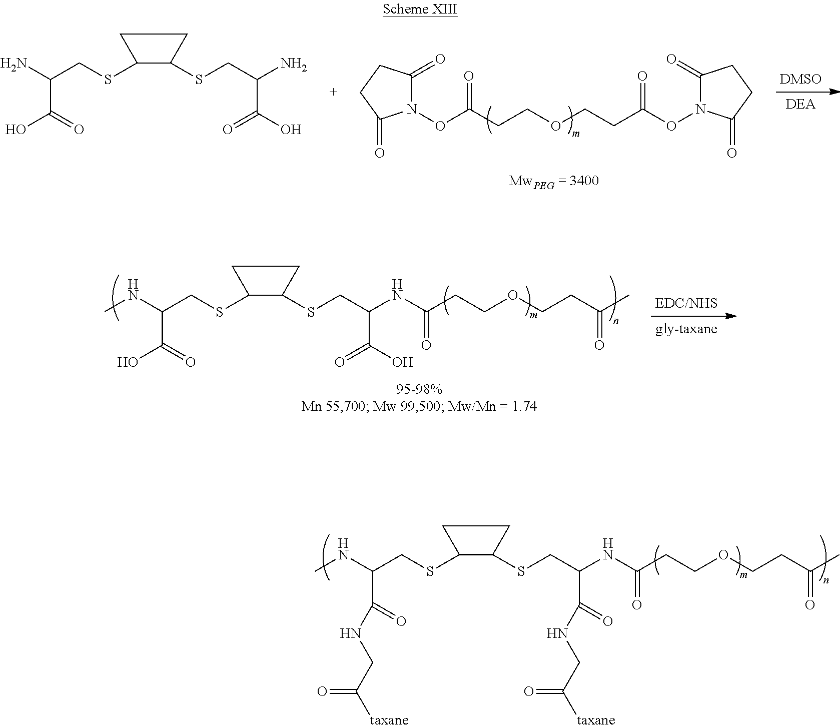

Scheme XIII, as provided above, includes embodiments where gly-taxane is absent in one or more positions as provided above. This can be achieved, for example, when less than 100% yield is achieved when coupling the taxane to the polymer and/or when less than an equivalent amount of taxane is used in the reaction. Accordingly, the loading of the taxane, by weight of the polymer, can vary.

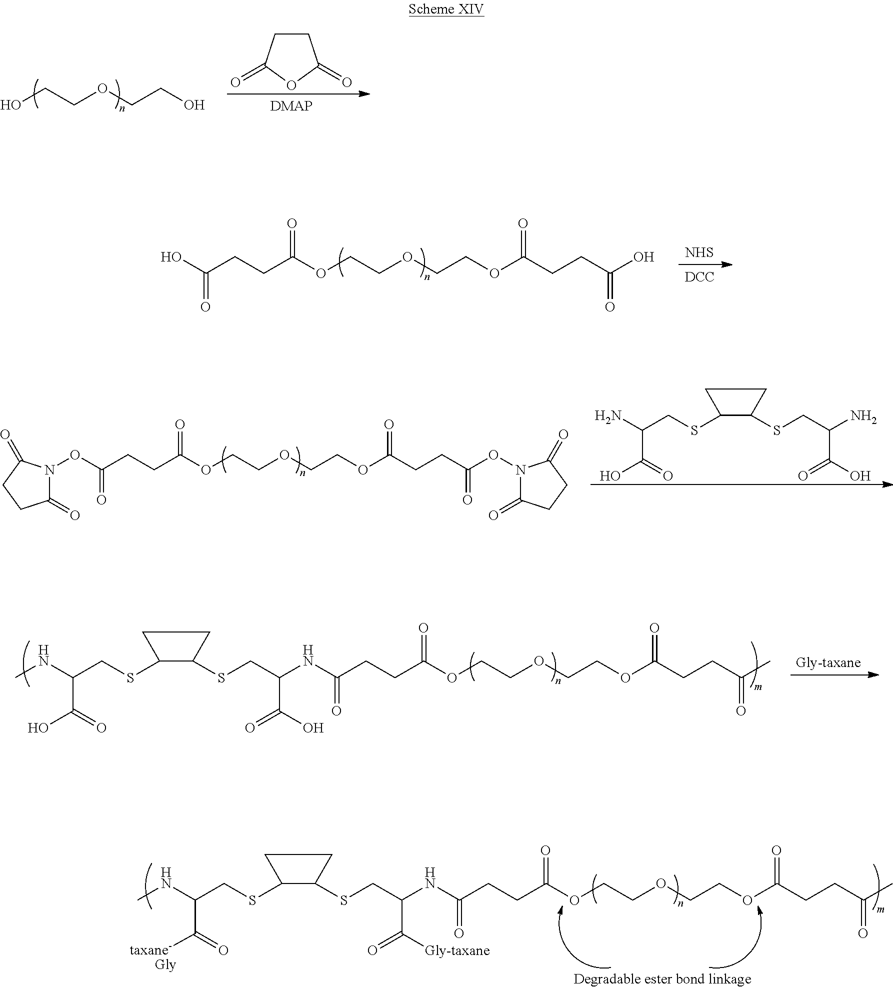

Scheme XIV, as provided above, includes embodiments where gly-taxane is absent in one or more positions as provided above. This can be achieved, for example, when less than 100% yield is achieved when coupling the taxane to the polymer and/or when less than an equivalent amount of taxane is used in the reaction. Accordingly, the loading of the taxane, by weight of the polymer, can vary.

In some embodiments, a CDP-taxane conjugate can be made by providing a CDP comprising cyclodextrin moieties and comonomers which do not contain cyclodextrin moieties (comonomers), wherein the cyclodextrin moieties and comonomers alternate in the CDP and wherein the CDP comprises at least four, five, six, seven, eight, etc. cyclodextrin moieties and at least four, five, six, seven, eight, etc. comonomers; and attaching a taxane to the CDP.

In some embodiments, one or more of the taxane moieties in the CDP-taxane conjugate can be replaced with another therapeutic agent, e.g., another anticancer agent or anti-inflammatory agent.

In some embodiments, the taxane is attached via a linker. In some embodiments, the taxane is attached to the water soluble linear polymer through an attachment that is cleaved under biological conditions to release the taxane. In some embodiments, the taxane is attached to the water soluble linear polymer at a cyclodextrin moiety or a comonomer. In some embodiments, the taxane is attached to the water soluble linear polymer via an optional linker to a cyclodextrin moiety or a comonomer.

In some embodiments, the cyclodextrin moieties comprise linkers to which therapeutic agents are linked. In some embodiments, the cyclodextrin moieties comprise linkers to which therapeutic agents are linked via a second linker In some embodiments, the CDP is made by a process comprising: providing cyclodextrin moiety precursors, providing comonomer precursors, and copolymerizing said cyclodextrin moiety precursors and comonomer precursors to thereby make a CDP comprising cyclodextrin moieties and comonomers. In some embodiments, the CDP is conjugated with a taxane to provide a CDP-taxane conjugate.

In some embodiments, the method includes providing cyclodextrin moiety precursors modified to bear one reactive site at each of exactly two positions, and reacting the cyclodextrin moiety precursors with comonomer precursors having exactly two reactive moieties capable of forming a covalent bond with the reactive sites under polymerization conditions that promote reaction of the reactive sites with the reactive moieties to form covalent bonds between the comonomers and the cyclodextrin moieties, whereby a CDP comprising alternating units of a cyclodextrin moiety and a comonomer is produced.

In some embodiments, the taxane is attached to the CDP via a linker. In some embodiments, the linker is cleaved under biological conditions.

In some embodiments, the taxane makes up at least 5%, 10%, 15%, 20%, 25%, 30%, or even 35% by weight of the CDP-taxane conjugate. In some embodiments, at least about 50% of available positions on the CDP are reacted with a taxane and/or a linker taxane (e.g., at least about 55%, 60%, 65%, 70%, 75%, 80%, 85%, 90%, or 95%).

In some embodiments, the comonomer comprises polyethylene glycol of molecular weight 3,400 Da, the cyclodextrin moiety comprises beta-cyclodextrin, the theoretical maximum loading of taxane on the CDP-taxane conjugate is 19%, and taxane is 17-21% by weight of the CDP-taxane conjugate. In some embodiments, about 80-90% of available positions on the CDP are reacted with a taxane and/or a linker taxane.

In some embodiments, the comonomer precursor is a compound containing at least two functional groups through which reaction and thus linkage of the cyclodextrin moieties is achieved. In some embodiments, the functional groups, which may be the same or different, terminal or internal, of each comonomer precursor comprise an amino, acid, imidazole, hydroxyl, thio, acyl halide, —HC═CH—, —C≡C— group, or derivative thereof. In some embodiments, the two functional groups are the same and are located at termini of the comonomer precursor. In some embodiments, a comonomer contains one or more pendant groups with at least one functional group through which reaction and thus linkage of a therapeutic agent is achieved. In some embodiments, the functional groups, which may be the same or different, terminal or internal, of each comonomer pendant group comprise an amino, acid, imidazole, hydroxyl, thiol, acyl halide, ethylene, ethyne group, or derivative thereof. In some embodiments, the pendant group is a substituted or unsubstituted branched, cyclic or straight chain C1-C10 alkyl, or arylalkyl optionally containing one or more heteroatoms within the chain or ring.

In some embodiments, the cyclodextrin moiety comprises an alpha, beta, or gamma cyclodextrin moiety.

In some embodiments, the taxane is poorly soluble in water.

In some embodiments, the solubility of the taxane is <5 mg/ml at physiological pH.

In some embodiments, the taxane is a hydrophobic compound with a log P>0.4, >0.6, >0.8, >1, >2, >3, >4, or >5. In some embodiments, the taxane is hydrophobic and is attached via a second compound.

In some embodiments, administration of the CDP-taxane conjugate to a subject results in release of the taxane over a period of at least 6 hours. In some embodiments, administration of the CDP-taxane conjugate to a subject results in release of the taxane over a period of 6 hours to a month. In some embodiments, upon administration of the CDP-taxane conjugate to a subject the rate of taxane release is dependent primarily upon the rate of hydrolysis as opposed to enzymatic cleavage.

In some embodiments, the CDP-taxane conjugate has a molecular weight of 10,000-500,000.

In some embodiments, the cyclodextrin moieties make up at least about 2%, 5%, 10%, 20%, 30%, 50% or 80% of the polymer by weight.

In some embodiments, a the CDP includes a comonomer selected from the group consisting of: an alkylene chain, polysuccinic anhydride, poly-L-glutamic acid, poly(ethyleneimine), an oligosaccharide, and an amino acid chain. In some embodiments, a comonomer comprises a polyethylene glycol chain. In some embodiments, a comonomer comprises a polyglycolic acid or polylactic acid chain. In some embodiments, a comonomer comprises a hydrocarbylene group wherein one or more methylene groups is optionally replaced by a group Y (provided that none of the Y groups are adjacent to each other), wherein each Y, independently for each occurrence, is selected from, substituted or unsubstituted aryl, heteroaryl, cycloalkyl, heterocycloalkyl, or —O—, C(═X) (wherein X is NR₁, O or S), —OC(O)—, —C(═O)O, —NR₁—, —NRiCO—, —C(O)NR₁—, —S(O)ₙ— (wherein n is 0, 1, or 2), —OC(O)—NR₁, —NR₁—C(O)—NR₁—, —NR₁—C(NR₁)—NR₁—, and —B(ORi)—; and R₁, independently for each occurrence, represents H or a lower alkyl.

In some embodiments, a CDP-polymer conjugate of the following formula can be made as follows:

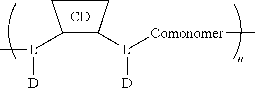

providing a polymer of the formula below:

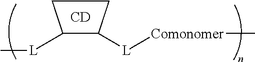

and coupling the polymer with a plurality of D moieties, wherein each D is independently absent or a taxane, to provide:

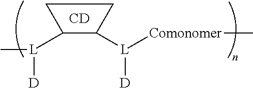

wherein the comonomer has a Mw of 2000 to 5000 Da (e.g., 3000 to 4000 Da, e.g., 3200 kDa to about 3.8 kDa, e.g., about 3.4 kDa) and n is at least 4, 5, 6, 7, 8, 9, 10, 11, 12, 13, 14, 15, 16, 17, 18, 19 or 20.

In some embodiments, one or more of the taxane moieties in the CDP-taxane conjugate can be replaced with another therapeutic agent, e.g., another anticancer agent or anti-inflammatory agent.

In some embodiments, a CDP-polymer conjugate of the following formula can be made as follows:

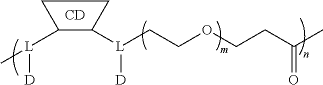

providing a polymer of the formula below:

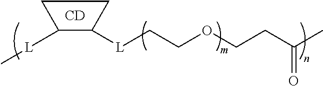

and coupling the polymer with a plurality of D moieties, wherein each D is independently absent or a taxane, to provide:

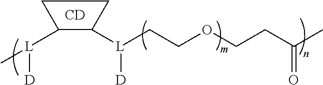

wherein the group

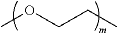

has a Mw of 4.0 kDa or less, e.g., 3.2 to 3.8 kDa, e.g., 3.4 kDa and n is at least 4, 5, 6, 7, 8, 9, 10, 11, 12, 13, 14, 15, 16, 17, 18, 19 or 20.

In some embodiments, one or more of the taxane moieties in the CDP-taxane conjugate can be replaced with another therapeutic agent, e.g., another anticancer agent or anti-inflammatory agent.

The reaction scheme as provided above includes embodiments where D is absent in one or more positions as provided above. This can be achieved, for example, when less than 100% yield is achieved when coupling the taxane to the polymer (e.g., 80-90%) and/or when less than an equivalent amount of taxane is used in the reaction. Accordingly, the loading of the taxane, by weight of the polymer, can vary, for example, the loading of the taxane can be at least about 3% by weight, e.g., at least about 5%, at least about 8%, at least about 10%, at least about 13%, at least about 15%, or at least about 20%.

In some embodiments, a CDP-polymer conjugate of the following formula can be made as follows:

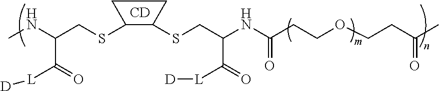

providing a polymer below:

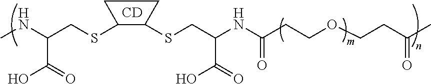

and coupling the polymer with a plurality of L-D moieties, wherein L is a linker or absent and D is a taxane, to provide:

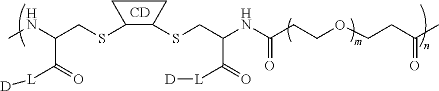

wherein the group

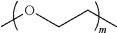

has a Mw of 4.0 kDa or less, e.g., 3.2 to 3.8 kDa, e.g., 3.4 kDa and n is at least 4, 5, 6, 7, 8, 9, 10, 11, 12, 13, 14, 15, 16, 17, 18, 19 or 20.

In some embodiments, one or more of the taxane moieties in the CDP-taxane conjugate can be replaced with another therapeutic agent, e.g., another anticancer agent or anti-inflammatory agent.

The reaction scheme as provided above includes embodiments where L-D is absent in one or more positions as provided above. This can be achieved, for example, when less than 100% yield is achieved when coupling the taxane-linker to the polymer (e.g., 80-90%) and/or when less than an equivalent amount of taxane-linker is used in the reaction. Accordingly, the loading of the taxane, by weight of the polymer, can vary, for example, the loading of the taxane can be at least about 3% by weight, e.g., at least about 5%, at least about 8%, at least about 10%, at least about 13%, at least about 15%, or at least about 20%.

In some embodiments, at least a portion of the L moieties of L-D is absent. In some embodiments, each L is independently an amino acid or derivative thereof (e.g., glycine).

In some embodiments, the coupling of the polymer with the plurality of L-D moieties results in the formation of a plurality of amide bonds.

In certain instances, the CDPs are random copolymers, in which the different subunits and/or other monomeric units are distributed randomly throughout the polymer chain. Thus, where the formula Xₘ—Yₙ—Zₒ appears, wherein X, Y and Z are polymer subunits, these subunits may be randomly interspersed throughout the polymer backbone. In part, the term “random” is intended to refer to the situation in which the particular distribution or incorporation of monomeric units in a polymer that has more than one type of monomeric units is not directed or controlled directly by the synthetic protocol, but instead results from features inherent to the polymer system, such as the reactivity, amounts of subunits and other characteristics of the synthetic reaction or other methods of manufacture, processing, or treatment.

### Pharmaceutical Compositions

In another aspect, the present invention provides a composition, e.g., a pharmaceutical composition, comprising a CDP-taxane conjugate and a pharmaceutically acceptable carrier or adjuvant.

In some embodiments, a pharmaceutical composition may include a pharmaceutically acceptable salt of a compound described herein, e.g., a CDP-taxane conjugate. Pharmaceutically acceptable salts of the compounds described herein include those derived from pharmaceutically acceptable inorganic and organic acids and bases. Examples of suitable acid salts include acetate, adipate, benzoate, benzenesulfonate, butyrate, citrate, digluconate, dodecylsulfate, formate, fumarate, glycolate, hemisulfate, heptanoate, hexanoate, hydrochloride, hydrobromide, hydroiodide, lactate, maleate, malonate, methanesulfonate, 2-naphthalenesulfonate, nicotinate, nitrate, palmoate, phosphate, picrate, pivalate, propionate, salicylate, succinate, sulfate, tartrate, tosylate and undecanoate. Salts derived from appropriate bases include alkali metal (e.g., sodium), alkaline earth metal (e.g., magnesium), ammonium and N-(alkyl)₄⁺ salts. This invention also envisions the quaternization of any basic nitrogen-containing groups of the compounds described herein. Water or oil-soluble or dispersible products may be obtained by such quaternization.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gailate, alpha-tocopherol, and the like; and (3) metal chelating agents, such as citric acid, ethylenediamine tetraacetic acid (EDTA), sorbitol, tartaric acid, phosphoric acid, and the like.

A composition may include a liquid used for suspending a CDP-taxane conjugate, which may be any liquid solution compatible with the CDP-taxane conjugate, which is also suitable to be used in pharmaceutical compositions, such as a pharmaceutically acceptable nontoxic liquid. Suitable suspending liquids including but are not limited to suspending liquids selected from the group consisting of water, aqueous sucrose syrups, corn syrups, sorbitol, polyethylene glycol, propylene glycol, and mixtures thereof.

A composition described herein may also include another component, such as an antioxidant, antibacterial, buffer, bulking agent, chelating agent, an inert gas, a tonicity agent and/or a viscosity agent.

In one embodiment, the CDP-taxane conjugate is provided in lyophilized form and is reconstituted prior to administration to a subject. The lyophilized CDP-taxane conjugate can be reconstituted by a diluent solution, such as a salt or saline solution, e.g., a sodium chloride solution having a pH between 6 and 9, lactated Ringer's injection solution, or a commercially available diluent, such as PLASMA-LYTE A Injection pH 7.4® (Baxter, Deerfield, Ill.).

In one embodiment, a lyophilized formulation includes a lyoprotectant or stabilizer to maintain physical and chemical stability by protecting the CDP-taxane conjugate from damage from crystal formation and the fusion process during freeze-drying. The lyoprotectant or stabilizer can be one or more of polyethylene glycol (PEG), a PEG lipid conjugate (e.g., PEG-ceramide or D-alpha-tocopheryl polyethylene glycol 1000 succinate), poly(vinyl alcohol) (PVA), poly(vinylpyrrolidone) (PVP), polyoxyethylene esters, poloxomers, Tweens, lecithins, saccharides, oligosaccharides, polysaccharides and polyols (e.g., trehalose, mannitol, sorbitol, lactose, sucrose, glucose and dextran), salts and crown ethers.

In some embodiments, the lyophilized CDP-taxane conjugate is reconstituted with a mixture of equal parts by volume of Dehydrated Alcohol, USP and a nonionic surfactant, such as a polyoxyethylated castor oil surfactant available from GAF Corporation, Mount Olive, N.J., under the trademark, Cremophor EL. The lyophilized product and vehicle for reconstitution can be packaged separately in appropriately light-protected vials. To minimize the amount of surfactant in the reconstituted solution, only a sufficient amount of the vehicle may be provided to form a solution having a concentration of about 2 mg/mL to about 4 mg/mL of the CDP-taxane conjugate. Once dissolution of the drug is achieved, the resulting solution is further diluted prior to injection with a suitable parenteral diluent. Such diluents are well known to those of ordinary skill in the art. These diluents are generally available in clinical facilities. It is, however, within the scope of the present invention to package the subject CDP-taxane conjugate with a third vial containing sufficient parenteral diluent to prepare the final concentration for administration. A typical diluent is Lactated Ringer's Injection.

The final dilution of the reconstituted CDP-taxane conjugate may be carried out with other preparations having similar utility, for example, 5% Dextrose Injection, Lactated Ringer's and Dextrose Injection, Sterile Water for Injection, and the like. However, because of its narrow pH range, pH 6.0 to 7.5, Lactated Ringer's Injection is most typical. Per 100 mL, Lactated Ringer's Injection contains Sodium Chloride USP 0.6 g, Sodium Lactate 0.31 g, Potassium chloride USP 0.03 g and Calcium Chloride2H2O USP 0.02 g. The osmolarity is 275 mOsmol/L, which is very close to isotonicity.

The compositions may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

### Routes of Administration

The pharmaceutical compositions described herein may be administered orally, parenterally (e.g., via intravenous, subcutaneous, intracutaneous, intramuscular, intraarticular, intraarterial, intrasynovial, intrasternal, intrathecal, intralesional or intracranial injection), topically, mucosally (e.g., rectally or vaginally), nasally, buccally, ophthalmically, via inhalation spray (e.g., delivered via nebulization, propellant or a dry powder device) or via an implanted reservoir.

Pharmaceutical compositions suitable for parenteral administration comprise one or more CDP-taxane conjugate(s) in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the agent from subcutaneous or intramuscular injection. This may be accomplished by the use of a liquid suspension of crystalline or amorphous material having poor water solubility. The rate of absorption of the CDP-taxane conjugate then depends upon its rate of dissolution which, in turn, may depend upon crystal size and crystalline form. Alternatively, delayed absorption of a parenterally administered drug form is accomplished by dissolving or suspending the CDP-taxane conjugate in an oil vehicle.

Pharmaceutical compositions suitable for oral administration may be in the form of capsules, cachets, pills, tablets, gums, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouthwashes and the like, each containing a predetermined amount of an agent as an active ingredient. A compound may also be administered as a bolus, electuary or paste.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered peptide or peptidomimetic moistened with an inert liquid diluent.

Tablets, and other solid dosage forms, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner Examples of embedding compositions which can be used include polymeric substances and waxes. The active ingredient can also be in micro-encapsulated form, if appropriate, with one or more of the above-described excipients.

Liquid dosage forms for oral administration include pharmaceutically acceptable emulsions, microemulsions, solutions, suspensions, syrups and elixirs. In addition to the CDP-taxane conjugate, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the CDP-taxane conjugate may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Pharmaceutical compositions suitable for topical administration are useful when the desired treatment involves areas or organs readily accessible by topical application.

For application topically to the skin, the pharmaceutical composition should be formulated with a suitable ointment containing the active components suspended or dissolved in a carrier. Carriers for topical administration of the a particle described herein include, but are not limited to, mineral oil, liquid petroleum, white petroleum, propylene glycol, polyoxyethylene polyoxypropylene compound, emulsifying wax and water. Alternatively, the pharmaceutical composition can be formulated with a suitable lotion or cream containing the active particle suspended or dissolved in a carrier with suitable emulsifying agents. Suitable carriers include, but are not limited to, mineral oil, sorbitan monostearate, polysorbate 60, cetyl esters wax, cetearyl alcohol, 2-octyldodecanol, benzyl alcohol and water. The pharmaceutical compositions described herein may also be topically applied to the lower intestinal tract by rectal suppository formulation or in a suitable enema formulation. Topically-transdermal patches are also included herein.

The pharmaceutical compositions described herein may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

The pharmaceutical compositions described herein may also be administered in the form of suppositories for rectal or vaginal administration. Suppositories may be prepared by mixing one or more CDP-taxane conjugate described herein with one or more suitable non-irritating excipients which is solid at room temperature, but liquid at body temperature. The composition will therefore melt in the rectum or vaginal cavity and release the CDP-taxane conjugate. Such materials include, for example, cocoa butter, polyethylene glycol, a suppository wax or a salicylate. Compositions of the present invention which are suitable for vaginal administration also include pessaries, tampons, creams, gels, pastes, foams or spray formulations containing such carriers as are known in the art to be appropriate.

Ophthalmic formulations, eye ointments, powders, solutions and the like, are also contemplated as being within the scope of the invention.

### Dosages and Dosage Regimens

The CDP-taxane conjugate can be formulated into pharmaceutically acceptable dosage forms by conventional methods known to those of skill in the art.

Actual dosage levels of the active ingredients in the pharmaceutical compositions of this invention may be varied so as to obtain an amount of the active ingredient which is effective to achieve the desired therapeutic response for a particular subject, composition, and mode of administration, without being toxic to the subject.

In one embodiment, the CDP-taxane conjugate is administered to a subject at a dosage of, e.g., about 0.1 to 300 mg/m², about 5 to 275 mg/m², about 10 to 250 mg/m², e.g., about 15, 20, 25, 30, 35, 40, 45, 50, 55, 60, 65, 70, 80, 90, 100, 110, 120, 130, 140, 150, 160, 170, 180, 190, 200, 210, 220, 230, 240, 250, 260, 270, 280, 290 mg/m² of the taxane. Administration can be at regular intervals, such as every 1, 2, 3, 4, or 5 days, or weekly, or every 2, 3, 4, 5, 6, or 7 or 8 weeks. The administration can be over a period of from about 10 minutes to about 6 hours, e.g., from about 30 minutes to about 2 hours, from about 45 minutes to 90 minutes, e.g., about 30 minutes, 45 minutes, 1 hour, 2 hours, 3 hours, 4 hours, 5 hours or more. In one embodiment, the CDP-taxane conjugate is administered as a bolus infusion or intravenous push, e.g., over a period of 15 minutes, 10 minutes, 5 minutes or less. In one embodiment, the CDP-taxane is administered in an amount such the desired dose of the agent is administered. Preferably the dose of the CDP-taxane conjugate is a dose described herein.

In one embodiment, the subject receives 1, 2, 3, up to 10 treatments, or more, or until the disorder or a symptom of the disorder is cured, healed, alleviated, relieved, altered, remedied, ameliorated, palliated, improved or affected. For example, the subject receive an infusion once every 1, 2, 3 or 4 weeks until the disorder or a symptom of the disorder are cured, healed, alleviated, relieved, altered, remedied, ameliorated, palliated, improved or affected. Preferably, the dosing schedule is a dosing schedule described herein.

The CDP-taxane conjugate can be administered as a first line therapy, e.g., alone or in combination with an additional agent or agents. In other embodiments, a CDP-taxane is administered after a subject has developed resistance to, has filed to respond to or has relapsed after a first line therapy. The CDP-taxane conjugate can be administered in combination with a second agent. Preferably, the CDP-taxane is administered in combination with a second agent described herein.

### Kits

A CDP-taxane described herein may be provided in a kit. The kit includes a CDP-taxane conjugate described herein and, optionally, a container, a pharmaceutically acceptable carrier and/or informational material. The informational material can be descriptive, instructional, marketing or other material that relates to the methods described herein and/or the use of the CDP-taxane conjugate for the methods described herein.

The informational material of the kits is not limited in its form. In one embodiment, the informational material can include information about production of the CDP-taxane conjugate, physical properties of the CDP-taxane conjugate, concentration, date of expiration, batch or production site information, and so forth. In one embodiment, the informational material relates to methods for administering the CDp-taxane.

In one embodiment, the informational material can include instructions to administer a CDP-taxane conjugate described herein in a suitable manner to perform the methods described herein, e.g., in a suitable dose, dosage form, or mode of administration (e.g., a dose, dosage form, or mode of administration described herein). In another embodiment, the informational material can include instructions to administer a CDP-taxane conjugate described herein to a suitable subject, e.g., a human, e.g., a human having or at risk for a disorder described herein. In another embodiment, the informational material can include instructions to reconstitute a CDP-taxane conjugate described herein into a pharmaceutically acceptable composition.

In one embodiment, the kit includes instructions to use the CDP-taxane conjugate, such as for treatment of a subject. The instructions can include methods for reconstituting or diluting the CDP-taxane conjugate for use with a particular subject or in combination with a particular chemotherapeutic agent. The instructions can also include methods for reconstituting or diluting the CDP-taxane conjugate for use with a particular means of administration, such as by intravenous infusion.

In another embodiment, the kit includes instructions for treating a subject with a particular indication, such as a particular cancer, or a cancer at a particular stage. For example, the instructions can be for a cancer or cancer at stage described herein. The instructions may also address first line treatment of a subject who has a particular cancer, or cancer at a stage described herein. The instructions can also address treatment of a subject who has been non-responsive to a first line therapy or has become sensitive (e.g., has one or more unacceptable side effect) to a first line therapy, such as a taxane, an anthracycline, an alkylating agent, a platinum based agent, a vinca alkaloid. In another embodiment, the instructions will describe treatment of selected subjects with the CDP-taxane conjugate. For example, the instructions can describe treatment of one or more of: a subject who has received an anticancer agent (e.g., a taxane) and has a neutrophil count less than a standard; a subject who has moderate to severe neutropenia; a subject who has experienced one or more symptom of neuropathy from treatment with an anticancer agent, e.g., a taxane, a vinca alkaloid, an alkylating agent, an anthracycline, a platinum-based agent or an epothilone; a subject who has experienced an infusion site reaction or has or is at risk for having hypersensitivity to treatment with an anticancer agent (e.g., a taxane); a subject having hepatic impairment, e.g., having transaminase (ALT and/or AST levels) greater than the upper limit of normal (ULN) and/or bilirubin levels greater than ULN; a subject having hepatic impairment, e.g., ALP levels greater than the upper limit of normal (ULN), SGOT and/or SGPT levels greater the upper limit of normal (ULN) and/or bilirubin levels greater than the ULN; a subject who is currently being administered or will be administered a cytochrome P450 isoenzyme inhibitor; a subject who has experienced or is at risk for renal impairment, a subject who has or is at risk of having a gastroinstinal disorder (e.g., vomiting, nausea and/or diarrhea, e.g., associated with the administration of a chemotherapeutic agent (e.g., a taxane)), and a subject who has or is at risk for having fluid retention and/or effusion.

The informational material of the kits is not limited in its form. In many cases, the informational material, e.g., instructions, is provided in printed matter, e.g., a printed text, drawing, and/or photograph, e.g., a label or printed sheet. However, the informational material can also be provided in other formats, such as Braille, computer readable material, video recording, or audio recording. In another embodiment, the informational material of the kit is contact information, e.g., a physical address, email address, website, or telephone number, where a user of the kit can obtain substantive information about a CDP-taxane conjugate described herein and/or its use in the methods described herein. The informational material can also be provided in any combination of formats.

In addition to a CDP-taxane conjugate described herein, the composition of the kit can include other ingredients, such as a surfactant, a lyoprotectant or stabilizer, an antioxidant, an antibacterial agent, a bulking agent, a chelating agent, an inert gas, a tonicity agent and/or a viscosity agent, a solvent or buffer, a stabilizer, a preservative, a flavoring agent (e.g., a bitter antagonist or a sweetener), a fragrance, a dye or coloring agent, for example, to tint or color one or more components in the kit, or other cosmetic ingredient, a pharmaceutically acceptable carrier and/or a second agent for treating a condition or disorder described herein. Alternatively, the other ingredients can be included in the kit, but in different compositions or containers than a CDP-taxane described herein. In such embodiments, the kit can include instructions for admixing a CDP-taxane conjugate described herein and the other ingredients, or for using a CDP-taxane conjugate described herein together with the other ingredients.

In another embodiment, the kit includes a second therapeutic agent, such as a second chemotherapeutic agent, e.g., a chemotherapeutic agent or combination of chemotherapeutic agents described herein. In one embodiment, the second agent is in lyophilized or in liquid form. In one embodiment, the CDP-taxane conjugate and the second therapeutic agent are in separate containers, and in another embodiment, the CDP-taxane conjugate and the second therapeutic agent are packaged in the same container.

In some embodiments, a component of the kit is stored in a sealed vial, e.g., with a rubber or silicone enclosure (e.g., a polybutadiene or polyisoprene enclosure). In some embodiments, a component of the kit is stored under inert conditions (e.g., under Nitrogen or another inert gas such as Argon). In some embodiments, a component of the kit is stored under anhydrous conditions (e.g., with a desiccant). In some embodiments, a component of the kit is stored in a light blocking container such as an amber vial.

A CDP-taxane described herein can be provided in any form, e.g., liquid, frozen, dried or lyophilized form. It is preferred that a particle described herein be substantially pure and/or sterile. When a CDP-taxane conjugate described herein is provided in a liquid solution, the liquid solution preferably is an aqueous solution, with a sterile aqueous solution being preferred. In one embodiment, the CDP-taxane conjugate is provided in lyophilized form and, optionally, a diluent solution is provided for reconstituting the lyophilized agent. The diluent can include for example, a salt or saline solution, e.g., a sodium chloride solution having a pH between 6 and 9, lactated Ringer's injection solution, D5W, or PLASMA-LYTE A Injection pH 7.4® (Baxter, Deerfield, Ill.).

The kit can include one or more containers for the composition containing a CDP-taxane conjugate described herein. In some embodiments, the kit contains separate containers, dividers or compartments for the composition and informational material. For example, the composition can be contained in a bottle, vial, IV admixture bag, IV infusion set, piggyback set or syringe, and the informational material can be contained in a plastic sleeve or packet. In other embodiments, the separate elements of the kit are contained within a single, undivided container. For example, the composition is contained in a bottle, vial or syringe that has attached thereto the informational material in the form of a label. In some embodiments, the kit includes a plurality (e.g., a pack) of individual containers, each containing one or more unit dosage forms (e.g., a dosage form described herein) of a CDP-taxane conjugate described herein. For example, the kit includes a plurality of syringes, ampules, foil packets, or blister packs, each containing a single unit dose of a particle described herein. The containers of the kits can be air tight, waterproof (e.g., impermeable to changes in moisture or evaporation), and/or light-tight.

The kit optionally includes a device suitable for administration of the composition, e.g., a syringe, inhalant, pipette, forceps, measured spoon, dropper (e.g., eye dropper), swab (e.g., a cotton swab or wooden swab), or any such delivery device. In one embodiment, the device is a medical implant device, e.g., packaged for surgical insertion.

### Combination Therapy

The CDP-taxane conjugate may be used in combination with other known therapies. Administered “in combination”, as used herein, means that two (or more) different treatments are delivered to the subject during the course of the subject's affliction with the disorder, e.g., the two or more treatments are delivered after the subject has been diagnosed with the disorder and before the disorder has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery”. In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

The CDP-taxane conjugate and the at least one additional therapeutic agent can be administered simultaneously, in the same or in separate compositions, or sequentially. For sequential administration, the CDP-taxane conjugate can be administered first, and the additional agent can be administered second, or the order of administration can be reversed.

In some embodiments, the CDP-taxane conjugate is administered in combination with other therapeutic treatment modalities, including surgery, radiation, cryosurgery, and/or thermotherapy. Such combination therapies may advantageously utilize lower dosages of the administered agent and/or other chemotherapeutic agent, thus avoiding possible toxicities or complications associated with the various monotherapies. The phrase “radiation” includes, but is not limited to, external-beam therapy which involves three dimensional, conformal radiation therapy where the field of radiation is designed to conform to the volume of tissue treated; interstitial-radiation therapy where seeds of radioactive compounds are implanted using ultrasound guidance; and a combination of external-beam therapy and interstitial-radiation therapy.

In some embodiments, the CDP-taxane conjugate is administered with at least one additional therapeutic agent, such as a chemotherapeutic agent. In certain embodiments, the CDP-taxane is administered in combination with one or more additional chemotherapeutic agent, e.g., with one or more chemotherapeutic agents described herein. Exemplary classes of chemotherapeutic agents include, e.g., the following:

alkylating agents (including, without limitation, nitrogen mustards, ethylenimine derivatives, alkyl sulfonates, nitrosoureas and triazenes): uracil mustard (Aminouracil Mustard®, Chlorethaminacil®, Demethyldopan®, Desmethyldopan®, Haemanthamine®, Nordopan®, Uracil nitrogen Mustard®, Uracillost®, Uracilmostaza®, Uramustin®, Uramustine®, chlormethine (Mustargen®), cyclophosphamide (Cytoxan®, Neosar®, Clafen®, Endoxan®, Procytox®, Revimmune™), ifosfamide (Mitoxana®), melphalan (Alkeran®), Chlorambucil (Leukeran®), pipobroman (Amedel®, Vercyte®), triethylenemelamine (Hemel®, Hexylen®, Hexastat®), triethylenethiophosphoramine, Temozolomide (Temodar®), thiotepa (Thioplex®), busulfan (Busilvex®, Myleran®), carmustine (BiCNU®), lomustine (CeeNU®), streptozocin (Zanosar®), and Dacarbazine (DTIC-Dome®).

anti-EGFR antibodies (e.g., cetuximab (Erbitux®), panitumumab (Vectibix®), and gefitinib (Iressa®)).

anti-Her-2 antibodies (e.g., trastuzumab (Herceptin®) and other antibodies from Genentech).

antimetabolites (including, without limitation, folic acid antagonists (also referred to herein as antifolates), pyrimidine analogs, purine analogs and adenosine deaminase inhibitors): methotrexate (Rheumatrex®, Trexall®), 5-fluorouracil (Adrucil®, Efudex®, Fluoroplex®), floxuridine (FUDF®), cytarabine (Cytosar-U®, Tarabine PFS), 6-mercaptopurine (Puri-Nethol®)), 6-thioguanine (Thioguanine Tabloid®), fludarabine phosphate (Fludara®), pentostatin (Nipent®), pemetrexed (Alimta®), raltitrexed (Tomudex®), cladribine (Leustatin®), clofarabine (Clofarex®, Clolar®), mercaptopurine (Puri-Nethol®), capecitabine (Xeloda®), nelarabine (Arranon®), azacitidine (Vidaza®) and gemcitabine (Gemzar®). Preferred antimetabolites include, e.g., 5-fluorouracil (Adrucil®, Efudex®, Fluoroplex®), floxuridine (FUDF®), capecitabine (Xeloda®), pemetrexed (Alimta®), raltitrexed (Tomudex®) and gemcitabine (Gemzar®).

vinca alkaloids: vinblastine (Velban®, Velsar®), vincristine (Vincasar®, Oncovin®), vindesine (Eldisine®), vinorelbine (Navelbine®).

platinum-based agents: carboplatin (Paraplat®, Paraplatin®), cisplatin (Platinol®), oxaliplatin (Eloxatin®).

anthracyclines: daunorubicin (Cerubidine®, Rubidomycin®), doxorubicin (Adriamycin®), epirubicin (Ellence®), idarubicin (Idamycin®), mitoxantrone (Novantrone®), valrubicin (Valstar®). Preferred anthracyclines include daunorubicin (Cerubidine®, Rubidomycin®) and doxorubicin (Adriamycin®).

topoisomerase inhibitors: topotecan (Hycamtin®), irinotecan (Camptosar®), etoposide (Toposar®, VePesid®), teniposide (Vumon®), lamellarin D, SN-38, camptothecin (e.g., CRLX101).

taxanes: paclitaxel (Taxol®), docetaxel (Taxotere®), larotaxel, cabazitaxel.

antibiotics: actinomycin (Cosmegen®), bleomycin (Blenoxane®), hydroxyurea (Droxia®, Hydrea®), mitomycin (Mitozytrex®, Mutamycin®).

immunomodulators: lenalidomide (Revlimid®), thalidomide (Thalomid®).

immune cell antibodies: alemtuzamab (Campath®), gemtuzumab (Myelotarg®), rituximab (Rituxan®), tositumomab (Bexxar®).

proteosome inhibitors: bortezomib (Velcade®).

interferons (e.g., IFN-alpha (Alferon®, Roferon-A®, Intron®-A) or IFN-gamma (Actimmune®))

interleukins: IL-1, IL-2 (Proleukin®), IL-24, IL-6 (Sigosix®), IL-12.

HSP90 inhibitors (e.g., geldanamycin or any of its derivatives). In certain embodiments, the HSP90 inhibitor is selected from geldanamycin, 17-alkylamino-17-desmethoxygeldanamycin (“17-AAG”) or 17-(2-dimethylaminoethyl)amino-17-desmethoxygeldanamycin (“17-DMAG”).

anti-androgens which include, without limitation nilutamide (Nilandron®) and bicalutamide (Caxodex®).

antiestrogens which include, without limitation tamoxifen (Nolvadex®), toremifene (Fareston®), letrozole (Femara®), testolactone (Teslac®), anastrozole (Arimidex®), bicalutamide (Casodex®), exemestane (Aromasin®), flutamide (Eulexin®), fulvestrant (Faslodex®), raloxifene (Evista®, Keoxifene®) and raloxifene hydrochloride.

anti-hypercalcaemia agents which include without limitation gallium (III) nitrate hydrate (Ganite®) and pamidronate disodium (Aredia®).

apoptosis inducers which include without limitation ethanol, 2-[[3-(2,3-dichlorophenoxy)propyl]amino]-(9Cl), gambogic acid, embelin and arsenic trioxide (Trisenox®).

Aurora kinase inhibitors which include without limitation binucleine 2.

Bruton's tyrosine kinase inhibitors which include without limitation terreic acid.

calcineurin inhibitors which include without limitation cypermethrin, deltamethrin, fenvalerate and tyrphostin 8.

CaM kinase II inhibitors which include without limitation 5-Isoquinolinesulfonic acid, 4-[{2S)-2-[(5-isoquinolinylsulfonyl)methylamino]-3-oxo-3-4-phenyl-1-piperazinyl)propyl]phenyl ester and benzenesulfonamide.

CD45 tyrosine phosphatase inhibitors which include without limitation phosphonic acid.

CDC25 phosphatase inhibitors which include without limitation 1,4-naphthalene dione, 2,3-bis[(2-hydroxyethyl)thio]-(9Cl).

CHK kinase inhibitors which include without limitation debromohymenialdisine.

cyclooxygenase inhibitors which include without limitation 1H-indole-3-acetamide, 1-(4-chlorobenzoyl)-5-methoxy-2-methyl-N-(2-phenylethyl)-(9Cl), 5-alkyl substituted 2-arylaminophenylacetic acid and its derivatives (e.g., celecoxib (Celebrex®), rofecoxib (Vioxx®), etoricoxib (Arcoxia®), lumiracoxib (Prexige®), valdecoxib (Bextra®) or 5-alkyl-2-arylaminophenylacetic acid).

cRAF kinase inhibitors which include without limitation 3-(3,5-dibromo-4-hydroxybenzylidene)-5-iodo-1,3-dihydroindol-2-one and benzamide, 3-(dimethylamino)-N-[3-[(4-hydroxybenzoyl)amino]-4-methylphenyl]-(9Cl).

cyclin dependent kinase inhibitors which include without limitation olomoucine and its derivatives, purvalanol B, roascovitine (Seliciclib®), indirubin, kenpaullone, purvalanol A and indirubin-3′-monooxime.

cysteine protease inhibitors which include without limitation 4-morpholinecarboxamide, N-[(1S)-3-fluoro-2-oxo-1-(2-phenylethyl)propyl]amino]-2-oxo-1-(phenylmethyl)ethyl]-(9Cl).

DNA intercalators which include without limitation plicamycin (Mithracin®) and daptomycin (Cubicin®).

DNA strand breakers which include without limitation bleomycin (Blenoxane®).

E3 ligase inhibitors which include without limitation N-((3,3,3-trifluoro-2-trifluoromethyl)propionyl)sulfanilamide.

EGF Pathway Inhibitors which include, without limitation tyrphostin 46, EKB-569, erlotinib (Tarceva®), gefitinib (Iressa®), lapatinib (Tykerb®) and those compounds that are generically and specifically disclosed in WO 97/02266, EP 0 564 409, WO 99/03854, EP 0 520 722, EP 0 566 226, EP 0 787 722, EP 0 837 063, U.S. Pat. No. 5,747,498, WO 98/10767, WO 97/30034, WO 97/49688, WO 97/38983 and WO 96/33980.

farnesyltransferase inhibitors which include without limitation A-hydroxyfarnesylphosphonic acid, butanoic acid, 2-[(25)-2-[[(2S,3S)-2-[[(2R)-2-amino-3-mercaptopropyl]amino]-3-methylpentyl]oxy]-1-oxo-3-phenylpropyl]amino]-4-(methylsulfonyl)-1-methylethylester (2S)-(9Cl), and manumycin A.

Flk-1 kinase inhibitors which include without limitation 2-propenamide, 2-cyano-3-[4-hydroxy-3,5-bis(1-methylethyl)phenyl]-N-(3-phenylpropyl)-(2E)-(9Cl).

glycogen synthase kinase-3 (GSK3) inhibitors which include without limitation indirubin-3′-monooxime

histone deacetylase (HDAC) inhibitors which include without limitation suberoylanilide hydroxamic acid (SAHA), [4-(2-amino-phenylcarbamoyl)-benzyl]-carbamic acid pyridine-3-ylmethylester and its derivatives, butyric acid, pyroxamide, trichostatin A, oxamflatin, apicidin, depsipeptide, depudecin, trapoxin and compounds disclosed in WO 02/22577.

I-kappa B-alpha kinase inhibitors (IKK) which include without limitation 2-propenenitrile, 3-[(4-methylphenyl)sulfonyl]-(2E)-(9Cl).

imidazotetrazinones which include without limitation temozolomide (Methazolastone®, Temodar® and its derivatives (e.g., as disclosed generically and specifically in U.S. Pat. No. 5,260,291) and Mitozolomide.

insulin tyrosine kinase inhibitors which include without limitation hydroxyl-2-naphthalenylmethylphosphonic acid.

c-Jun-N-terminal kinase (JNK) inhibitors which include without limitation pyrazoleanthrone and epigallocatechin gallate.

mitogen-activated protein kinase (MAP) inhibitors which include without limitation benzenesulfonamide, N-[2-[[[3-(4-chlorophenyl)-2-propenyl]methyl]amino]methyl]phenyl]-N-(2-hydroxyethyl)-4-methoxy-(9Cl).

MDM2 inhibitors which include without limitation trans-4-iodo, 4′-boranyl-chalcone.

MEK inhibitors which include without limitation butanedinitrile, bis[amino[2-aminophenyl)thio]methylene]-(9Cl).

MMP inhibitors which include without limitation Actinonin, epigallocatechin gallate, collagen peptidomimetic and non-peptidomimetic inhibitors, tetracycline derivatives marimastat (Marimastat®), prinomastat, incyclinide (Metastat®), shark cartilage extract AE-941 (Neovastat®), Tanomastat, TAA211, MMI270B or AAJ996.

mTor inhibitors which include without limitation rapamycin (Rapamune®), and analogs and derivatives thereof, AP23573 (also known as ridaforolimus, deforolimus, or MK-8669), CCI-779 (also known as temsirolimus) (Torisel®) and SDZ-RAD.

NGFR tyrosine kinase inhibitors which include without limitation tyrphostin AG 879.

p38 MAP kinase inhibitors which include without limitation Phenol, 4-[4-(4-fluorophenyl)-5-(4-pyridinyl)-1H-imidazol-2-yl]-(9Cl), and benzamide, 3-(dimethylamino)-N-[3-[(4-hydroxylbenzoyl)amino]-4-methylphenyl]-(9Cl).

p56 tyrosine kinase inhibitors which include without limitation damnacanthal and tyrphostin 46.

PDGF pathway inhibitors which include without limitation tyrphostin AG 1296, tyrphostin 9, 1,3-butadiene-1,1,3-tricarbonitrile, 2-amino-4-(1H-indol-5-yl)-(9Cl), imatinib (Gleevec®) and gefitinib (Iressa®) and those compounds generically and specifically disclosed in European Patent No.: 0 564 409 and PCT Publication No.: WO 99/03854.

phosphatidylinositol 3-kinase inhibitors which include without limitation wortmannin, and quercetin dihydrate.

phosphatase inhibitors which include without limitation cantharidic acid, cantharidin, and L-leucinamide.

protein phosphatase inhibitors which include without limitation cantharidic acid, cantharidin, L-P-bromotetramisole oxalate, 2(5H)-furanone, 4-hydroxy-5-(hydroxymethyl)-3-(1-oxohexadecyl)-(5R)-(9Cl) and benzylphosphonic acid.

PKC inhibitors which include without limitation 1-H-pyrollo-2,5-dione,3-[1-[3-(dimethylamino)propyl]-1H-indol-3-yl]-4-(1H-indol-3-yl)-(9Cl), Bisindolylmaleimide IX, Sphinogosine, staurosporine, and Hypericin.

PKC delta kinase inhibitors which include without limitation rottlerin.

polyamine synthesis inhibitors which include without limitation DMFO.

proteasome inhibitors which include, without limitation aclacinomycin A, gliotoxin and bortezomib (Velcade®).

PTP1B inhibitors which include without limitation L-leucinamide.

protein tyrosine kinase inhibitors which include, without limitation tyrphostin Ag 216, tyrphostin Ag 1288, tyrphostin Ag 1295, geldanamycin, genistein and 7H-pyrollo[2,3-d]pyrimidine derivatives of formula I as generically and specifically described in PCT Publication No.: WO 03/013541 and U.S. Publication No.: 2008/0139587:

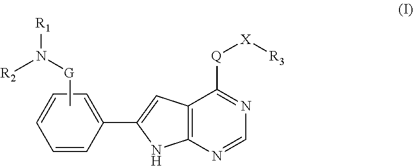

Publication No.: 2008/0139587 discloses the various substituents, e.g., R₁, R₂, etc.

SRC family tyrosine kinase inhibitors which include without limitation PP1 and PP2.

Syk tyrosine kinase inhibitors which include without limitation piceatannol.

Janus (JAK-2 and/or JAK-3) tyrosine kinase inhibitors which include without limitation tyrphostin AG 490 and 2-naphthyl vinyl ketone.

retinoids which include without limitation isotretinoin (Accutane®, Amnesteem®, Cistane®, Claravis®, Sotret®) and tretinoin (Aberel®, Aknoten®, Avita®, Renova®, Retin-A®, Retin-A MICRO®, Vesanoid®).

RNA polymerase II elongation inhibitors which include without limitation 5,6-dichloro-1-beta-D-ribofuranosylbenzimidazole.

serine/Threonine kinase inhibitors which include without limitation 2-aminopurine.

sterol biosynthesis inhibitors which include without limitation squalene epoxidase and CYP2D6.

VEGF pathway inhibitors, which include without limitation anti-VEGF antibodies, e.g., bevacizumab, and small molecules, e.g., sunitinib (Sutent®), sorafinib (Nexavar®), ZD6474 (also known as vandetanib) (Zactima™), SU6668, CP-547632 and AZD2171 (also known as cediranib) (Recentin™).

Examples of chemotherapeutic agents are also described in the scientific and patent literature, see, e.g., Bulinski (1997) J. Cell Sci. 110:3055-3064; Panda (1997) Proc. Natl. Acad. Sci. USA 94:10560-10564; Muhlradt (1997) Cancer Res. 57:3344-3346; Nicolaou (1997) Nature 387:268-272; Vasquez (1997) Mol. Biol. Cell. 8:973-985; Panda (1996) J. Biol. Chem. 271:29807-29812.

In some embodiment, the CDP-taxane conjugate is administered instead of another microtubule affecting agent, e.g., instead of a microtubule affecting agent as a first line therapy or a second line therapy. For example, the CDP-taxane conjugate can be used instead of any of the following microtubule affecting agents allocolchicine (NSC 406042), halichondrin B (NSC 609395), colchicine (NSC 757), colchicine derivatives (e.g., NSC 33410), dolastatin 10 (NSC 376128), maytansine (NSC 153858), rhizoxin (NSC 332598), paclitaxel (Taxol®, NSC 125973), taxol derivatives (e.g., derivatives (e.g., NSC 608832), thiocolchicine (NSC 361792), trityl cysteine (NSC 83265), vinblastine sulfate (NSC 49842), vincristine sulfate (NSC 67574).

In some cases, a hormone and/or steriod can be administered in combination with a CDP-taxane conjugate. Examples of hormones and steroids include: 17a-ethinylestradiol (Estinyl®, Ethinoral®, Feminone®, Orestralyn®), diethylstilbestrol (Acnestrol®, Cyren A®, Deladumone®, Diastyl®, Domestrol®, Estrobene®, Estrobene®, Estrosyn®, Fonatol®, Makarol®, Milestrol®, Milestrol®, Neo-Oestronol I®, Oestrogenine®, Oestromenin®, Oestromon®, Palestrol®, Stilbestrol®, Stilbetin®, Stilboestroform®, Stilboestrol®, Synestrin®, Synthoestrin®, Vagestrol®), testosterone (Delatestryl®, Testoderm®, Testolin®, Testostroval®, Testostroval-PA®, Testro AQ®), prednisone (Delta-Dome®, Deltasone®, Liquid Pred®, Lisacort®, Meticorten®, Orasone®, Prednicen-M®, Sk-Prednisone®, Sterapred®), Fluoxymesterone (Android-F®, Halodrin®, Halotestin®, Ora-Testryl®, Ultandren®), dromostanolone propionate (Drolban®, Emdisterone®, Masterid®, Masteril®, Masteron®, Masterone®, Metholone®, Permastril®), testolactone (Teslac®), megestrolacetate (Magestin®, Maygace®, Megace®, Megeron®, Megestat®, Megestil®, Megestin®, Nia®, Niagestin®, Ovaban®, Ovarid®, Volidan®), methylprednisolone (Depo-Medrol®, Medlone 21®, Medrol®, Meprolone®, Metrocort®, Metypred®, Solu-Medrol®, Summicort®), methyl-testosterone (Android®, Testred®, Virilon®), prednisolone (Cortalone®, Delta-Cortef®, Hydeltra®, Hydeltrasol®, Meti-derm®, Prelone®), triamcinolone (Aristocort®), chlorotrianisene (Anisene®, Chlorotrisin®, Clorestrolo®, Clorotrisin®, Hormonisene®, Khlortrianizen®, Merbentul®, Metace®, Rianil®, Tace®, Tace-Fn®, Trianisestrol®), hydroxyprogesterone (Delalutin®, Gestiva™), aminoglutethimide (Cytadren®, Elipten®, Orimeten®), estramustine (Emcyt®), medroxyprogesteroneacetate (Provera®, Depo-Provera®), leuprolide (Lupron®, Viadur®), flutamide (Eulexin®), toremifene (Fareston®), and goserelin (Zoladex®).

In certain embodiments, the CDP-taxane conjugate is administered in combination with an anti-microbial (e.g., leptomycin B).

In another embodiment, the CDP-taxane conjugate is administered in combination with an agent or procedure to mitigate potential side effects from the agent compositions such as diarrhea, nausea and vomiting.

Diarrhea may be treated with antidiarrheal agents including, but not limited to opioids (e.g., codeine (Codicept®, Coducept®), oxicodeine, percocet, paregoric, tincture of opium, diphenoxylate (Lomotil®), diflenoxin), and loperamide (Imodium A-D®), bismuth subsalicylate, lanreotide, vapreotide (Sanvar®, Sanvar MC1), motiln antagonists, COX2 inhibitors (e.g., celecoxib (Celebrex®), glutamine (NutreStore®), thalidomide (Synovir®, Thalomid®), traditional antidiarrhea remedies (e.g., kaolin, pectin, berberine and muscarinic agents), octreotide and DPP-IV inhibitors.

DPP-IV inhibitors employed in the present invention are generically and specifically disclosed in PCT Publication Nos.: WO 98/19998, DE 196 16 486 A1, WO 00/34241 and WO 95/15309.

Nausea and vomiting may be treated with antiemetic agents such as dexamethasone (Aeroseb-Dex®, Alba-Dex®, Decaderm®, Decadrol®, Decadron®, Decasone®, Decaspray®, Deenar®, Deronil®, Dex-4®, Dexace®, Dexameth®, Dezone®, Gammacorten®, Hexadrol®, Maxidex®, Sk-Dexamethasone®), metoclopramide (Reglan®), diphenylhydramine (Benadryl®, SK-Diphenhydramine®), lorazepam (Ativan®), ondansetron (Zofran®), prochlorperazine (Bayer A 173®, Buccastem®, Capazine®, Combid®, Compazine®, Compro®, Emelent®, Emetiral®, Eskatrol®, Kronocin®, Meterazin®, Meterazin Maleate®, Meterazine®, Nipodal®, Novamin®, Pasotomin®, Phenotil®, Stemetil®, Stemzine®, Tementil®, Temetid®, Vertigon®), thiethylperazine (Norzine®, Torecan®), and dronabinol (Marinol®).

In some embodiments, the CDP-taxane conjugate is administered in combination with an immunosuppressive agent Immunosuppressive agents suitable for the combination include, but are not limited to natalizumab (Tysabri®), azathioprine (Imuran®), mitoxantrone (Novantrone®), mycophenolate mofetil (Cellcept®), cyclosporins (e.g., Cyclosporin A (Neoral®, Sandimmun®, Sandimmune®, SangCya®), cacineurin inhibitors (e.g., Tacrolimus (Prograf®, Protopic®), sirolimus (Rapamune®), everolimus (Afinitor®), cyclophosphamide (Clafen®, Cytoxan®, Neosar®), or methotrexate (Abitrexate®, Folex®, Methotrexate®, Mexate®)), fingolimod, mycophenolate mofetil (CellCept®), mycophenolic acid (Myfortic®), anti-CD3 antibody, anti-CD25 antibody (e.g., Basiliximab (Simulect®) or daclizumab (Zenapax®)), and anti-TNFα antibody (e.g., Infliximab (Remicade®) or adalimumab (Humira®)).

In some embodiments, a CDP-taxane conjugate is administered in combination with a CYP3A4 inhibitor (e.g., ketoconazole (Nizoral®, Xolegel®), itraconazole (Sporanox®), clarithromycin (Biaxin®), atazanavir (Reyataz®), nefazodone (Serzone®, Nefadar®), saquinavir (Invirase®), telithromycin (Ketek®), ritonavir (Norvir®), amprenavir (also known as Agenerase, a prodrug version is fosamprenavir (Lexiva®, Telzir®), indinavir (Crixivan®), nelfinavir (Viracept®), delavirdine (Rescriptor®) or voriconazole (Vfend®)).

When employing the methods or compositions, other agents used in the modulation of tumor growth or metastasis in a clinical setting, such as antiemetics, can also be administered as desired.

When formulating the pharmaceutical compositions featured in the invention the clinician may utilize preferred dosages as warranted by the condition of the subject being treated. For example, in one embodiment, a CDP-taxane conjugate may be administered at a dosing schedule described herein, e.g., once every one, two three four, five, or six weeks.

Also, in general, a CDP-taxane conjugate and an additional chemotherapeutic agent(s) do not have to be administered in the same pharmaceutical composition, and may, because of different physical and chemical characteristics, have to be administered by different routes. For example, the CDP-taxane conjugate may be administered intravenously while the chemotherapeutic agent(s) may be administered orally. The determination of the mode of administration and the advisability of administration, where possible, in the same pharmaceutical composition, is well within the knowledge of the skilled clinician. The initial administration can be made according to established protocols known in the art, and then, based upon the observed effects, the dosage, modes of administration and times of administration can be modified by the skilled clinician.

In one embodiment, a CDP-taxane conjugate is administered once every three weeks and an additional therapeutic agent (or additional therapeutic agents) may also be administered every three weeks for as long as treatment is required. Examples of other chemotherapeutic agents which are administered one every three weeks include: an antimetabolite (e.g., floxuridine (FUDF®), pemetrexed (ALIMTA®), 5FU (Adrucil®, Efudex®, Fluoroplex®)); an anthracycline (e.g., daunorubicin (Cerubidine®, Rubidomycin®), epirubicin (Ellence®), idarubicin (Idamycin®), mitoxantrone (Novantrone®), valrubicin (Valstar®)); a vinca alkaloid (e.g., vinblastine (Velban®, Velsar®), vincristine (Vincasar®, Oncovin®), vindesine (Eldisine®) and vinorelbine (Navelbine®)); a topoisomerase inhibitor (e.g., topotecan (Hycamtin®), irinotecan (Camptosar®), etoposide (Toposar®, VePesid®), teniposide (Vumon®), lamellarin D, SN-38, camptothecin (e.g., CRLX101)); and a platinum-based agent (e.g., cisplatin (Platinol®), carboplatin (Paraplat®, Paraplatin®), oxaliplatin (Eloxatin®)).

In another embodiment, the CDP-taxane conjugate is administered once every two weeks in combination with one or more additional chemotherapeutic agent that is administered orally. For example, the CDP-taxane conjugate can be administered once every two weeks in combination with one or more of the following chemotherapeutic agents: capecitabine (Xeloda®), estramustine (Emcyt®), erlotinib (Tarceva®), rapamycin (Rapamune®), SDZ-RAD, CP-547632; AZD2171, sunitinib (Sutent®), sorafenib (Nexavar®) and everolimus (Afinitor®).

The actual dosage of the CDP-taxane conjugate and/or any additional chemotherapeutic agent employed may be varied depending upon the requirements of the subject and the severity of the condition being treated. Determination of the proper dosage for a particular situation is within the skill of the art. Generally, treatment is initiated with smaller dosages which are less than the optimum dose of the compound. Thereafter, the dosage is increased by small amounts until the optimum effect under the circumstances is reached.

In some embodiments, when a CDP-taxane conjugate is administered in combination with one or more additional chemotherapeutic agent, the additional chemotherapeutic agent (or agents) is administered at a standard dose. For example, a standard dosage for cisplatin is 75-120 mg/m² administered every three weeks; a standard dosage for carboplatin is within the range of 200-600 mg/m² or an AUC of 0.5-8 mg/ml×min; e.g., at an AUC of 4-6 mg/ml×min; a standard dosage for irinotecan is within 100-125 mg/m², once a week; a standard dosage for gemcitabine is within the range of 80-1500 mg/m² administered weekly; a standard dose for UFT is within a range of 300-400 mg/m² per day when combined with leucovorin administration; a standard dosage for leucovorin is 10-600 mg/m² administered weekly.

The disclosure also encompasses a method for the synergistic treatment of cancer wherein a CDP-taxane conjugate is administered in combination with an additional chemotherapeutic agent or agents.

The particular choice of conjugate and anti-proliferative cytotoxic agent(s) or radiation will depend upon the diagnosis of the attending physicians and their judgment of the condition of the subject and the appropriate treatment protocol.

If the CDP-taxane conjugate and the chemotherapeutic agent(s) and/or radiation are not administered simultaneously or essentially simultaneously, then the initial order of administration of the CDp-taxane conjugate, and the chemotherapeutic agent(s) and/or radiation, may be varied. Thus, for example, the CDP-taxane conjugate may be administered first followed by the administration of the chemotherapeutic agent(s) and/or radiation; or the chemotherapeutic agent(s) and/or radiation may be administered first followed by the administration of the CDP-taxane conjugate. This alternate administration may be repeated during a single treatment protocol. The determination of the order of administration, and the number of repetitions of administration of each therapeutic agent during a treatment protocol, is well within the knowledge of the skilled physician after evaluation of the disease being treated and the condition of the subject.

Thus, in accordance with experience and knowledge, the practicing physician can modify each protocol for the administration of a component (CDP-taxane conjugate, anti-neoplastic agent(s), or radiation) of the treatment according to the individual subject's needs, as the treatment proceeds.

The attending clinician, in judging whether treatment is effective at the dosage administered, will consider the general well-being of the subject as well as more definite signs such as relief of disease-related symptoms, inhibition of tumor growth, actual shrinkage of the tumor, or inhibition of metastasis. Size of the tumor can be measured by standard methods such as radiological studies, e.g., CAT or MRI scan, and successive measurements can be used to judge whether or not growth of the tumor has been retarded or even reversed. Relief of disease-related symptoms such as pain, and improvement in overall condition can also be used to help judge effectiveness of treatment.

Indications

The disclosed CDP-taxane conjugates are useful in evaluating or treating proliferative disorders, e.g., treating a tumor and metastases thereof wherein the tumor or metastases thereof is a cancer described herein. The methods described herein can be used to treat a solid tumor, a soft tissue tumor or a liquid tumor. Exemplary solid tumors include malignancies (e.g., sarcomas and carcinomas (e.g., adenocarcinoma or squamous cell carcinoma)) of the various organ systems, such as those of brain, lung, breast, lymphoid, gastrointestinal (e.g., colon), and genitourinary (e.g., renal, urothelial, or testicular tumors) tracts, pharynx, prostate, and ovary. Exemplary adenocarcinomas include colorectal cancers, renal-cell carcinoma, liver cancer, non-small cell carcinoma of the lung, and cancer of the small intestine. The disclosed methods are also useful in evaluating or treating soft tissue tumors such as those of the tendons, muscles or fat, and liquid tumors.

The methods described herein can be used with any cancer, for example those described by the National Cancer Institute. The cancer can be a carcinoma, a sarcoma, a myeloma, a leukemia, a lymphoma or a mixed type. Exemplary cancers described by the National Cancer Institute include:

Digestive/gastrointestinal cancers such as anal cancer; bile duct cancer; extrahepatic bile duct cancer; appendix cancer; carcinoid tumor, gastrointestinal cancer; colon cancer; colorectal cancer, childhood; esophageal cancer; esophageal cancer, childhood; gallbladder cancer; gastric (stomach) cancer; gastric (stomach) cancer, childhood; hepatocellular (liver) cancer, adult (primary); hepatocellular (liver) cancer, childhood (primary); extrahepatic; pancreatic cancer; pancreatic cancer, childhood; sarcoma, rhabdomyosarcoma; pancreatic cancer, islet cell; rectal cancer; and small intestine cancer;

Endocrine cancers such as islet cell carcinoma (endocrine pancreas); adrenocortical carcinoma; adrenocortical carcinoma, childhood; gastrointestinal carcinoid tumor; parathyroid cancer; pheochromocytoma; pituitary tumor; thyroid cancer; thyroid cancer, childhood; multiple endocrine neoplasia syndrome, childhood; and carcinoid tumor, childhood;

Eye cancers such as intraocular melanoma; and retinoblastoma;

Musculoskeletal cancers such as Ewing's family of tumors; osteosarcoma/malignant fibrous histiocytoma of the bone; rhabdomyosarcoma, childhood; soft tissue sarcoma, adult; soft tissue sarcoma, childhood; clear cell sarcoma of tendon sheaths; and uterine sarcoma;

Breast cancer such as breast cancer and pregnancy; breast cancer, childhood; and breast cancer, male;

Neurologic cancers such as brain stem glioma, childhood; brain tumor, adult; brain stem glioma, childhood; cerebellar astrocytoma, childhood; cerebral astrocytoma/malignant glioma, childhood; ependymoma, childhood; medulloblastoma, childhood; pineal and supratentorial primitive neuroectodermal tumors, childhood; visual pathway and hypothalamic glioma, childhood; other childhood brain cancers; adrenocortical carcinoma; central nervous system lymphoma, primary; cerebellar astrocytoma, childhood; neuroblastoma; craniopharyngioma; spinal cord tumors; central nervous system atypical teratoid/rhabdoid tumor; central nervous system embryonal tumors; andsupratentorial primitive neuroectodermal tumors, childhood and pituitary tumor;

Genitourinary cancers such as bladder cancer; bladder cancer, childhood; kidney cancer; ovarian cancer, childhood; ovarian epithelial cancer; ovarian low malignant potential tumor; penile cancer; prostate cancer; renal cell cancer, childhood; renal pelvis and ureter, transitional cell cancer; testicular cancer; urethral cancer; vaginal cancer; vulvar cancer; cervical cancer; Wilms tumor and other childhood kidney tumors; endometrial cancer; and gestational trophoblastic tumor;

Germ cell cancers such as extracranial germ cell tumor, childhood; extragonadal germ cell tumor; ovarian germ cell tumor; and testicular cancer;

Head and neck cancers such as lip and oral cavity cancer; oral cancer, childhood; hypopharyngeal cancer; laryngeal cancer; laryngeal cancer, childhood; metastatic squamous neck cancer with occult primary; mouth cancer; nasal cavity and paranasal sinus cancer; nasopharyngeal cancer; nasopharyngeal cancer, childhood; oropharyngeal cancer; parathyroid cancer; pharyngeal cancer; salivary gland cancer; salivary gland cancer, childhood; throat cancer; and thyroid cancer;

Hematologic/blood cell cancers such as a leukemia (e.g., acute lymphoblastic leukemia, adult; acute lymphoblastic leukemia, childhood; acute myeloid leukemia, adult; acute myeloid leukemia, childhood; chronic lymphocytic leukemia; chronic myelogenous leukemia; and hairy cell leukemia); a lymphoma (e.g., AIDS-related lymphoma; cutaneous T-cell lymphoma; Hodgkin's lymphoma, adult; Hodgkin's lymphoma, childhood; Hodgkin's lymphoma during pregnancy; non-Hodgkin's lymphoma, adult; non-Hodgkin's lymphoma, childhood; non-Hodgkin's lymphoma during pregnancy; mycosis fungoides; sezary syndrome; T-cell lymphoma, cutaneous; Waldenstrom's macroglobulinemia; and primary central nervous system lymphoma); and other hematologic cancers (e.g., chronic myeloproliferative disorders; multiple myeloma/plasma cell neoplasm; myelodysplastic syndromes; and myelodysplastic/myeloproliferative disorders);

Lung cancer such as non-small cell lung cancer; and small cell lung cancer;

Respiratory cancers such as malignant mesothelioma, adult; malignant mesothelioma, childhood; malignant thymoma; thymoma, childhood; thymic carcinoma; bronchial adenomas/carcinoids; pleuropulmonary blastoma; non-small cell lung cancer; and small cell lung cancer;

Skin cancers such as Kaposi's sarcoma; Merkel cell carcinoma; melanoma; and skin cancer, childhood;

Other childhood cancers and cancers of unknown primary site;

and metastases of the aforementioned cancers can also be treated or prevented in accordance with the methods described herein.

The CDP-taxane conjugates described herein are particularly suited to treat accelerated or metastatic cancers of the bladder cancer, pancreatic cancer, prostate cancer, renal cancer, non-small cell lung cancer, ovarian cancer, melanoma, colorectal cancer, and breast cancer.

In one embodiment, a method is provided for a combination treatment of a cancer, such as by treatment with a CDP-taxane conjugate and a second therapeutic agent. Various combinations are described herein. The combination can reduce the development of tumors, reduces tumor burden, or produce tumor regression in a mammalian host.

In some embodiments, the proliferative disorder is a disease or disorder associated with inflammation. A CDP-taxane conjugate described herein may be administered prior to the onset of, at, or after the initiation of inflammation. When used prophylactically, the CDP-taxane is preferably provided in advance of any inflammatory response or symptom. Administration of the CDP-taxane conjugate may prevent or attenuate inflammatory responses or symptoms. Exemplary inflammatory conditions include, for example, multiple sclerosis, rheumatoid arthritis, psoriatic arthritis, degenerative joint disease, spondouloarthropathies, gouty arthritis, systemic lupus erythematosus, juvenile arthritis, rheumatoid arthritis, osteoarthritis, osteoporosis, diabetes (e.g., insulin dependent diabetes mellitus or juvenile onset diabetes), menstrual cramps, cystic fibrosis, inflammatory bowel disease, irritable bowel syndrome, Crohn's disease, mucous colitis, ulcerative colitis, gastritis, esophagitis, pancreatitis, peritonitis, Alzheimer's disease, shock, ankylosing spondylitis, gastritis, conjunctivitis, pancreatis (acute or chronic), multiple organ injury syndrome (e.g., secondary to septicemia or trauma), myocardial infarction, atherosclerosis, stroke, reperfusion injury (e.g., due to cardiopulmonary bypass or kidney dialysis), acute glomerulonephritis, vasculitis, thermal injury (i.e., sunburn), necrotizing enterocolitis, granulocyte transfusion associated syndrome, and/or Sjogren's syndrome. Exemplary inflammatory conditions of the skin include, for example, eczema, atopic dermatitis, contact dermatitis, urticaria, schleroderma, psoriasis, and dermatosis with acute inflammatory components.

The CDP-taxane conjugate can be administered to a subject undergoing or who has undergone angioplasty. In one embodiment, the CDP-taxane conjugate is administered to a subject undergoing or who has undergone angioplasty with a stent placement. In some embodiments, the CDP-taxane conjugate can be used as a strut of a stent or a coating for a stent.

The CDP-taxane can be used during the implantation of a stent, e.g., as a separate intravenous administration, as coating for a stent or as the strut of a stent.

Stent

The CDP-taxane conjugates described herein can be used as or be part of a stent. As used herein, the term “stent” refers to a man-made ‘tube’ inserted into a natural passage or conduit in the body to prevent or counteract localized flow constriction. Types of stents include, e.g., coronary stent, urinary tract stent, urethral/pro static stent, vascular stent (e.g., peripheral vascular stent, or stent graft), esophageal stent, duodenal stent, colonic stent, biliary stent, and pancreatic stent. Types of stents that can be used in coronary arteries include, e.g., bare-metal stent (BMS) and drug-eluting stent (DES). A coronary stent can be placed within the coronary artery during an angioplasty procedure.

Bare-Metal Stent (BMS)

In one embodiment, the CDP-taxane conjugate can be used in combination with a BMS. As used herein, BMS refers to a stent without a coating that is made or a metal or combination of metals. BMS can be made from, e.g., stainless steel (e.g., BxVelocity™ stent, Express2™ stent, R Stent™, and Matrix® coronary stent), cobalt-chromium alloy (e.g., Driver® coronary stent, ML Vision® stent, and Coronnium® stent), or nickel titanium (Nitinol® stent). A CDP-taxane conjugate described herein can be used as a coating of a BMS, e.g., to coat the luminal and/or abluminal surface of a BMS.

Drug-Eluting Stent (DES)

In one embodiment, the CDP-taxane conjugate can be a DES or can be part of a DES. As used herein, DES refers to a stent placed into a natural passage or conduit of the body (e.g., a narrowed coronary artery) that releases (e.g., slowly releases) one or more agents to treat one or more symptoms associated with the constricted flow to the passage or conduit and/or one or more effect caused by or associated with the stent. For example, the DES can release one (or more) agent that reduces or inhibits the migration and/or proliferation of vascular smooth muscle cells (SMCs), that promotes or increases epithelialization, that reduces or inhibits a hypersensitivity reaction, that reduces or inhibits inflammation, that reduces or inhibits thrombosis, that reduces the risk of restenosis, and/or that reduces or inhibits other unwanted effects due to the stent.

One type of DES includes a stent strut and a polymer, on which an agent is loaded. Thus, in one embodiment, a CDP-taxane conjugate described herein can be used in combination with other polymeric struts (e.g., other biocompatible or bioasorbable polymers). For example, a CDP-taxane conjugate described herein can be coated on a polymeric strut, e.g., on the luminal and/or abluminal surface of a polymeric strut.

In another embodiment, the CDP-taxane conjugates described herein can be used as a polymeric strut, with out without an additional polymer and/or agent.

In one embodiment, the rate of major adverse cardiac events (MACE) of a subject having a stent made of a CDP-taxane conjugate described herein or a strut coated with a CDP-taxane conjugate described herein is reduced by at least 10, 20, 30, 40, 50, 60, 70, 80, 90, 95% or more, as compared to the rate of MACE of a subject having a stent made of a different material (e.g., a metal or polymer) or a stent not coated or coated with a polymer and/or agent other than the CDP-taxane conjugate. In another embodiment, the need for target vessel revascularization (TVR) of a subject having a stent made of a CDP-taxane conjugate described herein or a strut coated with a CDP-taxane conjugate described herein is reduced by at least 10, 20, 30, 40, 50, 60, 70, 80, 90, 95% or more, compared to the TVR of a subject having a stent made of a different material (e.g., a metal or polymer) or a stent not coated or coated with a polymer and/or agent other than the CDP-taxane conjugate. In yet another embodiment, the rate for target lesion revascularization (TLR) of a subject having a stent made of a CDP-taxane conjugate described herein or a strut coated with a CDP-taxane conjugate described herein is reduced by at least 10, 20, 30, 40, 50, 60, 70, 80, 90, 95% or more, compared to the TLR of a subject having a stent made of a different material (e.g., a metal or polymer) or a stent not coated or coated with a polymer and/or agent other than the CDP-taxane conjugate.

Agents

Agents that can be loaded onto a DES include, for example, antiproliferative agents, e.g., anticancer agents (e.g., a taxane (e.g., docetaxel, paclitaxel, larotaxel and cabazitaxel) and an anthracycline (e.g., doxorubicin); pro-endothelial cell agents, anti-restenotic agents; anti-inflammatory agents; statins (e.g., simovastatin); immunosuppresants (e.g., mycophenolic acid); somatostatin receptor agonists (e.g., angiopeptin); and dimethyl sulfoxide.

Exemplary anti-proliferative agents include, e.g., an anticancer agent, e.g., a taxane (e.g., docetaxel, paclitaxel, larotaxel and cabazitaxel) and an anthracycline (e.g., doxorubicin); and an immunosuppressive agent, e.g., a rapamycin analogue (e.g., everolimus, zotarolimus, biolimus), pimecrolimus, or tacrolimus

One or more of the pro-endothelial agents can be loaded on the stents, e.g., to promote, accelerate or increase endothelial healing. Exemplary pro-endothelial agents include, e.g., agents that diminish platelet adhesion and/or fibrinogen binding (e.g., titanium-nitride-oxide or titanium-nitride), agents that capture endothelial progenitor cells (EPCs) (e.g., antibodies (e.g., anti-CD34 antibody) or peptides (e.g., integrin-binding cyclic Arg-Gly-Asp peptide)), or estradiol.

One or more of anti-restenotic agent can also be loaded on or in the stents, e.g., anti-inflammatory agents (e.g., dexamethasone), immunosuppressive agents (e.g., mycophenolic acid), antisense agents (e.g., an advanced six-ring morpholino backbone c-myc antisense (AVI-4126)), inhibitors of vascular smooth muscle cell proliferation and/or tissue factor expression (e.g., 3-hydroxy-3-methylglutaryl coenzyme A (HMG-CoA)-reductase-inhibitors (statins), simvastatin, angiopeptin or dimethyl sulfoxide (DMSO)), or anti-hyperlipidemic agents (e.g., probucol).

In one embodiment, the agent (or agents) is loaded on the luminal side of the stent. In another embodiment, the agent (or agents) is loaded on the abluminal side of the stent. In yet another embodiment, the agent (or agents) is loaded on both the luminal and abluminal sides of the stent. In another embodiment, an agent (or agents) is loaded on the luminal side of the stent and a different agent (or combination of agents) is loaded on the abluminal side of the stent. Thus, different agents (e.g., an anti-proliferation agent and a pro-endothelial agent) can be loaded on different sides (luminal or abluminal) of the stent, e.g., to allow for differential agent elution, or different agents can be loaded on the same side (luminal or abluminal side) of the stent, e.g., to allow for dual local agent elution.

In one embodiment, the agent is present at a concentration of at least about 1, 2, 3, 4, 5, 6, 7, 8, 9, 10, 20, 50, or 100 μg/mm. In one embodiment, more than about 50, 60, 70, 80, 90, 95, 99% of the agent is released over a period of one month. In one embodiment, the release of the agent (e.g., a pro-endothelial agent) is delayed for at least about 1, 2, 3, 4, 5, 6, 7, 8, 9, or 10 days. In one embodiment, the release of the agent sustains for at least 7, 14, 21, 28, 35, or 42 days.

Polymeric Stents

Stents described herein can be made of biocompatible and/or bioabsorbable polymers. A CDP-taxane conjugate described herein can be the stent, the strut of a stent or the CDP-taxane conjugate can coat a strut made of a polymeric material.

An example of a biocompatible stent is the Endeavor Rsolute® stent. This system is composed of three elements: one hydrophobic polymer (‘C10’) to retain the drug and control drug release, another polymer (‘C19’) to provide improved biocompatibility, and finally (on the outer-most side of the stent) a polyvinyl pyrrolidinone (PVP) hydrophilic polymer which increases the initial drug burst and further enhances biocompatibility. Thus, in one embodiment, the CDP-taxane conjugate can be coated on an Endeavor Rsolute® stent. In other embodiments, a CDP-taxane conjugate described herein can replace one or more of the elements of the Endeavor Rsolute® stent.

Bioabsorbable polymers (e.g., inert bioabsorbable polymer) can also be used in a DES, e.g., to reduce prothrombogenic potential and/or allow non-invasive imaging. In some embodiments, the bioabsorbable polymer has a degradation time of at least about 14, 21, 28, 35, 42, 49, 56, 63, 70 days.

Exemplary bioasorbable stents include, e.g., a polymeric stent (e.g., a poly-L-lactide stent, a tyrosine poly(desaminotyrosyl-tyrosine ethyl ester) carbonate stent, and a poly(anhydride ester) salicyclic acid stent). For example, Igaki-Tamai stent is constructed from a poly-L-lactic acid polymer and contains either the tyrosine kinase antagonist ST638 or paclitaxel. REVA® stent is a tyrosine poly(desaminotyrosyl-tyrosine ethyl ester) carbonate stent. It is radio-opaque and has slide and lock mechanism designed to allow for substantial reductions in stent-strut thickness. IDEAL™ stent is a poly(anhydride ester) salicyclic acid stent. Infinnium® stent is composed of two biodegradable polymers with different paclitaxel-release kinetics. Other exemplary bioasorbable stents include, e.g., BVS®, Sahajanand®, Infinnium®, BioMATRIX®, Champion®, and Infinnium®. In one embodiment, a CDP-taxane conjugate described herein can be coated onto any of these bioabsorbable stents. In other embodiments, a CDP-taxane conjugate described herein can replace one or more elements of one of these bioabsorbable stents.

Biosorbable Metallic Stents

The CDP-taxane conjugates described herein can be used to coat a bioabsorbable metallic stent. An exemplary bioabsorbable stent is the Absorbable Metal Stent (AMS®) which is an alloy stent made of 93% magnesium and 7% rare-earth metals.

Reservoir Stents

As described herein, reservoir stents can be used, e.g., to decrease the “thickness” of the stent or reduce the unwanted effect due to microfragmentation of the polymer and/or the agent. For example, the drug can be loaded in one or more reservoirs or wells in the stent, compared to, e.g., more or less uniformly spread over the stent.

In one embodiment, a CDP-taxane conjugate described herein is loaded in the reservoirs or wells located on the stent, e.g., the CDP-taxane conjugate described herein is loaded in the reservoirs or wells located on the luminal side or the abluminal side of the stent. In yet another embodiment, the CDP-taxane conjugate described herein is loaded in the reservoirs or wells located on both the luminal and abluminal sides of the stent.

In one embodiment, different agents (e.g., an anti-proliferation agent and a pro-endothelial agent) can be loaded into the reservoirs or wells on different sides (luminal or abluminal) of the stent, e.g., to allow for differential agent elution. In another embodiment, different agents can be loaded into adjacent reservoirs or wells of the same side (luminal or abluminal side) of the stent, e.g., to allow for dual local drug elution.

Strut

In one embodiment, the strut thickness is at least about 25, 50, 100, 150, 200, 250 μm. In another embodiment, the strut wideness is at least about 0.002, 0.004, 0.006, 0.008, or 0.01 inch. In yet another embodiment, the number of struts is at least about 4, 8, 12, 16, or 18 in its cross-section.

Various shapes of struts such as a zig zag coil, a ratchet log design, circumferential loops, etc. are known in the art and can be employed in the stents described herein.

In one embodiment, the strut can be made of a CDP-taxane conjugate described herein.

Unless otherwise defined, all technical and scientific terms used herein have the same meaning as commonly understood by one of ordinary skill in the art to which this invention belongs. All publications, patent applications, patents, and other references mentioned herein are incorporated by reference in their entirety. In case of conflict, the present specification, including definitions, will control. In addition, the materials, methods, and examples are illustrative only and not intended to be limiting.

### EXAMPLES

### Example 1

### Synthesis 2′-(6-(carbobenzyloxyamino)caproyl)docetaxel

A 500-mL round-bottom flask equipped with a magnetic stirrer was charged with 6-(carbobenzyloxyamino) caproic acid (4.13 g, 15.5 mmol), docetaxel (12.0 g, 14.8 mmol), and dichloromethane (240 mL). The mixture was stirred for 5 min to produce a clear solution, to which 1-ethyl-3-(3-dimethyllaminopropyl)carbodiimide hydrochloride (EDC.HCl) (3.40 g, 17.6 mmol) and 4 dimethylaminopyridine (DMAP) (2.15 g, 17.6 mmol) were added. The mixture was stirred at ambient temperature for 3 h at which time, IPC analysis showed a 57% conversion along with 34% residual docetaxel. An additional 0.2 equivalents of EDC.HCl and DMAP were added and the reaction was stirred for 3 h, at which time IPC analysis showed 63% conversion. An additional 0.1 equivalents of 6-(carbobenzyloxyamino) caproic acid along with 0.2 equivalents of EDC.HCl and DMAP were added. The reaction was stirred for 12 h and IPC analysis indicated 74% conversion and 12% residual docetaxel. To further increase the conversion, an additional 0.1 equivalents of 6-(carbobenzyloxyamino) caproic acid and 0.2 equivalents of EDC.HCl and DMAP were added. The reaction was continued for another 3 h at which time, IPC analysis revealed 82% conversion and the residual docetaxel dropped to 3%. The reaction was diluted with DCM (200 mL) and washed with 0.01% HCl (2×150 mL) and brine (150 mL). The organic layer was separated, dried over sodium sulfate, and filtered. The filtrate was concentrated to a residue and dissolved in ethyl acetate (25 mL). The solution was divided into two portions, each of which was passed through a 120-g silica column (Biotage F40). The flow rate was adjusted to 20 mL/min and 2000 mL of 55:45 ethyl acetate/heptanes was consumed for each of the column purifications. The fractions containing minor impurities were combined, concentrated, and passed through a column a third time. The fractions containing product (shown as a single spot by TLC analysis) from all three column purifications were combined, concentrated to a residue, vacuum-dried at ambient temperature for 16 h to afford the product, 2′-(6-(carbobenzyloxyamino) caproyl) docetaxel as a white powder [10 g, yield: 64%]. The ¹H NMR analysis was consistent with the assigned structure of the desired product; however, HPLC analysis (AUC, 227 nm) indicated only a 97% purity along with 3% of bis-adducts. To purify the 2′-(6-(carbobenzyloxyamino) caproyl) docetaxel product, ethyl acetate (20 mL) was added to dissolve the batch to produce a clear solution. The solution was divided into two portions, each of which was passed through a 120-g silica column. The fractions containing product were combined, concentrated to a residue, vacuum-dried at ambient temperature for 16 h to afford the desired product (2′-(6-(carbobenzyloxyamino) caproyl) docetaxel) as a white powder [8.6 g, recovery yield: 86%]. HPLC analysis (AUC, 227 nm) indicated >99% purity.

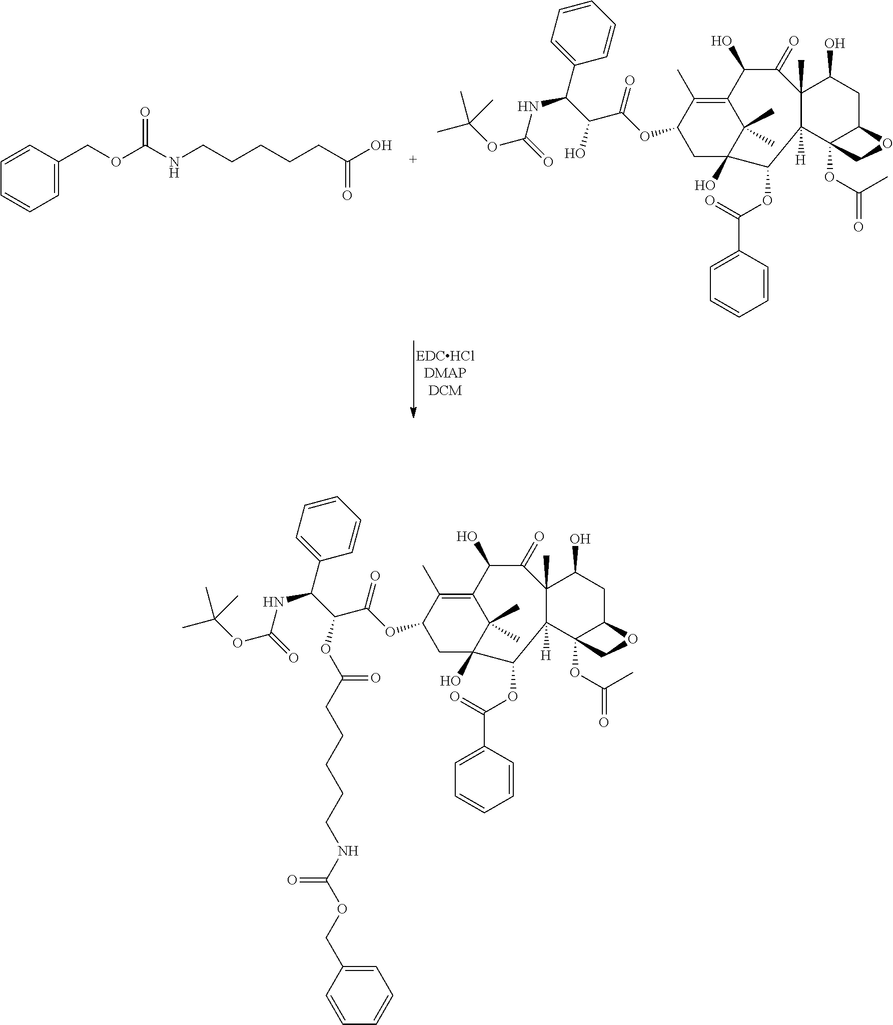

### Example 2

### Synthesis of 2′-(6-amino caproyl) docetaxel.MeSO₃H

A 1000-mL round-bottom flask equipped with a magnetic stirrer was charged with 2′-(6-(carbobenzyloxyamino) caproyl) docetaxel product [5.3 g, 5.02 mmol] and THF (250 mL). To the resultant clear solution, MeOH (2.5 mL) and 5% Pd/C (1.8 g, 10 mol % of Pd) were added. The mixture was cooled to 0° C. and methanesulfonic acid (316 μL, 4.79 mmol) was added. The flask was evacuated for 10 seconds and filled with hydrogen using a balloon. After 3 h, IPC analysis indicated 62% conversion. The ice-bath was removed and the reaction was allowed to warm up to ambient temperature. After an additional 3 h, IPC analysis indicated that the reaction was complete. The solution was filtered through a Celite® pad and the filtrate was black in appearance. To remove the possible residual Pd, charcoal (5 g, Darco®) was added and the mixture was placed in a fridge overnight and filtered through a Celite® pad to produce a clear colorless solution. This was concentrated at <20° C. under reduced pressure to a volume of ˜100 mL, to which methyl tert-butyl ether (MTBE) (100 mL) was added. The resultant solution was added to a solution of cold MTBE (1500 mL) with vigorous stirring over 0.5 h. The suspension was left at ambient temperature for 16 h, the upper clear supernatant was decanted off and the bottom layer was filtered through a 0.45 μm filter membrane. The filter cake was vacuum-dried at ambient temperature for 16 h to afford the desired product 2′-(6-amino caproyl) docetaxel.MeSO₃H as a white solid [4.2 g, yield: 82%]. HPLC analysis indicated >99% purity and the ¹H NMR analysis indicated the desired product.

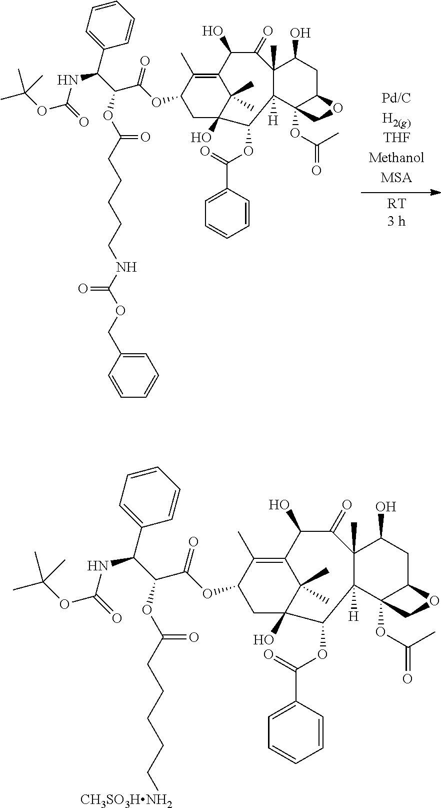

### Example 3

### Synthesis of CDP-hexanoate-docetaxel

CDP (4.9 g, 1.0 mmol) was dissolved in dry N,N-dimethylformamide (DMF, 49 mL) 2′-(6-aminohexanoyl) docetaxel MeSO₃H (2.0 g, 2.2 mmol), N,N-Diisopropylethylamine (290 mg, 2.2 mmol), N-(3-Dimethylaminopropyl)-N′-ethylcarbodiimide hydrochloride (580 mg, 3.0 mmol), and N-Hydroxysuccinimide (250 mg, 2.2 mmol) were added to the polymer solution and stirred for 4 h. The polymer was precipitated with acetone (500 mL). It was then rinsed with acetone (100 mL). The product contained CD-hexanoate-docetaxel and could contain free CDP and traces of free docetaxel.

The CDP hexanoate-docetaxel was dissolved in water (490 mL). The solution was dialyzed using a tangential flow filtration system (30 kDa MW cutoff, membrane area=50 cm²). It was then concentrated to 20 mg of CDP-hexanoate-docetaxel/mL. It was then formulated with mannitol and filtered through 0.2 μm filters (Nalgene) and lyophilized to yield white solid.

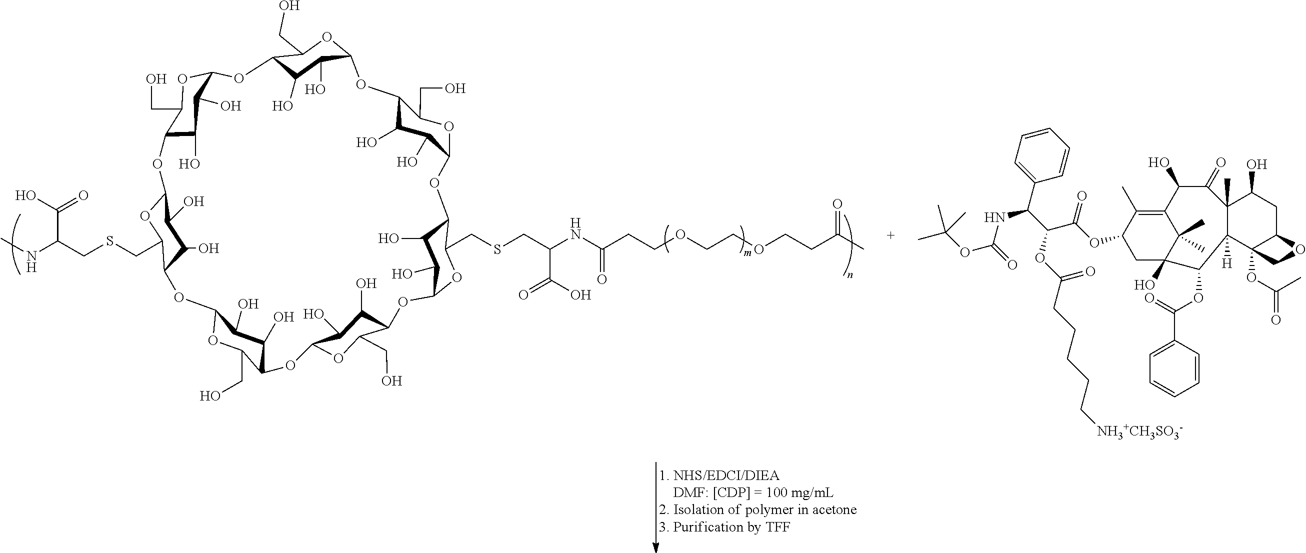

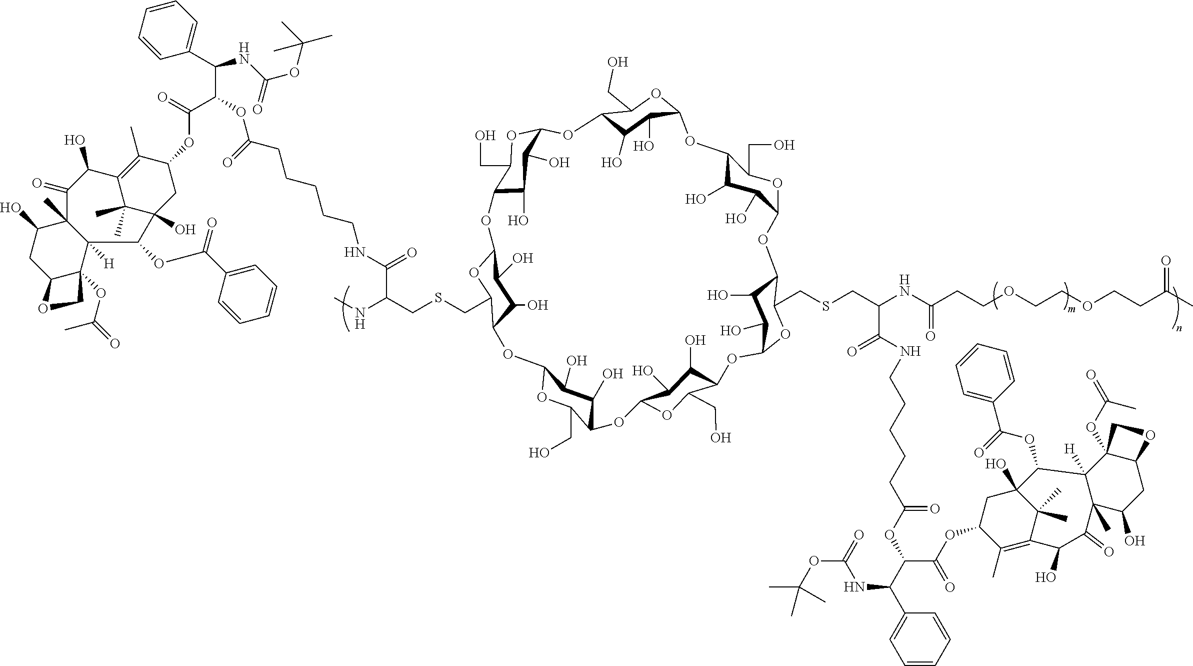

### Example 4

### Formulation of CDP-Hexanoate-Docetaxel Nanoparticles

CDP-hexanoate-docetaxel (100 mg) as prepared in example 3 above was dissolved in water (10 mL). Particle solution properties were characterized by dynamic light scattering (DLS) spectrometer.

Particle properties, evaluated by using the resulting plurality of particles made in the method above:
Zavg=47.0 nm
Particle PDI=0.587
Dv50=11.2 nm
Dv90=18.2 nm

### Example 5

### Synthesis of 2-(2-(pyridin-2-yl)disulfanyl)ethylamine

In a 25 mL round bottom flask, 2,2′-dithiodipyridine (2.0 g, 9.1 mmol) was dissolved in methanol (8 mL) with acetic acid (0.3 mL). Cysteamine hydrochloride (520 mg, 4.5 mmol) was dissolved in methanol (5 mL) and added dropwise into the mixture over 30 minutes. The mixture was then stirred overnight. It was then reduced under vacuum to yield a yellow oil. The oil was dissolved in methanol (5 mL) and then precipitated into diethyl ether (100 mL). The precipitate was filtered off and dried. It was then redissolved in methanol (5 mL) and reprecipitated in diethyl ether (100 mL). This procedure was repeated twice. The pale yellow solid was filtered off and dried to produce the final product, 2-(2-(pyridin-2-yl)disulfanyl)ethylamine (0.74 g, 74% yield) which was used without further purification.

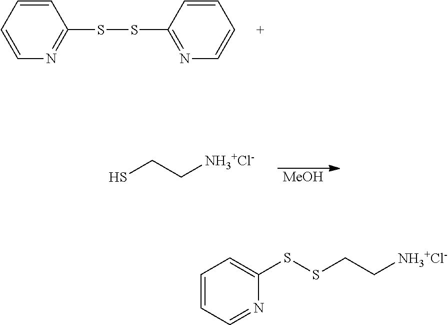

### Example 6

### Synthesis of 2-(2-(pyridin-2-yl)disulfanyl)ethanol

In a 50 mL round bottom flask, 2,2′-dithiodipyridine (0.50 g, 2.3 mmol) was dissolved in dichloromethane (5 mL) 2-Mercaptoethanol (90 mg, 1.1 mmol) was dissolved in dichloromethane (5 mL) and added to the mixture dropwise over 30 minutes. The mixture was stirred for an additional 30 minutes. It was then concentrated under vacuum to yield a yellow oil (200 mg, 91%). The oil was then used without further purification.

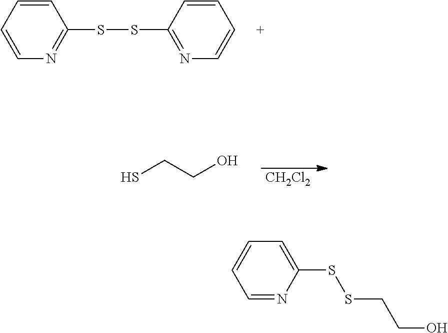

### Example 7

### Synthesis of 2-(2-(Pyridin-2-yl)disulfanyl)ethanol (Alternate Route)

In a 250 mL round bottom flask, methoxycarbonylsulfenyl chloride (7.0 g, 55 mmol) was dissolved in dichloromethane (50 mL) and stirred in ice bath. To the mixture, 2-mercaptoethanol (4.5 g, 55 mmol) was added dropwise over 30 minutes. 2-Mercaptopyridine (6.1 g, 55 mmol) was dissolved in dichloromethane (80 mL) and it was added dropwise to the mixture over 1 h in an ice bath. It was then brought to room temperature and stirred for one additional hour. The mixture was concentrated down to approximately. 60 mL of dichloromethane in which a precipitate started to form. The precipitate was filtered off and washed with dichloromethane (25 mL) twice. It was then dried under vacuum to produce a yellow solid (9.6 g, 78% yield).

In a 50 mL round bottom flask, the crude yellow solid (2.5 g, 11 mmol) and 4-(dimethylamino)pyridine (1.4 g, 11 mmol) was dissolved in dichloromethane (20 mL). It was then purified by flash columnchromatography (dichloromethane:acetone=15:1) to produce a yellow oil (1.9 g, 90% yield).

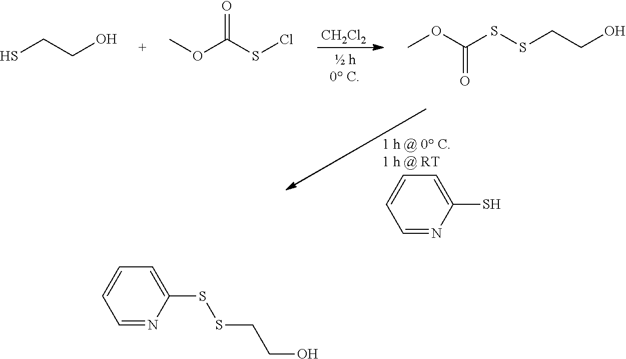

### Example 8

### Synthesis of 4-nitrophenyl 2-(2-(Pyridin-2-yl)disulfanyl)ethyl carbonate

In a 250 mL round bottom flask, 4-nitrophenyl chloroformate (2.0 g, 10 mmol) was dissolved in dichloromethane (20 mL) 2-(2-(Pyridin-2-yl)disulfanyl)ethanol (1.9 g, 10 mmol) and N,N-diisopropylethylamine (1.0 g, 10 mmol) were dissolved in dichloromethane (100 mL) and added dropwise to the mixture and stirred overnight. The solution was then pumped down to dryness to yield a yellow oil. The crude product was purified by flash column chromatography (dichloromethane:acetone=30:1) to produce a yellow oil (2.9 g, 81% yield).

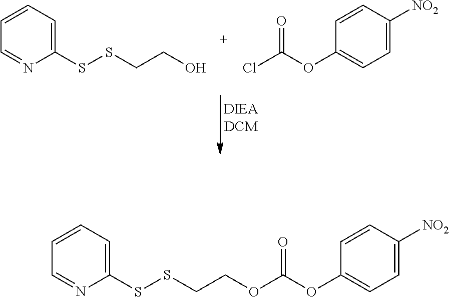 [SMILES: OCCSSc1ccccn1 ; O=C(Cl)Oc1ccc([N+](=O)[O-])cc1 ; O=C(OCCSSc1ccccn1)Oc1ccc([N+](=O)[O-])cc1]

### Example 9

### Synthesis of 2′-(2-(2-(Pyridin-2-yl)disulfanyl)ethylcarbonate) Docetaxel

In a 50 mL round bottom flask, 4-nitrophenyl 2-(2-(pyridin-2-yl)disulfanyl)ethyl carbonate (200 mg, 0.56 mmol), docetaxel (500 mg, 0.62 mmol) and 4-(dimethylamino)pyridine (140 mg, 1.1 mmol) were dissolved in dichloromethane (50 mL) and stirred overnight. It was washed with 0.1N hydrochloric acid (10 mL) twice, dried over magnesium sulfate, and pumped down to yield a white solid. It was then purified by column chromatography (dichloromethane:methanol=15:1) to yield a light yellow solid (210 mg, 36% yield).

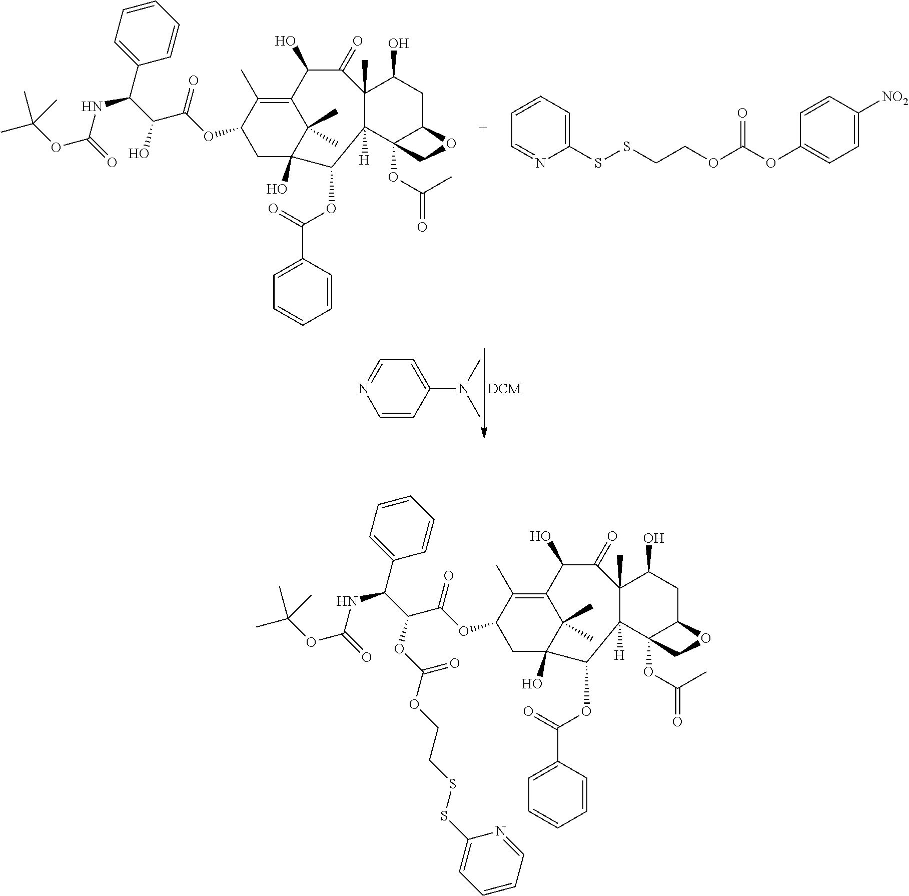

### Example 10

### Synthesis of CDP-NHEtSSPyridine

In a 25 mL round bottom flask, CDP(CDP, 0.50 g, 0.10 mmol) was dissolved in N,N-dimethylformamide (5 mL). To the solution, the following was added: 2-(2-(pyridin-2-yl)disulfanyl)ethylamine (51 mg, 0.23 mmol), N-hydroxysuccinimide (26 mg, 0.23 mmol), N-(3-dimethylaminopropyl)-N′-ethylcarbodiimide hydrochloride (60 mg, 0.31 mmol) and N,N-diisopropylethylamine (29 mg, 0.23 mmol). The mixture was stirred for 4 h. Isopropanol (10 mL) was added followed by diethyl ether (50 mL) to precipitate out the polymer. The polymer was then rinsed with acetone (20 mL) and dissolved in water (50 mL) The product was purified by dialysis against water by using dialysis tube membrane (25k MWCO) for 24 h. It was then filtered through a 0.2 μm filter and lyophilized to yield a white solid polymer (360 mg, 72% yield).

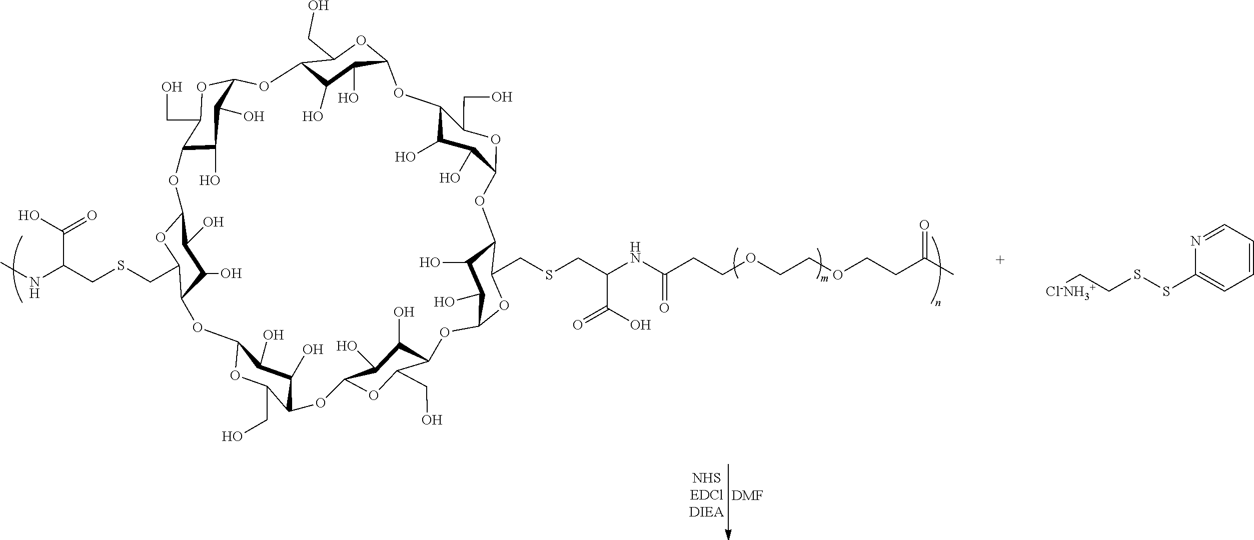

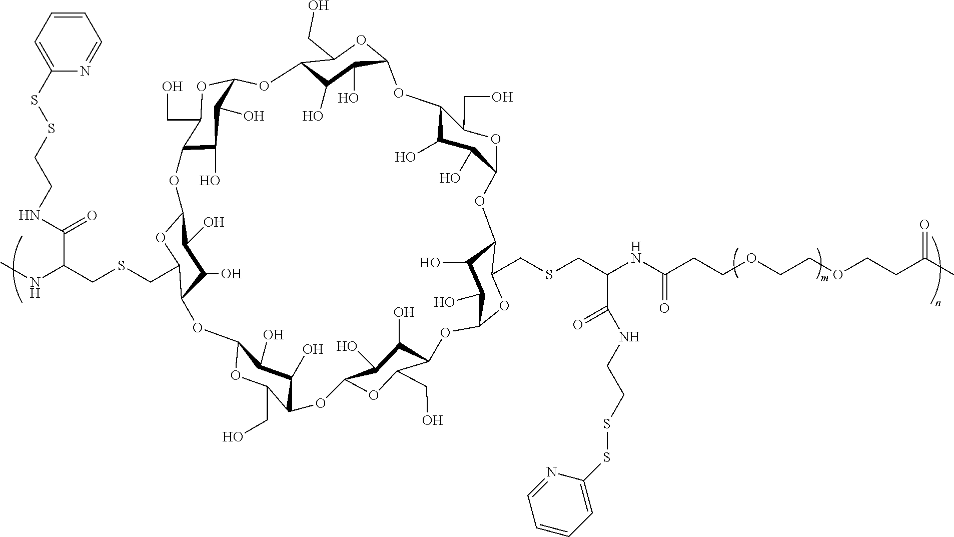

### Example 11

### Synthesis of CDP-NHEtSH

In a 10 mL round bottom flask, CDP-NHEtSSPyridine (120 mg, 0.023 mmol) was dissolved in methanol (2 mL) D,L-Dithiothreitol (36 mg, 0.23 mmol) was added to the mixture and stirred at room temperature for 1 h. The polymer was then precipitated out in diethyl ether (20 mL). It was then dried under vacuum for 2 min. The polymer was then redissolved in methanol (2 mL) and precipitated out in diethyl ether (20 mL). This reprecipitation procedure was repeated once more. It was then dried under vacuum for 1 h to yield a white solid (88 mg, 73% yield).

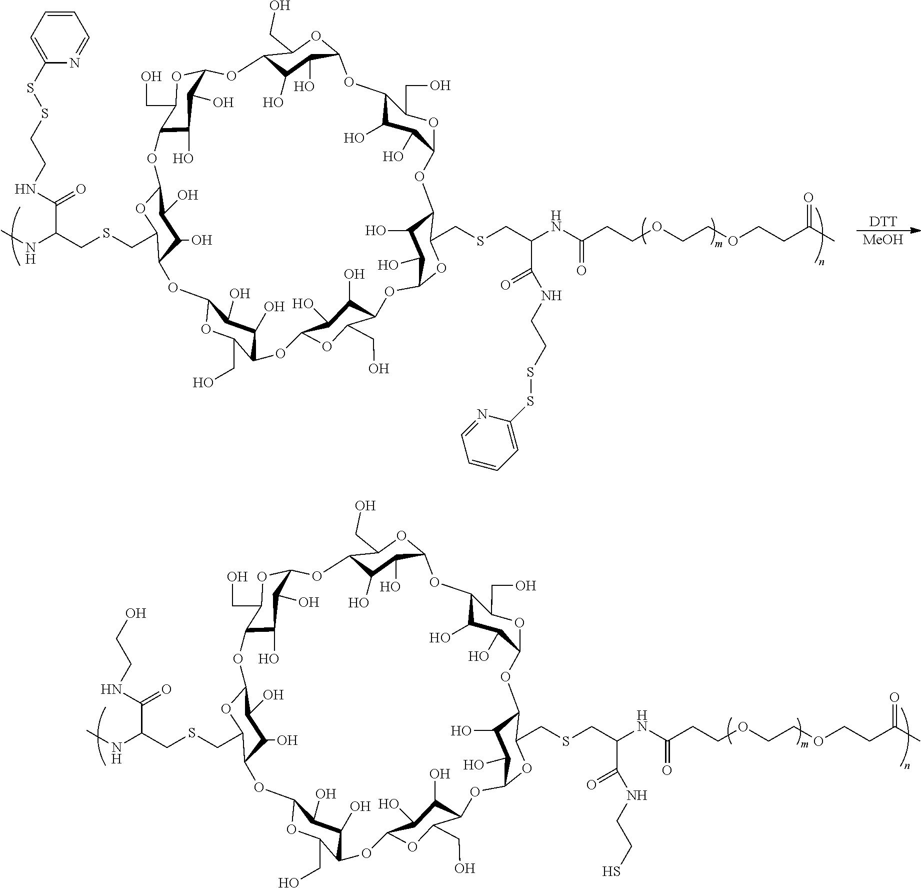

### Example 12

### Synthesis of CDP-NHEtSSEtOCO-2′-O-docetaxel

In a 10 mL round bottom flask, CDP-NHEtSH (88 mg, 0.018 mmol) was dissolved in methanol (1.8 mL). The solution was then mixed with 2′-(2-(2-(pyridin-2-yl)disulfanyl)ethylcarbonate) docetaxel (32 mg, 0.031 mmol) and stirred at room temperature for 1 h. N-Ethylmaleimide (4.4 mg, 0.035 mmol) was added to the mixture and stirred for an additional hour. The polymer was then precipitated out in diethyl ether (20 mL). It was then rinsed with acetone (10 mL). The polymer was dissolved in water (9 mL) and then purified by dialysis against water by using dialysis tube membrane (25k MWCO) for 24 h. It was then filtered through 0.2 μm and lyophilized to yield a white solid polymer (CDP-NHEtSSEtOCO-2′-O-docetaxel). The product could also contain free CDP and some traces of free docetaxel.

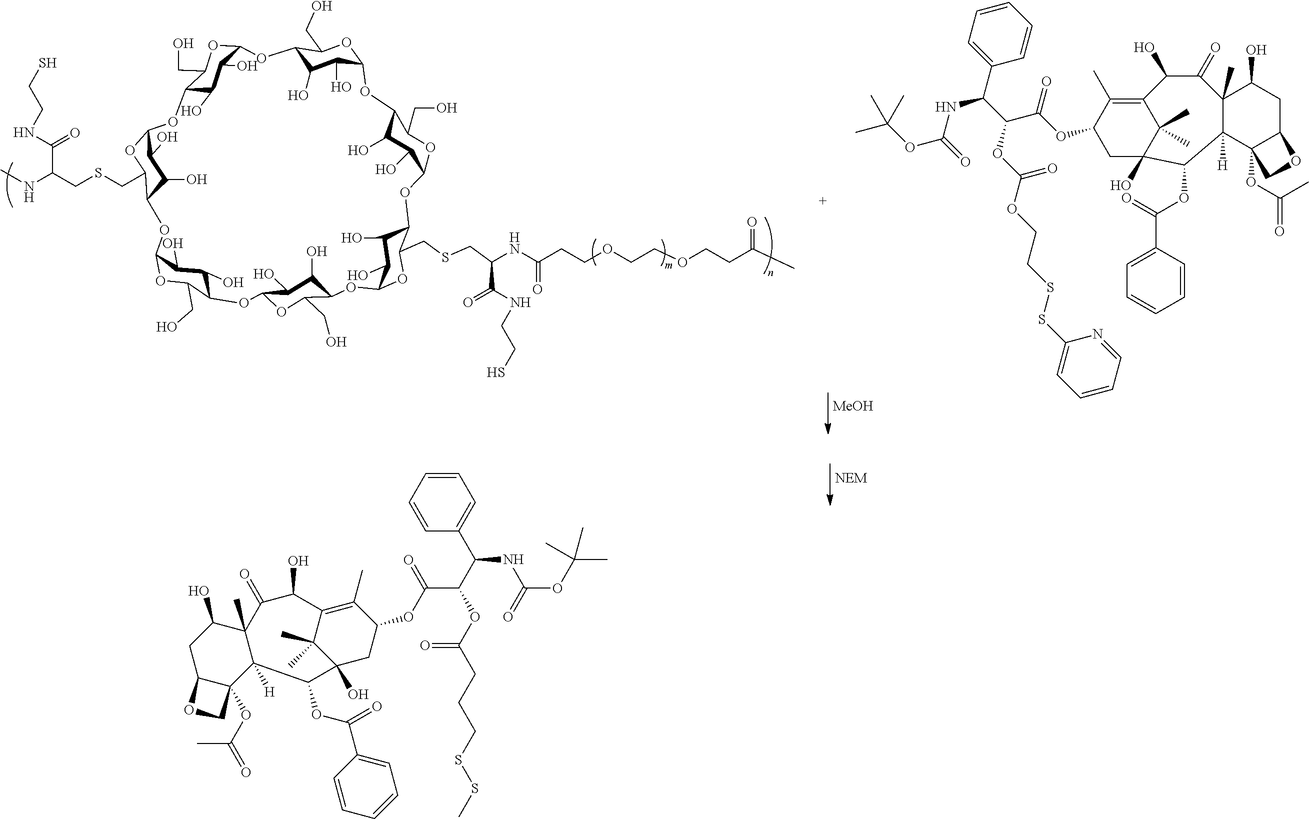

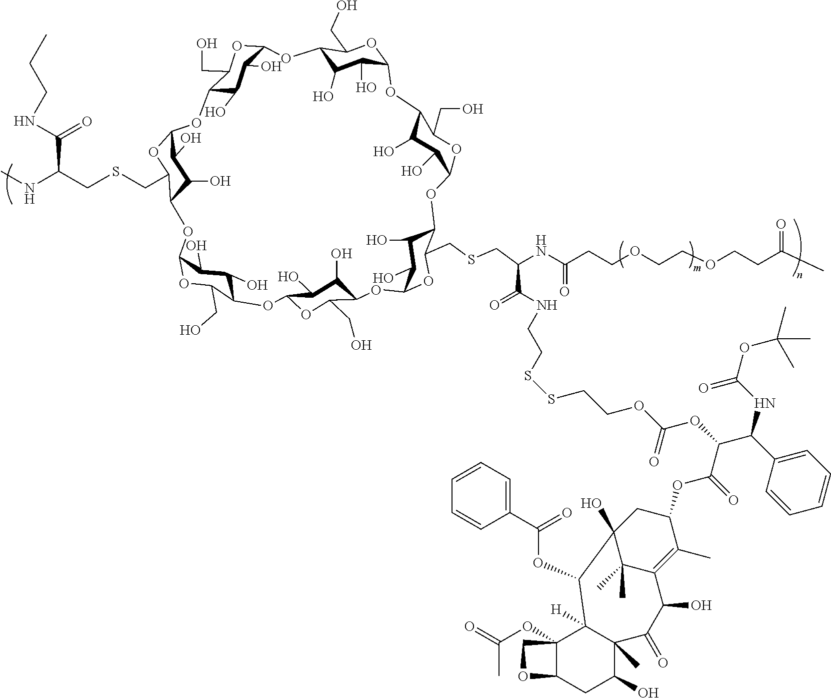

### Example 13

### Formulation of CDP-NHEtSSEtOCO-2′-O-docetaxel nanoparticles

CDP-NHEtSSEtOCO-2′-O-docetaxel (100 mg) as prepared in example 12 above was dissolved in water (10 mL). Particle solution properties were characterized by dynamic light scattering (DLS) spectrometer.

Particle properties, evaluated by using the resulting plurality of particles made in the method above:
Zavg=16.4 nm
Particle PDI=0.507
Dv50=4.41 nm
Dv90=8.30 nm

### Example 14

### Synthesis of Docetaxel Aminoethyldithioethyl Carbonate

Triethylamine (15.0 mL, 108 mmol) was added to a mixture of cystamine.2HCl (5.00 g, 22.2 mmol) and MMTCl (14.1 g, 45.6 mmol, 2.05 equiv) in CH₂Cl₂ (200 mL) at ambient temperature. The mixture was stirred for 90 h and 200 mL of 25% saturated NaHCO₃ was added, stirred for 30 min, and removed. The mixture was washed with brine (200 mL) and concentrated to produce a brown oil (19.1 g). The oil was dissolved in 20-25 mL CH₂Cl₂ and purified by flash chromatography to yield a white foam (diMMT-cyteamine, 12.2 g, 79% yield)

Bis(2-hydroxyethyldisulfide) (11.5 mL, 94 mmol, 5.4 equiv) and 2-mercaptoethanol (1.25 mL, 17.8 mmol, 1.02 equiv) were added to a solution of diMMT-cyteamine (12.2 g, 17.5 mmol) in 1:1 CH₂Cl₂/MeOH (60 mL) and the mixture was stirred at ambient temperature for 42.5 h. The mixture was concentrated to an oil, dissolved in EtOAc (150 mL), washed with 10% saturated NaHCO3 (3×150 mL) and brine (150 mL), dried over Na2SO4, and concentrated to an oil (16.4 g). The oil was dissolved in 20 mL CH₂Cl₂ and purified by flash chromatography to yield clear thick oil (MMT-aminoethyldithioethanol, 5.33 g, 36% yield).

A 250 mL round bottom flask equipped with a magnetic stirrer was charged with MMT-aminoethyldithioethanol (3.6 g, 8.5 mmol) and acetonitrile (60 mL). Disuccinimidyl carbonate (2.6 g) was added and the reaction was stirred at ambient temperature for 3 h. It was used for the next reaction without isolation. Succinimidyl MMT-aminoethyldithioethyl carbonate was transferred to a cooled solution of docetaxel (6.14 g, 7.61 mmol) and DMAP (1.03 g) in DCM (60 mL) at 0-5° C. with stirring for 16 h. It was then purified by column chromatography.

A 1000 mL round bottom flask equipped with a magnetic stirrer was charged with docetaxel Cbz-aminoethyldithioethyl carbonate (12.6 g) and DCM (300 mL). Anisole (10.9 mL, 10 equiv.) was added to this clear solution and stirred for a few minutes. Dichloroacetic acid (8.3 mL, 10 equiv.) was added over 5 min and the reaction was stirred at ambient temperature for 1 h. The mixture was concentrated down to ˜100 mL, to which heptanes (800 mL) was slowly added resulting in a suspension. The suspension was stirred for 15 min and the supernatant was decanted. The orange residue was washed with heptanes (200 mL) and vacuum-dried at ambient temperature for 1 h. THF (30 mL) was added to dissolve the orange residue producing a red solution. Heptanes (500 mL) was slowly added to precipitate out the product. The resulting suspension was stirred at ambient temperature for 1 h and filtered. The filter cake was washed with heptanes (300 mL) and dried under vacuum to yield docetaxel aminoethyldithioethyl carbonate.

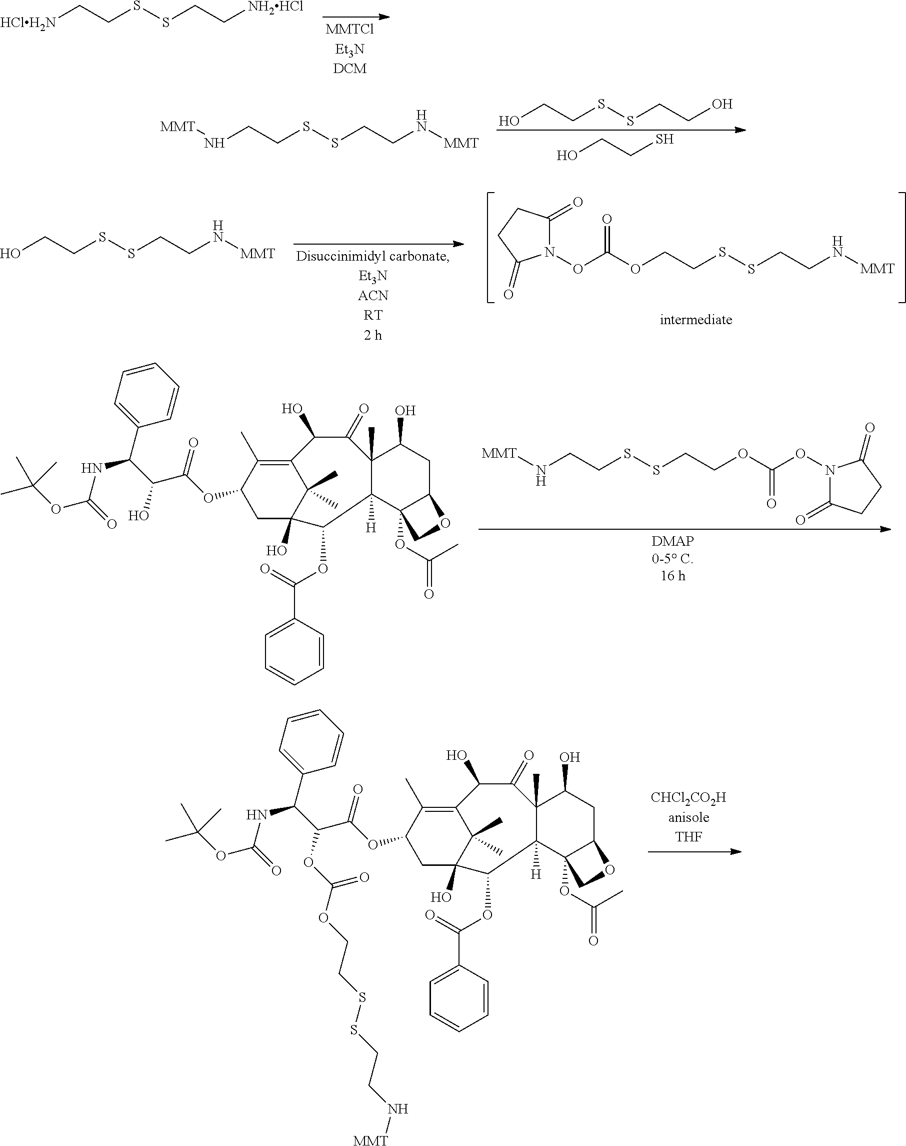

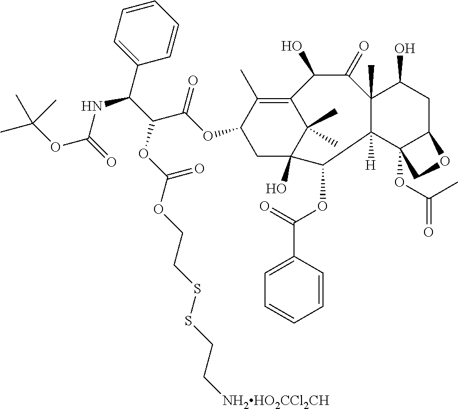

### Example 15

### Synthesis of CDP-NHEtSSEtOCO-2′-O-docetaxel

CDP (1.5 g, 0.31 mmol) was dissolved in dry N,N-dimethylformamide (DMF, 15 mL). Docetaxel aminoethyldithioethyl carbonate (760 mg, 0.68 mmol), N,N-Diisopropylethylamine (88 mg, 0.68 mmol), N-(3-Dimethylaminopropyl)-N′-ethylcarbodiimide hydrochloride (130 mg, 0.68 mmol), and N-Hydroxysuccinimide (79 mg, 0.68 mmol) were added to the polymer solution and stirred for 2 h. The polymer was precipitated with isopropanol (225 mL) and then rinsed with acetone (150 mL). The precipitate was dissolved in nanopure water (150 mL). It was purified by TFF with nanopure water (1.5 L). It was filtered through 0.2 μm filter and kept frozen.

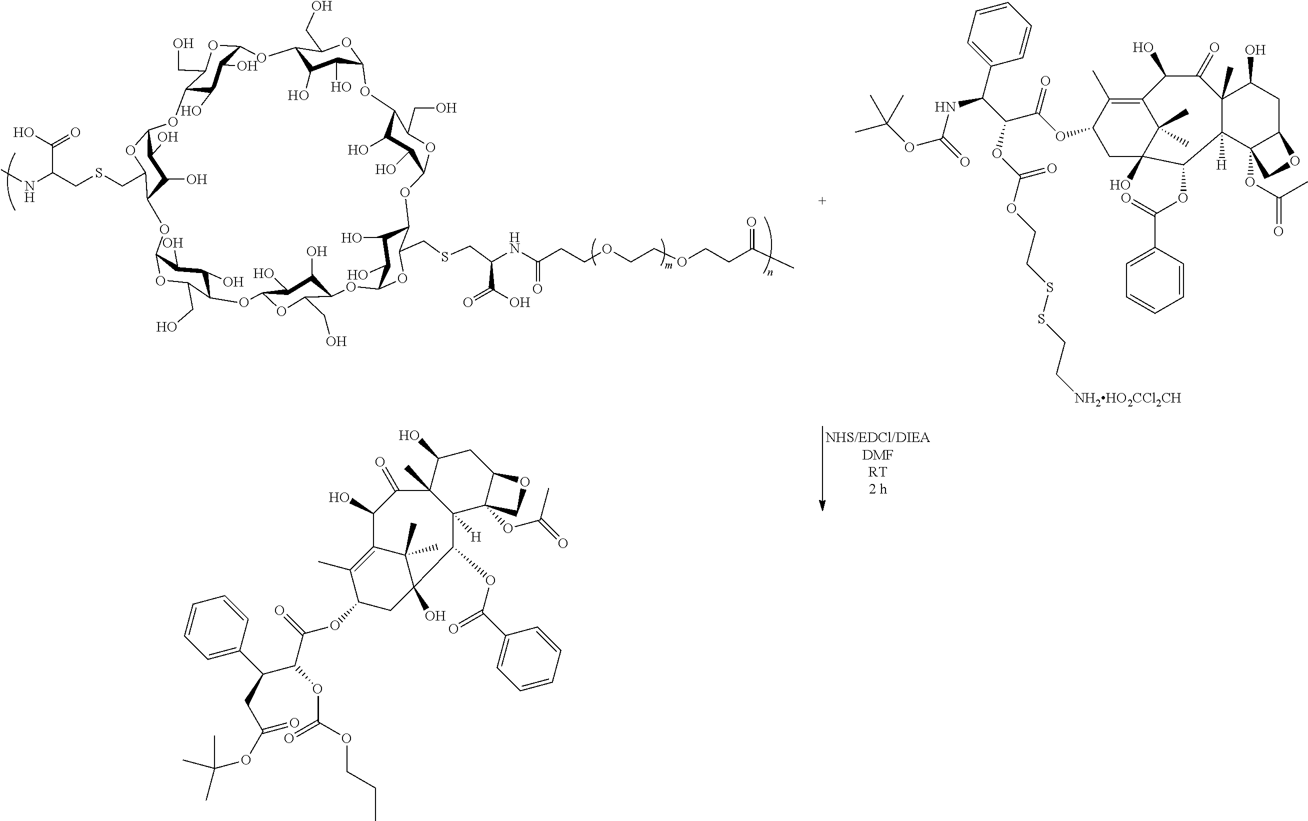

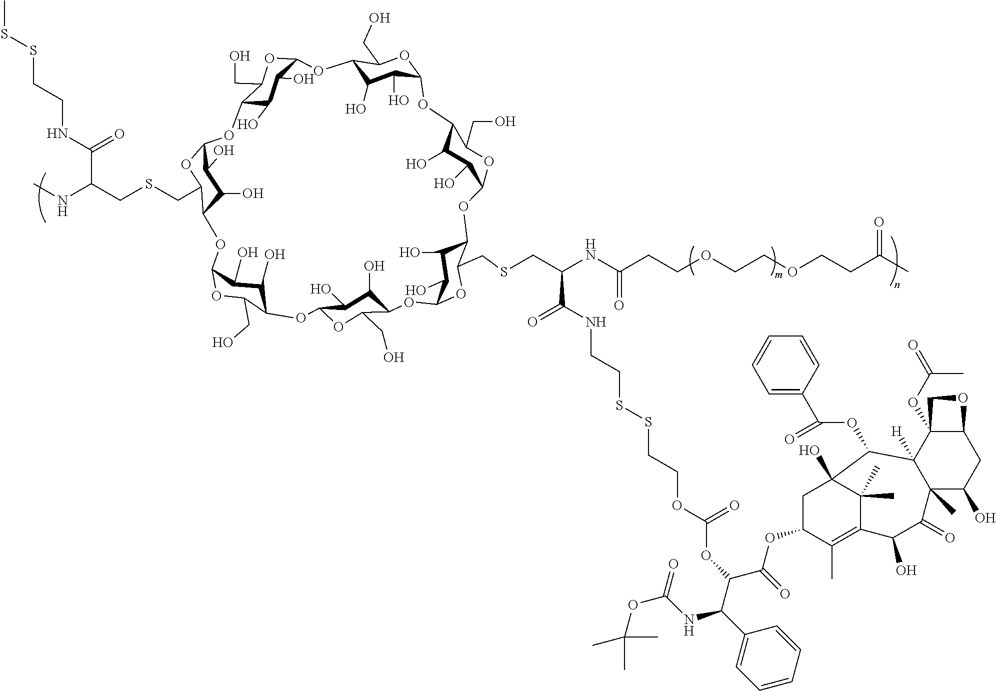

### Example 16

### Formulation of CDP-NHEtSSEtOCO-2′-O-docetaxel Nanoparticles

CDP-NHEtSSEtOCO-2′-O-docetaxel as prepared in Example 15 above (1 mg) was dissolved in water (1 mL). Particle solution properties were characterized by dynamic light scattering (DLS) spectrometer.

Particle properties, evaluated by using the resulting plurality of particles made in the method above:
Zavg=26.67 nm
Particle PDI=0.486
Dv50=8.55 nm
Dv90=14.6 nm

### Example 17

### Synthesis of docetaxel-2′-glycine bsmoc

A 50 ml round-bottom flask was charged with a solution of docetaxel (1 g, 1.23 mmol), BsmocGlycine (0.4184 g, 1.4 mmol) and 4-dimethylaminopyridine (0.0487 g, 0.398 mmol) in anhydrous methylene chloride (20 mL) under nitrogen. The solution was cooled to 10° C. and EDC.HCl (0.3589 g, 1.87 mmol) was added to the solution, while stiffing. The reaction was stirred for 1 h at 10° C., resulting in a clear solution. The reaction was stirred for an additional hour at ambient temperature. TLC analysis in CHCl₃ and MeOH (14:1) showed a presence of small amount of unreacted docetaxel. The reaction was continued to stir for another 30 minutes and then washed with 0.1 M hydrochloric acid (2×200 mL) and water (200 mL). The organic layer was dried over anhydrous magnesium sulfate and filtered. The organic solvent was then evaporated under reduced pressure to give a white powder (1.38 g). HPLC and LC/MS analysis of the final product showed a mixture of compounds—docetaxel, docetaxel-2′-glycine Bsmoc, docetaxel-7-glycine Bsmoc, docetaxel-2′,7-bis(glycine Bsmoc) and another bis(Glycine Bsmoc) derivative of docetaxel. The crude product was separated by silica gel column chromatography. The products were eluted with CHCl₃/MeOH and with increasing MeOH concentration from 2% (200 ml) to 3% (600 ml). The TLC was monitored in CHCl₃ and MeOH (14:1). The fractions containing docetaxel-2′-Glycine Bsmoc were collected and concentrated to provide 93% pure product with docetaxel-7-glycine Bsmoc as an impurity. ¹H NMR and LC/MS analysis confirmed the desired product.

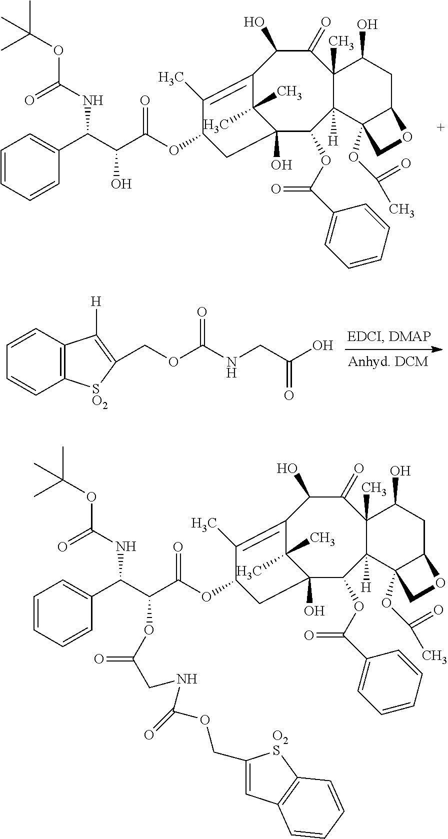

### Example 18

### Synthesis and Formulation of CDP-Glycine-Docetaxel Nanoparticles

To a solution of docetaxel-2′-glycine Bsmoc (0.052 g, 0.0478 mmol) in anhydrous DMF (2 mL), 4-piperidinopiperidine (0.008 g, 0.0478 mmol) was added and the reaction mixture was stirred at ambient temperature. 4-piperidinopiperidine was dried under vacuum before use. The TLC was monitored CHCl₃ and MeOH (14:1) and after ˜2 h of stiffing, no starting material was observed. A mass of 0.106 g (0.0217 mmol) of CDP polymer was then added to the reaction mixture and stirring was continued until the polymer dissolved, i.e., for approx. 15 min. The reagents EDC.HCl (0.0126 g, 0.0651 mmol) and NHS (0.0059 g, 0.0477 mmol) were added followed by the addition of DIEA (0.0062 g, 0.0477 mmol) and the stirring was continued for another 4 h. The polymer was precipitated in 5 volumes of acetone (10 ml), which resulted in a turbid solution. The acetone-DMF solution was then transferred into 5 volumes of diethyl ether (˜60 ml). The polymer precipitated together as a lump Diethyl ether was then decanted and the precipitated polymer product was washed with acetone. The product could contain some amounts of free CDP and trace amounts of drug present.

After decanting the acetone, the polymer was dissolved in 10 ml of water to make ˜10 mg/mL polymer solution. The solution was then dialyzed against 4 L water using 25 kDa MWCO dialysis tube. The sample was dialyzed for 72 h and the water was changed once on the third day. A small amount of precipitate was observed in the dialysis bag. The solution, ˜13 mL volume, was filtered through a 0.22 μm filter. The filtered solution was then analyzed for size by dynamic light scattering (DLS) spectrometer.

Particle properties, evaluated by using the resulting plurality of particles made in the method above:
Zavg=55.11 nm
Particle PDI=0.706
Dv50=13.2 nm
Dv90=23.9 nm

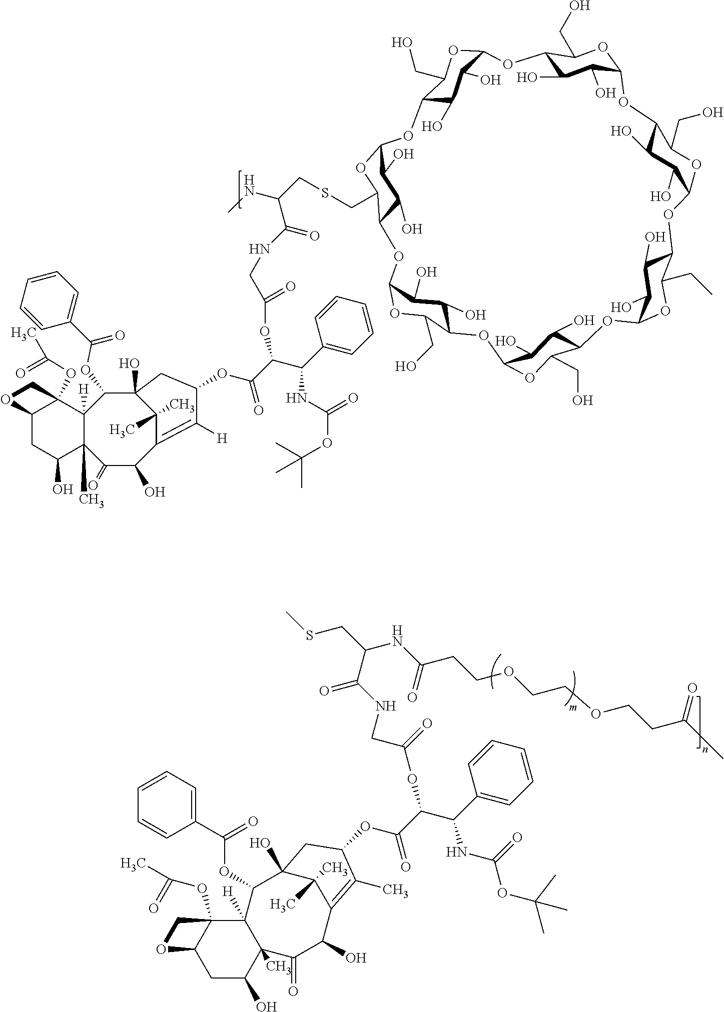

### Example 19

### Synthesis of Docetaxel-2′-Glycinate.Methanesulfonic Acid

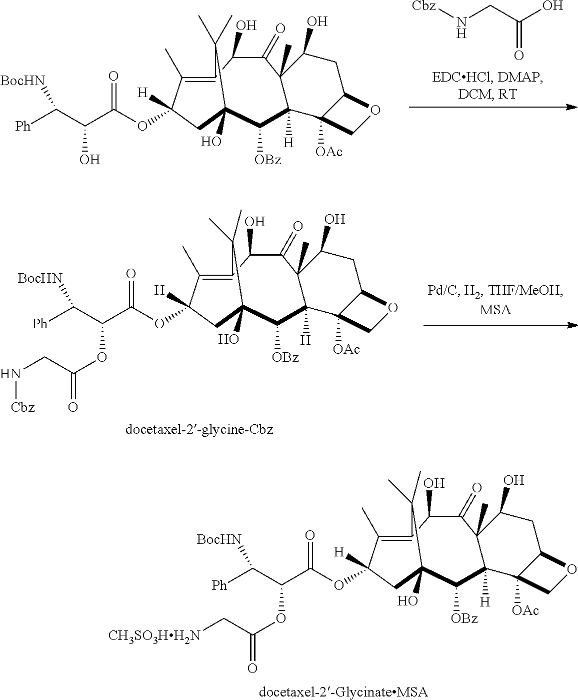

Docetaxel (15.0 g, 18.6 mmol) and dichloromethane (CH₂Cl₂, 300 mL) were added to a 1 litre round bottom flask and the mixture was stirred for 5 min using an overhead stirrer. N-Carbobenzyloxy-glycine (N-Cbz-glycine, 2.92 g, 13.9 mmol, 0.75 equiv), 4-(dimethylamino)pyridine (DMAP, 1.82 g, 15.0 mmol, 0.80 equiv) and N-(3-dimethylaminopropyl)-N′-ethylcarbodiimide hydrochloride (EDC.HCl, 2.87 g, 14.9 mmol, 0.80 equiv) were then added. The mixture was stirred at ambient temperature for 3 h and an additional amount of N-Cbz-glycine (1.57 g, 7.5 mmol, 0.40 equiv), DMAP (1.04 g, 8.5 mmol, 0.46 equiv), and EDC.HCl (1.62 g, 8.4 mol, 0.45 equiv) were added. After stirring the mixture for an additional 2.75 h, it was washed twice with 0.5% HCl (2×150 mL) and brine (150 mL). The organics were dried over sodium sulfate, and the supernatant was concentrated to a residue (21.6 g). The residue was dissolved in 60 mL of chloroform and purified by flash chromatography to produce docetaxel-2′-glycine-Cbz [12.3 g, 66% yield, 98.5%] as a white solid.

In a 1 litre round bottom flask, 5% palladium on activated carbon (Pd/C, 4.13 g) was slurried in a mixture of tetrahydrofuran (THF, 60 mL), methanol (MeOH, 12.5 mL), and methanesulfonic acid (MSA, 0.75 mL, 11.5 mmol, 0.93 equiv). The mixture was stirred under hydrogen (balloon pressure) at ambient temperature for 1 h. A solution of docetaxel-2′-glycine-Cbz (12.3 g, 12.3 mmol) in THF (60 mL) was added with an additional 60 mL THF wash. The mixture was stirred for 2.5 h, then the hydrogen was removed and the mixture was filtered using a 40 mL THF wash. The filtrate was concentrated and then diluted to about 80 mL with THF. Heptanes (700 mL) were then added drop wise over 20 min. The resulting slurry was filtered using a 150 mL heptanes wash and dried under vacuum to produce docetaxel-2′-glycinate.MSA as a white solid [11.05 g, 94%, 95.8% AUC by HPLC].

### Example 20

### Synthesis and Formulation of CDP-Glycine-Docetaxel Nanoparticles

CDP polymer (1 g, 0.207 mmol) was dissolved in anhydrous dimethylformamide

(DMF, 10 mL) and stirred for 30 min to dissolve the polymer. Docetaxel-2′-glycinate.methanesulfonic acid (0.430 g, 0.455 mmol), 1-ethyl-3-(3-dimethylaminopropyl) carbodiimide (EDCI, 0.0597 g, 0.311 mmol) and N-Hydroxysuccinimide (NHS, 0.0263 g, 0.228 mmol) was added to the polymer solution. While stirring, N,N-diisopropylethylamine (DIEA, 0.0294 g, 0.228 mmol) was added and the stirring was continued for 2 h.

The reaction was worked up by precipitating the polymer in 15 volumes of acetone (150 mL). The polymer precipitated out immediately as a lump. The solution was stirred for 15 minutes and then the slightly turbid supernatant was decanted. The polymer precipitate was stirred in 10 volumes of acetone (100 mL) for 30 min and then added into 50 mL of water to produce an approximate 20 mg/mL polymer concentration. The solution was then dialyzed against 4 litres of water using a 25 kDa MWCO dialysis tube for 24 h. The water was changed once during that period. The final solution (volume ˜52 mL) was filtered through a 0.22 μm filter and the filtered solution was analyzed for particle size.

Particle properties, evaluated by using the resulting plurality of particles made in the method above:
Zavg=13.34 nm
Particle PDI=0.332
Dv50=4.82 nm
Dv90=9.57 nm

### Example 21

### Synthesis of Docetaxel-2′-β-Alanine Glycolate

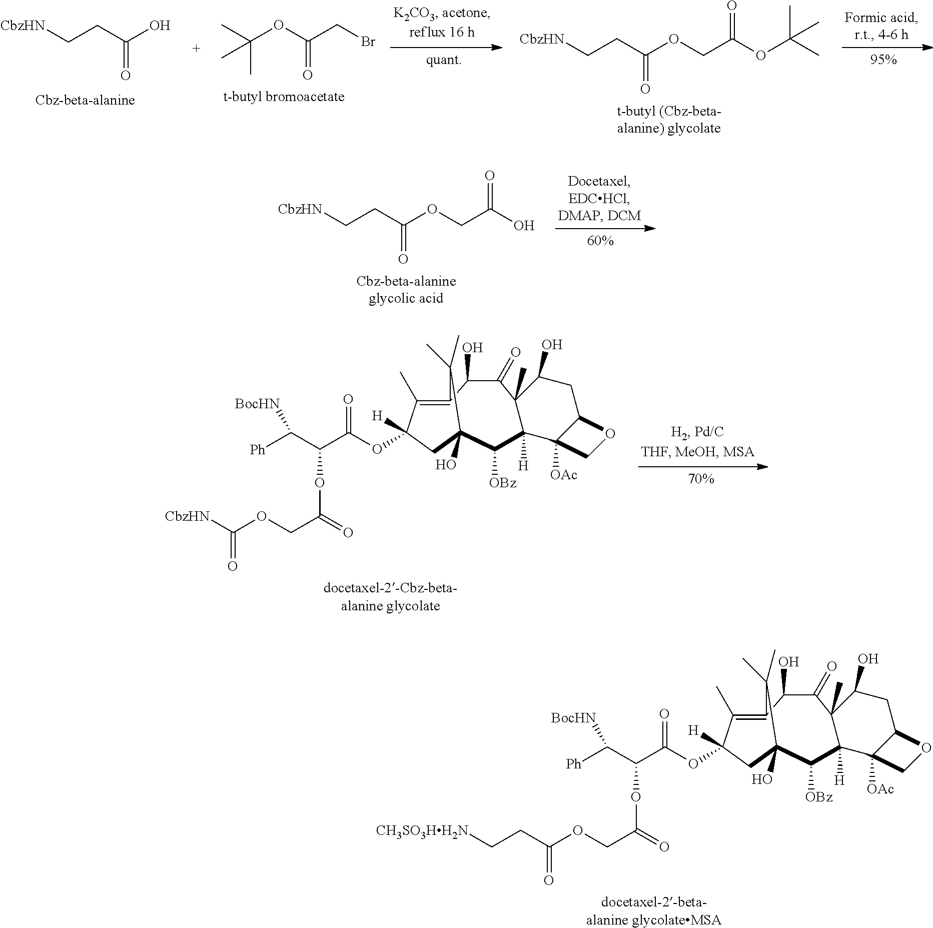

A 1000 mL round-bottom flask equipped with a magnetic stirrer was charged with carbobenzyloxy-β-alanine (Cbz-β-alanine, 15.0 g, 67.3 mmol), tert-butyl bromoacetate (13.1 g, 67.3 mmol), acetone (300 mL), and potassium carbonate (14 g, 100 mmol). The mixture was heated to reflux at 60° C. for 16 h, cooled to ambient temperature and then the solid was removed by filtration. The filtrate was concentrated to a residue, dissolved in ethyl acetate (EtOAc, 300 mL), and washed with 100 mL of water (three times) and 100 mL of brine. The organic layer was separated, dried over sodium sulfate and filtered. The filtrate was concentrated to clear oil [22.2 g, yield: 99%]. HPLC analysis showed 97.4% purity (AUC, 227 nm) and ¹H NMR analysis confirmed the desired intermediate product, t-butyl (carbobenzyloxy-β-alanine) glycolate.

To prepare the intermediate product, carbobenzyloxy-β-alanine glycolic acid (Cbz-β-alanine glycolic acid), a 100 mL round-bottom flask equipped with a magnetic stirrer was charged with t-butyl (Cbz-β-alanine) glycolate [7.5 g, 22.2 mmol] and formic acid (15 mL, 2 vol). The mixture was stirred at ambient temperature for 3 h to give a red-wine color and HPLC analysis showed 63% conversion. The reaction was continued stirring for an additional 2 h, at which point HPLC analysis indicated 80% conversion. An additional portion of formic acid (20 mL, 5 vol in total) was added and the reaction was stirred overnight, at which time HPLC analysis showed that the reaction was complete. The reaction was concentrated under vacuum to a residue and redissolved in ethyl acetate (7.5 mL, 1 vol.). The solution was added to the solvent heptanes (150 mL, 20 vol.) and this resulted in the slow formation of the product in the form of a white suspension. The mixture was filtered and the filter cake was vacuum-dried at ambient temperature for 24 h to afford the desired product, Cbz-β-alanine glycolic acid as a white powder [5.0 g, yield: 80%]. HPLC analysis showed 98% purity. The ¹H NMR analysis in DMSO-d6 was consistent with the assigned structure of Cbz-β-alanine glycolic acid [δ 10.16 (s, 1H), 7.32 (bs, 5H), 5.57 (bs, 1H), 5.14 (s, 2H), 4.65 (s, 2H), 3.45 (m, 2H), 2.64 (m, 2H)].

To prepare the intermediate, docetaxel-2′-carbobenzyloxy-β-alanine glycolate (docetaxel-2′-Cbz-β-alanine glycolate), a 250-mL round-bottom flask equipped with a magnetic stirrer was charged with docetaxel (5.03 g, 6.25 mmol), Cbz-β-alanine glycolic acid [1.35 g, 4.80 mmol] and dichloromethane (DCM, 100 mL). The mixture was stirred for 5 min to produce a clear solution, to which N-(3-Dimethylaminopropyl)-N′-ethylcarbodiimide hydrochloride (EDC.HCl, 1.00 g, 5.23 mmol) and 4-(dimethylamino)pyridine (DMAP, 0.63 g, 5.23 mmol) were added. The mixture was stirred at ambient temperature for 3 h, at which point HPLC analysis showed 48% conversion along with 46% of residual docetaxel. A second portion of Cbz-β-alanine glycolic acid (0.68 g, 2.39 mmol), EDC.HCl (0.50 g, 1.04 mmol) and DMAP (0.13 g, 1.06 mmol) were added and the reaction was allowed to stirred overnight. At this point, HPLC analysis showed 69% conversion along with 12% of residual docetaxel. The solution was diluted to 200 mL with DCM and then washed with 80 mL of water (twice) and 80 mL of brine. The organic layer was separated, dried over sodium sulfate, and then filtered. The filtrate was concentrated to a residue, re-dissolved in 10 mL of chloroform, and purified using a silica gel column. The fractions containing product (shown as a single spot by TLC analysis) were combined, concentrated to a residue, vacuum-dried at ambient temperature for 16 h to produce docetaxel-2′-Cbz-β-alanine glycolate as a white powder [3.5 g, yield: 52%]. HPLC analysis (AUC, 227 nm) indicated >99.5% purity. The ¹H NMR analysis confirmed the corresponding peaks.

To prepare the intermediate, docetaxel-2′-β-alanine glycolate.methanesulfonic acid, a 250 mL round-bottom flask equipped with a magnetic stirrer was charged with docetaxel-2′-Cbz-β-alanine glycolate [3.1 g, 2.9 mmol] and tetrahydrofuran (THF, 100 mL). To the clear solution methanol (MeOH, 4 mL), methanesulfonic acid (172 μL, 2.6 mmol), and 5% palladium on activated carbon (Pd/C, 1.06 g, 10 mol % of Pd) were added. The mixture was evacuated for 15 seconds and filled with hydrogen using a balloon. After 3 h, HPLC analysis indicated that the reaction was complete. Charcoal (3 g, Aldrich, Darco®#175) was then added and the mixture was stirred for 15 min and filtered through a Celite® pad to produce a clear colorless solution. It was concentrated under reduced pressure at <20° C. to ˜5 mL, to which 100 mL of heptanes was added slowly resulting in the formation of a white gummy solid. The supernatant was decanted and the gummy solid was vacuum-dried for 0.5 h to produce a white solid. A volume of 100 mL of heptanes were added and the mixture was triturated for 10 min and filtered. The filter cake was vacuum-dried at ambient temperature for 16 h to produce docetaxel-2′-β-alanine glycolate.MSA as a white powder [2.5 g, yield: 83%]. The HPLC analysis indicated >99% purity (AUC, 230 nm). MS analysis revealed the correct molecular mass (m/z: 936.5).

### Example 22

### Synthesis and Formulation of CDP-Alanine Glycolate-Docetaxel Nanoparticles

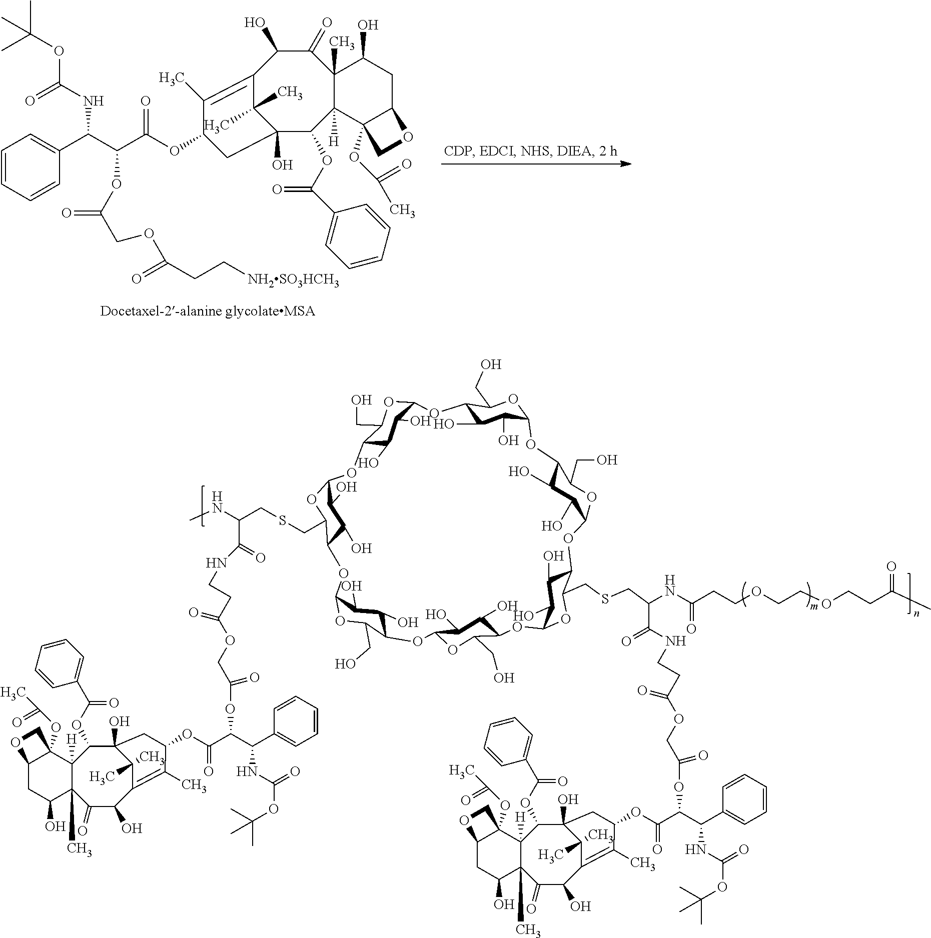

CDP (0.3 g, 0.062 mmol) was dissolved in anhydrous dimethylformamide (DMF, 3 mL) for 30 min with stirring. Docetaxel-2′-alanine glycolate.methanesulfonic acid (0.141 g, 0.137 mmol), 1-ethyl-3-(3-dimethylaminopropyl) carbodiimide (EDCI, 0.036 g, 0.186 mmol) and N-Hydroxysuccinimide (NHS, 0.016 g, 0.137 mmol) was then added to the polymer solution. While stirring, N,N-diisopropylethylamine (DIEA, 0.0177 g, 0.137 mmol) was added and the stirring was continued for 2 h.

The reaction was worked up by precipitating the polymer in 15 volumes of acetone (45 mL), which occurred immediately in the form of a lump. The solution was stirred for 15 minutes and then a slightly turbid supernatant was decanted. The polymer precipitate was stirred in 10 volumes (30 mL) of acetone for 30 min and then added into added into 50 mL of water to produce an approximate 20 mg/mL polymer concentration. The solution was then dialyzed against 4 litres of water using a 25 kDa MWCO dialysis tube for 24 h. During this period, the water was changed once. The resulting solution (˜16.5 mL), was filtered through a 0.22 μm filter and the filtered solution was analyzed for particle size.

Particle properties, evaluated by using the resulting plurality of particles made in the method above:
Zavg=35.81 nm
Particle PDI=0.280
Dv50=12.9 nm
Dv90=26.1 nm

### Example 23

### Synthesis of Docetaxel-2-(2-(2-aminoethoxy)ethoxy)acetic acetate.Methanesulfonic Acid

As used herein, the linker “2-(2-(2-aminoethoxy)ethoxy)acetic acetate” can also be referred to shorthand as “aminoethoxyethoxy”

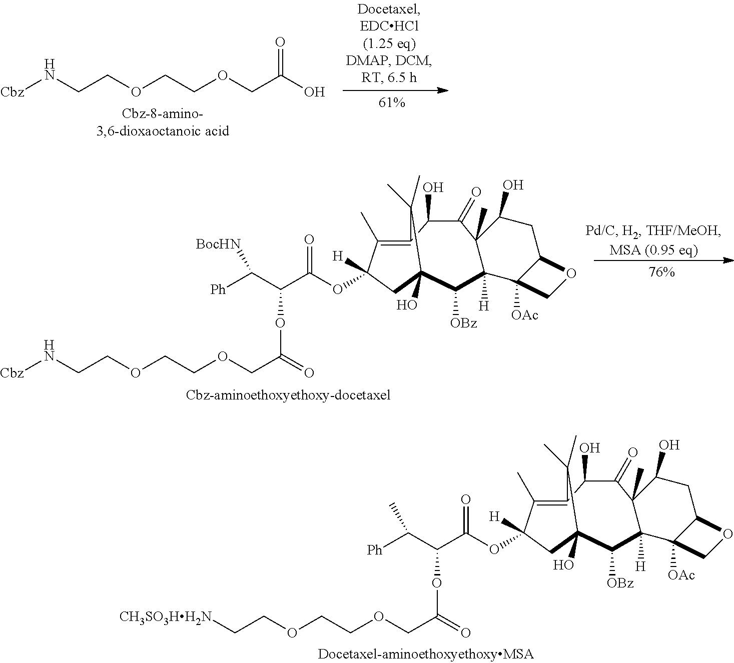

Carbobenzyloxy-8-amino-3,6-dioxaoctanoic acid (3.97 g, 13.3 mmol, 1.19 equiv) was dissolved in dichloromethane (CH₂Cl₂, 10 mL). A portion of this solution (9 mL, about 8.6 mmol, 0.77 equiv) was added to a solution of docetaxel (9.03 g, 11.2 mmol) in CH₂Cl₂ (180 mL) at ambient temperature. 4-(dimethylamino)pyridine (DMAP, 1.23 g, 10.1 mmol, 0.90 equiv) and N-(3-dimethylaminopropyl)-N′-ethylcarbodiimide hydrochloride (EDC.HCl, 1.94 g, 10.1 mmol, 0.91 equiv) were added to the mixture and the contents were stirred at ambient temperature for 2.75 h. An additional amount of cbz-8-amino-3,6-dioxaoctanoic acid (5 mL, about 4.7 mmol, 0.42 equiv), DMAP (830 mg, 6.80 mmol, 0.61 equiv), and EDC.HCl (1.28 g, 6.67 mmol, 0.60 equiv) were added to the mixture and stirred for an additional 4.75 h. The mixture was then washed twice with 0.1% HCl (2×100 mL) and brine (100 mL). The organic layer was dried over sodium sulfate and concentrated to a residue (16.6 g). The residue was dissolved in chloroform (CHCl₃, 40 mL) and purified by flash chromatography to produce carbobenzyloxy-aminoethoxyethoxy-docetaxel as a white solid in two portions [4.2 g, 35%, 97.0% AUC by HPLC] and [1.4 g, 12%, 97.2% AUC by HPLC].

In a 250 mL flask, 5% palladium on activated carbon (Pd/C, 1.95 g) was slurried in tetrahydrofuran (THF, 25 mL) with overhead stirring. The slurry was stirred under hydrogen at ambient temperature for 45 min. A solution of Cbz-aminoethoxyethoxy-docetaxel (5.6 g, 5.2 mmol) in THF (25 mL) and MeOH (5 mL) was added with an additional 25 mL THF wash. After 4.25 h, 5.0 g of activated carbon was added and stirred under nitrogen for 15 min. The slurry was filtered using a 25 mL THF wash and the filtrate was concentrated to about 20 mL. The solution was added drop wise into 200 mL heptanes to form a sticky precipitate. Both THF and MeOH solvents were added until dissolution of the precipitate occurred. A solvent swap into THF was then performed and the solution was concentrated to about 40 mL. Heptanes (500 mL) were subsequently added drop wise. The resulting slurry was filtered using a 250 mL heptanes wash and dried under vacuum overnight to produce docetaxel; -aminoethoxyethoxy.MSA as a white solid [4.55 g, 84%, 97.9% AUC by HPLC]. Pd analysis showed 69 ppm of residual Pd.

### Example 24

### Synthesis and Formulation of CDP-2′-aminoethoxyethoxy-Docetaxel Nanoparticles

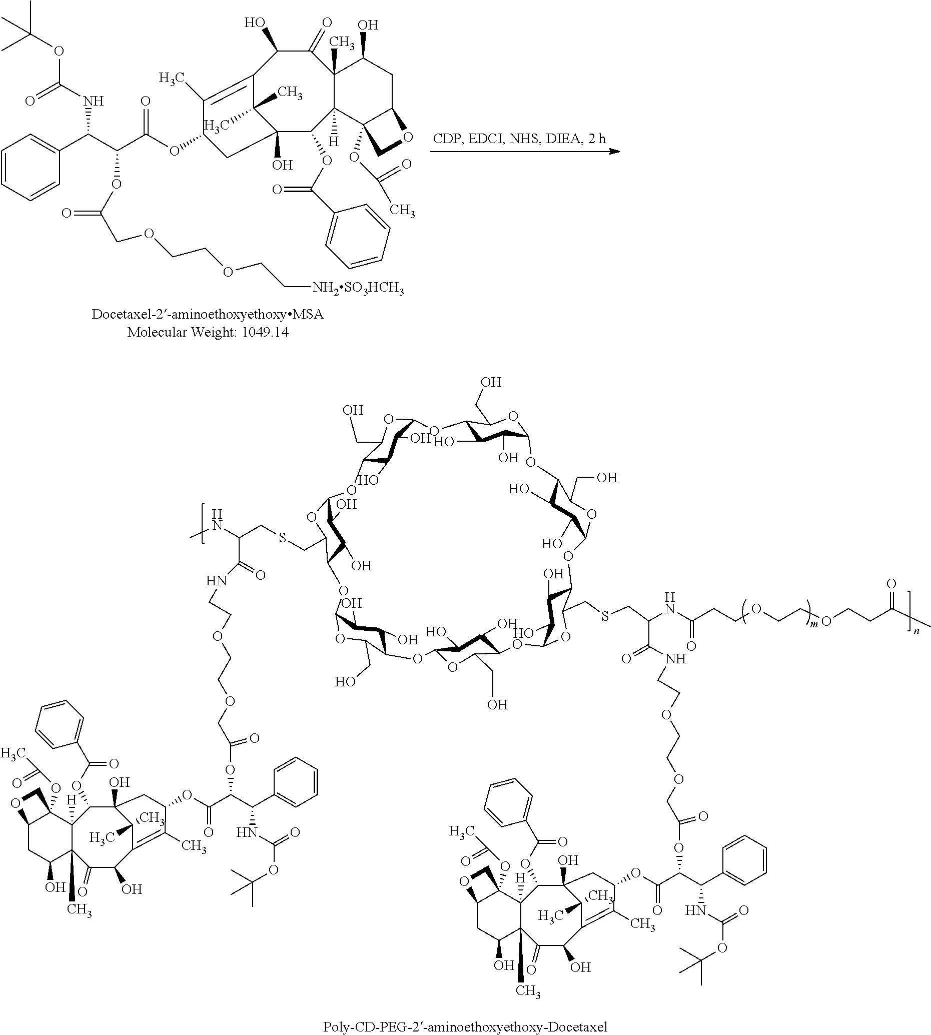

CDP (2 g, 0.414 mmol) was dissolved in anhydrous dimethylformamide (20 mL) and stirred for 30 minutes to dissolve the polymer. Docetaxel-2′-aminoethoxyethoxy.MSA (0.955 g, 0.911 mmol), 1-ethyl-3-(3-dimethylaminopropyl)carbodiimide (EDCI, 0.174 g, 0.911 mmol) and N-hydroxysuccinimide (NHS, 0.1048 g, 0.911 mmol) were added to the polymer solution. While stirring, N,N-diisopropylethylamine (DIEA, 0.117 g, 0.911 mmol) was added and the stirring was continued for 2 h.

The reaction was worked up by precipitating the polymer in 15 volumes of acetone (300 mL). The polymer precipitated out immediately as a lump. The solution was stirred for 30 min. and then the slightly turbid supernatant was decanted. The polymer precipitate was stirred in 10 additional volumes of acetone (200 mL) for 30 min and then poured into 200 mL of water to prepare a ˜10 mg/mL polymer concentration. The polymer dissolved smoothly in water and the polymer solution was then filtered through a 0.22 μm PES membrane. This solution was then washed using TFF (3×30K capsules) using 10 volumes of ultrapure water. After diafiltration, the solution was concentrated down to approximately half the volume and the concentrated solution was filtered with a 0.22 μm cellulose nitrate membrane. The filtered solution was analyzed for particle size using a particle sizer and docetaxel concentration using HPLC.

Particle properties, evaluated by using the resulting plurality of particles made in the method above:
Zavg=18.85 nm
Particle PDI=0.510
Dv50=8.78 nm
Dv90=15.4 nm

### Example 25

### Cytotoxicity of Nanoparticles Formed from CDP-Linker-Docetaxel Compounds

To measure the cytotoxic effect of CDP-linker-docetaxel compounds, the CellTiter-Glo Luminescent Cell Viability Assay (CTG) was used. Briefly, ATP and oxygen in viable cells reduce luciferin to oxyluciferin in the presence of luciferase to produce energy in the form of light. B16.F10 cells, grown to 85-90% confluency in 150 cm2 flasks (passage <30), were resuspended in media (MEM-alpha, 10% HI-FBS, 1×antibiotic-antimycotic solution) and added to 96-well opaque-clear bottom plates at a concentration of 1500 cells/well in 200 μL/well. The cells were incubated at 37° C. with 5% CO₂ for 24 hours. The following day, serial dilutions of 2× concentrated particles and 2× concentrated free drug were made in 12-well reservoirs with media to specified concentrations. The media in the plates was replaced with 1000_, of fresh media and 100 μL of the corresponding serially diluted drug. Three sets of plates were prepared with duplicate treatments. Following 24, 48 and 72 hours of incubation at 37° C. with 5% CO2, the media in the plates was replaced with 100 μL of fresh media and 100 μL of CTG solution, and then incubated for 5 minutes on a plate shaker at room temperature set to 450 rpm and allowed to rest for 15 minutes. Viable cells were measured by luminescence using a microtiter plate reader. The data was plotted as % viability vs. concentration and standardized to untreated cells. The CDP-linker-docetaxel compounds inhibited the growth of B16.F10 cells in a dose and time dependent manner. Also, in comparison to the corresponding free drug, the CDP-linker-docetaxel compounds exhibited a slower release profile. IC₅₀: IC₅₀ values 72 hours after treatment are shown in the table below

| Group | IC₅₀ (nM) |
|---|---|
| Free docetaxel | 0.2-2 |
| CDP-2′-hexanoate-docetaxel | 325-440 |
| CDP-2′-glycine-docetaxel | 1.2-3.7 |
| CDP-dithiolethyloxy-carbonate-docetaxel | 23 |
| CDP-2′-alanine glycolate-docetaxel | 0.4-2.0 |
| CDP-2′-aminoethoxyethoxys-Docetaxel | NA |

### Example 26

### Drug Release and Stability Method for the CDP-Linker-Docetaxel Compounds

The drug release and stability method experiment was run using the following

CDP-linker-docetaxel nanoparticles: CDP-2′-glycine-docetaxel (CDP-Gly-DTX), CDP-2′-alanine glycolate-docetaxel (CDP-Ala Gly-DTX), CDP-2′-hexanoate-docetaxel (CDP-Hex-DTX), CDP-dithiolethyloxy-carbonate-docetaxel (CDP-ethane-S—S-ethane-DTX) and CDP-2′-aminoethoxyethoxy-Docetaxel (CDP-aminoethoxyethoxy-DTX).

A 10 mg/mL (with regard to polymer) solution of each CDP-linker-DTX nanoparticle was prepared in water (pH<5) or in 0.1×PBS buffer (pH=7.4). An aliquot of 100 μL was transferred into corresponding HPLC vials. A vial containing each CDP-linker-DTX nanoparticle in water for each designated time point was placed in both: 1) a water bath at 37° C. and 2) kept at room temperature at 25° C. Samples were mixed using a water bath shaker at 100 rpm during the experiments. At each designated time point, a vial was removed for each CDP-linker-DTX nanoparticle and processed for HPLC using a sample preparation procedure.

To prepare a sample for HPLC analysis, each vial containing 100 μL of sample was mixed with 25 μL of 0.1% formic acid in ACN, which is a good solvent for both docetaxel and the CDP polymer. If there was any precipitated material in the vial, the contents were also stirred to dissolve the precipitate. If the sample was still opaque, an additional 25 μL of 0.1% formic acid in ACN was added. HPLC analysis was used to determine the amount of free docetaxel and the amount of conjugated docetaxel in the sample for a given time point.

For the HPLC analysis at each time point, the peak areas of all relevant peaks from the chromatograms were retrieved and the concentration of free and conjugated docetaxel was calculated. The sample degradation was calculated based on the percentage of the amount of conjugated drug with regard to the initial starting point of the experiment (at t=0). The drug release was calculated based on the sum of free docetaxel and docetaxel main degradants at each time point. The drug release and degradation of given conjugate at 37° C. in 0.1×PBS after 24 h are presented in Table 1.

TABLE 1
Drug Release for Different CDP-linker-Docetaxel products at 37° C. in
0.1x PBS at pH = 7.4

| | In vitro | |
|---|---|---|
| | release of free | In vitro degradation |
| | drug (24 hrs in | of conjugate (24 hrs in |
| CPX# | PBS at 37° C.) | PBS at 37° C.) |
| CDP-Glycine-DTX | 88% | 84% |
| CDP-Ala Gly-DTX | 95% | 96% |
| CDP-Hex-DTX | 8% | 7% |
| CDP-Ethane-S—S-Ethane-Doce | 7% | 4% |
| CDP-aminoethoxyethoxy-Doce | 71% | 74% |

The data indicates that the hexanoate linker and the disulfide linker are relatively stable toward hydrolysis in vitro, whereas the glycine linker, alanine-glycolate linker, and aminoethoxyethoxylinker are more susceptible to hydrolysis.

Relative stability of different CDP-linker-DTX nanoparticles:

CDP-hex-DTX, CDP-ethane-S—S-ethane-DTX>>CDP-aminoethoxyethoxy-DTX>CDP-Gly-DTX, CDP-Ala Gly-DTX

### Example 27

### Efficacy and Tolerability of CDP—Docetaxel Nanoparticles in a Murine Melanoma Model

B16.F10 cells were grown in culture to 85-90% confluency in MEM-alpha medium supplemented with 10% fetal bovine serum (FBS) and 1% penicillin/streptomycin. Cells were removed from the flask using 0.05% trypsin (passage=4), re-suspended in PBS (density=10×10⁶ cells/mL) and were implanted subcutaneously (1×10⁶ cells in 100 μL of PBS/mouse) into the right flank of male C57BL/6 mice on day 1.

The six treatment groups that were administered to the mice included: 1)

Docetaxel formulation prepared at 10 mg/mL stock solution (with 20 mg of docetaxel, 0.2 mL ethanol, 0.5 mL Tween 80 and 1.3 mL water, added in that specific order and vortexed to ensure proper mixing) and diluted further with PBS to 1.5 and 3 mg/mL concentrations for a corresponding dose of 15 and 30 mg/kg respectively. 2) CDP-2′-glycine-docetaxel (CDP-Gly-DTX) nanoparticle formulation administered at 15 and 30 mg/kg. 3) CDP-2′-alanine glycolate-docetaxel (CDP-Ala Gly-DTX) nanoparticle formulation administered at 15 and 30 mg/kg. 4) CDP -2′-hexanoate-docetaxel (CDP-Hex-DTX) nanoparticle formulation administered at 30 mg/kg. (5) CDP-dithiolethyloxy-carbonate-docetaxel (CDP-ethane-S—S-ethane-DTX) nanoparticle formulation administered at 15 and 30 mg/kg. (6) CDP -2′-aminoethoxyethoxy-docetaxel (CDP-aminoethoxyethoxy-DTX) nanoparticle formulation administered at 15 and 30 mg/kg.

The treatments were administered IV into the tail vein at a dose volume of 10 mL/kg, beginning on post-implantation day 5, when the mean tumor volume was ca. 60 mm³. Animals were monitored for any morbidity and adverse effect three times a week. In addition, body weight and tumor volume were also measured three times a week.

Tumor volume was calculated with a (width×width×length)/2 mm³ formula. Efficacy was determined by tumor growth inhibition (TGI), tumor growth delay (TGD) and survival. Tumor growth inhibition (TGI) was represented as % and calculated as (1−(treated tumor volume/control tumor volume))×100 when the control group mean tumor volume reached ≧3000 mm³. Tumor growth delay (TGD) was calculated by subtracting the day when the vehicle treated group reached the maximum tumor size 3000 mm³ from the day when the treatment group tumor size reached 3000 mm³. The criterion at which a mouse was removed from the study was tumor volume ≧3000 mm³.

Tolerability was determined by changes in body weight, expressed as a percent of the initial body weight on post-implantation day 5. Health monitoring was conducted three times a week to evaluate lethargy, tremors, hypothermia, ataxia, hind limb paralysis etc. The criteria at which a mouse was removed from the study were >20% body weight loss or severe morbidity or hind limb paralysis. When one of these criteria are seen, such as greater than or equal to 20% body weight loss, the method of administering the CDP-taxane conjugates to the subject can be modified by, for example, decreasing the dose of the CDP-taxane conjugates to the subject, or increasing the interval between doses of the CDP-taxane conjugates to the subject.

1. CDP -2′-glycine-docetaxel (CDP-Gly-DTX) Nanoparticle Formulation

1.1. The CDP-Gly-DTX formulation was administered at a dose of 15 mg/kg with a schedule of three injections over 2 weeks at a dosing frequency of twice per week. Free docetaxel administered at the same dose and schedule of CDP-Gly-DTX formulation showed similar TGI. At 15 mg/kg, the TGI was 97% for the free docetaxel group and 98% for the CDP-Gly-DTX formulation group. CDP-Gly-DTX formulation showed better TGD as compared to the free docetaxel group. The free docetaxel group reached the mean tumor volume endpoint (≧3000 mm³) on day 34 and exhibited 15 days TGD (79% increase in TGD). In comparison, the CDP-Gly-DTX formulation had mean tumor volumes of 233 mm³ and 374 mm³ on day 33 and day 36 respectively and the group continued beyond day 36 whereas the free docetaxel group ended because the mean tumor volume reached the endpoint (≧3000 mm³). On day 52, the mean tumor volume of the CDP-Gly-DTX formulation group was 1556 mm³ and the TGD was greater than 33 days as the mean tumor volume of the group did not reach the endpoint (≧3000 mm³) on day 52. For the free docetaxel group, 50% survival was observed on day 33 and 0% survival on day 40 compared to the CDP-Gly-DTX formulation which showed 86% survival on day 40, 50% on day 94 and 43% survival on day 115. Both the free docetaxel and CDP-Gly-DTX nanoparticle formulation did not cause any significant body weight loss.

| | | | | Maximum |
|---|---|---|---|---|
| | | Tumor growth | Tumor | body weight |
| | Dose | inhibition | growth delay | loss |
| Formulation | (mg/kg) | (% TGI) | (TGD) | (%) |
| Free docetaxel | 15 | 97% | 15 days | 3% |
| CDP-Gly-DTX | 15 | 98% | >33 days | 7% |
| formulation | | | | |

1.2. The CDP-Gly-DTX formulation was administered at a dose of 30 mg/kg with a schedule of three injections over 2 weeks at a dosing frequency of twice per week. Free docetaxel administered at a dose of 15 mg/kg, on a biweekly schedule for 3 injections showed similar TGI as compared to CDP-Gly-DTX formulation. At 15 mg/kg, the TGI was 97% for the free docetaxel group whereas TGI was 98% for the CDP-Gly-DTX formulation group at 30 mg/kg. CDP-Gly-DTX formulation showed better TGD as compared to the free docetaxel group. The free docetaxel group reached the mean tumor volume endpoint (≧3000 mm³) on day 34 and exhibited 15 days TGD (79% increase in TGD). In comparison, the CDP-Gly-DTX formulation had mean tumor volumes of 63 mm³ on both day 33 and day 36 and the group continued beyond day 36 whereas the free docetaxel group ended because the mean tumor volume reached the endpoint (≧3000 mm³). On day 82, the mean tumor volume of the CDP-Gly-DTX formulation was 1979 mm³ and the TGD was greater than 63 days as the mean tumor volume of the group did not reach the endpoint (≧3000 mm³) on day 82.50% survival was observed on day 33 in the free docetaxel group and 0% survival on day 40 compared to the CDP-Gly-DTX formulation which showed 100% survival on day 40 and 50% survival on day 115. The CDP-Gly-DTX formulation caused 20% body weight loss.

| | | | | Maximum |
|---|---|---|---|---|
| | | Tumor growth | Tumor | body weight |
| | Dose | inhibition | growth delay | loss |
| Formulation | (mg/kg) | (% TGI) | (TGD) | (%) |
| Free docetaxel | 15 | 97% | 15 days | 3% |
| CDP-Gly-DTX | 30 | 98% | >63 days | 20% |
| formulation | | | | |

1.3. The CDP-Gly-DTX formulation was administered at a dose of 15 mg/kg, on a weekly schedule for 3 injections. The free docetaxel group administered at the same dose and schedule was less efficacious than the CDP-Gly-DTX formulation. At 15 mg/kg, the TGI was 68% for the free docetaxel group compared to 82% TGI for the CDP-Gly-DTX formulation. The free docetaxel group reached the mean tumor volume endpoint (≧3000 mm³) on day 26 and exhibited 7 days TGD (37% increase in TGD). In contrast, the CDP-Gly-DTX formulation reached mean tumor volume endpoint on day 31 and exhibited 12 days TGD (63% increase in TGD). Both free docetaxel and CDP-Gly-DTX formulation groups did not cause any body weight loss.

| | | | | Maximum |
|---|---|---|---|---|
| | | Tumor growth | Tumor | body weight |
| | Dose | inhibition | growth delay | loss |
| Formulation | (mg/kg) | (% TGI) | (TGD) | (%) |
| Free docetaxel | 15 | 68% | 7 days | 0% |
| CDP-Gly-DTX | 15 | 82% | 12 days | 0% |
| formulation | | | | |

1.4 The CDP-Gly-DTX formulation was administered at a dose of 30 mg/kg, on a weekly schedule for 3 injections. The free docetaxel group administered at the same dose and schedule was less efficacious than the CDP-Gly-DTX formulation. At 30 mg/kg, the TGI was 84% for the free docetaxel group compared to 96% TGI for the CDP-Gly-DTX formulation. The free docetaxel group reached the mean tumor volume endpoint (≧3000 mm³) on day 31 and exhibited 12 days TGD (63% increase in TGD). In comparison, the CDP-Gly-DTX formulation reached the mean tumor volume endpoint on day 47 and exhibited 28 days TGD (147% increase in TGD). For the free docetaxel group, 50% survival was observed on day 29 and 0% survival on day 38 compared to the CDP-Gly-DTX formulation which showed 50% survival on day 47 and 25% survival on day 59. Both free docetaxel and CDP-Gly-DTX formulation groups did not cause any significant body weight loss.

| | | | | Maximum |
|---|---|---|---|---|
| | | Tumor growth | Tumor | body weight |
| | Dose | inhibition | growth delay | loss |
| Formulation | (mg/kg) | (% TGI) | (TGD) | (%) |
| Free docetaxel | 30 | 84% | 12 days | 8% |
| CDP-Gly-DTX | 30 | 96% | 28 days | 14% |
| formulation | | | | |

1.5 The CDP-Gly-DTX formulation was administered at a dose of 30 mg/kg, on a weekly schedule for 3 injections. The free docetaxel group administered at the same dose and schedule was less efficacious than the CDP-Gly-DTX formulation. At 30 mg/kg, the TGI was 92% for the free docetaxel group compared to 99% TGI for the CDP-Gly-DTX formulation. The free docetaxel group reached the mean tumor volume endpoint (≧3000 mm³) on day 41 and exhibited 21 days TGD (105% increase in TGD). In comparison, the CDP-Gly-DTX formulation did not yet reach the mean tumor volume endpoint (≧3000 mm³) on day 80 and exhibited >60 days TGD (>300% increase in TGD). For the free docetaxel group, 50% survival was observed on day 40 and 0% survival on day 45 compared to the CDP-Gly-DTX formulation which showed 62.5% survival on day 127, which was the last day of the experiment.

| | | | | Maximum |
|---|---|---|---|---|
| | | Tumor growth | Tumor | body weight |
| | Dose | inhibition | growth delay | loss |
| Formulation | (mg/kg) | (% TGI) | (TGD) | (%) |
| Free docetaxel | 30 | 92% | 21 days | 12% |
| CDP-Gly-DTX | 30 | 99% | >60 days | 15% |
| formulation | | | | |

1.6. The CDP-Gly-DTX formulation was administered at a dose of 30 mg/kg on a biweekly schedule for 3 injections. The free docetaxel group administered at 30 mg/kg on a biweekly schedule for 2 injections was less efficacious than CDP-Gly-DTX formulation. At 30 mg/kg, the TGI was 73% for the free docetaxel in contrast to 93% TGI for the CDP-Gly-DTX formulation. The free docetaxel group reached the mean tumor volume endpoint (≧3000 mm³) on day 26 and exhibited 7 days TGD (37% increase in TGD). In comparison, the CDP-Gly-DTX formulation reached the mean tumor volume endpoint on day 43 and exhibited 24 days TGD (126% increase in TGD). The free docetaxel group did not receive the 3^{rd} injection (on day 33) because the group exited on day 26.50% survival was observed on day 24 in the free docetaxel group and 0% survival on day 31 whereas the CDP-Gly-DTX formulation showed 50% survival on day 40 and 13% survival on day 59. Both the free docetaxel and CDP-Gly-DTX formulation groups did not cause any significant body weight loss.

| | | | | Maximum |
|---|---|---|---|---|
| | | Tumor growth | Tumor | body weight |
| | Dose | inhibition | growth delay | loss |
| Formulation | (mg/kg) | (% TGI) | (TGD) | (%) |
| Free docetaxel | 30 | 73% | 7 days | 5% |
| CDP-Gly-DTX | 30 | 93% | 24 days | 4% |
| formulation | | | | |

2. CDP-2′-alanine glycolate-docetaxel (CDP-Ala Gly-DTX) Nanoparticle Formulation

2.1. The CDP-Ala Gly-DTX formulation was administered at 15 mg/kg, three injections over a 2 week schedule. Free docetaxel administered at the same dose and schedule of CDP-Ala Gly-DTX formulation showed similar TGI. At 15 mg/kg, the TGI was 97% for the free docetaxel group and 98% for the CDP-Ala Gly-DTX formulation group. CDP-Ala Gly-DTX formulation however showed better TGD as compared to the free docetaxel group. The mean tumor volume endpoint (≧3000 mm³) was reached at day 35 for the free docetaxel group compared to day 43 for CDP-Ala Gly-DTX formulation. The free docetaxel group exhibited 15 days TGD (79% increase in TGD) whereas CDP-Ala Gly-DTX formulation showed 24 days TGD (126% increase in TGD). 50% survival was observed on day 33 in the free docetaxel group and 0% survival on day 40 whereas CDP-Ala Gly-DTX formulation showed 75% survival on day 40 and 38% survival on day 43. Both free docetaxel and CDP-Ala Gly-DTX formulation groups did not cause any significant body weight loss.

| | | | Tumor | Maximum |
|---|---|---|---|---|
| | | Tumor growth | growth | body weight |
| | Dose | inhibition | delay | loss |
| Formulation | (mg/kg) | (% TGI) | (TGD) | (%) |
| Free docetaxel | 15 | 97% | 15 days | 3% |
| CDP-Ala Gly-DTX | 15 | 98% | 24 days | 6% |
| formulation | | | | |

2.2. The CDP-Ala Gly-DTX formulation was administered at a dose of 15 mg/kg on a weekly schedule for 3 injections. The free docetaxel group administered at the same dose and schedule was less efficacious than CDP-Ala Gly-DTX formulation. The free docetaxel and CDP-Ala Gly-DTX formulation groups resulted in 68% TGI and 85% TGI respectively. On day 26, the free docetaxel group reached the mean tumor volume endpoint (≧3000 mm³) and exhibited 7 days TGD (37% increase in TGD). In comparison, on day 33, CDP-Ala Gly-DTX formulation reached the mean tumor volume endpoint on day 33 and exhibited 14 days TGD (74% increase in TGD). Both free docetaxel and CDP-Ala Gly-DTX formulation groups did not cause any significant body weight loss.

| | | | Tumor | Maximum |
|---|---|---|---|---|
| | | Tumor growth | growth | body weight |
| | Dose | inhibition | delay | loss |
| Formulation | (mg/kg) | (% TGI) | (TGD) | (%) |
| Free docetaxel | 15 | 68% | 7 days | 0% |
| CDP-Ala Gly-DTX | 15 | 85% | 14 days | 3% |
| formulation | | | | |

2.3. The CDP-Ala Gly-DTX formulation was administered at 30 mg/kg on a weekly schedule for 3 injections. Free docetaxel administered at the same dose and schedule was less efficacious than CDP-Ala Gly-DTX formulation. At 30 mg/kg, the free docetaxel and CDP-Ala Gly-DTX formulation groups caused 84% TGI and 96% TGI respectively. On day 31, the free docetaxel group reached the mean tumor volume endpoint (≧3000 mm³) and exhibited 12 days TGD (63% increase in TGD). In comparison, on day 43, CDP-Ala Gly-DTX formulation reached the mean tumor volume endpoint and showed 24 days TGD (126% increase in TGD). 50% survival was observed on day 29 in the free docetaxel group and 0% survival on day 38 whereas CDP-Ala Gly-DTX formulation showed 50% survival on day 40 and 0% survival on day 54. Both free docetaxel and CDP-Ala Gly-DTX formulation groups did not cause any significant body weight loss.

| | | | Tumor | Maximum |
|---|---|---|---|---|
| | | Tumor growth | growth | body weight |
| | Dose | inhibition | delay | loss |
| Formulation | (mg/kg) | (% TGI) | (TGD) | (%) |
| Free docetaxel | 30 | 84% | 12 days | 8% |
| CDP-Ala Gly-DTX | 30 | 96% | 24 days | 7% |
| formulation | | | | |

2.4. The CDP-Ala Gly-DTX formulation was administered at 30 mg/kg on a biweekly schedule for 2 injections. Free docetaxel administered at the same dose and schedule showed similar TGI but less TGD as compare to CDP-Ala Gly-DTX formulation. Free docetaxel caused 73% TGI whereas CDP-Ala Gly-DTX formulation caused 77% TGI. The free docetaxel group reached the mean tumor volume endpoint (>3000 mm³) on day 26 and exhibited 7 days TGD (37% increase in TGD) whereas CDP-Ala Gly-DTX formulation reached the mean tumor volume endpoint on day 29 and exhibited 10 days TGD (53% increase in TGD). For the free docetaxel, 50% survival was observed on day 24 and 0% survival on day 31. In comparison, CDP-Ala Gly-DTX formulation showed 50% survival on day 29 and 0% survival on day 36. Both free docetaxel and CDP-Ala Gly-DTX formulation groups did not cause any significant body weight loss.

| | | | Tumor | Maximum |
|---|---|---|---|---|
| | | Tumor growth | growth | body weight |
| | Dose | inhibition | delay | loss |
| Formulation | (mg/kg) | (% TGI) | (TGD) | (%) |
| Free docetaxel | 30 | 73% | 7 days | 5% |
| CDP-Ala Gly-DTX | 30 | 77% | 10 days | 2% |
| formulation | | | | |

3. CDP-2′-hexanoate-docetaxel (CDP-Hex-DTX) Nanoparticle Formulation

3.1. The CDP-Hex-DTX formulation was administered at a dose of 30 mg/kg, three injections over a 2 week schedule. Free docetaxel administered at 15 mg/kg, three injections over a 2 week schedule was more efficacious than CDP-Hex-DTX formulation. At 15 mg/kg, the free docetaxel resulted in a 97% TGI compared to CDP-Hex-DTX formulation at 30 mg/kg, resulted in a 66% TGI. On day 34, the free docetaxel group reached the mean tumor volume endpoint (≧3000 mm³) and exhibited 15 days TGD (79% increase in TGD). The CDP-Hex-DTX formulation showed 10 days TGD (53% increase in TGD). Both free docetaxel and CDP-Hex-DTX formulation groups did not cause any significant body weight loss.

| | | | Tumor | Maximum |
|---|---|---|---|---|
| | | Tumor growth | growth | body weight |
| | Dose | inhibition | delay | loss |
| Formulation | (mg/kg) | (% TGI) | (TGD) | (%) |
| Free docetaxel | 15 | 97% | 15 days | 3% |
| CDP-Hex-DTX | 30 | 66% | 10 days | 0% |
| formulation | | | | |

4. CDP-dithiolethyloxy-carbonate-docetaxel (CDP-ethane-S—S-ethane-DTX) Nanoparticle Formulation

4.1. The CDP-ethane-S—S-ethane-DTX formulation was administered at a dose of 15 mg/kg, on a weekly schedule for 3 injections. Free docetaxel administered at the same dose and schedule was found to be more efficacious than CDP-ethane-S—S-ethane-DTX formulation. Free docetaxel caused 68% TGI whereas CDP-ethane-S—S-ethane-DTX formulation caused 24% TGI. On day 26, the free docetaxel group reached the mean tumor volume endpoint (≧3000 mm³) and exhibited 7 days TGD (37% increase in TGD) compared to CDP-ethane-S—S-ethane-DTX formulation which reached the mean tumor volume endpoint on day 21 and exhibited 2 days TGD (11% increase in TGD). Both free docetaxel and CDP-ethane-S—S-ethane-DTX formulation groups did not cause any body weight loss.

| | | Tumor | Tumor | Maximum |
|---|---|---|---|---|
| | | growth | growth | body weight |
| | Dose | inhibition | delay | loss |
| Formulation | (mg/kg) | (% TGI) | (TGD) | (%) |
| Free docetaxel | 15 | 68% | 7 days | 0% |
| CDP-ethane-S-S-ethane- | 15 | 24% | 2 days | 0% |
| DTX formulation | | | | |

4.2. The CDP-ethane-S—S-ethane-DTX formulation was administered at a dose of 30 mg/kg, on a weekly schedule for 3 injections. The free docetaxel administered at the same dose and schedule was less efficacious than CDP-ethane-S—S-ethane-DTX formulation. At 30 mg/kg, free docetaxel resulted in 84% TGI compared to 46% TGI for the CDP-ethane-S—S-ethane-DTX formulation. On day 31, the free docetaxel group reached the mean tumor volume endpoint (≧3000 mm³) and showed 12 days TGD (63% increase in TGD) compared to CDP-ethane-S—S-ethane-DTX formulation which reached the mean tumor volume endpoint on day 24 and exhibited 5 days TGD (26% increase in TGD). Both free docetaxel and CDP-ethane-S—S-ethane-DTX formulation groups did not cause any significant body weight loss.

| | | Tumor | Tumor | Maximum |
|---|---|---|---|---|
| | | growth | growth | body weight |
| | Dose | inhibition | delay | loss |
| Formulation | (mg/kg) | (% TGI) | (TGD) | (%) |
| Free docetaxel | 30 | 84% | 12 days | 8% |
| CDP-ethane-S-S-ethane- | 30 | 46% | 5 days | 0% |
| DTX formulation | | | | |

5. CDP-2′-aminoethoxyethoxy-Docetaxel (CDP-aminoethoxyethoxy-DTX) Nanoparticle Formulation

5.1. The CDP-aminoethoxyethoxy-DTX formulation was administered at a dose of 15 mg/kg on a weekly schedule for 3 injections. Free docetaxel administered at the same dose and schedule was less efficacious than the CDP-aminoethoxyethoxy-DTX formulation. Free docetaxel resulted in 68% TGI compared to CDP-aminoethoxyethoxy-DTX formulation which resulted in a 87% TGI. On day 26, the free docetaxel group reached the mean tumor volume endpoint (≧3000 mm³) and showed 7 days TGD (37% increase in TGD). In comparison, CDP-aminoethoxyethoxy-DTX formulation reached the mean tumor volume endpoint on day 33 and exhibited 14 days TGD (74% increase in TGD). Both free docetaxel and CDP-aminoethoxyethoxy-DTX formulation groups did not cause any significant body weight loss.

| | | Tumor | Tumor | Maximum |
|---|---|---|---|---|
| | | growth | growth | body weight |
| | Dose | inhibition | delay | loss |
| Formulation | (mg/kg) | (% TGI) | (TGD) | (%) |
| Free docetaxel | 15 | 68% | 7 days | 0% |
| CDP-aminoethoxyethoxy- | 15 | 87% | 14 days | 7% |
| DTX formulation | | | | |

5.2. The CDP-aminoethoxyethoxy-DTX formulation was administered at a dose of 30 mg/kg on a weekly schedule for 3 injections. Free docetaxel administered at the same dose and schedule was less efficacious than the CDP-aminoethoxyethoxy-DTX formulation. At 30 mg/kg, free docetaxel resulted in a 84% TGI compared to 97% TGI for CDP-aminoethoxyethoxy-DTX formulation. The free docetaxel group reached the mean tumor volume endpoint (≧3000 mm³) on day 31 and exhibited 12 days TGD (63% increase in TGD) whereas the mean tumor volume of the CDP-aminoethoxyethoxy-DTX formulation was 1442 mm³ on day 59 and the TGD was more than 40 days. 50% survival was observed on day 29 for the free docetaxel group and 0% survival on day 38. In comparison, CDP-aminoethoxyethoxy-DTX formulation showed 88% survival on day 59. The CDP-aminoethoxyethoxy-DTX formulation caused 23% body weight loss.

| | | Tumor | Tumor | Maximum |
|---|---|---|---|---|
| | Dose | growth | growth | body |
| | (mg/ | inhibition | delay | weight loss |
| Formulation | kg) | (% TGI) | (TGD) | (%) |
| Free docetaxel | 30 | 84% | 12 days | 8% |
| CDP-aminoethoxyethoxy- | 30 | 97% | >40 days | 23% |
| DTX formulation | | | | |

### Example 28

### Synthesis of Larotaxel Glycinate

A 1000 mL, three-neck jacketed reactor equipped with an addition funnel, overhead stirrer, J-KEM probe, and N₂ inlet will be charged with larotaxel (22.3 g, 26.7 mmol), N-Cbz-glycine (5.6 g, 26.7 mmol), DMAP (3.3 g, 26.7 mmol) and DCM (150 mL). The mixture will be stirred for a few minutes to produce a clear solution. It will be cooled from −2 to 2° C. with a TCM. A suspension of EDCI (10.2 g, 53.4 mmol) and DMAP (1.6 g, 13.3 mmol) in DCM (100 mL) will be added dropwise over 2 h. The reaction will be stirred from −2 to 2° C. for 12 h and subsequently the temperature will be lowered to −5° C. Additional N-Cbz-glycine (2.2 g, 10.7 mmol) will be added, followed by addition of EDCI (5.1 g, 26.7 mmol) and DMAP (1.6 g, 13.3 mmol) in DCM (50 mL) over 1 h. The reaction will be stirred at −5° C. for 16 h and then at 0° C. for 4 h, at which time IPC analysis will be done to check for the consumption of larotaxel. Once the reaction completion is confirmed, the reaction mixture will be diluted with DCM to 500 mL and washed with 1% HCl (2×150 mL), saturated NaHCO₃ (2×100 mL) and brine (150 mL). The organic layer will be separated, dried over Na₂SO₄, and filtered. The filtrate will be concentrated to a residue to produce a crude product. The crude product will then be purified by column chromatography to yield pure Cbz-glycinate larotaxel.

A 1000 mL round-bottom flask equipped with a magnetic stirrer will be charged with THF (160 mL), methanesulfonic acid (980 μL), and 5% Pd/C (5.9 g). The suspension will be evacuated and back filled with H₂ three times and stirred under H₂ for 0.5 h. A solution of Cbz-glycinate larotaxel (17.5 g, 17.0 mmol) in THF (170 mL) and MeOH (10 mL) will be added. The reaction will be monitored by HPLC. After the reaction is completed, charcoal (10 g) will be added to the reaction and the mixture will be stirred for 10 min and filtered through a Celite pad to produce a clear solution. It will be concentrated to −50 mL, to which heptanes (500 mL) will be added to precipitate out the product. It will then be dried under vacuum to yield larotaxel glycinate.

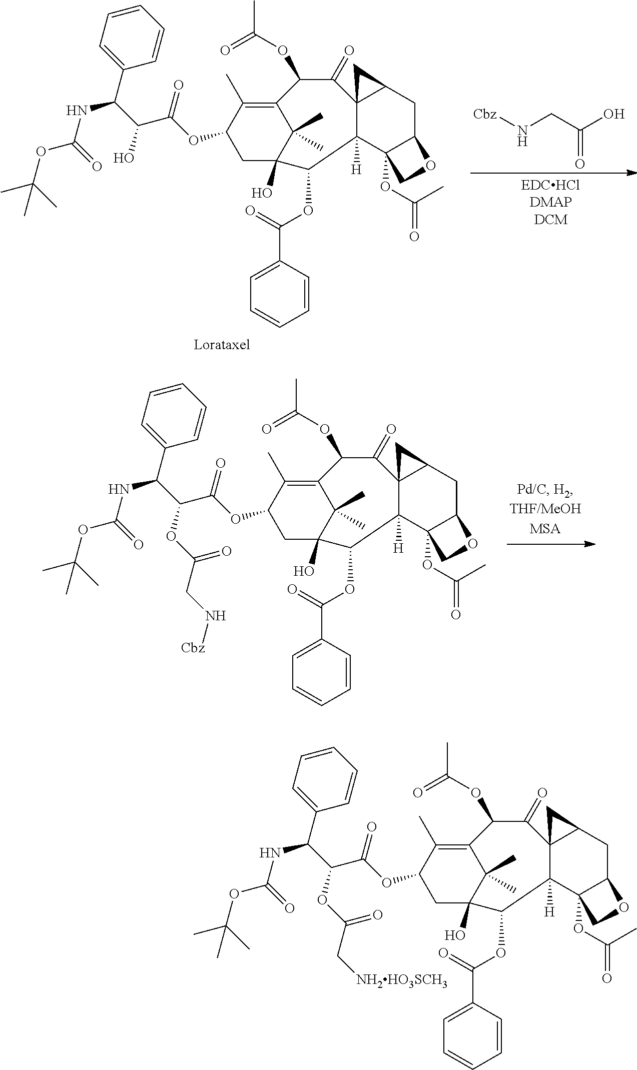

### Example 29

### Synthesis of CDP Larotaxel Glycinate Conjugate

CDP (1.0 g, 0.21 mmol) will be dissolved in dry N,N-dimethylformamide (DMF, 10 mL). Larotaxel glycinate (400 mg, 0.46 mmol), N,N-Diisopropylethylamine (59 mg, 0.46 mmol), N-(3-Dimethylaminopropyl)-N′-ethylcarbodiimide hydrochloride (87 mg, 0.46 mmol), and N-Hydroxysuccinimide (52 mg, 0.46 mmol) will then be added to the polymer solution and stirred for 2 h. The polymer will be precipitated with isopropanol (150 mL) and then rinsed with acetone (100 mL). The precipitate will be dissolved in nanopure water (100 mL). It will be purified by TFF with nanopure water (1 L). Finally it will be filtered through 0.2 μm filter and kept frozen.

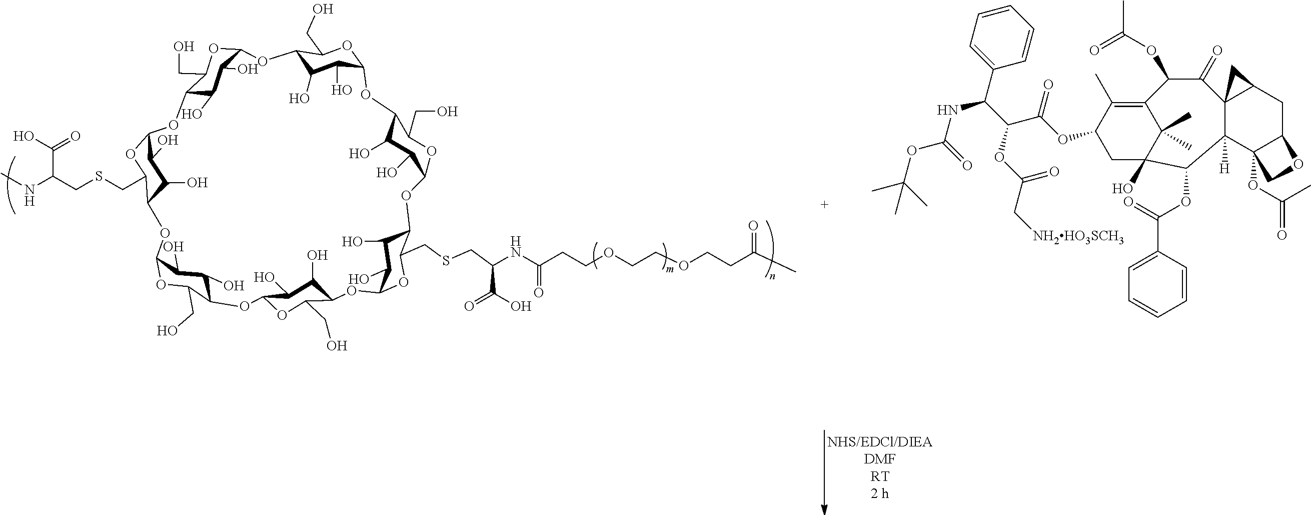

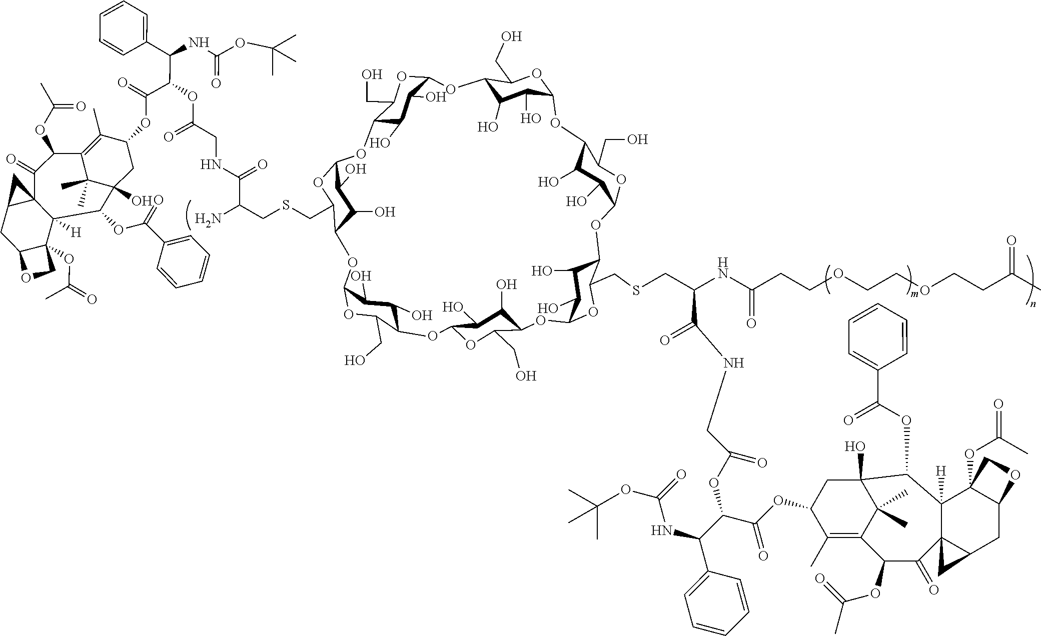

### Example 30

### Synthesis of Larotaxel β-Alanine Glycolate

N-Cbz-β-alanine (15.0 g, 67.3 mmol), tert-butyl bromoacetate (13.1 g, 67.3 mmol), acetone (300 mL), and K₂CO₃ (14 g, 100 mmol) was added to a 1000 mL round bottom flask equipped with a magnetic stirrer. The mixture was heated to reflux (60° C.) for 16 h. The mixture was cooled to ambient temperature and the solid was filtered. The filtrate was concentrated to a residue, dissolved in EtOAc (300 mL), and washed with water (3×100 mL) and brine (100 mL). The organic layer was separated, dried over Na₂SO₄, and filtered. The filtrate was concentrated to produce a clear oil, tert-butyl N-Cbz-β-alanine glycolate (22.2 g, yield: 99%) with 97.4% purity.

A 100 mL round-bottom flask equipped with a magnetic stirrer was charged with tert-butyl N-Cbz-β3-alanine glycolate (7.5 g, 22.2 mmol) and formic acid (35 mL). The mixture was stirred at ambient temperature overnight. The reaction was concentrated under vacuum to a residue and redissolved in EtOAc (7.5 mL). The solution was added to heptanes (150 mL). The product slowly precipitated out to give a white suspension. The mixture was filtered and the filter cake was vacuum-dried at ambient temperature for 24 h to produce the desired product as a white powder, N-Cbz-β-alanine glycolate (5.0 g, yield: 80%) with 98% purity.

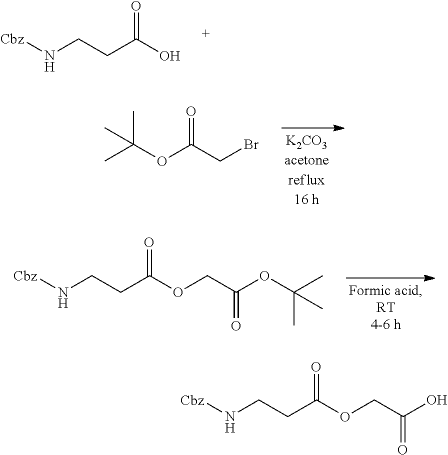

N-Cbz-β-alanine glycolate (1.8 g, 6.5 mmol), DMAP (850 mg, 6.9 mmol) and

EDCI (1.4 g, 7.1 mmol) will be added to a solution of larotaxel (7.2 g, 8.7 mmol) in dichloromethane (140 mL) and the mixture will be stirred at ambient temperature for 2.5 h. N-Cbz-β-alanine glycolate (1.1 g, 3.9 mmol), DMAP (480 mg, 3.9 mmol), and EDCI (1.2 g, 6.1 mmol) will be added and the mixture will be stirred for an additional 2.5 h. The mixture will be washed twice with 1% HCl (2×100 mL) and brine (100 mL). The organics will be dried over sodium sulfate and concentrated under vacuum. The crude product will be purified by column chromatography.

5% Pd/C (2.80 g) will be slurried in 40 mL THF and 4 mL MeOH in a 250 mL flask with overhead stirring. Methanesulfonic acid (0.46 mL, 7.0 mmol) will be added and the slurry will be stirred under hydrogen at ambient temperature for 30 min. A solution of larotaxel Cbz-β-alanine glycolate (8.5 g, 7.7 mmol) in THF (40 mL) will be added (10 mL THF wash). After 2.0 h, the slurry will be filtered (50 mL THF wash) and the filtrate will be concentrated to a minimum volume, diluted with THF (100 mL) and concentrated to about 40 mL. Heptanes (400 mL) will be added dropwise to this mixture over 15 min and stirred 20 min. The resulting slurry will be filtered (100 mL heptanes wash) and the solid will be dried under vacuum to yield larotaxel β-alanine glycolate.

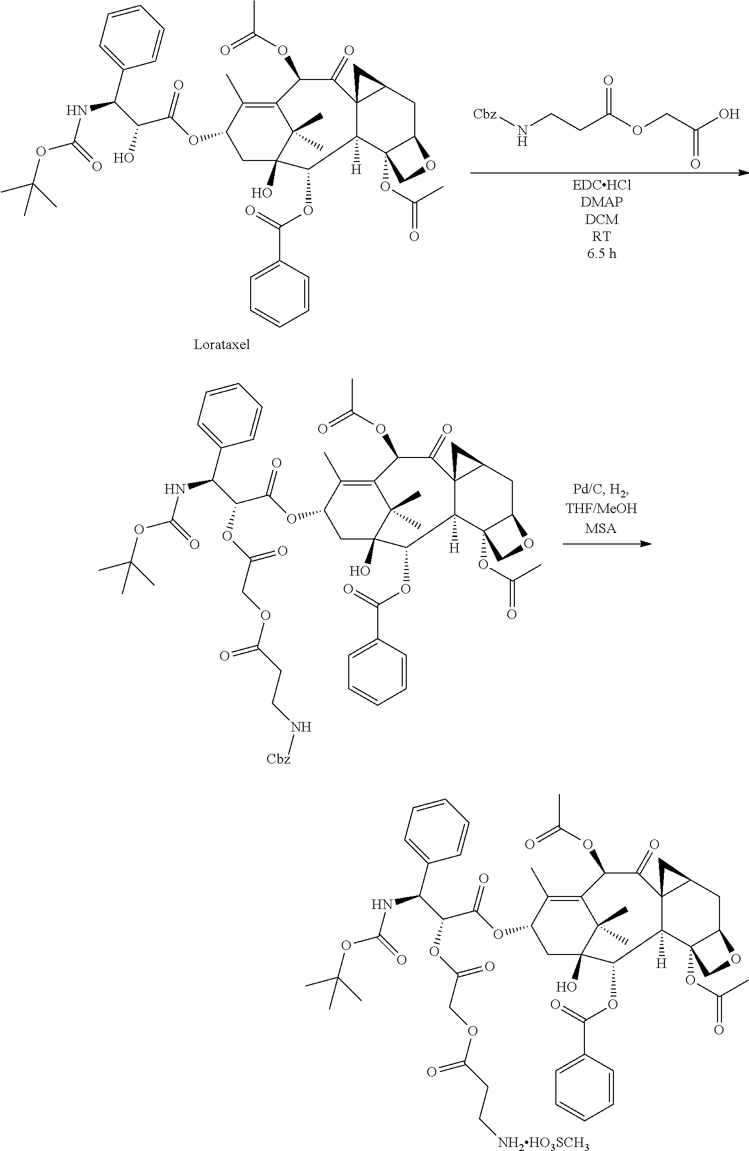

### Example 31

### Synthesis of CDP Larotaxel β-alanine Glycolate

CDP (1.0 g, 0.21 mmol) will be dissolved in dry N,N-dimethylformamide (DMF, 10 mL). Larotaxel β-alanine glycolate (440 mg, 0.46 mmol), N,N-Diisopropylethylamine (59 mg, 0.46 mmol), N-(3-Dimethylaminopropyl)-N′-ethylcarbodiimide hydrochloride (87 mg, 0.46 mmol), and N-Hydroxysuccinimide (52 mg, 0.46 mmol) will then be added to the polymer solution and stirred for 2 h. The polymer will be precipitated with isopropanol (150 mL) and then rinsed with acetone (100 mL). The precipitate will be dissolved in nanopure water (100 mL). It will be purified by TFF with nanopure water (1 L). Consequently, it will be filtered through 0.2 μm filter and kept frozen.

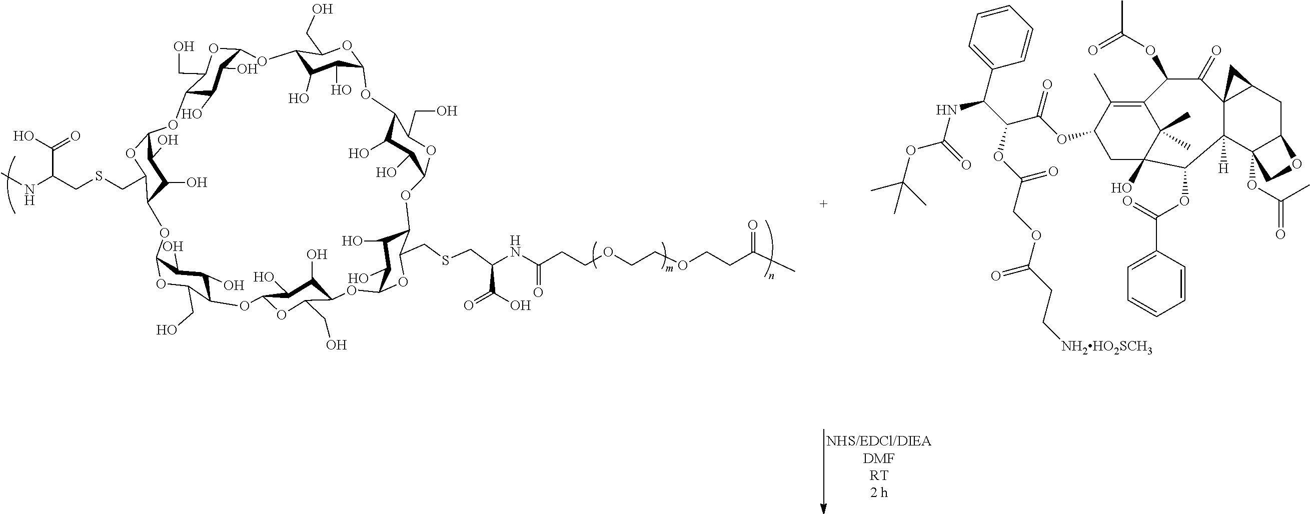

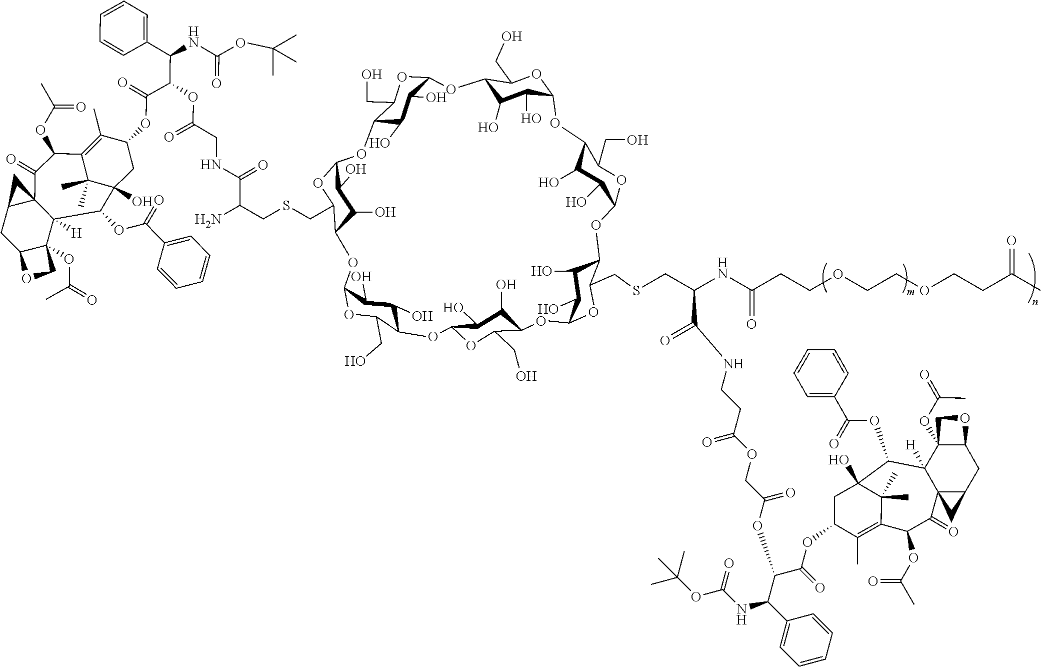

### Example 32

### Synthesis of Larotaxel Aminoethoxyethoxy Acetate

Cbz-aminoethoxyethoxy acetic acid (3.97 g, 13.3 mmol) will be dissolved in dichloromethane (10 mL). A portion of this solution (9 mL, about 8.6 mmol) will be added to a solution of larotaxel (9.36 g, 11.2 mmol) in dichloromethane (180 mL) at ambient temperature. DMAP (1.23 g, 10.1 mmol) and EDCI (1.94 g, 10.1 mmol) will be added and the mixture will be stirred at ambient temperature for 2.75 h. The remaining solution of Cbz-aminoethoxyethoxy acetic acid (5 mL, about 4.7 mmol), DMAP (830 mg, 6.80 mmol), and EDCI (1.28 g, 6.67 mmol, 0.60 equiv) will be added. The mixture will be stirred for approximately 5 hours, and the mixture will be washed twice with 0.1% HCl (2×100 mL) and brine (100 mL). The organic layer will be dried over sodium sulfate and concentrated to a residue. The crude product will be purified by column chromatography to yield larotaxel Cbz-aminoethoxyethoxy acetate.

5% Pd/C (2.0 g) will be slurried in 25 mL THF in a 250 mL flask with overhead stirring. The slurry will be stirred under hydrogen at ambient temperature for 45 min. A solution of larotaxel Cbz-aminoethoxyethoxy acetate (5.8 g, 5.2 mmol) in THF (25 mL) and MeOH (5 mL) will be added (25 mL THF wash). After 4.25 h, 5.0 g of activated carbon will be added and stirred under nitrogen for 15 min. The slurry will be filtered (25 mL THF wash) and the filtrate will be concentrated to about 20 mL. The solution will be added dropwise into 200 mL heptanes. THF and MeOH will be added until dissolution of the precipitate has occurred. A solvent exchange with THF will be performed and the solution concentrated to about 40 mL. Heptanes (500 mL) will be added dropwise to precipitate out the product. It will be filtered and dried under vacuum to yield the final product, larotaxel aminoethoxyethoxy acetate.

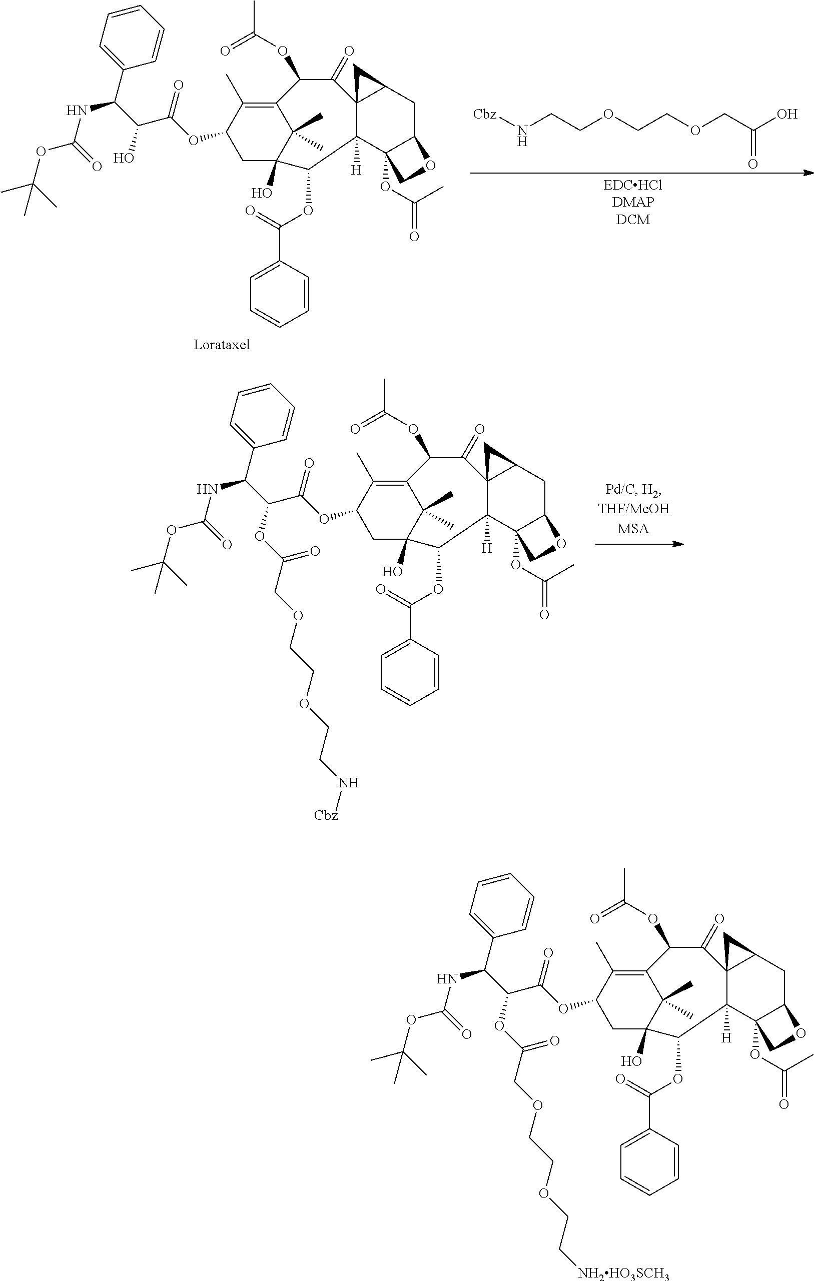

### Example 33

### Synthesis of CDP Larotaxel Aminoethoxyethoxy Acetate

CDP (1.0 g, 0.21 mmol) will be dissolved in dry N,N-dimethylformamide (DMF, 10 mL). Larotaxel aminoethoxyethoxy acetate (440 mg, 0.46 mmol), N,N-Diisopropylethylamine (59 mg, 0.46 mmol), N-(3-Dimethylaminopropyl)-N′-ethylcarbodiimide hydrochloride (87 mg, 0.46 mmol), and N-Hydroxysuccinimide (52 mg, 0.46 mmol) will then be added to the polymer solution and stirred for 2 h. The polymer will be precipitated with isopropanol (150 mL) and then rinsed with acetone (100 mL). The precipitate will be dissolved in nanopure water (100 mL). It will be purified by TFF with nanopure water (1 L). In addition, it will be filtered through 0.2 μm filter and kept frozen.

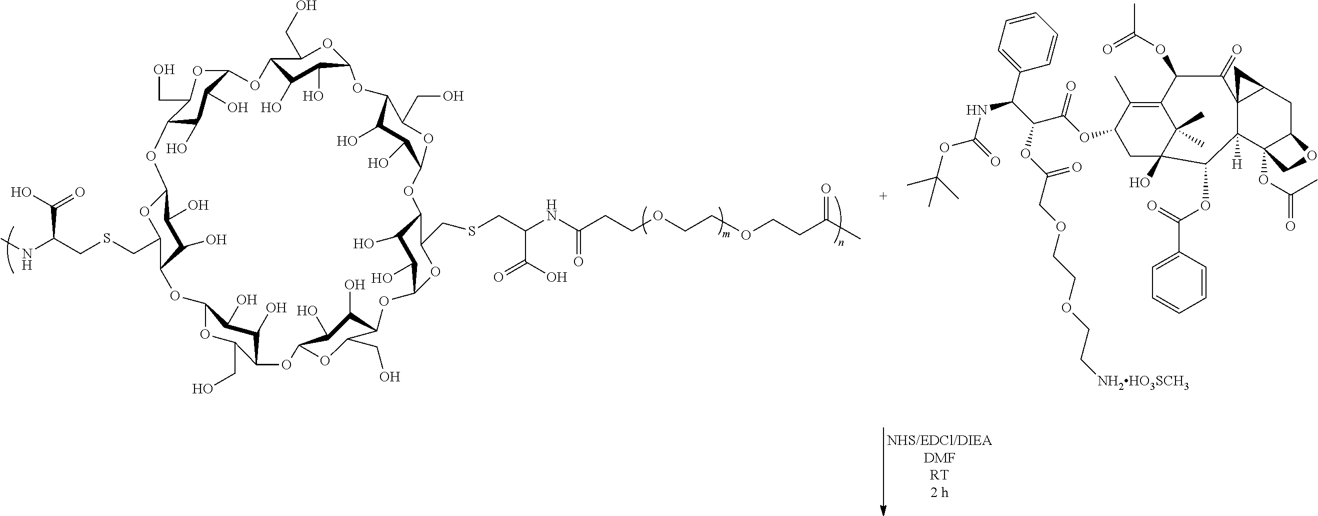

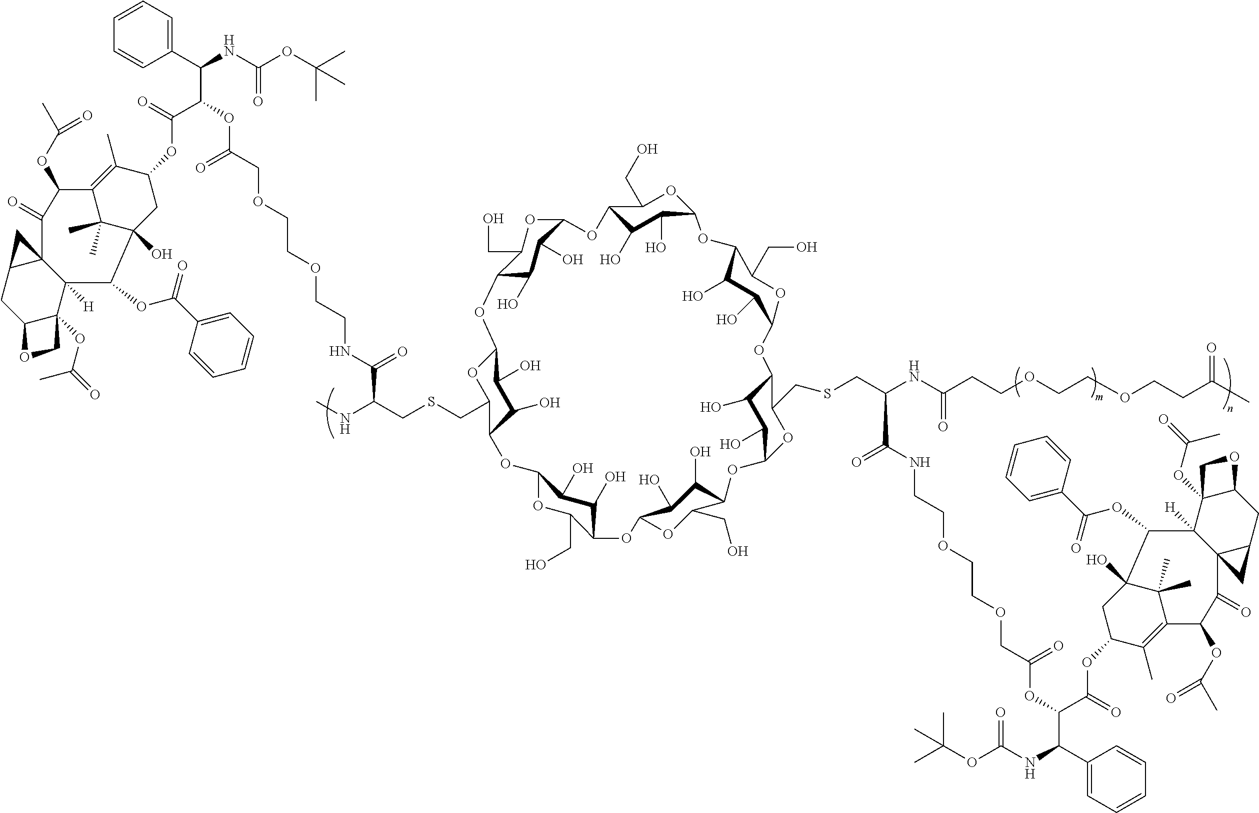

### Example 34

### Synthesis of Larotaxel Aminohexanoate

A 1000 mL, three-neck jacketed reactor equipped with an addition funnel, overhead stirrer, J-KEM probe, and N₂ inlet will be charged with larotaxel (22.3 g, 26.7 mmol), N-Cbz-aminohexanoic acid (7.08 g, 26.7 mmol), DMAP (3.3 g, 26.7 mmol) and DCM (150 mL). The mixture will be stirred for a few minutes to produce a clear solution. It will be cooled from −2 to 2° C. with a TCM. A suspension of EDCI (10.2 g, 53.4 mmol) and DMAP (1.6 g, 13.3 mmol) in DCM (100 mL) will be added dropwise over 2 h. The reaction will be stirred from −2 to 2° C. for 12 h and the temperature will be lowered to −5° C. Additional Cbz-aminohexanoic acid (2.83 g, 10.7 mmol) will be added, followed by addition of EDCI (5.1 g, 26.7 mmol) and DMAP (1.6 g, 13.3 mmol) in DCM (50 mL) over 1 h. The reaction will be stirred at −5° C. for 16 h and then at 0° C. for 4 h, at which time IPC analysis will be done to check for the consumption of larotaxel. Once the reaction completion is confirmed, the reaction mixture will be diluted with DCM to 500 mL and washed with 1% HCl (2×150 mL), saturated NaHCO₃ (2×100 mL) and brine (150 mL). The organic layer will be separated, dried over Na₂SO₄, and filtered. The filtrate will be concentrated to a residue to produce a crude product. Subsequently, the crude product will be purified by column chromatography to yield pure larotaxel Cbz-aminohexanoate.

A 1000 mL round-bottom flask equipped with a magnetic stirrer will be charged with THF (160 mL), methanesulfonic acid (980 μL), and 5% Pd/C (5.9 g). The suspension will be evacuated and back filled with H₂ three times and stirred under H₂ for 0.5 h. A solution of larotaxel Cbz-aminohexanoate (18.4 g, 17.0 mmol) in THF (170 mL) and MeOH (10 mL) will be added. The reaction will be monitored by HPLC. After the reaction is completed, charcoal (10 g) will be added to the reaction and the mixture will be stirred for 10 min and filtered through a Celite pad to produce a clear solution. It will be concentrated to ˜50 mL, to which heptanes (500 mL) will be added to precipitate out the product. It will then be dried under vacuum to yield larotaxel aminohexanoate.

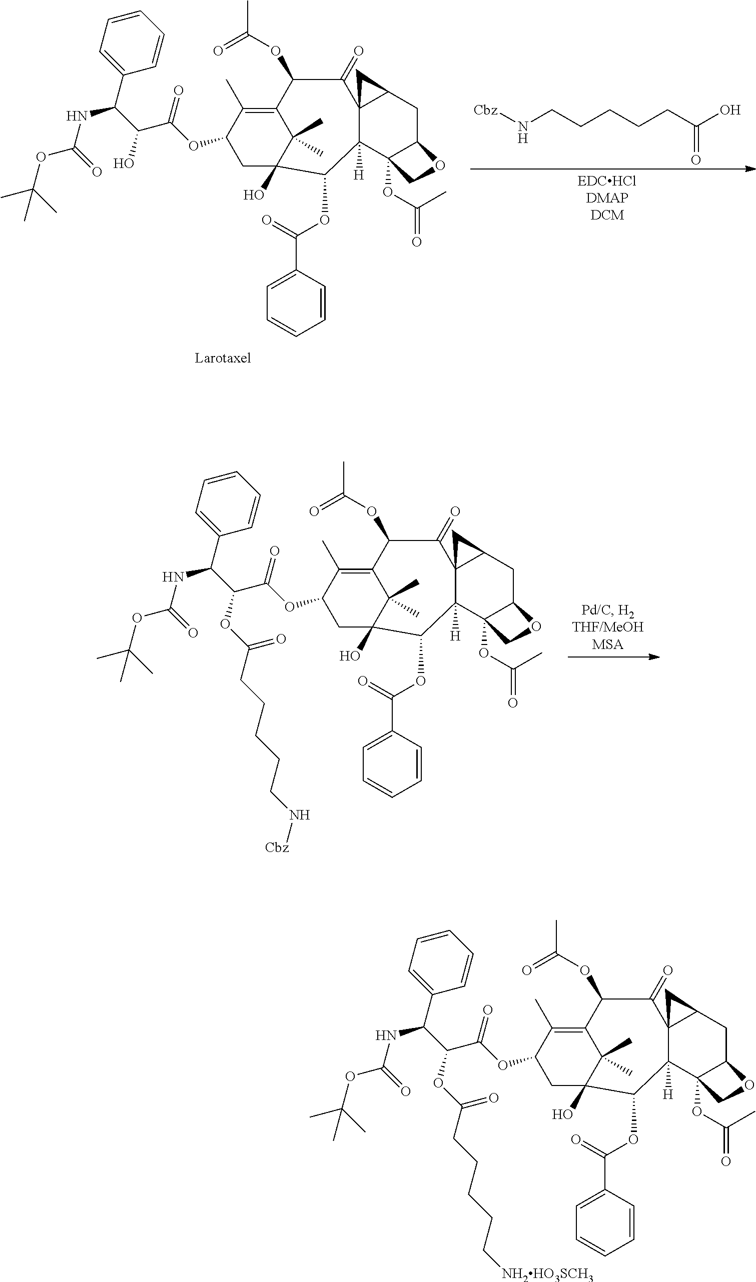

### Example 35

### Synthesis of CDP Larotaxel Aminohexanoate Conjugate

CDP (1.0 g, 0.21 mmol) will be dissolved in dry N,N-dimethylformamide (DMF, 10 mL). Larotaxel aminohexanoate (430 mg, 0.46 mmol), N,N-Diisopropylethylamine (59 mg, 0.46 mmol), N-(3-Dimethylaminopropyl)-N′-ethylcarbodiimide hydrochloride (87 mg, 0.46 mmol), and N-Hydroxysuccinimide (52 mg, 0.46 mmol) will then be added to the polymer solution and stirred for 2 h. The polymer will be precipitated with isopropanol (150 mL) and then rinsed with acetone (100 mL). The precipitate will be dissolved in nanopure water (100 mL). Then it will be purified by TFF with nanopure water (1 L). Followed by filtration through a 0.2 μm filter and kept frozen.

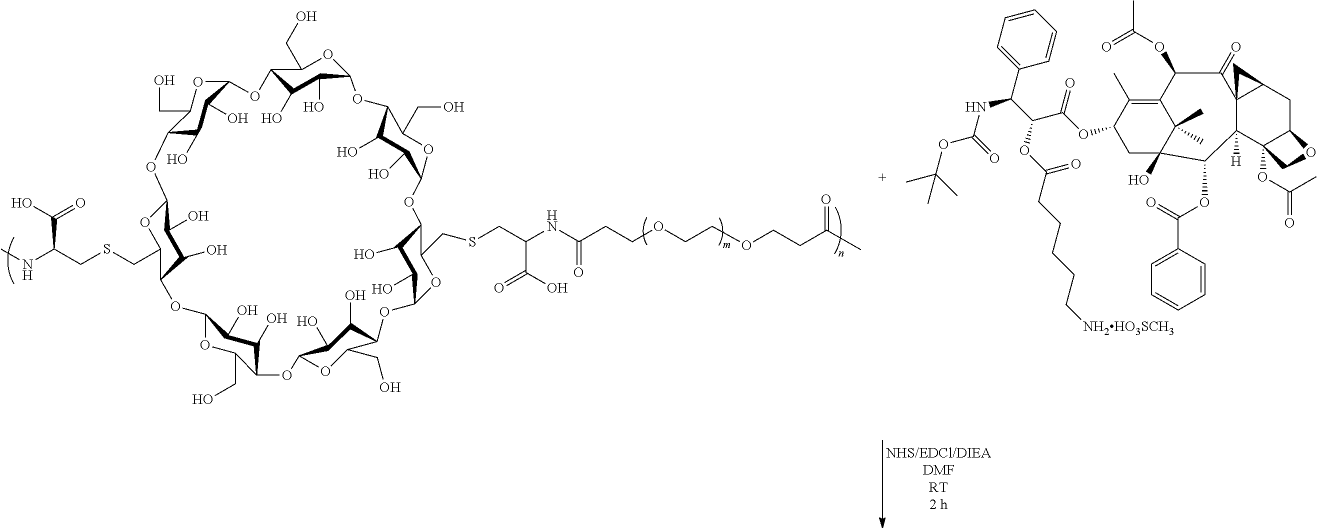

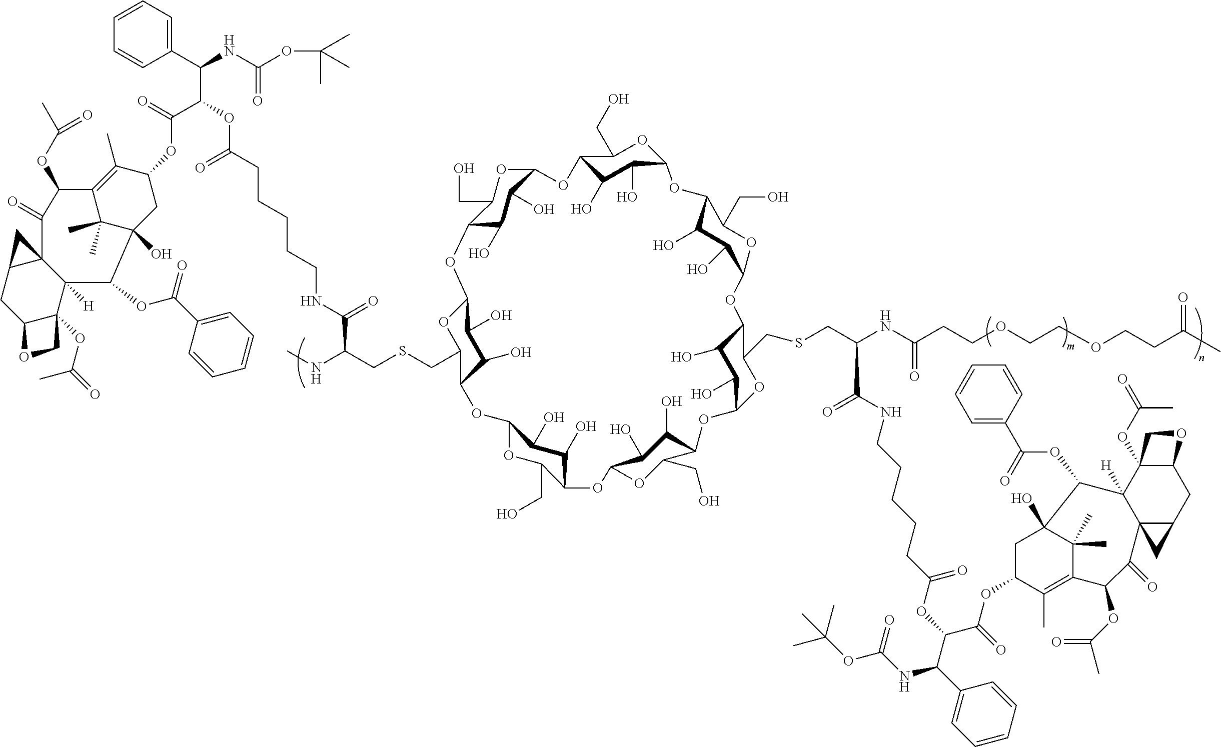

### Example 36

### Synthesis of Larotaxel Aminoethyldithioethyl Carbonate

Triethylamine (15.0 mL, 108 mmol) was added to a mixture of cystamine.2HCl (5.00 g, 22.2 mmol) and MMTCl (14.1 g, 45.6 mmol, 2.05 equiv) in CH₂Cl₂ (200 mL) at ambient temperature. The mixture was stirred for 90 h and 200 mL of 25% saturated NaHCO₃ was added, stirred for 30 min, and removed. The mixture was washed with brine (200 mL) and concentrated to produce a brown oil (19.1 g). The oil was dissolved in 20-25 mL CH₂Cl₂ and purified by flash chromatography to yield a white foam (diMMT-cyteamine, 12.2 g, Yield: 79%)

Bis(2-hydroxyethyldisulfide) (11.5 mL, 94 mmol, 5.4 equiv) and 2-mercaptoethanol (1.25 mL, 17.8 mmol, 1.02 equiv) were added to a solution of diMMT-cyteamine (12.2 g, 17.5 mmol) in 1:1 CH₂Cl₂/MeOH (60 mL) and the mixture was stirred at ambient temperature for 42.5 h. The mixture was concentrated to an oil, dissolved in EtOAc (150 mL), washed with 10% saturated NaHCO3 (3·150 mL) and brine (150 mL), dried over Na2SO4, and concentrated to an oil (16.4 g). The oil was dissolved in 20 mL CH₂Cl₂ and purified by flash chromatography to yield clear thick oil (MMT-aminoethyldithioethanol, 5.33 g, Yield: 36%).

A 250 mL round bottom flask equipped with a magnetic stirrer was charged with MMT-aminoethyldithioethanol (3.6 g, 8.5 mmol) and acetonitrile (60 mL). Disuccinimidyl carbonate (2.6 g) was added and the reaction was stirred at ambient temperature for 3 h. It will be used for the next reaction without isolation. Succinimidyl MMT-aminoethyldithioethyl carbonate from Scheme 9(a) will be transferred to a cooled solution of larotaxel (6.36 g, 7.61 mmol) and DMAP (1.03 g) in DCM (60 mL) at 0-5° C. with stirring for 16 h. It will be then purified by column chromatography.

A 1000 mL round bottom flask equipped with a magnetic stirrer will be charged with larotaxel Cbz-aminoethyldithioethyl carbonate (12.6 g) and DCM (300 mL). Anisole (10.9 mL, 10 equiv.) will be added to this clear solution and stirred for a few minutes. Dichloroacetic acid (8.3 mL, 10 equiv.) will be added over 5 min and the reaction will be stirred at ambient temperature for 1 h. The mixture will be concentrated down to ˜100 mL, to which heptanes (800 mL) will be slowly added resulting in a suspension. The suspension will be stirred for 15 min and the supernatant will be decanted. The orange residue will be washed with heptanes (200 mL) and vacuum-dried at ambient temperature for 1 h. THF (30 mL) will be added to dissolve the orange residue producing a red solution. Heptanes (500 mL) will be slowly added to precipitate out the product. The resulting suspension will be stirred at ambient temperature for 1 h and filtered. The filter cake will be washed with heptanes (300 mL) and dried under vacuum to yield larotaxel aminoethyldithioethyl carbonate.

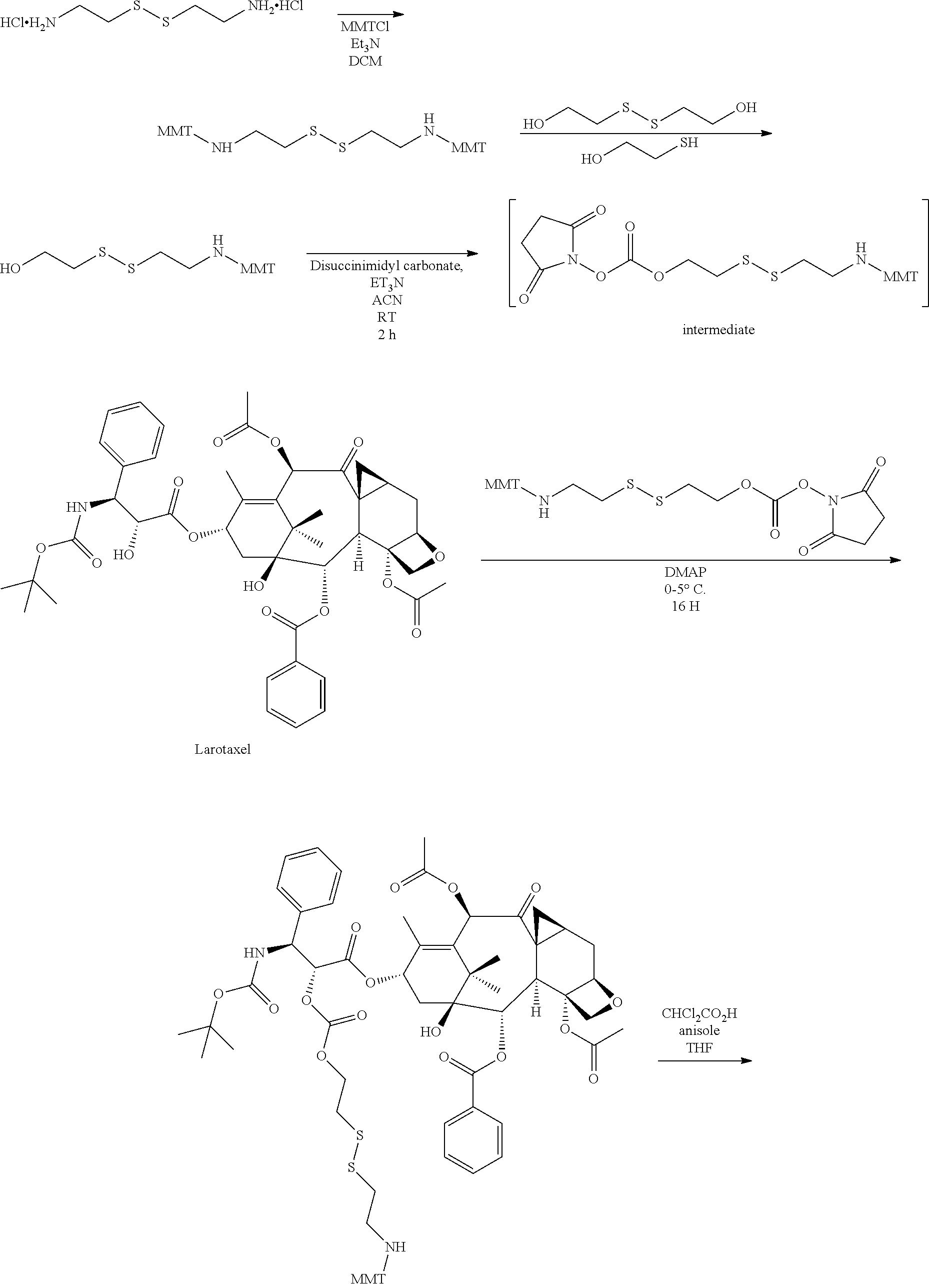

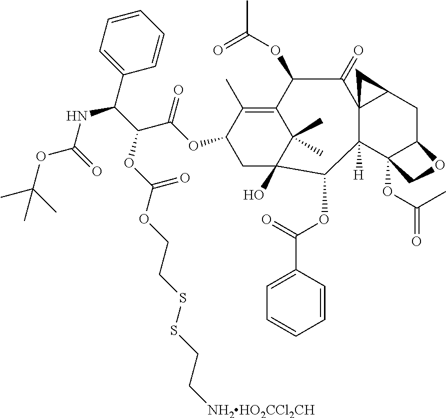

### Example 37

### Synthesis of CDP Larotaxel Aminoethyldithioethyl Carbonate

CDP (1.0 g, 0.21 mmol) will be dissolved in dry N,N-dimethylformamide (DMF, 10 mL). Larotaxel aminoethyldithioethyl carbonate (460 mg, 0.46 mmol), N,N-Diisopropylethylamine (59 mg, 0.46 mmol), N-(3-Dimethylaminopropyl)-N′-ethylcarbodiimide hydrochloride (87 mg, 0.46 mmol), and N-Hydroxysuccinimide (52 mg, 0.46 mmol) will then be added to the polymer solution and stirred for 2 h. The polymer will be precipitated with isopropanol (150 mL) and then rinsed with acetone (100 mL). The precipitate will be dissolved in nanopure water (100 mL). It will be purified by TFF with nanopure water (1 L). It will then be filtered through 0.2 μm filter and kept frozen.

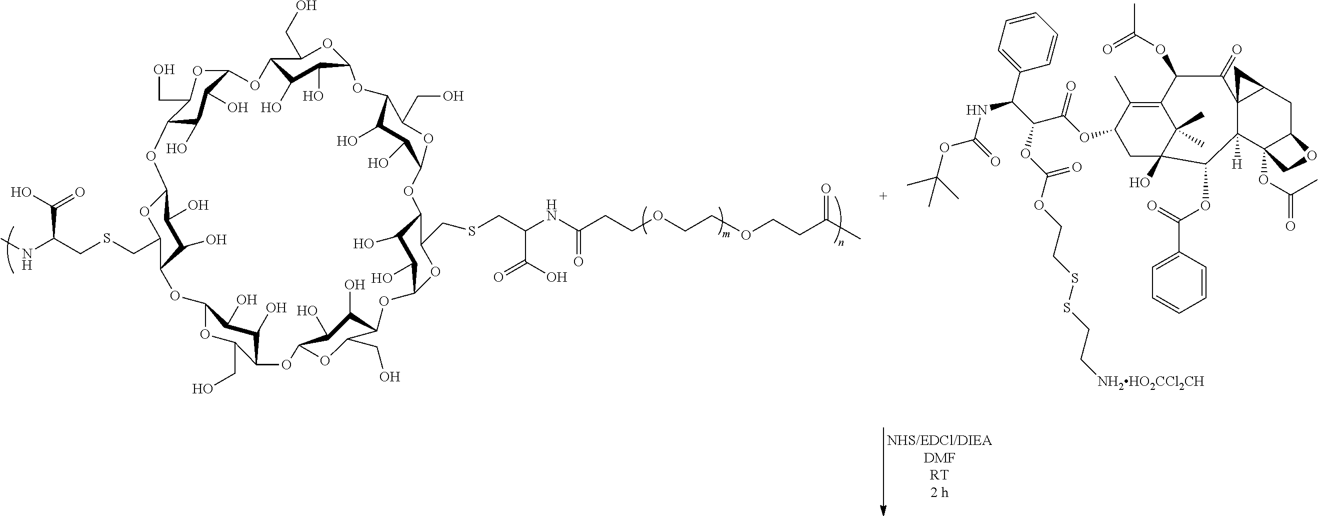

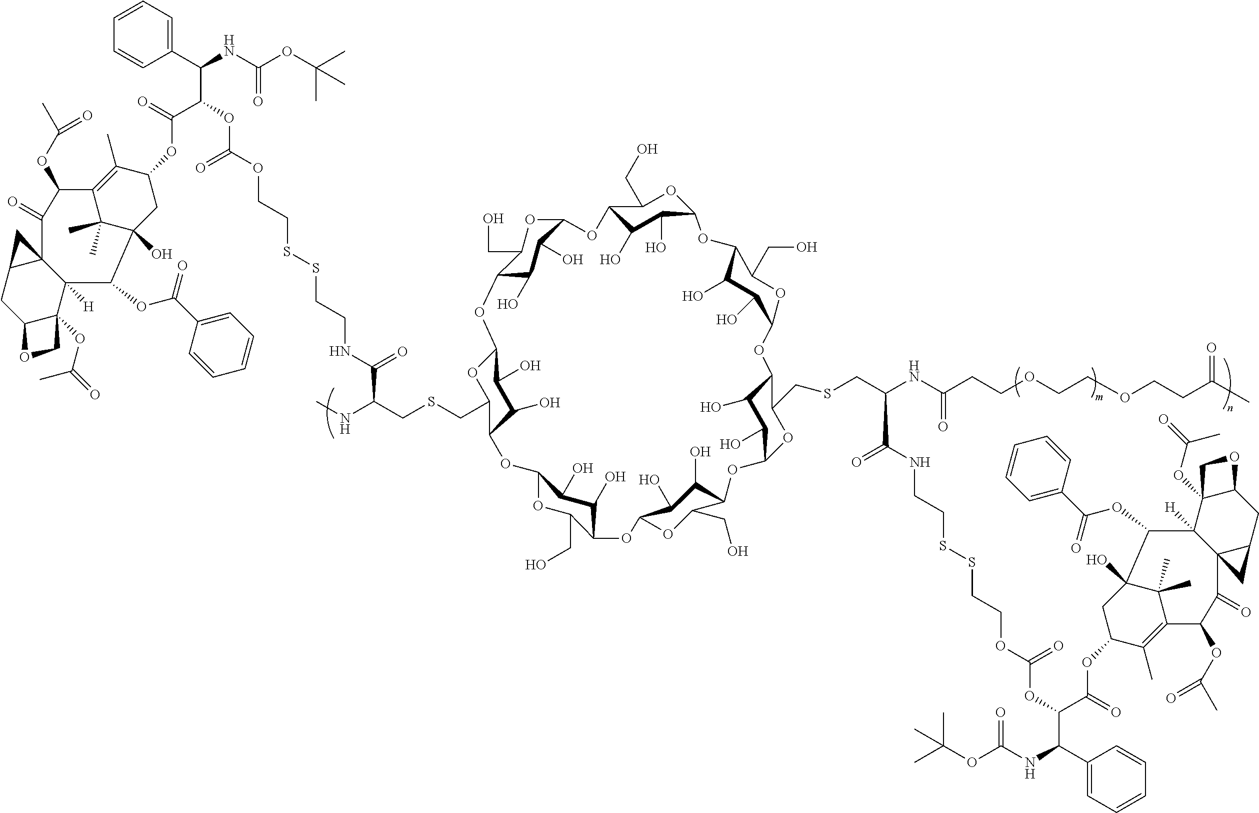

### Example 38

### Synthesis of Cabazitaxel Glycinate

A 1000 mL, three-neck jacketed reactor equipped with an addition funnel, overhead stirrer, J-KEM probe, and N₂ inlet will be charged with cabazitaxel (22.3 g, 26.7 mmol), N-Cbz-glycine (5.6 g, 26.7 mmol), DMAP (3.3 g, 26.7 mmol) and DCM (150 mL). The mixture will be stirred for a few minutes to produce a clear solution. It will be cooled from −2 to 2° C. with a TCM. A suspension of EDCI (10.2 g, 53.4 mmol) and DMAP (1.6 g, 13.3 mmol) in DCM (100 mL) will be added dropwise over 2 h. The reaction will be stirred at −2 to 2° C. for 12 h and the temperature will be lowered to −5° C. Additional N-Cbz-glycine (2.2 g, 10.7 mmol) will be added, followed by addition of EDCI (5.1 g, 26.7 mmol) and DMAP (1.6 g, 13.3 mmol) in DCM (50 mL) over 1 h. The reaction will be stirred at −5° C. for 16 h and then at 0° C. for 4 h, at which time IPC analysis will be done to check for the consumption of cabazitaxel. Once the reaction completion is confirmed, the reaction mixture will be diluted with DCM to 500 mL and washed with 1% HCl (2×150 mL), saturated NaHCO₃ (2×100 mL) and brine (150 mL). The organic layer will be separated, dried over Na₂SO₄, and filtered. The filtrate will be concentrated to a residue to produce a crude product. The crude product will then be purified by column chromatography to yield pure cabazitaxel Cbz-glycinate.

A 1000 mL round-bottom flask equipped with a magnetic stirrer will be charged with THF (160 mL), MSA (980 μL), and 5% Pd/C (5.9 g). The suspension will be evacuated and back filled with H₂ three times and stirred under H₂ for 0.5 h. A solution of cabazitaxel Cbz-glycinate (17.5 g, 17.0 mmol) in THF (170 mL) and MeOH (10 mL) will be added. The reaction will be monitored by HPLC. After the reaction is completed, charcoal (10 g) will be added to the reaction and the mixture will be stirred for 10 min and filtered through a Celite pad to produce a clear solution. It will be concentrated to ˜50 mL, to which heptanes (500 mL) will be added to precipitate out the product. It will then be dried under vacuum to yield cab azitaxel glycinate.

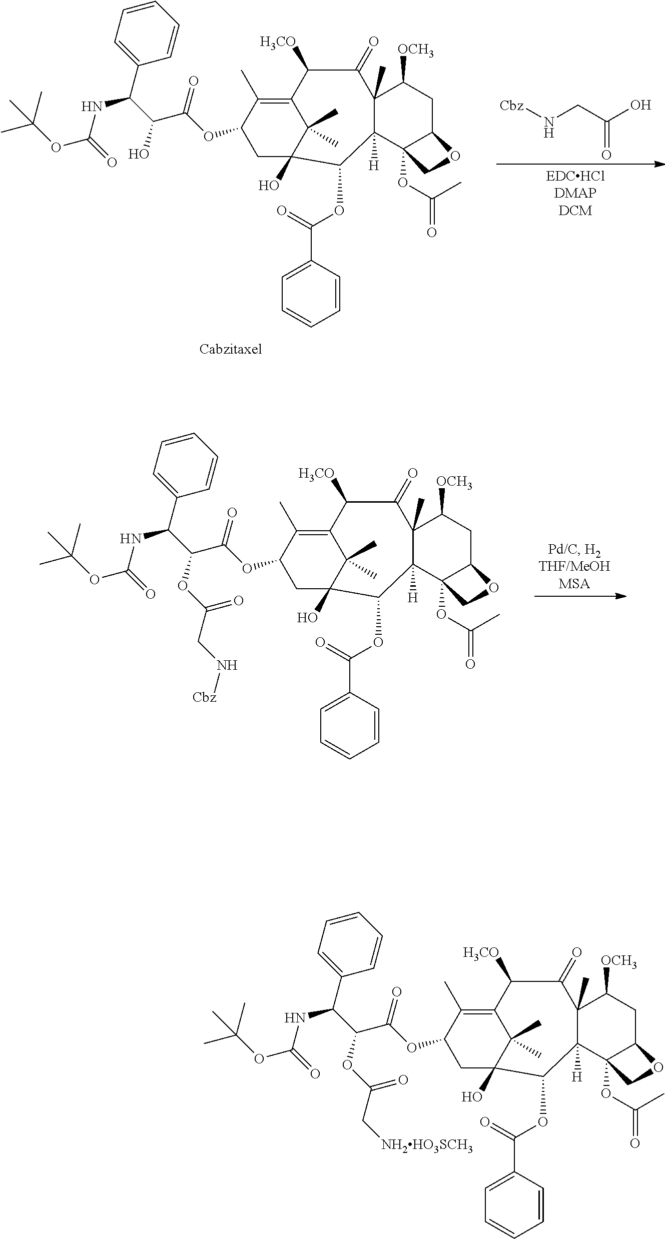

### Example 39

### Synthesis of CDP Cabazitaxel Glycinate Conjugate

CDP (1.0 g, 0.21 mmol) will be dissolved in dry N,N-dimethylformamide (DMF, 10 mL). Cabazitaxel glycinate (400 mg, 0.46 mmol), N,N-Diisopropylethylamine (59 mg, 0.46 mmol), N-(3-Dimethylaminopropyl)-N′-ethylcarbodiimide hydrochloride (87 mg, 0.46 mmol), and N-Hydroxysuccinimide (52 mg, 0.46 mmol) will then be added to the polymer solution and stirred for 2 h. The polymer will be precipitated with isopropanol (150 mL) and then rinsed with acetone (100 mL). The precipitate will be dissolved in nanopure water (100 mL). It will be purified by TFF with nanopure water (1 L). It will then be filtered through 0.2 μm filter and kept frozen.

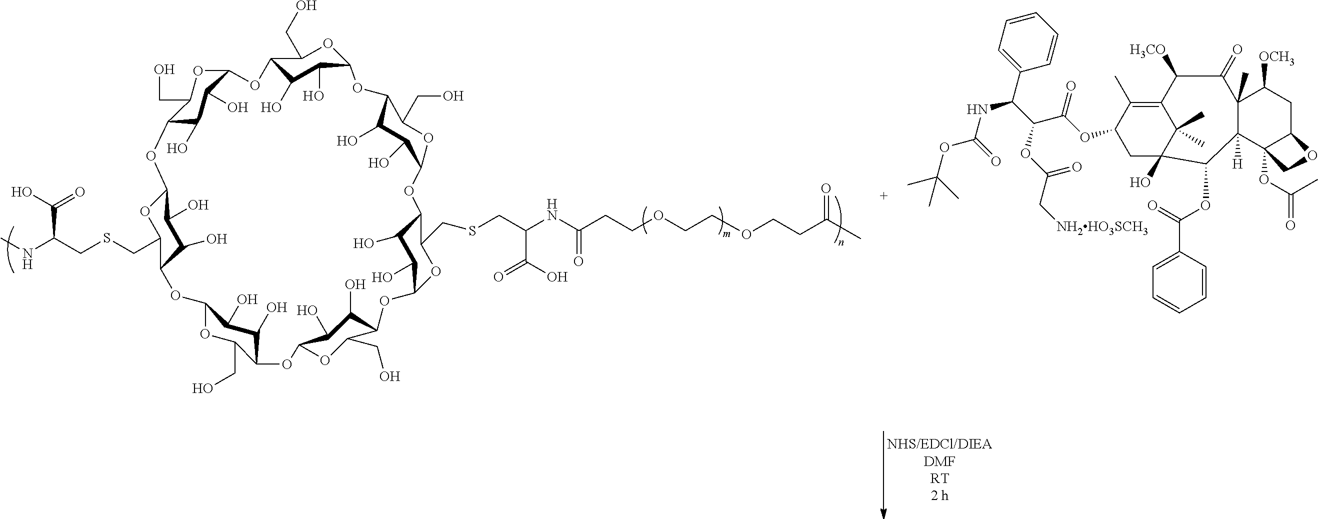

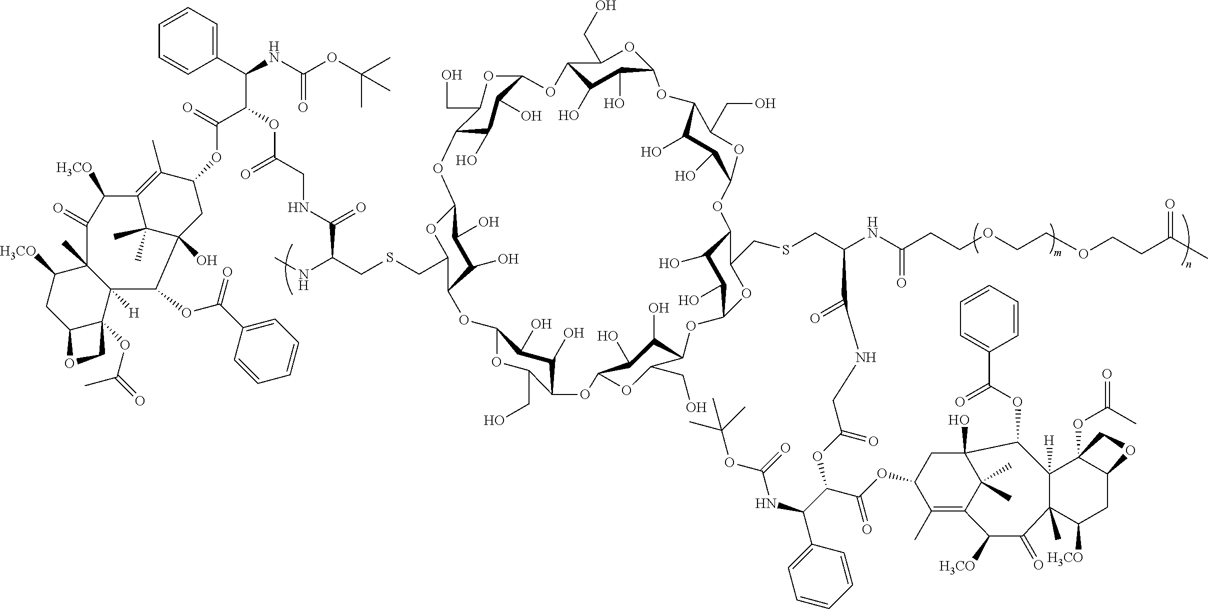

### Example 40

### Synthesis of Cabazitaxel β-alanine Glycolate

N-Cbz-β-alanine glycolate (1.8 g, 6.5 mmol), DMAP (850 mg, 6.9 mmol) and EDCI (1.4 g, 7.1 mmol) will be added to a solution of cabazitaxel (7.2 g, 8.7 mmol) in CH₂Cl₂ (140 mL) and the mixture will be stirred at ambient temperature for 2.5 h. N-Cbz-13-alanine glycolate (1.1 g, 3.9 mmol), DMAP (480 mg, 3.9 mmol), and EDCI (1.2 g, 6.1 mmol) will be added and the mixture was stirred for an additional 2.5 h. The mixture will be washed twice with 1% HCl (2×100 mL) and brine (100 mL). The organics will be dried over sodium sulfate and concentrated under vacuum. The crude product will be purified by column chromatography.

5% Pd/C (2.80 g) will be slurried in 40 mL THF and 4 mL MeOH in a 250 mL flask with overhead stirring. Methanesulfonic acid (0.46 mL, 7.0 mmol) will be added and the slurry will be stirred under hydrogen at ambient temperature for 30 min. A solution of cabazitaxel Cbz-β-alanine glycolate (8.5 g, 7.7 mmol) in THF (40 mL) will be added (10 mL THF wash). After 2.0 h, the slurry will be filtered (50 mL THF wash) and the filtrate will be concentrated to a minimum volume, diluted with THF (100 mL) and concentrated to about 40 mL. Heptanes (400 mL) will be added dropwise to this mixture over 15 min and stirred 20 min. The resulting slurry will be filtered (100 mL heptanes wash) and the solid will be dried under vacuum to yield cabazitaxel β-alanine glycolate.

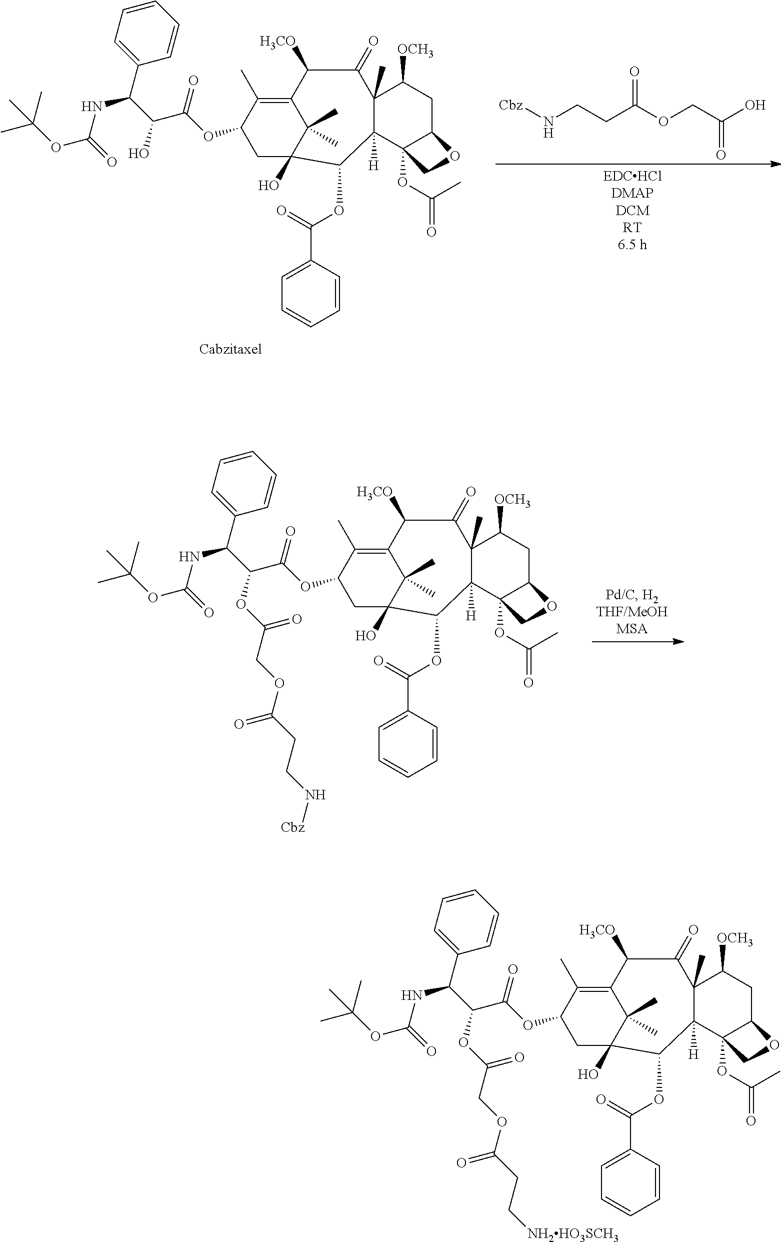

### Example 41

### Synthesis of CDP Cabazitaxel β-alanine Glycolate

CDP (1.0 g, 0.21 mmol) will be dissolved in dry N,N-dimethylformamide (DMF, 10 mL). Cabazitaxel β-alanine glycolate (440 mg, 0.46 mmol), N,N-Diisopropylethylamine (59 mg, 0.46 mmol), N-(3-Dimethylaminopropyl)-N′-ethylcarbodiimide hydrochloride (87 mg, 0.46 mmol), and N-Hydroxysuccinimide (52 mg, 0.46 mmol) will then be added to the polymer solution and stirred for 2 h. The polymer will be precipitated with isopropanol (150 mL) and then rinsed with acetone (100 mL). The precipitate will be dissolved in nanopure water (100 mL). It will be purified by TFF with nanopure water (1 L). It will then be filtered through 0.2 μm filter and kept frozen.

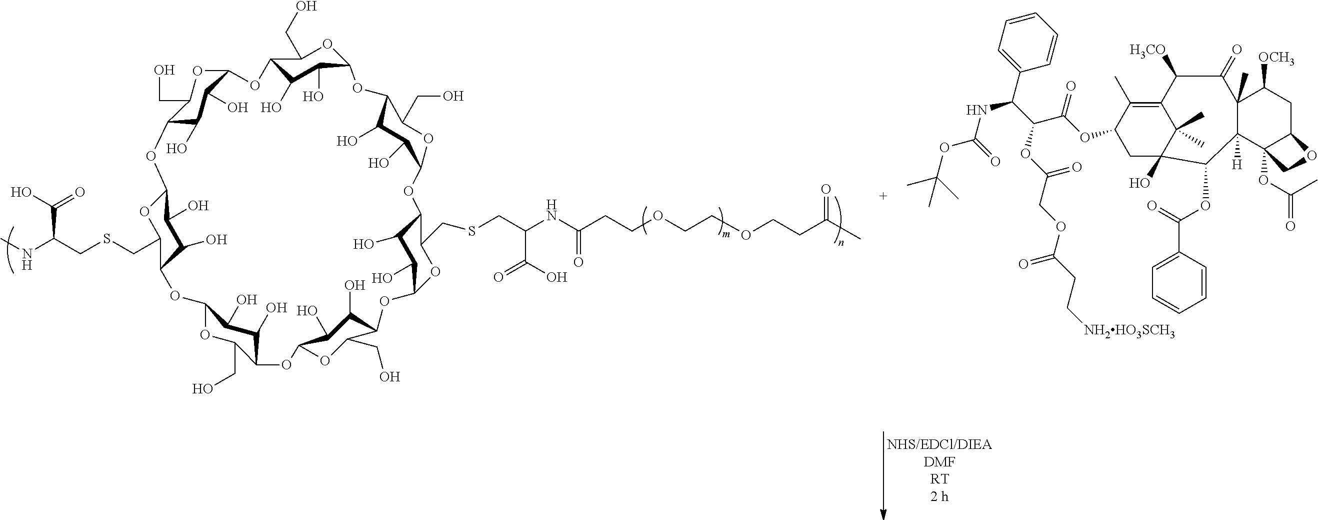

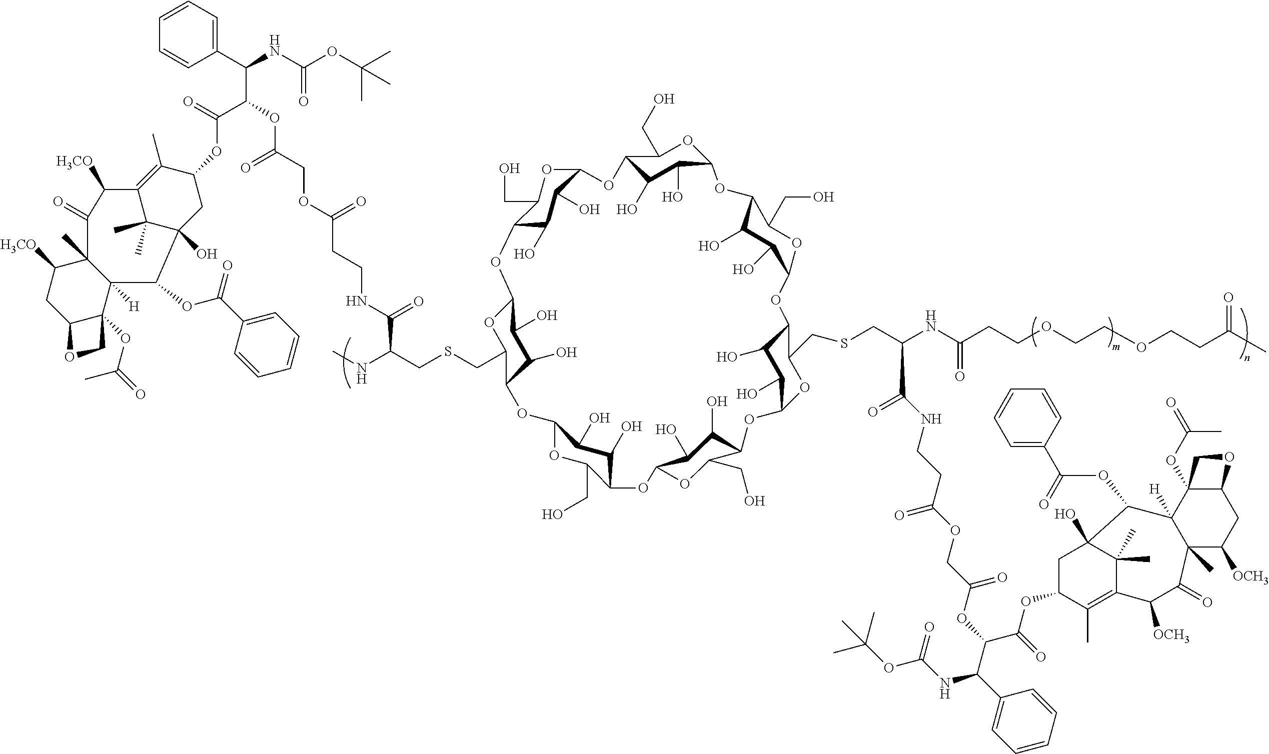

### Example 42

### Synthesis of Cabazitaxel Aminoethoxyethoxy Acetate

Cbz-aminoethoxyethoxy acetic acid (3.97 g, 13.3 mmol) will be dissolved in dichloromethane (10 mL). A portion of this solution (9 mL, about 8.6 mmol) will be added to a solution of cabazitaxel (9.36 g, 11.2 mmol) in CH₂Cl₂ (180 mL) at ambient temperature. DMAP (1.23 g, 10.1 mmol) and EDCI (1.94 g, 10.1 mmol) will be added and the mixture will be stirred at ambient temperature for 2.75 h. The remaining solution of Cbz-aminoethoxyethoxy acetic acid (5 mL, about 4.7 mmol), DMAP (830 mg, 6.80 mmol), and EDCI (1.28 g, 6.67 mmol, 0.60 equiv) will be added. The mixture will be stirred for an additional 4.75 h, and the mixture will be washed twice with 0.1% HCl (2×100 mL) and brine (100 mL). The organic layer will be dried over sodium sulfate and concentrated to a residue. The crude product will be purified by column chromatography to yield cabazitaxel Cbz-aminoethoxyethoxy acetate.

5% Pd/C (2.0 g) will be slurried in 25 mL THF in a 250 mL flask with overhead stirring. The slurry will be stirred under hydrogen at ambient temperature for 45 min. A solution of cabazitaxel Cbz-aminoethoxyethoxy acetate (5.8 g, 5.2 mmol) in THF (25 mL) and MeOH (5 mL) will be added (25 mL THF wash). After 4.25 h, 5.0 g of activated carbon will be added and stirred under nitrogen for 15 min. The slurry will be filtered (25 mL THF wash) and the filtrate will be concentrated to about 20 mL. The solution will be added dropwise into 200 mL heptanes. THF and MeOH will be added until dissolution of the precipitate has occurred. A solvent exchange with THF will be performed and concentrated to about 40 mL. Heptanes (500 mL) will be added dropwise to precipitate out the product. It will be filtered and dried under vacuum to yield the final product, cabazitaxel aminoethoxyethoxy acetate.

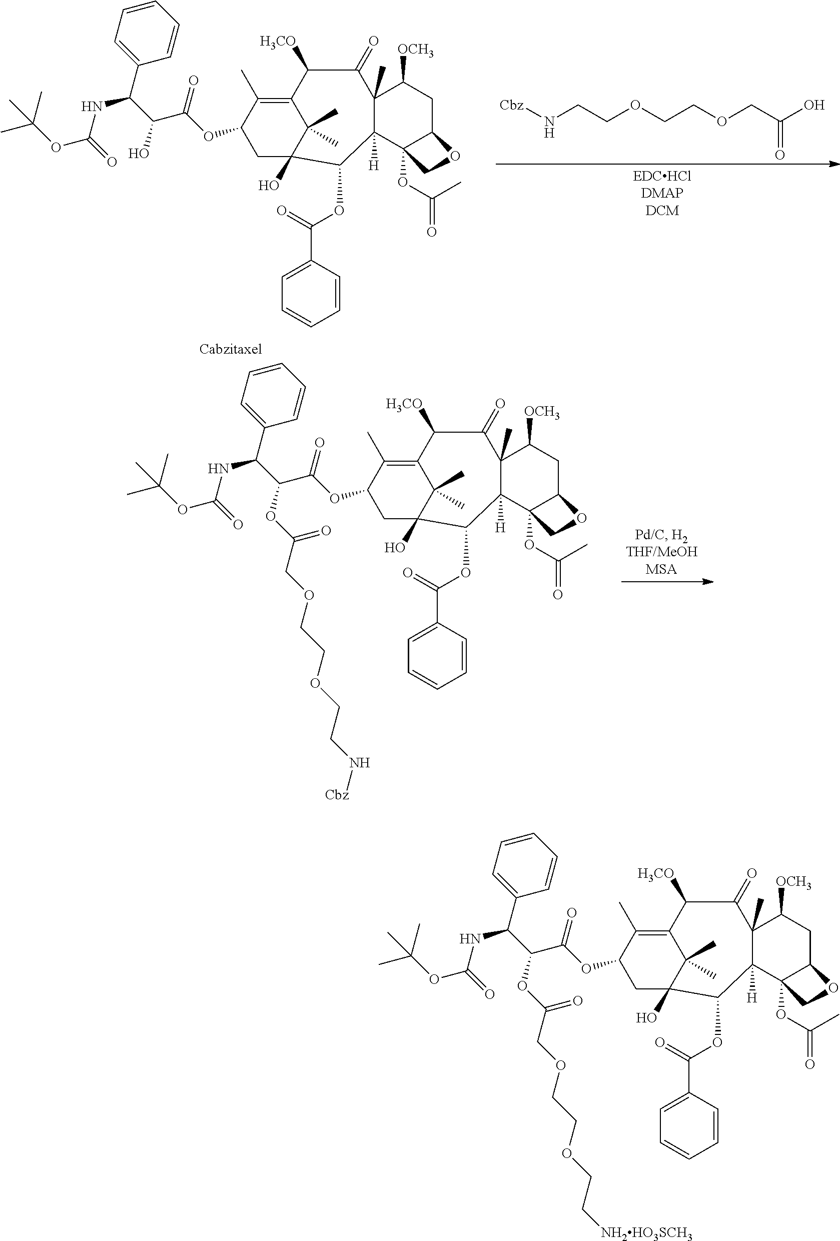

### Example 43

### Synthesis of CDP Cabazitaxel Aminoethoxyethoxy Acetate

CDP (1.0 g, 0.21 mmol) will be dissolved in dry N,N-dimethylformamide (DMF, 10 mL). Cabazitaxel aminoethoxyethoxy acetate (440 mg, 0.46 mmol), N,N-Diisopropylethylamine (59 mg, 0.46 mmol), N-(3-Dimethylaminopropyl)-N′-ethylcarbodiimide hydrochloride (87 mg, 0.46 mmol), and N-Hydroxysuccinimide (52 mg, 0.46 mmol) will then be added to the polymer solution and stirred for 2 h. The polymer will be precipitated with isopropanol (150 mL) and then rinsed with acetone (100 mL). The precipitate will be dissolved in nanopure water (100 mL). It will be purified by TFF with nanopure water (1 L). It will then be filtered through 0.2 μm filter and kept frozen.

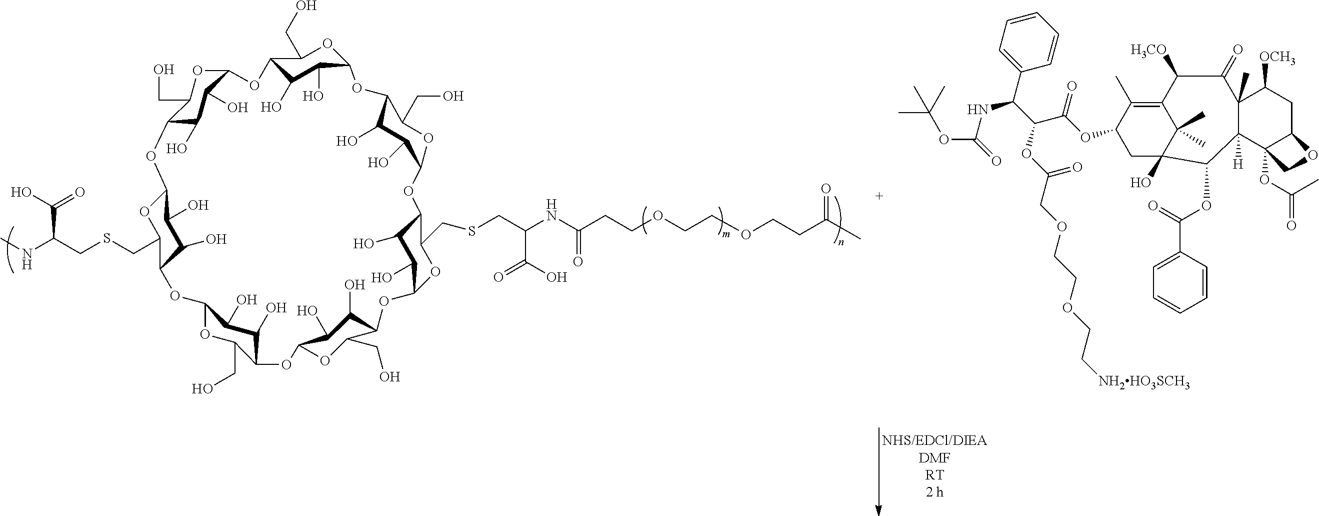

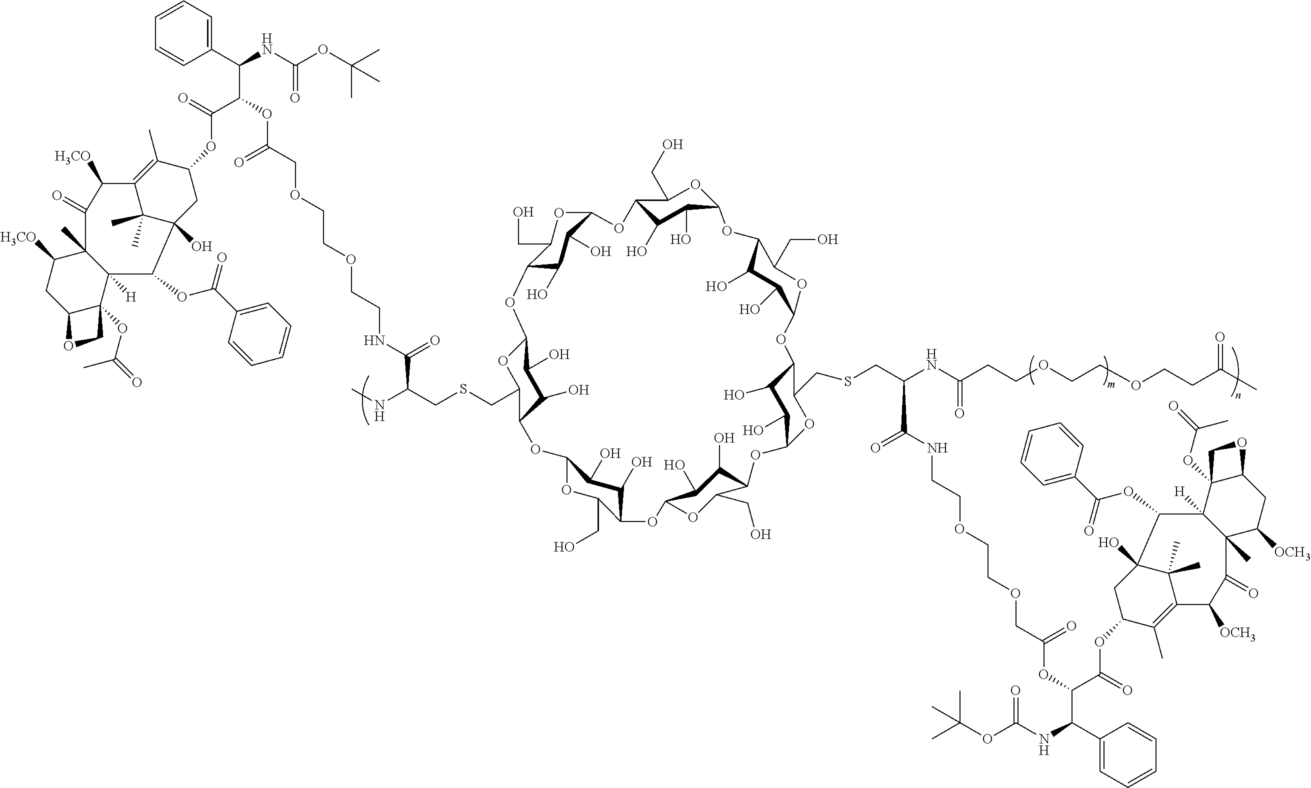

### Example 44

### Synthesis of Cabazitaxel Aminohexanoate

A 1000 mL, three-neck jacketed reactor equipped with an addition funnel, overhead stirrer, J-KEM probe, and N₂ inlet will be charged with cabazitaxel (22.3 g, 26.7 mmol), N-Cbz-aminohexanoic acid (7.08 g, 26.7 mmol), DMAP (3.3 g, 26.7 mmol) and DCM (150 mL). The mixture will be stirred for a few minutes to produce a clear solution. It will be cooled from −2 to 2° C. with a TCM. A suspension of EDCI (10.2 g, 53.4 mmol) and DMAP (1.6 g, 13.3 mmol) in DCM (100 mL) will be added dropwise over 2 h. The reaction will be stirred from −2 to 2° C. for 12 h and the temperature will be lowered to −5° C. Additional Cbz-aminohexanoic acid (2.83 g, 10.7 mmol) will be added, followed by addition of EDCI (5.1 g, 26.7 mmol) and DMAP (1.6 g, 13.3 mmol) in DCM (50 mL) over 1 h. The reaction will be stirred at −5° C. for 16 h and then at 0° C. for 4 h, at which time IPC analysis will be done to check for the consumption of cabazitaxel. Once the reaction completion is confirmed, the reaction mixture will be diluted with DCM to 500 mL and washed with 1% HCl (2×150 mL), saturated NaHCO₃ (2×100 mL) and brine (150 mL). The organic layer will be separated, dried over Na₂SO₄, and filtered. The filtrate will be concentrated to a residue to produce a crude product. The crude product will then be purified by column chromatography to yield pure cabazitaxel Cbz-aminohexanoate.

A 1000 mL round-bottom flask equipped with a magnetic stirrer will be charged with THF (160 mL), methanesulfonic acid (980 μL), and 5% Pd/C (5.9 g). The suspension will be evacuated and back filled with H₂ three times and stirred under H₂ for 0.5 h. A solution of cabazitaxel Cbz-aminohexanoate (18.4 g, 17.0 mmol) in THF (170 mL) and MeOH (10 mL) will be added. The reaction will be monitored by HPLC. After the reaction is completed, charcoal (10 g) will be added to the reaction and the mixture will be stirred for 10 min and filtered through a Celite pad to produce a clear solution. It will be concentrated to ˜50 mL, to which heptanes (500 mL) will be added to precipitate out the product. It will then be dried under vacuum to yield cabazitaxel aminohexanoate.

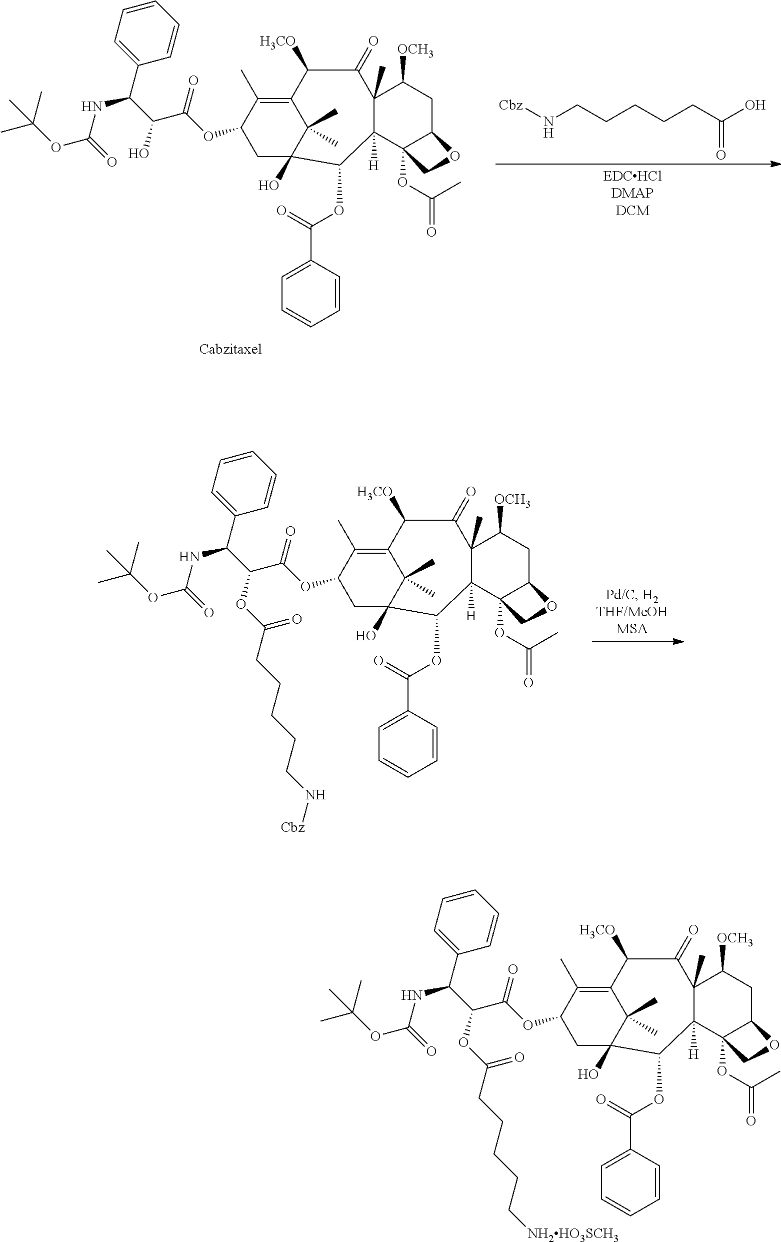

### Example 45

### Synthesis of CDP Cabazitaxel Aminohexanoate Conjugate

CDP (1.0 g, 0.21 mmol) will be dissolved in dry N,N-dimethylformamide (DMF, 10 mL). Cabazitaxel aminohexanoate (430 mg, 0.46 mmol), N,N-Diisopropylethylamine (59 mg, 0.46 mmol), N-(3-Dimethylaminopropyl)-N′-ethylcarbodiimide hydrochloride (87 mg, 0.46 mmol), and N-Hydroxysuccinimide (52 mg, 0.46 mmol) will then be added to the polymer solution and stirred for 2 h. The polymer will be precipitated with isopropanol (150 mL) and then rinsed with acetone (100 mL). The precipitate will be dissolved in nanopure water (100 mL). It will be purified by TFF with nanopure water (1 L). It will then be filtered through 0.2 μm filter and kept frozen.

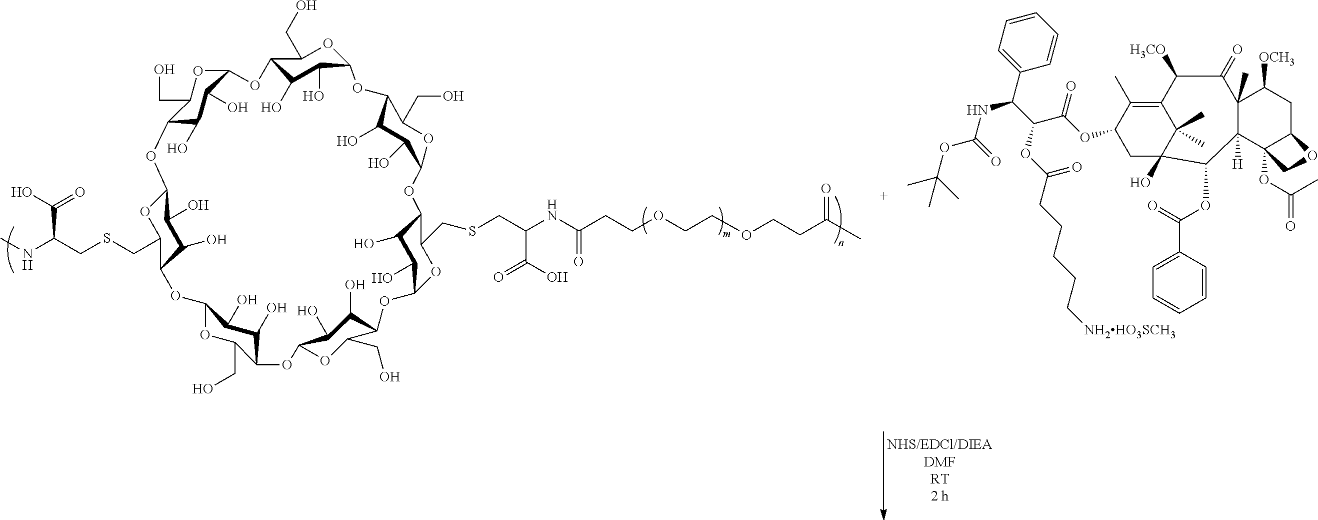

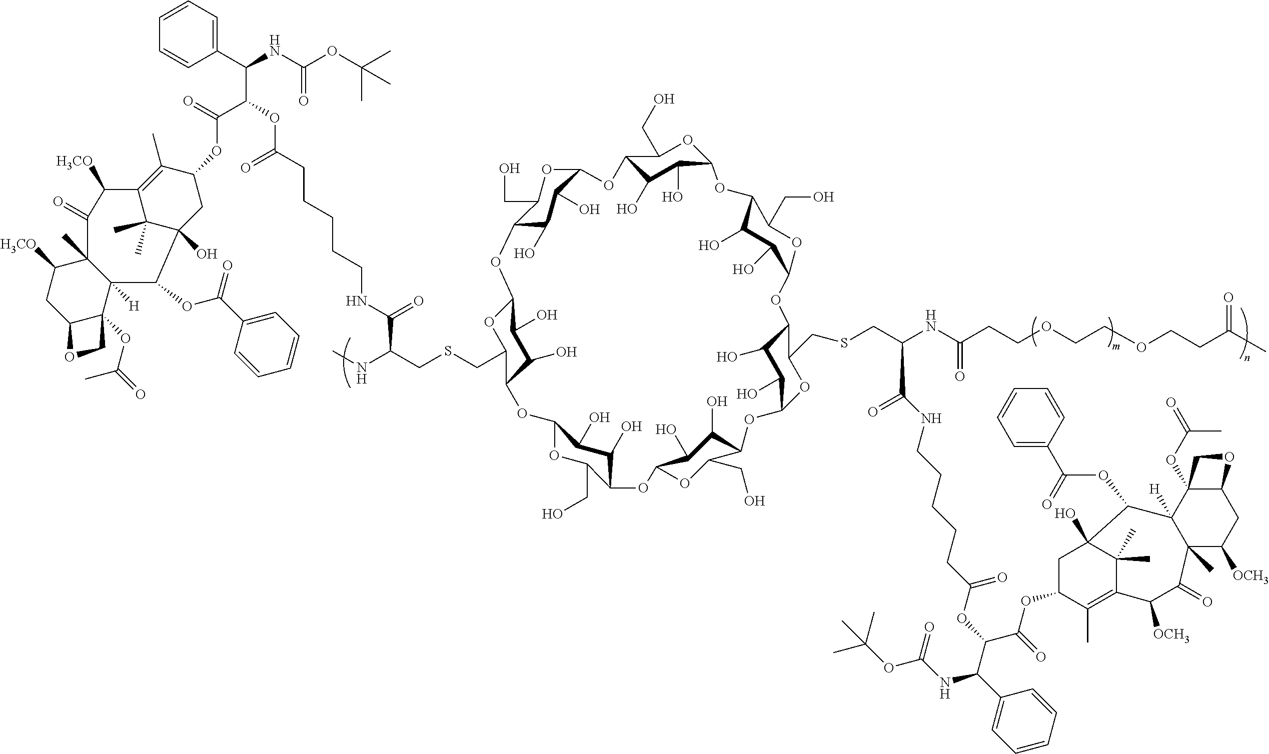

### Example 46

### Synthesis of Cabazitaxel Aminoethyldithioethyl Carbonate

Succinimidyl MMT-aminoethyldithioethyl carbonate from Scheme 9(a) will then be transferred to a cooled solution of cabazitaxel (6.36 g, 7.61 mmol) and DMAP (1.03 g) in DCM (60 mL) at 0-5° C. with stirring for 16 h. It will be purified by column chromatography.

A 1000 mL round bottom flask equipped with a magnetic stirrer will be charged with cabazitaxel Cbz-aminoethyldithioethyl carbonate (12.6 g) and DCM (300 mL). Anisole (10.9 mL, 10 equiv.) will be added to this clear solution and stirred for a few minutes. Dichloroacetic acid (8.3 mL, 10 equiv.) will be added over 5 min and the reaction will be stirred at ambient temperature for 1 h. The mixture will be concentrated down to ˜100 mL, to which heptanes (800 mL) will be slowly added resulting in a suspension. The suspension will be stirred for 15 min and the supernatant will be decanted off. The orange residue will be washed with heptanes (200 mL) and vacuum-dried at ambient temperature for 1 h. THF (30 mL) will be added to dissolve the orange residue producing a red solution. Heptanes (500 mL) will be slowly added to precipitate out the product. The resulting suspension will be stirred at ambient temperature for 1 h and filtered. The filter cake will be washed with heptanes (300 mL) and dried under vacuum to yield cabazitaxel aminoethyldithioethyl carbonate.

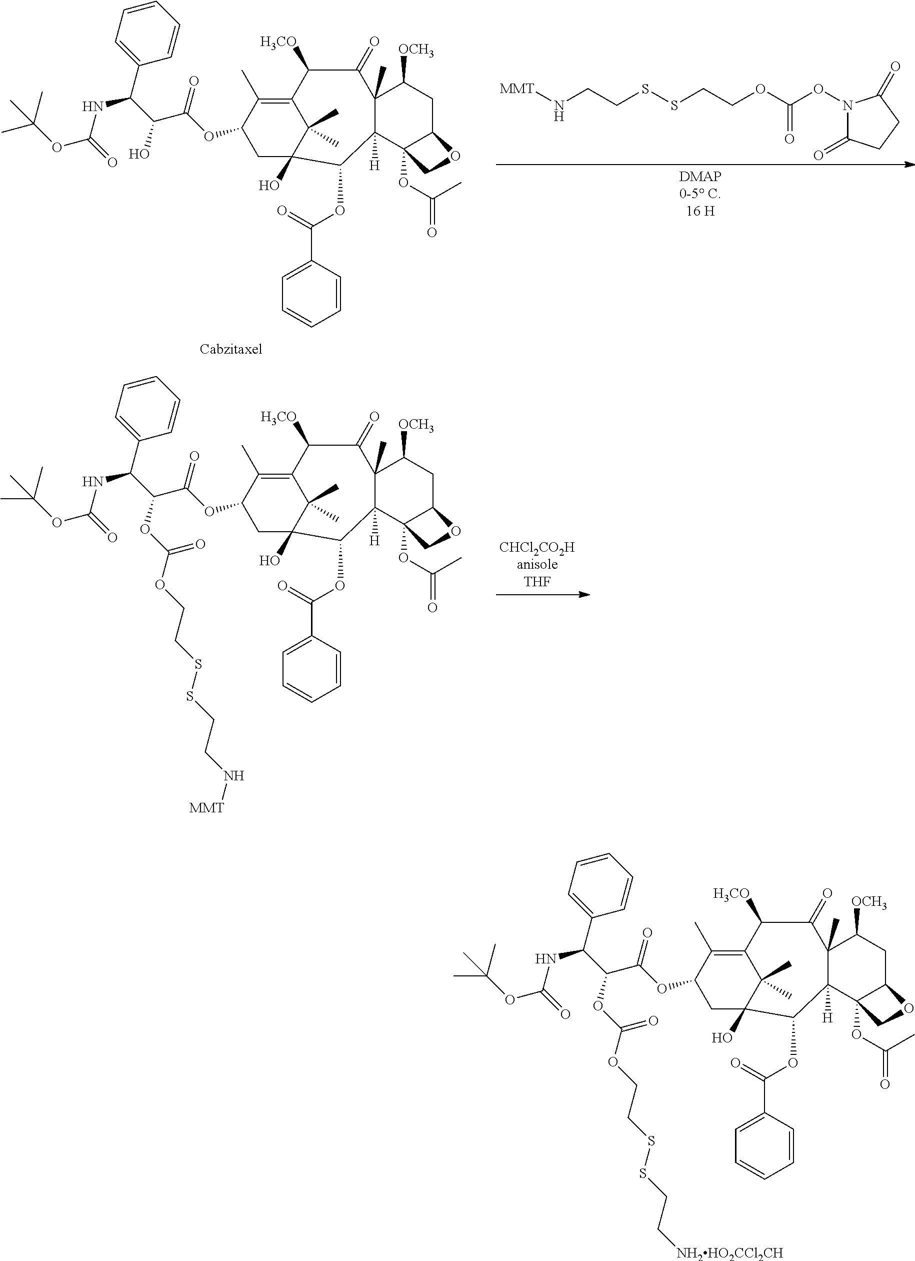

### Example 47

### Synthesis of CDP Cabazitaxel Aminoethyldithioethyl Carbonate

CDP (1.0 g, 0.21 mmol) will be dissolved in dry N,N-dimethylformamide (DMF, 10 mL). Cabazitaxel aminoethyldithioethyl carbonate (460 mg, 0.46 mmol), N,N-Diisopropylethylamine (59 mg, 0.46 mmol), N-(3-Dimethylaminopropyl)-N′-ethylcarbodiimide hydrochloride (87 mg, 0.46 mmol), and N-Hydroxysuccinimide (52 mg, 0.46 mmol) will then be added to the polymer solution and stirred for 2 h. The polymer will be precipitated with isopropanol (150 mL) and then rinsed with acetone (100 mL). The precipitate will be dissolved in nanopure water (100 mL). It will be purified by TFF with nanopure water (1 L). It will then be filtered through 0.2 μm filter and kept frozen.

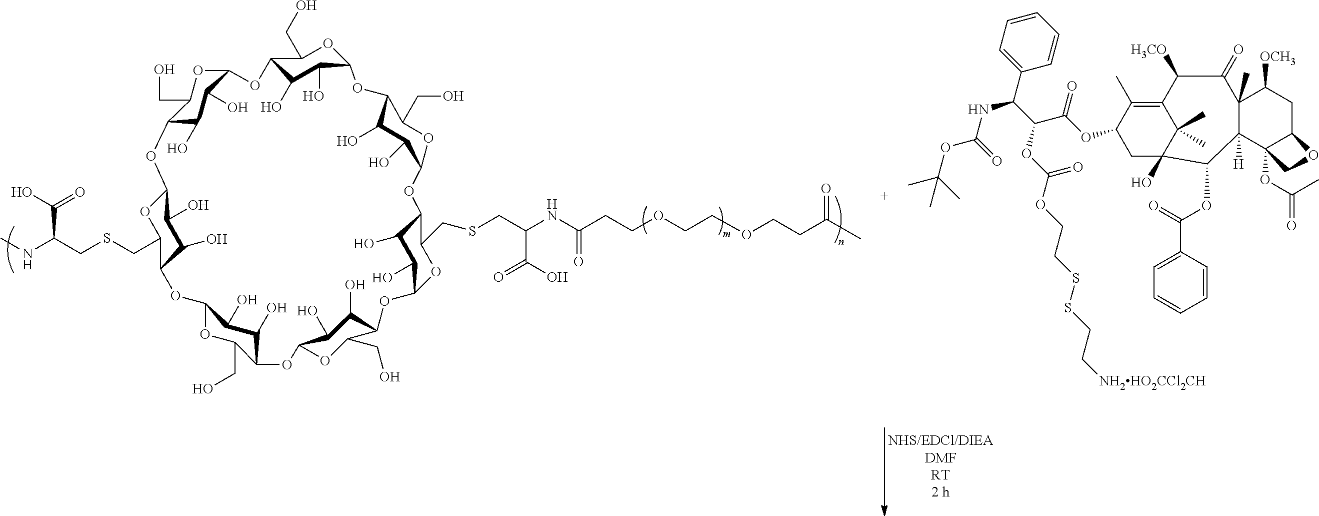

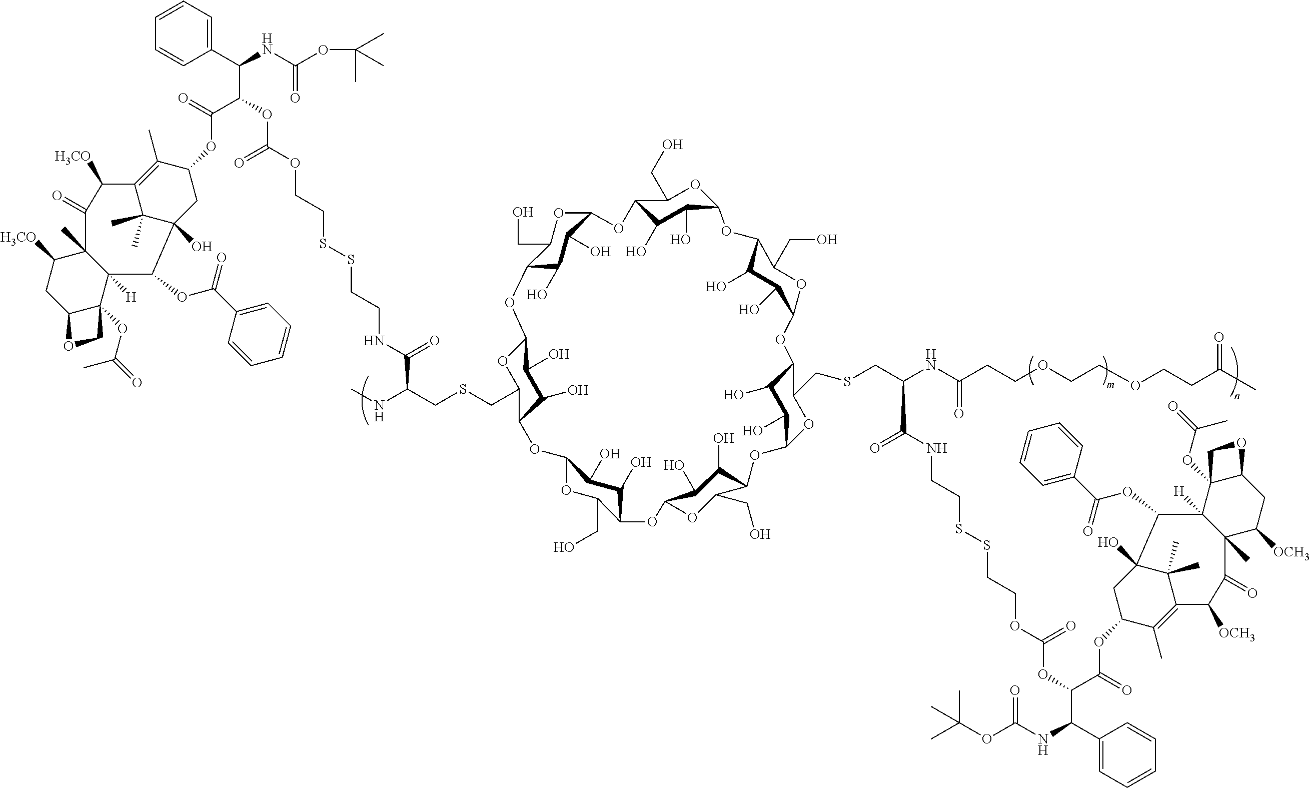

Other embodiments are in the claims.

We claim:

1. A method of treating cancer in a subject, wherein the subject has cancer and has received an anticancer agent, the method comprising administering to the subject a CDP-taxane conjugate in an amount effective to treat the cancer, to thereby treat the cancer.

2. The method of claim 1, wherein the taxane is docetaxel, larotaxel, or cabazitaxel.

3. The method of claim 1, wherein the method further comprises identifying a subject having cancer who has received an anticancer agent prior to administering the CDP-taxane conjugate.

4. The method of claim 1, wherein the cancer is a chemotherapeutic sensitive, a chemotherapeutic refractory, a chemotherapeutic resistant, and/or a relapsed cancer.

5. A method of treating cancer in a subject, the method comprising administering a CDP-taxane conjugate, wherein the CDP-taxane conjugate is of the following formula: 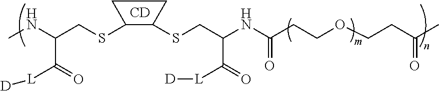
wherein each L is independently a linker or absent and each D is independently a taxane, a prodrug derivative thereof, or absent and wherein the group 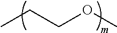
has a Mw of 3.4 kDa or less and n is at least 4, provided that the polymer comprises at least one taxane, to a subject in an amount effective to treat the cancer, to thereby treat the cancer.

6. The method of claim 5, wherein the taxane is docetaxel, larotaxel, or cabazitaxel.

7. The method of claim 5, wherein the breast cancer is selected from the group consisting of breast, prostate, lung, and ovarian cancer.

8. A method of treating metastatic or locally advanced breast cancer in a subject, the method comprising providing a subject who has metastatic or locally advanced breast cancer and has been treated with a chemotherapeutic agent which did not effectively treat the cancer or which had an unacceptable side effect, and
administering a CDP-taxane conjugate to a subject in an amount effective to treat the cancer, to thereby treat the cancer.

9. A method of treating hormone refractory prostate cancer in a subject, the method comprising:
providing a subject who has hormone refractory prostate cancer and has been treated with a chemotherapeutic agent that did not effectively treat the cancer or who had an unacceptable side effect, and
administering a CDP-taxane conjugate to a subject in an amount effective to treat the cancer, to thereby treat the cancer.

10. The method of claim 7, wherein the cancer is non-small cell lung cancer.

11. A method of treating a subject having cancer, the method comprising selecting a subject having cancer who has received an anticancer agent and has a neutrophil count less than a standard; and
administering a CDP-taxane conjugate to the subject in an amount effective to treat the cancer, to thereby treat the cancer.

12. The method of claim 11, wherein the method further comprises identifying a subject having cancer who has received an anticancer agent and has a neutrophil count less than a standard, and
Identifying the subject as suitable for treatment with a CDP-taxane conjugate.

13. A method for treating a subject having cancer, the method comprising:
selecting a subject with cancer who has moderate to severe neutropenia; and
administering a CDP-taxane conjugate to the subject in an amount effective to treat the cancer, to thereby treat the cancer.

14. The method of claim 13, further comprising determining whether a subject with a proliferative disorder has moderate to severe neutropenia; and
selecting a subject for treatment with a CDP-taxane conjugate on the basis that the subject has moderate to severe neutropenia.

15. A method for treating a subject having cancer, the method comprising:
selecting a subject with cancer who has experienced one or more symptom of neuropathy from treatment with a anticancer agent; and
administering a CDP-taxane conjugate, to the subject in an amount effective to treat the cancer, to thereby treat the cancer.

16. The method of claim 15, further comprising: determining whether a subject with cancer, has experienced neuropathy from treatment with an anticancer agent; and selecting a subject for treatment with a CDP-taxane conjugate, on the basis that the subject has experienced neuropathy from treatment with an anticancer agent

17. A method of treating a subject having cancer, the method comprising:
selecting a subject with cancer who has or is at risk of having hepatic impairment, e.g., a subject having alanine aminotransferase (ALT) and/or aspartate aminotransferase (AST) levels greater than 1.5 times the upper limit of normal (ULN) and/or bilirubin levels greater than 2 times the ULN; and
administering a CDP-taxane conjugate to the subject in an amount effective to treat the cancer, to thereby treat the cancer.

18. The method of claim 17, further comprising determining if a subject having cancer has or is at risk of having hepatic impairment, e.g., determining alanine aminotransferase (ALT), aspartate aminotransferase (AST) and/or bilirubin levels in a subject having cancer; and
selecting a subject having hepatic impairment, e.g., a subject having ALT and/or AST levels greater than 1.5 times the upper limit of normal (ULN) and/or bilirubin levels greater than 2 times the ULN, for treatment with a CDP-taxane conjugate.

19. A method of treating a subject having cancer, the method comprising:
selecting a subject with cancer who has or is at risk of having hepatic impairment, e.g., a subject having alkaline phosphatase (ALP) levels greater than 2.5 times the upper limit of normal (ULN), serum glutamate oxaloacetate transaminase (SGOT) and/or serum glutamate pyruvate transaminase (SGPT) levels greater than 1.5 times the ULN and/or bilirubin levels greater than the ULN; and
administering a CDP-taxane conjugate to the subject in an amount effective to treat the cancer, to thereby treat the cancer.

20. The method of claim 19, further comprising determining if a subject having cancer has or is at risk of having hepatic impairment, e.g., determining alkaline phosphatase (ALP), serum glutamate oxaloacetate transaminase (SGOT), serum glutamate pyruvate transaminase (SGPT) and/or bilirubin levels in the subject having cancer; and
selecting a subject having or at risk of having hepatic impairment, e.g., a subject having ALP levels greater than 2.5 times the upper limit of normal (ULN), SGOT and/or SGPT levels greater than 1.5 times the upper limit of normal (ULN) and/or bilirubin levels greater than the ULN for treatment with a CDP-taxane conjugate.

21. A method of treating a subject having cancer, the method comprising:
selecting a subject having cancer who is currently being administered or will be, administered a cytochrome P450 isoenzyme, and/or a CYP2C8 inhibitor; and administering a CDP-taxane conjugate to the subject at a dose described herein, to thereby treat the cancer.

22. The method of claim 21, further comprising: determining if a subject having cancer is currently being administered or will be administered a cytochrome P450 isoenzyme and/or a CYP2C8 inhibitor; and
selecting a subject with cancer who is currently being administered or will be administered a cytochrome P450 isoenzyme and/or a CYP2C8 inhibitor, for treatment with a CDP-taxane conjugate.

23. A method of selecting a subject having cancer, for treatment with a CDP-taxane conjugate, the method comprising:
determining if a subject with cancer is at risk for or has diarrhea or has experienced diarrhea from treatment with an anticancer agent, and
selecting a subject who is at risk for or has diarrhea or has experienced diarrhea from treatment with an anticancer agent for treatment with a CDP-taxane conjugate.

24. A CDP-taxane conjugate of the following formula: 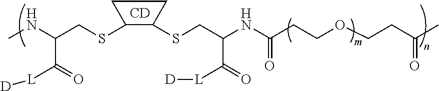
wherein each L is independently a linker or absent and each D is independently a taxane, a prodrug derivative thereof, or absent and wherein the group 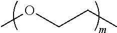
has a Mw of 3.4 kDa or less and n is at least 4, provided that the polymer comprises at least one taxane.

25. The CDP-taxane conjugate of claim 24 wherein the taxane is docetaxel, larotaxel, or cabazitaxel.

26. A composition comprising the CDP-taxane conjugate of claim 24.

27. A pharmaceutical composition comprising the CDP-taxane conjugate of claim 24.

28. A dosage form comprising the CDP-taxane conjugate of claim 24.

29. A kit comprising the CDP-taxane conjugate of claim 25.
